(19) United States
(12) Patent Application Publication
(10) Pub. No.: US 2024/0366157 A1
(43) Pub. Date: Nov. 7, 2024

(54) Methods And Devices For Real-Time Word And Speech Decoding From Neural Activity

(71) Applicant: The Regents of the University of California, Oakland, CA (US)

(72) Inventors: David A. Moses, Oakland, CA (US); Jessie Liu, San Francisco, CA (US); Sean Metzger, San Francisco, CA (US); Edward Chang, San Francisco, CA (US)

(21) Appl. No.: 18/561,981

(22) PCT Filed: May 26, 2022
(86) PCT No.: PCT/US2022/031101

§ 371 Date: Nov. 17, 2023

Related U.S. Application Data
(60) Provisional application No. 63/193,351, filed on May 26, 2021.

(51) Int. Cl. A61B 5/00; A61B 5/293; A61B 5/37; G10L 13/08; G10L 15/24

(52) U.S. Cl. CPC A61B 5/7264; A61B 5/293; A61B 5/37; G10L 13/08; G10L 15/24

(57) ABSTRACT

Methods, devices, and systems for assisting individuals with communication are provided. In particular, methods, devices, and systems are provided for decoding words and sentences directly from neural activity of an individual. Cortical activity from a region of the brain involved in speech processing is recorded while an individual attempts to say or spell out words. Deep learning computational models are used to detect and classify words from the recorded brain activity. Decoding of speech from brain activity is aided by use of a language model that predicts how likely certain sequences of words are to occur. In addition, decoding of attempted non-speech motor movements from neural activity can be used to further assist communication. This neurotechnology can be used to restore communication to patients who have lost the ability to speak and has the potential to improve autonomy and quality of life.

### CROSS-REFERENCE TO RELATED APPLICATION

This application claims benefit under 35 U.S.C. § 119(e) of provisional application 63/193,351, filed May 26, 2021, which application is hereby incorporated by reference in its entirety.

### STATEMENT OF GOVERNMENT SUPPORT

This invention was made with government support under grant number U01 NS098971-01 awarded by the National Institutes of Health (NIH). The government has certain rights in the invention.

### INTRODUCTION

Anarthria is the loss of the ability to articulate speech. It can result from a variety of conditions, including stroke, traumatic brain injury, and amyotrophic lateral sclerosis (Beukelman et al. (2007) Augmentative and Alternative Communication 23(3):230-242). For paralyzed individuals with severe movement impairment, it hinders communication with family, friends, and caregivers, reducing self-reported quality of life (Felgoise et al. (2016) Amyotrophic Lateral Sclerosis and Frontotemporal Degeneration 17(3-4):179-183). Neurotechnology designed to restore communication for paralyzed patients who have lost the ability to speak has the potential to improve autonomy and quality of life. However, most existing approaches are slow and tedious compared to natural speech. Thus, there remains a need for better methods for restoring the ability to communicate to patients with anarthria.

### SUMMARY

Methods, devices, and systems for assisting individuals with communication are provided. In particular, methods, devices, and systems are provided for decoding words and sentences directly from neural activity of an individual. In the disclosed methods, cortical activity from a region of the brain involved in speech processing is recorded while an individual attempts to say or spell out words (even if the words or spelled letters are not vocalized). Deep learning computational models are used to detect and classify words from the recorded brain activity. Decoding of speech from brain activity is aided by use of a language model that predicts how likely certain sequences of words are to occur. In addition, decoding of attempted non-speech motor movements from neural activity can be used to further assist communication. The neurotechnology described herein can be used to restore communication to patients who have lost the ability to speak and has the potential to improve autonomy and quality of life.

In one aspect, a method of assisting a subject with communication is provided, the method comprising: positioning a neural recording device comprising an electrode at a location in a sensorimotor cortex region of the brain of the subject to record brain electrical signal data associated with attempted speech by the subject; positioning an interface in communication with a computing device at a location on the head of the subject, wherein the interface is connected to the neural recording device; recording the brain electrical signal data associated with attempted speech by the subject using the neural recording device, wherein the interface receives the brain electrical signal data from the neural recording device and transmits the brain electrical signal data to a processor; and decoding a word, a phrase, or a sentence from the recorded brain electrical signal data using the processor.

In certain embodiments, the subject has difficulty with communication because of anarthria, a stroke, a traumatic brain injury, a brain tumor, or amyotrophic lateral sclerosis. In some embodiments, the subject is paralyzed.

In certain embodiments, the location of the neural recording device is in the ventral sensorimotor cortex. For example, the electrode can be positioned on a surface of the sensorimotor cortex region or within the sensorimotor cortex region. In some embodiments, the electrode is positioned on a surface of the sensorimotor cortex region of the brain in a subdural space.

In certain embodiments, the method comprises recording brain electrical signal data from a sensorimotor cortex region selected from a precentral gyrus, postcentral gyrus, posterior middle frontal gyrus, posterior superior frontal gyrus, or posterior inferior frontal gyrus region, or any combination thereof.

In certain embodiments, the neural recording device comprises a brain-penetrating electrode array or an electrocorticography (ECoG) electrode array.

In certain embodiments, the electrode is a depth electrode or a surface electrode.

In certain embodiments, the features used by the processor are high-gamma frequency content features contained in the electrical signal data. In some embodiments, the high-gamma frequency electrical signal data may comprise neural oscillations in a range from 70 Hz to 150 Hz.

In certain embodiments, the method further comprises mapping the brain of the subject to identify an optimal location for positioning the electrode for recording the brain electrical signals associated with the attempted speech by the subject.

In certain embodiments, the interface comprises a percutaneous pedestal connector attached to the subject's cranium. In some embodiments, the interface further comprises a removable headstage connected to the percutaneous pedestal connector.

In certain embodiments, the processor is provided by a computer or a handheld device (e.g., a cell phone or tablet).

In certain embodiments, the processor is programmed to automate speech detection, word classification, and sentence decoding using a machine learning algorithm based on identification of a neural activity pattern of electrical signals in the recorded brain electrical signal data associated with attempted word production by the subject. In some embodiments, the machine learning algorithm uses artificial neural network (ANN) models for the speech detection and the word classification and natural language processing techniques such as, but not limited to, a hidden Markov model (HMM) or a Viterbi decoding model for the sentence decoding.

In certain embodiments, the processor is programmed to automate detection of onset and offset of word production during the attempted speech by the subject. In some embodiments, the method further comprises assigning speech event labels for preparation, speech, and rest to time points during the recording of the brain electrical signal data. In some embodiments, the processor is programmed to use the recorded brain electrical signal data within a time window around the detected onset of word classification.

In certain embodiments, the subject is limited to a specified word set for the attempted speech.

In certain embodiments, the processor is programmed to calculate a probability that a word of the word set is an intended word that the subject tried to produce during the attempted speech. In some embodiments, the processor is programmed to calculate the probability that a word of the word set is an intended word that the subject tried to produce during the attempted speech for every word of the word set, and select the word of the word set having the highest probability of being the intended word that the subject tried to produce during the attempted speech.

In certain embodiments, the word set comprises: am, are, bad, bring, clean, closer, comfortable, coming, computer, do, faith, family, feel, glasses, going, good, goodbye, have, hello, help, here, hope, how, hungry, I, is, it, like, music, my, need, no, not, nurse, okay, outside, please, right, success, tell, that, they, thirsty, tired, up, very, what, where, yes, and you.

In certain embodiments, the subject may use the words of the word set without limitation to create sentences. In other embodiments, the subject is limited to a specified sentence set for the attempted speech.

In certain embodiments, the processor is programmed to calculate a probability that a sequence of words is an intended sentence that the subject tried to produce during the attempted speech. In some embodiments, the processor is programmed to calculate the probability that a sentence of the sentence set is an intended sentence that the subject tried to produce during the attempted speech for every sentence of the sentence set. In some embodiments, the processor is programmed to calculate the probability of many possible sentences composed entirely of words from the specified word set as being the intended sentence that the subject tried to produce during the attempted speech. In some embodiments, the processor is programmed to maintain the most likely sentence as well as other, less likely sentences composed entirely of words from the specified word set that the subject tried to produce during the attempted speech. In some embodiments, the processor is programmed to track the first, second, and third most likely sentence possibilities at any given point in time. When a new word event is processed, the most likely sentence may change. For example, the second most likely sentence based on processing of a word event could then become the most likely sentence after one or more additional word events are processed.

In certain embodiments, the sentence set includes sentences that can be selected to communicate with a caregiver regarding tasks the subject wishes the caregiver to perform. In some embodiments, the sentences that can be composed entirely of words from the specified word set include sentences that can be used to communicate with a caregiver regarding the tasks the subject wishes the caregiver to perform.

In certain embodiments, the sentence set comprises: Are you going outside; Are you tired; Bring my glasses here; Bring my glasses please; Do not feel bad; Do you feel comfortable; Faith is good; Hello how are you; Here is my computer; How do you feel; How do you like my music; I am going outside; I am not going; I am not hungry; I am not okay; I am okay; I am outside; I am thirsty; I do not feel comfortable; I feel very comfortable; I feel very hungry; I hope it is clean; I like my nurse; I need my glasses; I need you; It is comfortable; It is good; It is okay; It is right here; My computer is clean; My family is here; My family is outside; My family is very comfortable; My glasses are clean; My glasses are comfortable; My nurse is outside; My nurse is right outside; No; Please bring my glasses here; Please clean it; Please tell my family; That is very clean; They are coming here; They are coming outside; They are going outside; They have faith; What do you do; Where is it; Yes; and You are not right.

In certain embodiments, the processor is programmed to use a language model that provides next-word probabilities given a previous word or phrase in a sequence of words to aid the decoding by determining predicted word sequence probabilities. For example, words that occur more frequently are assigned more weight than words that occur less frequently according to the language model.

In certain embodiments, the processor is programmed to use a hidden Markov model (HMM) or a Viterbi decoding model to determine the most likely sequence of words in the intended speech of the subject given the brain electrical signal data associated with the attempted speech, the predicted word probabilities from the word classification using the machine learning algorithm, and the word sequence probabilities using the language model.

In certain embodiments, the method further comprises: recording brain electrical signal data associated with an attempted non-speech motor movement of the subject, wherein the subject performs the attempted non-speech motor movement to indicate the initiation or termination of the attempted speech or to control an external device; and analyzing the brain electrical signal data using a non-speech motor movement classification model that identifies patterns of electrical signals in the recorded brain electrical signal data associated with the attempted non-speech motor movement and calculates a probability that the subject attempted the non-speech motor movement. In some embodiments, the attempted non-speech motor movement comprises an attempted head, arm, hand, foot, or leg movement.

In certain embodiments, the processor is further programmed to automate detection of an attempted non-speech motor movement of the subject based on identification of a neural activity pattern of electrical signals in the recorded brain electrical signal data associated with the attempted non-speech motor movement. In some embodiments, the processor is further programmed to assign event labels for the attempted non-speech motor movement to time points during the recording of the brain electrical signal data.

In certain embodiments, the method further comprises assessing accuracy of the decoding.

In another aspect, a computer implemented method for decoding a sentence from recorded brain electrical signal data associated with attempted speech by a subject is provided, the computer performing steps comprising: a) receiving the recorded brain electrical signal data from the subject; b) analyzing the recorded brain electrical signal data using a speech detection model to calculate the probability that attempted speech is occurring at any time point during recording of the brain electrical signal data and detect onset and offset of word production during the attempted speech by the subject; c) analyzing the brain electrical signal data using a word classification model that identifies patterns of electrical signals in the recorded brain electrical signal data associated with attempted word production by the subject and calculates predicted word probabilities; d) performing sentence decoding by using the calculated word probabilities from the word classification model in combination with predicted word sequence probabilities in the sentence using a language model that provides next-word probabilities given a previous word or phrase in a sequence of words to calculate predicted word sequence probabilities and determining the most likely sequence of words in the sentence based on the predicted word probabilities determined using the word classification model and the language model; and e) displaying the sentence decoded from the recorded brain electrical signal data.

In certain embodiments, the processor is programmed to automate speech detection, word classification, and sentence decoding using a machine learning algorithm based on identification of a neural activity pattern of electrical signals in the recorded brain electrical signal data associated with attempted word production by the subject. In some embodiments, the machine learning algorithm uses artificial neural network (ANN) models for the speech detection and the word classification and natural language processing techniques such as, but not limited to, a hidden Markov model (HMM) or a Viterbi decoding model for the sentence decoding.

In certain embodiments, the subject is limited to a specified word set for the attempted speech. In some embodiments, the processor is further programmed to calculate a probability that a word of the word set is an intended word that the subject tried to produce during the attempted speech for every word of the word set and select the word of the word set having the highest probability of being the intended word that the subject tried to produce during the attempted speech.

In certain embodiments, the subject may use the words of the word set without limitation to create sentences. In other embodiments, the subject is limited to a specified sentence set for the attempted speech. In some embodiments, the processor is further programmed to calculate a probability that a sequence of words is an intended sentence that the subject tried to produce during the attempted speech. In some embodiments, the processor is further programmed to calculate a probability that a sentence of the sentence set is an intended sentence that the subject tried to produce during the attempted speech.

In certain embodiments, the computer implemented method further comprises assigning speech event labels for preparation, speech, and rest to time points during the recording of the brain electrical signal data.

In certain embodiments, the computer implemented method further comprises analyzing the recorded brain electrical signal data within a time window around the detected onset of word classification (e.g., from 1 second before the detected onset up to 3 seconds after the detected onset for word classification).

In certain embodiments, the computer implemented method further comprises assigning more weight to words that occur more frequently than words that occur less frequently according to the language model.

In certain embodiments, the computer implemented method further comprises: receiving recorded brain electrical signal data associated with an attempted non-speech motor movement of the subject, wherein the subject performs the attempted non-speech motor movement to indicate the initiation or termination of the attempted speech or to control an external device; and analyzing the brain electrical signal data using a non-speech motor movement classification model that identifies patterns of electrical signals in the recorded brain electrical signal data associated with the attempted non-speech motor movement and calculates a probability that the subject attempted the non-speech motor movement. In some embodiments, the attempted non-speech motor movement comprises an attempted head, arm, hand, foot, or leg movement. In some embodiments, the computer implemented method further comprises assigning event labels for the attempted non-speech motor movement to time points during the recording of the brain electrical signal data.

In certain embodiments, the computer implemented method further comprises storing a user profile for the subject comprising information regarding the patterns of electrical signals in the recorded brain electrical signal data associated with attempted word production by the subject.

In another aspect, a non-transitory computer-readable medium is provided comprising program instructions that, when executed by a processor in a computer, causes the processor to perform a computer implemented method described herein for decoding a sentence from recorded brain electrical signal data associated with attempted speech by a subject.

In another aspect, a kit comprising the non-transitory computer-readable medium and instructions for decoding brain electrical signal data associated with attempted speech by a subject is provided.

In another aspect, a system for assisting a subject with communication is provided, the system comprising: a neural recording device comprising an electrode adapted for positioning at a location in a sensorimotor cortex region of the brain of the subject to record brain electrical signal data associated with attempted speech by the subject; a processor programmed to decode a sentence from the recorded brain electrical signal data according to a computer implemented method described herein; an interface in communication with a computing device adapted for positioning at a location on the head of the subject, wherein the interface receives the brain electrical signal data from the neural recording device and transmits the brain electrical signal data to the processor; and a display component for displaying the sentence decoded from the recorded brain electrical signal data.

In certain embodiments, the subject has difficulty with communication because of anarthria, a stroke, a traumatic brain injury, a brain tumor, or amyotrophic lateral sclerosis.

In certain embodiments, the location of the neural recording device is in the ventral sensorimotor cortex.

In certain embodiments, the electrode is adapted for positioning on a surface of the sensorimotor cortex region or within the sensorimotor cortex region. In some embodiments, the electrode is adapted for positioning on a surface of the sensorimotor cortex region of the brain in a subdural space.

In certain embodiments, the neural recording device comprises a brain-penetrating electrode array or an electrocorticography (ECoG) electrode array.

In certain embodiments, the electrode is a depth electrode or a surface electrode.

In certain embodiments, the electrical signal data comprises high-gamma frequency content features. In some embodiments, the high-gamma frequency electrical signal data comprises neural oscillations in a range from 70 Hz to 150 Hz.

In certain embodiments, the interface comprises a percutaneous pedestal connector attached to the subject's cranium. In some embodiments, the interface further comprises a headstage that is connectable to the percutaneous pedestal connector.

In certain embodiments, the processor is provided by a computer or handheld device (e.g., a cell phone or tablet).

In certain embodiments, the processor is programmed to automate speech detection, word classification, and sentence decoding using a machine learning algorithm based on identification of a neural activity pattern of electrical signals in the recorded brain electrical signal data associated with attempted word production by the subject. In some embodiments, the machine learning algorithm uses artificial neural network (ANN) models for the speech detection and the word classification and natural language processing techniques such as, but not limited to, a hidden Markov model (HMM) or a Viterbi decoding model for the sentence decoding.

In certain embodiments, the processor is further programmed to assign speech event labels for preparation, speech, and rest to time points during the recording of the brain electrical signal data. In some embodiments, the processor is further programmed to use the recorded brain electrical signal data within a time window around the detected onset of word classification.

In certain embodiments, the subject is limited to a specified word set for the attempted speech. In some embodiments, the processor is further programmed to calculate a probability that a word of the word set is an intended word that the subject tried to produce during the attempted speech for every word of the word set, and select the word of the word set having the highest probability of being the intended word that the subject tried to produce during the attempted speech.

In certain embodiments, the word set comprises: am, are, bad, bring, clean, closer, comfortable, coming, computer, do, faith, family, feel, glasses, going, good, goodbye, have, hello, help, here, hope, how, hungry, I, is, it, like, music, my, need, no, not, nurse, okay, outside, please, right, success, tell, that, they, thirsty, tired, up, very, what, where, yes, and you.

In certain embodiments, the subject may use the words of the word set without limitation to create sentences. In other embodiments, the subject is limited to a specified sentence set for the attempted speech. In some embodiments, the processor is further programmed to calculate a probability that a sequence of words is an intended sentence that the subject tried to produce during the attempted speech. In some embodiments, the processor is further programmed to calculate a probability that a sentence of the sentence set is an intended sentence that the subject tried to produce during the attempted speech. In some embodiments, the sentence set includes sentences that can be selected to communicate with a caregiver regarding tasks the subject wishes the caregiver to perform.

In certain embodiments, the sentence set comprises: Are you going outside; Are you tired; Bring my glasses here; Bring my glasses please; Do not feel bad; Do you feel comfortable; Faith is good; Hello how are you; Here is my computer; How do you feel; How do you like my music; I am going outside; I am not going; I am not hungry; I am not okay; I am okay; I am outside; I am thirsty; I do not feel comfortable; I feel very comfortable; I feel very hungry; I hope it is clean; I like my nurse; I need my glasses; I need you; It is comfortable; It is good; It is okay; It is right here; My computer is clean; My family is here; My family is outside; My family is very comfortable; My glasses are clean; My glasses are comfortable; My nurse is outside; My nurse is right outside; No; Please bring my glasses here; Please clean it; Please tell my family; That is very clean; They are coming here; They are coming outside; They are going outside; They have faith; What do you do; Where is it; Yes; and You are not right.

In certain embodiments, the processor is further programmed to automate detection of an attempted non-speech motor movement of the subject based on identification of a neural activity pattern of electrical signals in the recorded brain electrical signal data associated with the attempted non-speech motor movement. In some embodiments, the processor is further programmed to assign event labels for the attempted non-speech motor movement to time points during the recording of the brain electrical signal data.

In another aspect, a kit comprising a system described herein for assisting a subject with communication and instructions for using the system for recording and decoding brain electrical signal data associated with attempted speech by a subject is provided.

In another aspect, a method of assisting a subject with communication is provided, the method comprising: positioning a neural recording device comprising an electrode at a location in a sensorimotor cortex region of the brain of the subject to record brain electrical signal data associated with attempted spelling of letters of words of an intended sentence by the subject; positioning an interface in communication with a computing device at a location on the head of the subject, wherein the interface is connected to the neural recording device; recording the brain electrical signal data associated with said attempted spelling by the subject using the neural recording device, wherein the interface receives the brain electrical signal data from the neural recording device and transmits the brain electrical signal data to a processor of the computing device; and decoding the spelled words of the intended sentence from the recorded brain electrical signal data using the processor.

In certain embodiments, the electrical signal data comprises high-gamma frequency content features (e.g., 70 Hz to 150 Hz) and low frequency content features (e.g., 0.3 Hz to 100 Hz).

In certain embodiments, recording the brain electrical signal data comprises recording the brain electrical signal data from a sensorimotor cortex region selected from a precentral gyrus, postcentral gyrus, posterior middle frontal gyrus, posterior superior frontal gyrus, or posterior inferior frontal gyrus region, or any combination thereof.

In certain embodiments, the method further comprising mapping the brain of the subject to identify an optimal location for positioning the electrode for recording the brain electrical signals associated with the attempted spelling of words by the subject.

In certain embodiments, the processor is programmed to automate detection of brain activity associated with the attempted spelling, letter classification, word classification, and sentence decoding based on identification of a neural activity pattern of electrical signals in the recorded brain electrical signal data associated with the attempted spelling of words by the subject.

In certain embodiments, the processor is programmed to use a machine learning algorithm for the speech detection, letter classification, word classification, and sentence decoding. In some embodiments, the machine learning algorithm may use natural language processing techniques.

In certain embodiments, the processor is further programmed to constrain word classification from sequences of letters decoded from neural activity associated with attempted spelling of words by the subject to only words within a vocabulary of a language used by the subject.

In certain embodiments, the processor is programmed to automate detection of onset and offset of letter production during the attempted spelling by the subject.

In certain embodiments, the processor is further programmed to assign speech event labels for preparation, speech, and rest to time points during the recording of the brain electrical signal data.

In certain embodiments, the processor is programmed to use the recorded brain electrical signal data within a time window around the detected onset of attempted spelling of a letter by the subject.

In certain embodiments, the method further comprises providing a series of go cues to the subject indicating when the subject should initiate attempted spelling of each letter of the words of the intended sentence. In some embodiments, the series of go cues are provided visually on a display. In some embodiments, each go cue is preceded by a countdown to the presentation of the go cue, wherein the countdown for the next spelled letter is provided visually on the display and automatically started after each go cue. In some embodiments, the series of go cues are provided with a set interval of time between each go cue. In some embodiments, the subject can control the set interval of time between each go cue. In some embodiments, the processor is programmed to use the recorded brain electrical signal data within a time window following the go cue.

In certain embodiments, the processor is programmed to calculate a probability that a sequence of decoded words from a sequence of decoded letters is an intended sentence that the subject tried to produce during the attempted spelling of letters of words of an intended sentence by the subject.

In certain embodiments, the processor is programmed to use a language model that provides next-word probabilities given a previous word or phrase in a sequence of words to aid the decoding by determining predicted word sequence probabilities. In some embodiments, words that occur more frequently are assigned more weight than words that occur less frequently according to the language model.

In certain embodiments, the processor is further programmed to use a sequence of predicted letter probabilities to compute potential sentence candidates and automatically insert spaces into letter sequences between predicted words in the sentence candidates.

In certain embodiments, the method further comprises: recording brain electrical signal data associated with an attempted non-speech motor movement of the subject, wherein the subject performs the attempted non-speech motor movement to indicate the initiation or termination of the attempted spelling of words of the intended sentence or to control an external device; and analyzing the brain electrical signal data using a classification model that identifies patterns of electrical signals in the recorded brain electrical signal data associated with the attempted non-speech motor movement and calculates a probability that the subject attempted non-speech motor movement.

In certain embodiments, the attempted non-speech motor movement comprises an attempted head, arm, hand, foot, or leg movement. In some embodiments, the attempted hand movement comprises an imagined hand gesture or an imagined hand squeeze.

In certain embodiments, the processor is programmed to automate detection of an attempted non-speech motor movement of the subject signaling the end of the attempted spelling by the subject based on identification of a neural activity pattern of electrical signals in the recorded brain electrical signal data associated with the attempted non-speech motor movement. In some embodiments, the processor is further programmed to assign event labels for the attempted non-speech motor movement to time points during the recording of the brain electrical signal data.

In certain embodiments, the method further comprises: recording brain electrical signal data associated with attempted speech by the subject using the neural recording device, wherein the interface receives the brain electrical signal data from the neural recording device and transmits the brain electrical signal data to the processor of the computing device; and decoding a word, a phrase, or a sentence from the recorded brain electrical signal data associated with attempted speech by the subject using the processor, as described herein.

In certain embodiments, the method further comprises assessing accuracy of the decoding.

In another aspect, a computer implemented method for decoding a sentence from recorded brain electrical signal data associated with attempted spelling of letters of words of an intended sentence by a subject is provided, the computer performing steps comprising: a) receiving the recorded brain electrical signal data associated with the attempted spelling of letters of words of an intended sentence by the subject; b) analyzing the recorded brain electrical signal data using a speech detection model to calculate the probability that attempted spelling is occurring at any time point and detect onset and offset of letter production during the attempted spelling by the subject; c) analyzing the brain electrical signal data using a letter classification model that identifies patterns of electrical signals in the recorded brain electrical signal data associated with attempted letter production by the subject and calculates a sequence of predicted letter probabilities; d) computing potential sentence candidates based on the sequence of predicted letter probabilities and automatically inserting spaces into the letter sequences between predicted words in the sentence candidates, wherein decoded words in the letter sequences are constrained to only words within a vocabulary of a language used by the subject; e) analyzing the potential sentence candidates using a language model that provides next-word probabilities given a previous word or phrase in a sequence of words to calculate predicted word sequence probabilities and determining the most likely sequence of words in a sentence; and f) displaying the sentence decoded from the recorded brain electrical signal data.

In certain embodiments, the recorded brain electrical signal data is only used within a time window around the detected onset of attempted spelling of a letter by the subject.

In certain embodiments, the method further comprises displaying a series of go cues to the subject indicating when the subject should initiate attempted spelling of each letter of the words of the intended sentence. In some embodiments, each go cue is preceded by displaying a countdown to the presentation of the go cue, wherein the countdown for the next spelled letter is automatically started after each go cue. In some embodiments, the series of go cues are provided with a set interval of time between each go cue. In some embodiments, the subject can control the set interval of time between each go cue. In some embodiments, the recorded brain electrical signal data within a time window following the go cue is used for letter classification.

In certain embodiments, the computer implemented method further comprises receiving recorded brain electrical signal data associated with an attempted non-speech motor movement of the subject, wherein the subject performs the attempted non-speech motor movement to indicate the initiation or termination of the attempted spelling of words of the intended sentence or to control an external device; and analyzing the brain electrical signal data using a motor movement classification model that identifies patterns of electrical signals in the recorded brain electrical signal data associated with the attempted non-speech motor movement and calculates a probability that the subject attempted the non-speech motor movement. In some embodiments, the attempted non-speech motor movement comprises an attempted head, arm, hand, foot, or leg movement. In some embodiments, the attempted hand movement comprises an imagined hand gesture or an imagined hand squeeze.

In certain embodiments, a machine learning algorithm is used for speech detection and letter classification.

In certain embodiments, the computer implemented method further comprises assigning more weight to words that occur more frequently than words that occur less frequently according to the language model.

In certain embodiments, the computer implemented method further comprises storing a user profile for the subject comprising information regarding the patterns of electrical signals in the recorded brain electrical signal data associated with letter production during attempted spelling by the subject.

In certain embodiments, the electrical signal data comprises high-gamma frequency content features (e.g., 70 Hz to 150 Hz) and low frequency content features (e.g., 0.3 Hz to 100 Hz).

In certain embodiments, the computer implemented method further comprises assessing accuracy of the decoding.

In certain embodiments, the computer implemented method further comprises decoding a sentence from recorded brain electrical signal data associated with attempted speech by the subject, the computer further performing steps comprising: a) receiving the recorded brain electrical signal data associated with the attempted speech by the subject; b) analyzing the recorded brain electrical signal data using a speech detection model to calculate the probability that attempted speech is occurring at any time point and detect onset and offset of word production during the attempted speech by the subject; c) analyzing the brain electrical signal data using a word classification model that identifies patterns of electrical signals in the recorded brain electrical signal data associated with attempted word production by the subject and calculates predicted word probabilities; d) performing sentence decoding by using the calculated word probabilities from the word classification model in combination with predicted word sequence probabilities in the sentence using a language model that provides next-word probabilities given a previous word or phrase in a sequence of words to calculate predicted word sequence probabilities and determining the most likely sequence of words in the sentence based on the predicted word probabilities determined using the word classification model and the language model; and e) displaying the sentence decoded from the recorded brain electrical signal data. In some embodiments, a machine learning algorithm is used for speech detection, word classification, and sentence decoding. In some embodiments, artificial neural network (ANN) models are used for the speech detection and the word classification and a hidden Markov model (HMM), a Viterbi decoding model, or other natural language processing techniques are used for the sentence decoding.

In another aspect, a non-transitory computer-readable medium is provided, the non-transitory computer-readable medium comprising program instructions that, when executed by a processor in a computer, causes the processor to perform a computer implemented method described herein.

In another aspect, a kit is provided, the kit comprising the non-transitory computer-readable medium and instructions for decoding brain electrical signal data associated with attempted spelling of letters of words of an intended sentence by a subject.

In another aspect, a system for assisting a subject with communication is provided, the system comprising: a neural recording device comprising an electrode adapted for positioning at a location in a sensorimotor cortex region of the brain of the subject to record brain electrical signal data associated with attempted speech, attempted spelling of letters of words of an intended sentence, or attempted non-speech motor movement by the subject, or a combination thereof, a processor programmed to decode a sentence from the recorded brain electrical signal data according to a computer implemented method described herein; an interface in communication with a computing device, said interface adapted for positioning at a location on the head of the subject, wherein the interface receives the brain electrical signal data from the neural recording device and transmits the brain electrical signal data to the processor; and a display component for displaying the sentence decoded from the recorded brain electrical signal data.

In certain embodiments, the electrode is adapted for positioning on a surface of the sensorimotor cortex region or within the sensorimotor cortex region.

In certain embodiments, the electrode is adapted for positioning on a surface of the sensorimotor cortex region of the brain in a subdural space.

In certain embodiments, the neural recording device comprises a brain-penetrating electrode array.

In certain embodiments, the neural recording device comprises an electrocorticography (ECoG) electrode array.

In certain embodiments, the electrode is a depth electrode or a surface electrode.

In certain embodiments, the electrical signal data comprises high-gamma frequency content features (e.g., 70 Hz to 150 Hz) and low frequency content features (e.g., 0.3 Hz to 100 Hz).

In certain embodiments, the interface comprises a percutaneous pedestal connector attached to the subject's cranium.

In certain embodiments, the interface further comprises a headstage that is connectable to the percutaneous pedestal connector.

In certain embodiments, the processor is provided by a computer or handheld device (e.g., a cell phone or tablet).

In another aspect, a kit comprising a system described herein and instructions for using the system for recording and decoding brain electrical signal data associated with attempted speech, attempted spelling of words, or attempted non-speech motor movement by a subject, or a combination thereof.

The methods of assisting a subject with communication through decoding of neural activity associated with attempted speech, attempted spelling of words, or attempted non-speech motor movement can be combined. The techniques are complementary. In some cases, decoding of attempted spelling may enable a larger vocabulary to be used than for decoding of attempted speech. However, decoding of attempted speech may be easier and more convenient for the subject, as it allows faster, direct word decoding, which may be preferred to express frequently used words. To assist decoding, attempted non-speech motor movements may be used to signal a subject is initiating or ending attempted speech or spelling out of an intended message.

### BRIEF DESCRIPTION OF THE DRAWINGS

FIG. 1. Schematic overview of the direct speech BCI. Neural activity acquired from an investigational electrocorticography (ECoG) electrode array implanted in a clinical trial participant with severe paralysis is used to directly decode words and sentences in real time. In a conversational demonstration, the participant is visually prompted with a question (A) and is instructed to attempt to respond using words from a predefined 50-word vocabulary. Simultaneously, cortical signals are acquired from the surface of the brain via the ECoG device (B) and processed in real time (C). A speech detection model analyzes the processed neural signals sample-by-sample to detect the participant's attempts to speak (D). A classifier computes word probabilities (across the 50 possible words) from each detected window of relevant neural activity (E). A Viterbi decoding algorithm uses these probabilities in conjunction with word sequence probabilities from a separately trained language model to decode the most likely sentence given the ECoG data (F). The predicted sentence, which is updated each time a word is decoded, is displayed as feedback to the participant (G).

FIGS. 2A-2E. Neural signal processing and language modeling enable decoding of a variety of sentences in real time. FIG. 2A shows word error rates of the word sequences decoded from the participant's cortical activity during sentence task blocks. The word error rates quantify how frequently decoding errors were made (lower word error rate indicates better performance). Word error rates were significantly lower than chance when decoding words with and without the language model (LM), and performance was significantly improved when using the LM during decoding (* all P<0.001, 3-way Holm-Bonferroni correction). FIG. 2B shows decoded words per minute values across all trials when either including or excluding words that were incorrectly decoded. Each violin distribution was created using kernel density estimation with Scott bandwidth estimation, accompanied by a thick horizontal line depicting the median and smaller horizontal lines depicting the range (excluding outliers that were more than 4 standard deviations below or above the mean). FIG. 2C shows a summary of the differences between the number of detected and actual words in each trial, with the percent of trials with correct sentence lengths shown in black and incorrect sentence lengths shown in dark red. FIG. 2D shows the edit distances (the number of decoding errors made) for the decoded sentences with and without the LM across all trials and all 50 sentence targets, sorted by ascending edit distance for the predictions with the LM (lower edit distance indicates better performance). Each small vertical dash represents the edit distance for a single trial (there are 3 trials per target sentence; marks for identical edit distances are staggered horizontally for visualization purposes). Each dot represents the mean edit distance for that target sentence. The histogram on the bottom shows the edit distance counts across all of the trials. FIG. 2E shows the target sentence and the decoded sentence with and without use of the LM for seven different trials. Correctly decoded words are shown in black and incorrect words are shown in red.

FIGS. 3A-3C. Distinct neural activity patterns underlie word production attempts. FIG. 3A shows the effect of the amount of training data on word classification accuracy using cortical activity recorded during the participant's isolated word production attempts. Each point depicts mean±standard deviation across 10 cross-validation folds. Chance accuracy is depicted as a horizontal dashed line. FIG. 3B shows the participant's brain reconstruction overlaid with the locations of the implanted electrodes and their contributions to the speech detection and word classification models. Plotted electrode size (area) and opacity are scaled by relative contribution (important electrodes appear larger and more opaque than other electrodes). Each set of contributions are normalized to sum to 1. For anatomical reference, the precentral gyrus is highlighted in light blue. FIG. 3C shows word confusions from the classification results, depicting how often the classifier predicted each of the 50 words given the identity of the target word that the participant was attempting to say (values along the diagonal correspond to correct classifications).

FIGS. 4A-4B. Neural activity recorded during attempted speech exhibits long-term stability. FIG. 4A shows neural activity from a single electrode across all of the participant's attempts to say the word “Goodbye” during the isolated word task, spanning over 18 months of recording. FIG. 4B shows word classification outcomes from training and testing the detector and classifier on subsets of isolated word data sampled from four non-overlapping date ranges. Each subset contains data from 20 attempted productions of each word. Each solid bar depicts results from cross-validated evaluation within a single subset, and each dotted bar depicts results from training on data from all of the subsets except for the one that is being evaluated. Each bar depicts mean±standard error across 10 evaluation folds. Chance accuracy is depicted as a horizontal dashed line. Also shown are significant differences between the four same-subset evaluations (*P<0.01, two-tailed Fisher's exact test, 10-way Holm-Bonferroni correction) and between the two evaluations for each test subset (*P<0.01, two-tailed exact McNemar's test, 10-way Holm-Bonferroni correction). Electrode contributions computed during cross-validated evaluation within a single subset are shown on top (oriented with the most dorsal and posterior electrode in the upper-right corner). Plotted electrode size (area) and opacity are scaled by relative contribution. Each set of contributions are normalized to sum to 1.

FIGS. 5A-5B. MRI results for the participant. FIG. 5A shows a sagittal MRI for the participant, who has encephalomalacia and brainstem atrophy (labeled in blue) caused by pontine stroke (labeled in red). FIG. 5B shows two additional MRI scans that indicate the absence of cerebral atrophy, suggesting that cortical neuron populations (including those recorded from in this study) should be relatively unaffected by the participant's pathology.

FIG. 6. Real-time neural data acquisition hardware infrastructure. Electrocorticography (ECoG) data acquired from the implanted array and percutaneous pedestal connector are processed and transmitted to the Neuroport digital signal processor (DSP). Simultaneously, microphone data are acquired, amplified, and transmitted to the DSP. Signals from the DSP are transmitted to the real-time computer. The real-time computer controls the task displayed to the participant, including any decoded sentences that are provided in real time as feedback. Speaker output from the real-time computer is also sent to the DSP and synchronized with the neural signals (not depicted). During earlier sessions, a human patient cable connected to the pedestal acquired the ECoG signals, which were then processed by a front-end amplifier before being transmitted to the DSP (the human patient cable and front-end amplifier are not shown here, but they replaced the digital headstage and digital hub in this pipeline when they were used).

FIG. 7. Real-time neural signal processing pipeline. Using the data acquisition headstage and rig, the participant's electrocorticography (ECoG) signals were acquired at 30 kHz, filtered with a wide-band filter, conditioned with a software-based line noise cancellation technique, low-pass filtered at 500 Hz, and streamed to the real-time computer at 1 kHz. On the real-time computer, custom software was used to perform common average referencing, multi-band high gamma band-pass filtering, analytic amplitude estimation, multi-band averaging, and running z-scoring on the ECoG signals. The resulting signals were then used as the measure of high gamma activity for the remaining analyses.

FIG. 8. Data collection timeline. Bars are stacked vertically if more than one data type was collected in a day (the height of the stacked bars for any given day is equal to the total number of trials collected that day). The irregularity of the data collection schedule was caused in part by external and clinical time constraints unrelated to the implanted device. The gap from 55-88 weeks was due to clinical guidelines concerning the COVID-19 pandemic.

FIG. 9. Speech detection model schematic. The z-scored high gamma activity across all electrodes is processed time point by time point by an artificial neural network consisting of a stack of three long short-term memory layers (LSTMs) and a single dense (fully connected) layer. The dense layer projects the latent dimensions of the last LSTM layer into probability space for three event classes: speech, preparation, and rest. The predicted speech event probability time series is smoothed and then thresholded with probability and time thresholds to yield onset (t*) and offset times of detected speech events. During sentence decoding, each time a speech event was detected, the window of neural activity spanning from −1 to 3 seconds relative to the detected onset (t*) was passed to the word classifier. The neural activity, predicted speech probability time series (upper right), and detected speech event (lower right) shown are the actual neural data and detection results across a 7-second time window for an isolated word trial in which the participant attempted to produce the word “family”.

FIG. 10. Word classification model schematic. For each classification, a 4-second time window of high gamma activity is processed by an ensemble of 10 artificial neural network (ANN) models. Within each ANN, the high gamma activity is processed by a temporal convolution followed by two bidirectional gated recurrent unit (GRU) layers. A dense layer projects the latent dimension from the final GRU layer into probability space, which contains the probability of each of the words from the 50-word set being the target word during the speech production attempt associated with the neural time window. The 10 probability distributions from the ensembled ANN models are averaged together to obtain the final vector of predicted word probabilities.

FIG. 11. Sentence decoding hidden Markov model. This hidden Markov model (HMM) describes the relationship between the words that the participant attempts to produce (the hidden states qᵢ) and the associated detected time windows of neural activity (the observed states yᵢ). The HMM emission probabilities p(y₀|q₀) can be simplified to p(wᵢ|yᵢ) (the word likelihoods provided by the word classifier), and the HMM transition probabilities p(qᵢ|qᵢ₋₁) can be simplified to p(wᵢ|cᵢ) (the word-sequence prior probabilities provided by the language model).

FIGS. 12A-12C. Auxiliary modeling results with isolated word data. FIG. 12A shows the effect of the amount of training data on word classification accuracy (left) and cross-entropy loss (right) using cortical activity recorded during the participant's isolated word production attempts. Lower cross entropy indicates better performance. Each point depicts mean standard deviation across 10 cross-validation folds (the error bars in the cross-entropy plot were typically too small to be seen alongside the circular markers). Chance performance is depicted as a horizontal dashed line in each plot (chance cross-entropy loss is computed as the negative log (base 2) of the reciprocal of the number of word targets). Performance improved more rapidly for the first four hours of training data and then less rapidly for the next 5 hours, although it did not plateau. When using all available isolated word data, the information transfer rate was 25.1 bits per minute (not depicted). FIG. 12B shows the effect of the amount of training data on the frequency of detection errors during speech detection and detected event curation with the isolated word data. Lower error rates indicate better performance. False positives are detected events that were not associated with a word production attempt and false negatives are word production attempts that were not associated with a detected event. Each point depicts mean±standard deviation across 10 cross-validation folds. Not all of the available training data was used to fit each speech detection model, but each model always used between 47 and 83 minutes of data (not depicted). FIG. 12C shows the distribution of onsets detected from neural activity across 9000 isolated word trials relative to the go cue (100 ms histogram bin size). This histogram was created using results from the final set of analyses in the learning curve scheme (in which all available trials were included in the cross-validated evaluation). The distribution of detected speech onsets had a mean of 308 ms after the associated go cues and a standard deviation of 1017 ms. This distribution was likely influenced to some degree by behavioral variability in the participant's response times. During detected event curation, 429 trials required curation to choose a detected event from multiple candidates (420 trials had 2 candidates and 9 trials had 3 candidates).

FIG. 13. Acoustic contamination investigation. Each blue curve depicts the average correlations between the spectrograms from a single electrode and the corresponding spectrograms from the time-aligned microphone signal as a function of frequency. The red curve depicts the average power spectral density (PSD) of the microphone signal. Vertical dashed lines mark the 60 Hz line noise frequency and its harmonics. Highlighted in green is the high gamma frequency band (70-150 Hz), which was the frequency band from which we extracted the neural features used during decoding. Across all frequencies, correlations between the electrode and microphone signals are small. There is a slight increase in correlation in the lower end of the high gamma frequency range, but this increase in correlation occurs as the microphone PSD decreases. Because the correlations are low and do not increase or decrease with the microphone PSD, the observed correlations are likely due to factors other than acoustic contamination, such as shared electrical noise. After comparing these results to those observed in the study describing acoustic contamination (which informed the contamination analysis we used here) [39], we conclude that our decoding performance was not artificially improved by acoustic contamination of our electrophysiological recordings.

FIGS. 14A-14C. Long-term stability of speech-evoked signals. FIG. 14A shows neural activity from a single electrode across all of the participant's attempts to say the word “Goodbye” during the isolated word task, spanning 81 weeks of recording. FIG. 14B shows the participant's brain reconstruction overlaid with electrode locations. The electrode shown in Panel A is filled in with black. For anatomical reference, the precentral gyrus is highlighted in light blue. FIG. 14C shows word classification outcomes from training and testing the detector and classifier on subsets of isolated word data sampled from four non-overlapping date ranges. Each subset contains data from 20 attempted productions of each word. Each solid bar depicts results from cross-validated evaluation within a single subset, and each dotted bar depicts results from training on data from all of the subsets except for the one that is being evaluated. Each error bar shows the 95% confidence interval of the mean, computed across cross-validation folds. Chance accuracy is depicted as a horizontal dashed line. Electrode contributions computed during cross-validated evaluation within a single subset are shown on top (oriented with the most dorsal and posterior electrode in the upper-right corner). Plotted electrode size (area) and opacity are scaled by relative contribution. Each set of contributions is normalized to sum to 1. These results suggest that speech-evoked cortical responses remained relatively stable throughout the study period, although model recalibration every 2-3 months may still be beneficial for decoding performance.

FIG. 15. Schematic depiction of the spelling pipeline. A. At the start of a sentence-spelling trial, the participant attempts to silently say a word to volitionally activate the speller. B. Neural features (high-gamma activity and low-frequency signals) are extracted in real time from the recorded cortical data throughout the task. The features from a single electrode (electrode 0 as shown in FIG. 19A) are depicted. For visualization, the traces were smoothed via convolution with a Gaussian kernel with a standard deviation of 150 milliseconds. The microphone signal shows that there is no vocal output during the task. C. The speech-detection model, consisting of a recurrent neural network (RNN) and thresholding operations, processes the neural features sample-by-sample to detect a silent-speech attempt. Once an attempt is detected, the detection model becomes inactive and the spelling procedure begins. D. During the spelling procedure, the participant spells out the intended message throughout letter-decoding cycles that occur every 2.5 seconds. Each cycle, the participant is visually presented with a countdown and eventually a go cue. At the go cue, the participant attempts to silently say the code word that represents the desired letter. E. High-gamma activity and low-frequency signals are computed throughout the spelling procedure for all electrode channels and parceled into 2.5-second non-overlapping time windows corresponding to the letter-decoding cycles. F. An RNN-based letter-classification model processes each of these neural time windows to predict the probability that the participant was attempting to silently say each of the 26 possible code words or attempting to perform a hand-motor command (see G). If the classifier predicts that the participant was performing the hand-motor command with at least 80% probability, the spelling procedure ends and the sentence is finalized (see I). Otherwise, the predicted letter probabilities are processed by a beam-search algorithm in real time and the most likely sentence is displayed to the participant. G. After the participant spells out his intended message, he attempts to squeeze his right hand during the next letter-decoding cycle to end the spelling procedure and finalize the sentence. H. The neural time window associated with the hand-motor command is passed to the classification model. I. If the classifier confirms that the participant attempted the hand-motor command, a neural network-based language model (“DistilGPT-2”) rescores the sentences composed solely of complete words, and the system uses the most likely sentence after rescoring as the final prediction.

FIGS. 16A-16F. Performance summary of the spelling system during the copy-typing task. FIG. 16A. Character error rates (CERs) observed during real-time sentence spelling (denoted as ‘+LM (Real-time results)’) and offline simulations in which portions of the spelling system were omitted. In the ‘Chance’ condition, sentences were created by replacing the outputs from the neural classifier with randomly generated letter probabilities without altering the remainder of the spelling pipeline. In the ‘Only neural decoding’ condition, sentences were created solely by concatenating together the most likely character from each of the classifier's predictions during a sentence trial (which did not include any whitespace characters). In the ‘+Vocab. constraints’ condition, the predicted letter probabilities from the neural classifier were used with a beam search that constrained the predicted character sequences to form words from within the 1,152-word vocabulary. The final condition labeled ‘+LM (Real-time results)’ shows the real-time results during testing with the participant, incorporating language modeling during the beam search and after the sentence is finalized. The sentences decoded with the full system in real time exhibited lower CERs than sentences decoded in the other conditions (*** P<0.0001, two-sided Wilcoxon Rank-Sum test with 6-way Holm-Bonferroni correction). FIG. 16B. Word error rates (WERs) for real-time results and corresponding offline omission simulations from FIG. 16A. FIG. 16C. The decoded characters per minute during real-time testing. FIG. 16D. The decoded words per minute during real-time testing. In FIGS. 16A-16D, the distribution described each boxplot was computed across n=34 real-time blocks (in each block, the participant attempted to spell between 2-5 sentences), and each boxplot depicts the quartiles of the data with whiskers extending to show the rest of the distribution except for data points that are 1.5 times the interquartile range. In FIG. 16A and FIG. 16B, each boxplot corresponds to n=34 blocks (in each of these blocks, the participant attempted to spell between two to five sentences). In FIG. 16C, each boxplot corresponds to n=9 blocks (in each of these blocks, the participant attempted to spell between two to four conversational responses). FIG. 16E. Number of excess characters in each decoded sentence. A decoded sentence with 0 excess characters indicates that a hand-motor command (to disengage the speller) was successfully identified from the participant's neural activity immediately after he spelled the final letter in that sentence. FIG. 16F. Example sentence-spelling trials with decoded sentences from each non-chance condition. Incorrect letters are colored red. 1 and 2 mark trials in which the sentence decoded in real time contained at least one error. The target sentences for these two trials are given at the bottom of the panel. All other example sentences did not contain any real-time decoding errors.

FIGS. 17A-17H. Characterization of high-gamma activity (HGA) and low-frequency signals (LFS) during silent-speech attempts. FIG. 17A. 10-fold cross-validated classification accuracy on silently attempted NATO code words when using HGA alone, LFS alone, and both HGA+LFS simultaneously. Classification accuracy using only LFS is significantly higher than using only HGA, and using both HGA+LFS results in significantly higher accuracy than using either feature type alone (** P<0.001, two-sided Wilcoxon Rank-Sum test with 3-way Holm-Bonferroni correction). Chance accuracy is 3.7%. Each boxplot depicts the quartiles of the data with whiskers extending to show the remainder of the distribution except for data points that are 1.5 times the interquartile range. Each boxplot corresponds to n=10 cross-validation folds. FIG. 17B. Electrode contributions from a classification model trained using only HGA features. Plotted electrode size and opacity are scaled by relative contribution; electrodes that appear larger and more opaque provide more important features to the classification model. FIG. 17C. Electrode contributions associated with HGA features from a classification model trained using the combined HGA+LFS feature set. FIG. 17D. Electrode contributions from a classification model trained using only LFS features. FIG. 17E. Electrode contributions associated with LFS features from a classification model trained using the combined HGA+LFS feature set. In FIGS. 17B-17E, plotted electrode size and opacity are scaled by relative contribution; electrodes that appear larger and more opaque provided more important features to the classification model. FIG. 17F. Minimum number of principal components (PCs) required to explain more than 80% of the variance in the spatial dimension for each feature set over 100 bootstrap iterations. The number of PCs required were significantly different for each feature set (*** P<0.0001, two-sided Wilcoxon Rank-Sum test with 3-way Holm-Bonferroni correction, * P<0.01 two-sided Wilcoxon Rank-Sum test with 3-way Holm-Bonferroni correction). FIG. 17G. Minimum number of PCs required to explain more than 80% of the variance in the temporal dimension for each feature set over 100 bootstrap iterations. In FIG. 17F and FIG. 17G, the number of PCs required for each feature set is depicted as a histogram, where the x-axis is the percent of the bootstrap iterations that required a certain number of PCs. FIG. 17H. Effect of temporal smoothing on classification accuracy. Each point represents the median and error bars represent the 99% confidence interval around bootstrapped estimations of the median.

FIGS. 18A-18C. Comparison of neural signals during attempts to silently say English letters and NATO code words. FIG. 18A. Classification accuracy (across n=10 cross-validation folds) using models trained with HGA+LFS features is significantly higher for NATO code words than for English letters (** P<0.001, two-sided Wilcoxon Rank-Sum test). The dotted horizontal line represents chance accuracy. FIG. 18B. Nearest-class distance for the combined HGA+LFS feature set is significantly larger for NATO code words than for letters (boxplots show values across the n=26 code words or letters; * P<0.01, two-sided Wilcoxon Rank-Sum test). In FIG. 18A and FIG. 18B, each boxplot depicts the quartiles of the data with whiskers extending to show the rest of the distribution except for data points that are 1.5 times the interquartile range. FIG. 18C. The nearest-class distance is greater for the majority of code words than for the corresponding letters. In FIG. 18B and FIG. 18C, nearest-class distances are computed as the Frobenius norm between trial-averaged HGA+LFS features.

FIGS. 19A-19D. Differences in neural signals and classification performance between overt- and silent-speech attempts. FIG. 19A. MRI reconstruction of the participant's brain overlaid with implanted electrode locations. The locations of the electrodes used in FIG. 19B and FIG. 19C are bolded and numbered in the overlay. FIG. 19B. High-gamma activity (HGA) event-related potentials during silent (orange) and overt (green) attempts to say the NATO code word “kilo”. FIG. 19C. High-gamma activity (HGA) event-related potentials during silent (orange) and overt (green) attempts to say the NATO code word “tango”. Evoked responses in FIGS. 19B and C are aligned to the go cue, which is marked as a vertical dashed line at time 0. Each curve depicts the mean±standard error across n=100 speech attempts. FIG. 19D. Code-word classification accuracy (across 10 cross-validation folds) with various model-training schemes. All comparisons revealed significant differences between the result pairs (P<0.01, two-sided Wilcoxon Rank-Sum with 28-way Holm-Bonferroni correction) except for those marked as ‘ns’. Each boxplot corresponds to n=10 cross-validation folds. Chance accuracy is 3.84%.

FIGS. 20A-20D. The spelling approach can generalize to larger vocabularies and conversational settings. FIG. 20A. Simulated character error rates from the copy-typing task with different vocabularies, including the original vocabulary used during real-time decoding.

FIG. 20B. Word error rates from the corresponding simulations in FIG. 20A. FIG. 20C. Character and word error rates across the volitionally chosen responses and messages decoded in real time during the conversational task condition. In FIGS. 20A-20C, each boxplot depicts the quartiles of the data with whiskers extending to show the rest of the distribution except for data points that are 1.5 times the interquartile range. FIG. 20D. Examples of presented questions from trials of the conversational task condition (left) along with corresponding responses decoded from the participant's brain activity (right). In the final example, the participant spelled out his intended message without being prompted with a question.

FIG. 21. Data collection timeline. Each bar depicts the total number of trials collected on each day of recording. The participant and implant date are the same as in our previous work [2]. If more than one type of dataset was collected in a single day, the bar is colored by the proportion of each dataset collected. Each color represents a specific dataset (as specified in the legend). Datasets vary in task type (isolated-target or real-time sentence spelling), utterance set (English letters, NATO code words (which included the attempted hand squeeze), copy-typing sentences, or conversational sentences), and, for the real-time sentence-spelling datasets, the purpose of the data (for hyperparameter optimization or for performance evaluation). All speech-related trials were associated with silent-speech attempts, except for the dataset with “(overt)” in its legend label. Additionally, 3.06% of trials in this overt dataset were actually recorded during a version of the copy-typing sentence-spelling task in which the participant attempted to overtly produce the code words (see Section S3 for more details). Datasets were collected on an irregular schedule due to external and clinical time constraints that were unrelated to the neural implant. The gap from 55-88 weeks was specifically due to clinical guidelines during the start of the COVID-19 pandemic that limited or prevented in-person recording sessions.

FIG. 22. Real-time signal-processing pipeline. A detachable data-acquisition headstage (CerePlex E, Blackrock Microsystems) attached to the percutaneous pedestal connector applied a hardware-based wide-band Butterworth filter (between 0.3 Hz and 7.5 kHz) to the ECoG signals, digitized them with 16-bit, 250-nV per bit resolution, and transmitted them at 30 kHz through additional connections to a Neuroport system (Blackrock Microsystems), which processed the signals using software-based line noise cancellation and an anti-aliasing low-pass filter (at 500 Hz). Afterwards, the processed signals were streamed at 1 kHz to a separate computer for further real-time processing and analysis, where we applied a common average reference (across all electrode channels) to each time sample of the ECoG data. The re-referenced signals were then processed in two parallel streams to extract high-gamma activity (HGA) and low-frequency signal (LFS) features. To compute the HGA features, we applied eight 390^{th}-order band-pass finite impulse response (FIR) filters to the re-referenced signals (filter center frequencies were within the high-gamma band at 72.0, 79.5, 87.8, 96.9, 107.0, 118.1, 130.4, and 144.0 Hz). Then, for each channel and band, we used a 170^{th}-order FIR filter to approximate the Hilbert transform. Specifically, for each channel and band, we set the real component of the analytic signal equal to the original signal delayed by 85 samples (half of the filter order) and set the imaginary component equal to the Hilbert transform of the original signal (approximated by this FIR filter) [25]. We then computed the magnitude of each analytic signal at every fifth time sample, yielding analytic amplitude signals at 200 Hz. For each channel, we averaged the analytic amplitude values across the eight bands at each time point to obtain a single high-gamma analytic amplitude measure for that channel. To compute the LFS features, we downsampled the re-referenced signals to 200 Hz after applying a 130^{th}-order anti-aliasing low-pass FIR filter with a cutoff frequency of 100 Hz. We then combined the time-synchronized values from the two feature streams (high-gamma analytic amplitudes and downsampled signals) into a single feature stream. Next, we z-scored the values for each channel and each feature type using Welford's method with a 30-second sliding window [26]. Finally, we implemented a simple artifact-rejection approach to prevent samples with uncommonly large z-score magnitudes from interfering with the running z-score statistics or downstream decoding processes. We adapted this figure from our previous works [2, 27], which implemented similar preprocessing pipelines to compute high-gamma features.

FIG. 23. Speech-detection model schematic. To detect silent-speech attempts from the participant's neural activity during real-time sentence spelling, first the z-scored low-frequency signals (LFS) and high-gamma activity (HGA) for each electrode are processed continuously by a stack of 3 long short-term memory (LSTM) layers. Next, a single dense (fully connected) layer projects the latent dimensions of the final LSTM onto the 4 possible classes: speech, speech preparation, rest, and motor. The stream of speech probabilities is then temporally smoothed, probability thresholded, and time thresholded to yield onsets and offsets of full speech events. Once the participant attempts to silently say something and that speech attempt is detected, the spelling system is engaged and the paced spelling procedure begins. The depicted neural features, predicted speech-probability time series (upper right), and detected speech event (lower right) are the actual neural data and detection results for a 5-second time window at the beginning of a trial of the real-time sentence copy-typing task. This figure was adapted from our previous work [2], which implemented a similar speech-detection architecture.

FIGS. 24A-24B. Effects of feature selection on code-word classification accuracy. FIG. 24A. Classification accuracy improves for each code word when using high-gamma activity (HGA) and low-frequency signals (LFS) together (the combined HGA+LFS feature set) instead of only HGA features. FIG. 24B. Classification accuracy improves for almost every code word when using HGA+LFS instead of LFS alone. In both FIG. 24A and FIG. 24B, code words are represented as lower-case letters and the Spearman rank correlations are shown. The associated p-value was computed via permutation testing, where one group of observations (code-word accuracies for either HGA, LFS, or HGA+LFS) was shuffled before re-computing the correlation between that group of observations and the other group. 2000 iterations were used during permutation testing for each of the two comparisons.

FIG. 25. Confusion matrix from isolated-target trial classification. Confusion values, computed during offline classification of neural data (using both high-gamma activity and low-frequency signals) recorded during isolated-target trials, are shown for each NATO code word and the attempted hand squeeze. Each row corresponds to a target code word or the attempted hand squeeze, and the value in each column for that row corresponds to the percent of isolated-target task trials that were correctly classified as the target (if the value is along the diagonal) or misclassified (“confused”) as another potential target (if the value is not along the diagonal). The values in each row sum to 100%. In general, silent-speech and hand-squeeze attempts were reliably classified.

FIGS. 26A-26B. Neural-activation characteristics during overt- and silent-speech attempts. FIG. 26A. Each image shows an MRI reconstruction of the participant's brain overlaid with electrode locations and the maximum neural activations for each electrode, type of speech attempt (overt or silent), and feature type (high-gamma activity (HGA) or low-frequency signals (LFS)), measured as maximum peak code-word average magnitudes. To calculate these values, the trial-averaged neural-feature time series was computed for each code word, electrode, type of speech attempt, and feature type using the isolated-target dataset (for each trial, the 2.5-second time window after the go cue was used). Then, the peak magnitude (maximum of the absolute value) of each of these trial-averaged time series was determined. The maximum peak code-word average magnitude for each electrode, type of speech attempt, and feature type was then computed as the maximum value of these peak magnitudes across code words for each combination. The two columns show the values for each type of speech attempt (overt then silent), and the two rows show the values for each feature type (HGA then LFS). FIG. 26B. The standard deviation of peak code-word average magnitudes. Here, the standard deviation (instead of the maximum used in FIG. 26A) of the peak average magnitudes across the code words for each electrode, type of speech attempt, and feature type is computed and plotted, depicting how much the magnitudes varied across speech targets for that combination. For FIG. 26A and FIG. 26B, the color of each plotted electrode indicates the true associated value for that electrode, and the size of each electrode depicts the associated value for that electrode relative to the values for the other electrodes (for a given type of speech attempt and feature type).

### DETAILED DESCRIPTION

Methods, devices, and systems for assisting a subject with communication are provided. In particular, methods, devices, and systems are provided for decoding words and sentences directly from neural activity of an individual. In the disclosed methods, cortical activity from a region of the brain involved in speech processing is recorded while an individual attempts to say or spell out words of a sentence. Deep learning computational models are used to detect and classify words from the recorded brain activity. Decoding of speech from brain activity is aided by use of a language model that predicts how likely certain sequences of words are to occur. In addition, decoding of attempted non-speech motor movements from neural activity can be used to further assist communication.

The methods, devices, and systems disclosed herein may be used to assist individuals who have difficulty with communication caused by conditions and diseases including, without limitation, strokes, traumatic brain injuries, brain tumors, amyotrophic lateral sclerosis, multiple sclerosis, Huntington's disease, Niemann-Pick disease, Friedreich's ataxia, Wilson's disease, cerebral palsy, Guillain-Barre syndrome, Tay-Sachs disease, encephalopathy, central pontine myelinolysis, and other conditions causing dysfunction or paralysis of the muscles of the head, neck, or chest resulting in anarthria. The methods disclosed herein may be used to restore communication to such individuals and improve autonomy and quality of life.

Before exemplary embodiments of the present invention are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials may now be described. Any and all publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “an electrode” or “the electrode” includes a plurality of such electrodes and reference to “a signal” or “the signal” includes reference to one or more signals, and so forth.

It is further noted that the claims may be drafted to exclude any element which may be optional. As such, this statement is intended to serve as antecedent basis for use of such exclusive terminology as “solely”, “only” and the like in connection with the recitation of claim elements, or the use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed. To the extent such publications may set out definitions of a term that conflicts with the explicit or implicit definition of the present disclosure, the definition of the present disclosure controls.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Definitions

The term “communication disorders” is used herein to refer to a group of conditions that affect the ability of a subject to speak. Communication disorders include, without limitation, anarthria, strokes, traumatic brain injuries, brain tumors, amyotrophic lateral sclerosis, multiple sclerosis, Huntington's disease, Niemann-Pick disease, Friedreich's ataxia, Wilson's disease, cerebral palsy, Guillain-Barre syndrome, Tay-Sachs disease, encephalopathy, central pontine myelinolysis, and other conditions causing dysfunction or paralysis of the muscles of the head, neck, or chest resulting in anarthria.

The term “communication” includes word-based communication such as verbal communication including spoken speech, spelling of words, and production of text (e.g., controlling a personal device to generate email or text via attempts to speak) as well as action-based communication such as through attempted non-speech motor movement. Attempted speech may include vocalized speech, which may or may not be intelligible, or non-vocalized speech. Silent-speech attempts are volitional attempts to articulate speech without vocalizing. Silent-spelling attempts are volitional attempts to spell alphabetical characters or numbers without vocalizing. Attempted non-speech motor movement may include imagined movement without any detectable physical movement. Attempted non-speech motor movements may include, without limitation, imagined head, arm, hand, foot, and leg movements. Attempted non-speech motor movements may be used to indicate the initiation or termination of attempted speech or spelling or to control an external device (e.g., for communication with a personal device or software applications or to turn on or off a device). In the disclosed methods, neural activity is recorded during attempts to communicate whether or not the individual produces any vocal output or detectable motor movement.

The terms “subject”, “individual”, “patient”, and “participant” are used interchangeably herein and refer to a patient having a communication disorder. The patient is preferably human, e.g., a child, an adolescent, an adult, such as a young, middle-aged, or elderly human who may benefit from the systems, devices, and methods disclosed herein for restoring communication. The patient may have been diagnosed as having anarthria.

The term “user” as used herein refers to a person that interacts with a device and/system disclosed herein for performing one or more steps of the presently disclosed methods. The user may be the patient receiving treatment. The user may be a health care practitioner, such as, the patient's physician.

### Methods

The present disclosure provides methods for assisting a subject with communication. Methods are provided for decoding words and sentences directly from neural activity of an individual. In the disclosed methods, cortical activity from a region of the brain involved in speech processing is recorded while an individual attempts to say or spell out words of a sentence. Attempts to say or spell out words can include or exclude vocalizations. That is, neural activity is recorded during attempts to say or spell out words whether or not the individual produces any vocal output. In some cases, the vocal output may be unintelligible when the individual attempts to say or spell out words. Deep learning computational models are used to detect and classify words and/or spelled letters from the recorded brain activity. Decoding of speech from brain activity is aided by use of a language model that predicts how likely certain sequences of words occur. The neurotechnology described herein can be used to restore communication to patients who have lost the ability to speak and has the potential to improve autonomy and quality of life. Various steps and aspects of the methods will now be described in greater detail below.

The method includes positioning a neural recording device comprising one or more electrodes at a location in a sensorimotor cortex region of the brain of the subject to record brain electrical signal data associated with attempted speech and/or attempted spelling by the subject; and positioning an interface in communication with a computing device at a location on the head of the subject. Brain electrical signal data associated with attempted speech and/or attempted spelling by the subject is recorded using the neural recording device, wherein the interface receives the brain electrical signal data from the neural recording device and transmits the brain electrical signal data to a processor programmed to detect attempted speech and/or spelling by the subject and decode spelled letters, words, phrases, or sentences from the recorded brain electrical signal data.

The recording device may comprise non-brain penetrating surface electrodes or brain-penetrating depth electrodes. The electrical signals may be recorded using a single electrode, electrode pairs, or an electrode array. In some embodiments, the brain activity is recorded from more than one site. In certain embodiments, brain electrical signal data is recorded from a sensorimotor cortex region of the brain involved in speech processing such as the precentral gyrus, postcentral gyrus, posterior middle frontal gyrus, posterior superior frontal gyrus, or posterior inferior frontal gyrus region, or any combination thereof. In some embodiments, the electrode is positioned on a surface of the sensorimotor cortex region of the brain in a subdural space.

Positioning an electrode for recording brain activity at specified region(s) of the brain may be carried out using standard surgical procedures for placement of intra-cranial electrodes. As used herein, the phrases “an electrode” or “the electrode” refer to a single electrode or multiple electrodes such as an electrode array. As used herein, the term “contact” as used in the context of an electrode in contact with a region of the brain refers to a physical association between the electrode and the region. In other words, an electrode that is in contact with a region of the brain is physically touching the region of the brain. An electrode in contact with a region of the brain can be used to detect electrical signals corresponding to neural activity associated with attempted speech and/or spelling. Electrodes used in the methods disclosed herein may be monopolar (cathode or anode) or bipolar (e.g., having an anode and a cathode).

In certain embodiments, one or more electrodes are used to record electrical signals for neural activity associated with attempted speech and/or attempted spelling in one or more brain regions. An electrode may be placed, for example, in a region of the sensorimotor cortex involved in speech processing such as the precentral gyrus, postcentral gyrus, posterior middle frontal gyrus, posterior superior frontal gyrus, or posterior inferior frontal gyrus region of the brain. In certain cases, placing the electrode may involve positioning the electrode on the surface of the specified region(s) of the brain. For example, electrodes may be placed on the surface of the brain at the precentral gyrus, postcentral gyrus, posterior middle frontal gyrus, posterior superior frontal gyrus, or posterior inferior frontal gyrus region, or any combination thereof. The electrode may contact at least a portion of the surface of the brain at the precentral gyrus, postcentral gyrus, posterior middle frontal gyrus, posterior superior frontal gyrus, or posterior inferior frontal gyrus regions. In some embodiments, the electrode may contact substantially the entire surface area at the precentral gyrus, postcentral gyrus, posterior middle frontal gyrus, posterior superior frontal gyrus, or posterior inferior frontal gyrus regions. In some embodiments, the electrode may additionally contact area(s) adjacent to the precentral gyrus, postcentral gyrus, posterior middle frontal gyrus, posterior superior frontal gyrus, or posterior inferior frontal gyrus regions.

In some embodiments, an electrode array arranged on a planar support substrate may be used for detecting electrical signals for neural activity from one or more of the brain regions specified herein. The surface area of the electrode array may be determined by the desired area of contact between the electrode array and the brain. An electrode for implanting on a brain surface, such as, a surface electrode or a surface electrode array may be obtained from a commercial supplier. A commercially obtained electrode/electrode array may be modified to achieve a desired contact area. In some cases, the non-brain penetrating electrode (also referred to as a surface electrode) that may be used in the methods disclosed herein may be an electrocorticography (ECoG) electrode or an electroencephalography (EEG) electrode.

In certain cases, placing the electrode at a target area or site (e.g., a neural recording device electrode) may involve positioning a brain penetrating electrode (also referred to as depth electrode) in the specified region(s) of the brain. For example, a depth electrode may be placed in a selected region of the sensorimotor cortex involved in speech processing (e.g., the precentral gyrus, postcentral gyrus, posterior middle frontal gyrus, posterior superior frontal gyrus, or posterior inferior frontal gyrus region). In some embodiments, the electrode may additionally contact area(s) adjacent to the selected region of the sensorimotor cortex involved in speech processing (e.g., adjacent to the precentral gyrus, postcentral gyrus, posterior middle frontal gyrus, posterior superior frontal gyrus, or posterior inferior frontal gyrus region). In some embodiments, an electrode array may be used for recording electrical signals at the selected region of the sensorimotor cortex involved in speech processing (e.g., the precentral gyrus, postcentral gyrus, posterior middle frontal gyrus, posterior superior frontal gyrus, or posterior inferior frontal gyrus region) as specified herein.

The depth to which an electrode is inserted into the brain may be determined by the desired level of contact between the electrode array and the brain and the types of neural populations that the electrode would have access to for recording electrical signals. A brain-penetrating electrode array may be obtained from a commercial supplier. A commercially obtained electrode array may be modified to achieve a desired depth of insertion into the brain tissue.

The precise number of electrodes contained in an electrode array (e.g., for recording of neural activity associated with attempted speech) may vary. In certain aspects, an electrode array may include two or more electrodes, such as 3 or more, 10 or more, 50 or more, 100 or more, 200 or more, 500 or more, including 4 or more, e.g., about 3 to 6 electrodes, about 6 to 12 electrodes, about 12 to 18 electrodes, about 18 to 24 electrodes, about 24 to 30 electrodes, about 30 to 48 electrodes, about 48 to 72 electrodes, about 72 to 96 electrodes, about 96 to 128 electrodes, about 128 to 196 electrodes, about 196 to 294 electrodes, or more electrodes. The electrodes may be arranged into a regular repeating pattern (e.g., a grid, such as a grid with about 1 cm spacing between electrodes), or no pattern. An electrode that conforms to the target site for optimal recording of electrical signals from neural activity associated with attempted speech and/or spelling by a subject may be used. One such example, is a single multi contact electrode with eight contacts separated by 2½ mm. Each contact would have a span of approximately 2 mm. Another example is an electrode with two 1 cm contacts with a 2 mm intervening gap. Yet further, another example of an electrode that can be used in the present methods is a 2 or 3 branched electrode to cover the target site. Each one of these three-pronged electrodes has four 1-2 mm contacts with a center-to-center separation of 2 to 2.5 mm and a span of 1.5 mm.

In some embodiments, a high-density ECoG electrode array is used to record electrical signals from neural activity associated with attempted speech and/or spelling by a subject. For example, a high-density ECoG electrode array may comprise at least 100 electrodes, at least 128 electrodes, at least 196 electrodes, at least 256 electrodes, at least 294 electrodes, at least 500 electrodes, or at least 1000 electrodes, or more. In some embodiments, the electrode center-to-center spacing in a high-density ECoG electrode array ranges from 250 μm to 4 mm, including any electrode center-to-center spacing within this range such as 250 μm, 300 μm, 350 m, 400 μm, 500 μm, 550 μm, 600 μm, 650 μm, 700 μm, 800 μm, 900 μm, 1 mm, 1.5 mm, 2 mm, 2.5 mm, 3 mm, 3.5 mm, or 4 mm. In some embodiments, a high-density ECoG micro-electrode array is used. ECoG micro-electrode arrays may comprise electrodes having a diameter of 250 μm or less, 230 μm or less, or 200 μm or less, including electrodes having a diameter ranging from 150 μm to 250 μm, including any diameter within this range such as 150, 160, 170, 180, 190, 200, 210, 220, 230, 240, or 250 μm. For a description of high-density ECoG electrode arrays and micro-electrode arrays, see, e.g., Muller et al. (2015) Annu Int Conf IEEE Eng Med Biol Soc 2016:1528-1531; Chiang et al (2020) J. Neural Eng. 17:046008; Escabi et al. (2014) J. Neurophysiol. 112(6): 1566-1583; herein incorporated by reference.

The size of each electrode may also vary depending upon such factors as the number of electrodes in the array, the location of the electrodes, the material, the age of the patient, and other factors. In certain aspects, each electrode has a size (e.g., a diameter) of about 5 mm or less, such as about 4 mm or less, including 4 mm-0.25 mm, 3 mm-0.25 mm, 2 mm-0.25 mm, 1 mm-0.25 mm, or about 3 mm, about 2 mm, about 1 mm, about 0.5 mm, or about 0.25 mm.

In certain embodiments, the method further comprises mapping the brain of the subject to optimize positioning of an electrode. Positioning of an electrode is optimized to detect brain activity features associated with attempted speech by the subject and to achieve optimal decoding of attempted speech. For example, patterns of electrical signals in specific frequency ranges (e.g., alpha, delta, beta, gamma, and/or high gamma) may be used for detecting attempted speech and/or spelling and decoding words, phrases, or sentences intended by the subject. Thus, electrodes may be positioned to optimize detection and/or decoding of brain activity in specific frequency ranges to restore communication to a subject who has a communication disorder.

In certain aspects, the methods and systems of the present disclosure may include recording brain activity, for example, electrical activity in the ventral sensorimotor cortex, where patterns of gamma-frequency neural activity associated with words, phrases, and sentences of attempted speech may be detected. In certain cases, electrical activity from a plurality of locations in the ventral sensorimotor cortex may be measured. In some embodiments, electrical activity in the high gamma frequency range (such as 70 Hz to 150 Hz) or the low frequency range (such as 0.3 Hz to 100 Hz) may be measured from the precentral gyrus, postcentral gyrus, posterior middle frontal gyrus, posterior superior frontal gyrus, or posterior inferior frontal gyrus region, or any combination thereof. In some embodiments, electrical activity in the high gamma frequency range (such as 70 Hz to 150 Hz) and the low frequency range (such as 0.3 Hz to 100 Hz) may be measured from the precentral gyrus, postcentral gyrus, posterior middle frontal gyrus, posterior superior frontal gyrus, or posterior inferior frontal gyrus region, or any combination thereof.

Detection of brain activity may be performed by any method known in the art. For example, functional brain imaging of neural activity may be carried out by electrical methods such as electrocorticography (ECoG), electroencephalography (EEG), stereoelectroencephalography (sEEG), magnetoencephalography (MEG), single photon emission computed tomography (SPECT), as well as metabolic and blood flow studies such as functional magnetic resonance imaging (fMRI), positron emission tomography (PET), functional near-infrared spectroscopy (fNIRS), and time-domain functional near-infrared spectroscopy. In some embodiments, the precentral gyrus, postcentral gyrus, posterior middle frontal gyrus, posterior superior frontal gyrus, or posterior inferior frontal gyrus region are mapped to determine optimal positioning for electrodes to detect neural activity associated with attempted speech and/or attempted spelling. One or more of these regions may be implanted with a neural recording device comprising electrodes to measure electrical signals from neural activity associated with attempted speech and/or attempted spelling.

In some cases, electrical activity in one or more locations in the brain may be measured not only during attempted speech or attempted spelling but also during a period extending from just prior to attempted speech or attempted spelling (i.e., period of preparation for speech or spelling) to a period just after attempted speech or spelling (i.e., rest period after attempted speech or spelling). Assessment of the accuracy of the decoding of speech or spelling from neural activity at a particular site may be determined by comparing decoded words to the intended words of the patient. For example, the patient may communicate the correct intended words using an assistive typing device. Both detection of the onset and offset of speech events and word/letter classification accuracy from decoding neural activity may be evaluated. False positives include detected speech events that are not associated with a true word or letter production attempt and false negatives include word/letter production attempts that are not associated with a detected speech event. Lower error rates in detection of speech events and decoding of words or spelled letters from neural activity indicate better performance. In certain cases, the placement of electrodes or the number of electrodes may be altered to improve detection of electrical signals and decoding of attempted speech and/or spelling by the subject.

Application of the method may include a prior step of selecting a patient for implantation with a neural recording device based on need as determined by clinical assessment of the severity of the communication disorder and the desire for assistance with communication, and may also include cognitive assessment, anatomical assessment, behavioral assessment and/or neurophysiological assessment. Patients who have difficulty with communication may be implanted with a neural recording device to assist communication, as described herein.

An interface capable of communication with a computing device is implanted in the cranium or placed on the head of the subject to provide an externally accessible platform through which brain electrical signals can be acquired from the neural recording device and transmitted to a data processor for decoding. In some embodiments, the interface comprises a percutaneous pedestal connector anchored in the cranium of the subject. The interface can be connected, for example, to a computing device such as a computer or a handheld computing device (e.g., cell phone or tablet) with a detachable digital connector and cable. Alternatively, the interface may be connected to a computing device wirelessly. In some embodiments, the interface comprises a first wireless communication unit in communication with a computing device comprising a second wireless communication unit. In some embodiments, the first wireless communication unit utilizes a wireless communication protocol using an electromagnetic carrier wave (e.g., a radio wave, microwave, or an infrared carrier wave) or ultrasound to transfer data from the interface to the computing device comprising the second wireless communication unit. Brain-computer interfaces are commercially available, including the Neuroport™ system from Blackrock Microsystems (Salt Lake City, Utah), See also, e.g., Weiss et al. (2019) Brain-Computer Interfaces 6:106-117; herein incorporated by reference.

The processor may be provided by a computer or a handheld computing device (e.g., cell phone or tablet) programmed to decode the attempted speech and/or attempted spelling from the recorded brain electrical signal data.

Analyzing the recorded brain electrical activity may comprise the use of an algorithm or classifier. In some embodiments, a machine learning algorithm is used to automate speech detection, letter classification (in the case of attempted spelling), word classification, and sentence decoding from analysis of recorded brain activity during attempted speech or spelling. The machine learning algorithm may comprise a supervised learning algorithm. Examples of supervised learning algorithms may include Average One-Dependence Estimators (AODE), Artificial neural network (e.g., artificial neural network comprising a stack of long short-term memory (LSTM) layers), Bayesian statistics (e.g., Naive Bayes classifier, Bayesian network, Bayesian knowledge base), Case-based reasoning, Decision trees, Inductive logic programming, Gaussian process regression, Group method of data handling (GMDH), Learning Automata, Learning Vector Quantization, Minimum message length (decision trees, decision graphs, etc.), Lazy learning, Instance-based learning Nearest Neighbor Algorithm, Analogical modeling, Probably approximately correct (PAC) learning, Ripple down rules, a knowledge acquisition methodology, Symbolic machine learning algorithms, Subsymbolic machine learning algorithms, Support vector machines, Random Forests, Ensembles of classifiers, Bootstrap aggregating (bagging), and Boosting. Supervised learning may comprise ordinal classification such as regression analysis and Information fuzzy networks (IFN). Alternatively, supervised learning methods may comprise statistical classification, such as AODE, Linear classifiers (e.g., Fisher's linear discriminant, Logistic regression, Naive Bayes classifier, Perceptron, and Support vector machine), quadratic classifiers, k-nearest neighbor, Boosting, Decision trees (e.g., C4.5, Random forests), Bayesian networks, and Hidden Markov models.

The machine learning algorithms may also comprise an unsupervised learning algorithm. Examples of unsupervised learning algorithms may include artificial neural network, Data clustering, Expectation-maximization algorithm, Self-organizing map, Radial basis function network, Vector Quantization, Generative topographic map, Information bottleneck method, and IBSEAD. Unsupervised learning may also comprise association rule learning algorithms such as Apriori algorithm, Eclat algorithm and FP-growth algorithm. Hierarchical clustering, such as Single-linkage clustering and Conceptual clustering, may also be used. Alternatively, unsupervised learning may comprise partitional clustering such as K-means algorithm and Fuzzy clustering.

In some instances, the machine learning algorithms comprise a reinforcement learning algorithm. Examples of reinforcement learning algorithms include, but are not limited to, temporal difference learning, Q-learning and Learning Automata. Alternatively, the machine learning algorithm may comprise Data Pre-processing.

In some instances, the machine learning algorithm may use deep learning. Deep learning (e.g., deep neural networks, deep belief networks, graph neural networks, recurrent neural networks and convolutional neural networks) may be supervised, semi-supervised or unsupervised.

In some embodiments, the machine learning algorithm uses artificial neural network (ANN) models for the speech detection and the word/letter classification and natural language processing techniques such as, but not limited to, a hidden Markov model (HMM) or a Viterbi decoding model for the sentence decoding.

In some embodiments, the processor is programmed to use a speech detection model to determine the probability that attempted speech or spelling is occurring at any time point during recording of neural activity and/or detect onset and offset of attempted speech or spelling during recording of the neural activity. Linear models or non-linear (e.g., artificial neural network (ANN)) models may be used to automate speech detection. In some embodiments, a deep learning model is used for speech detection, in particular, to automate detection of onset and offset of word production during attempted speech by the subject or letter production during attempted spelling by the subject. The processor may be programmed to further assign speech event labels for preparation, speech/spelling, and rest to time points during the recording of the brain electrical signal data. In some embodiments, the recorded brain electrical signal data within a time window around the detected onset of attempted speech/spelling (e.g., from 1 second before the detected onset of speech up to 3 seconds after the detected onset of speech) is used for word classification or letter classification.

Word classification may utilize a machine learning algorithm to automate identification of neural activity patterns of electrical signals in the recorded brain electrical signal data associated with attempted word production during attempted speech by the subject. Letter classification may utilize a machine learning algorithm to automate identification of neural activity patterns of electrical signals in the recorded brain electrical signal data associated with attempted letter production during attempted spelling by the subject.

In certain embodiments, a series of go cues is provided to the subject indicating when the subject should initiate attempted spelling of each letter of the words of an intended sentence. In some embodiments, the series of go cues are provided visually on a display. Each go cue may be preceded by a countdown to the presentation of the go cue, wherein the countdown for the next spelled letter is provided visually on the display and automatically started after each go cue. For example, during the spelling procedure, the participant spells out the intended message throughout letter-decoding cycles. In each cycle, the participant is visually presented with a countdown and eventually a go cue. At the go cue, the participant attempts to silently say a desired letter. In some embodiments, the series of go cues are provided with a set interval of time between each go cue, which may be adjustable by the user. In certain embodiments, the processor is programmed to use the recorded brain electrical signal data within a time window following a go cue.

In some embodiments, the processor is programmed to use a word classification model to decode words in a detected time window of neural activity (e.g., time window identified by the speech detection model as occurring during attempted speech or spelling). The word classification model is used to determine the probability that the subject intended a particular word in the attempted speech across possible speech/text targets. For example, for each word in a vocabulary of possible words that the user can say, the word classification model determines probabilities that the neural activity was collected as the user attempted to say that word. The word classification model may use linear models or non-linear (e.g., ANN) models.

In some embodiments, the processor is programmed to use a letter classification model to determine the probability that the subject intended a particular letter during the attempted spelling across all possible characters (i.e., letters of an alphabet or numbers) of the language used by a subject. In certain embodiments, the processor is further programmed to constrain word classification from sequences of letters decoded from neural activity associated with attempted spelling of words by the subject to only words within a vocabulary of a language used by the subject.

In some embodiments, the processor is programmed to use a word sequence decoding model to decode sentences based on word-sequence probabilities to determine the most likely sequence of words associated with detected speech events from the corresponding neural activity of the subject during attempted speech or spelling. The word sequence decoding model uses the sequence of probabilities from the classification model to construct a decoded sequence. This can involve using language models to incorporate a priori character-sequence or word-sequence probabilities into the neural decoding pipeline. It can also involve hidden Markov modeling (HMM) or Viterbi decoding models to handle incorporation of probabilities from the language model(s). This can use linear models or non-linear (e.g. ANN) models. In some embodiments, the processor is also programmed to use a language model that provides next-word probabilities given a previous word or phrase in a sequence of words to aid the decoding by determining predicted word sequence probabilities, wherein words that occur more frequently are assigned more weight than words that occur less frequently according to the language model. In addition, decoded information from previous detected speech events may be used to aid decoding. See Examples for a detailed discussion of the speech detection model, word classification model, and language model used to decode attempted speech from neural activity.

The subject may be instructed to limit attempted speech to words from a predefined vocabulary (i.e., word set). The number of words included is preferably large enough to create a meaningful variety of sentences but small enough to enable satisfactory neural-based classification performance. For word classification from neural activity, the subject is instructed to attempt to produce each word contained in the word set to determine the pattern of electrical signals associated with each word. Exploratory, preliminary assessments with the subject following device implantation may be used to evaluate the selection of words and the size of the word set that can be readily decoded and used to assist communication by the methods described herein.

In some embodiments, the word set comprises up to 50 words, up to 100 words, up to 200 words, up to 300 words, up to 400 words, or up to 500 words, or more. For example, the word set may include 50 words, 55 words, 60 words, 65 words, 70 words, 75 words, 80 words, 85 words, 90 words, 95 words, 100 words, 125 words, 150 words, 175 words, 200 words, 225 words, 250 words, 275 words, 300 words, 325 words, 350 words, 375 words, 400 words, 500 words, 600 words, 700 words, 800 words, 900 words, 1000 words, or any number of words in between. In some embodiments, the word set comprises: am, are, bad, bring, clean, closer, comfortable, coming, computer, do, faith, family, feel, glasses, going, good, goodbye, have, hello, help, here, hope, how, hungry, I, is, it, like, music, my, need, no, not, nurse, okay, outside, please, right, success, tell, that, they, thirsty, tired, up, very, what, where, yes, and you.

In some embodiments, the attempted speech of the subject may include any chosen sequence of words of the selected word set. In other embodiments, the attempted speech of the subject is further limited to a predefined sentence set that uses only words of the selected word set. The word set and sentence set may be selected to include sentences that can be used to communicate with a caregiver regarding tasks the subject wishes the caregiver to perform. For sentence classification from neural activity, the subject is instructed to attempt to produce each sentence contained in the sentence set while the neural activity of the subject is processed and decoded into text. A processor connected to the interface is programmed to calculate the probability that a sequence of words is an intended sentence that the subject tried to produce during the attempted speech. In some embodiments, the processor is programmed to calculate the probability of many possible sentences composed entirely of words from the specified word set as being the intended sentence that the subject tried to produce during the attempted speech. In some embodiments, the processor is programmed to maintain the most likely sentence as well as other, less likely sentences composed entirely of words from the specified word set that the subject tried to produce during the attempted speech. In some embodiments, the processor is programmed to maintain the first, second, and third most likely sentence possibilities at any given point in time. When a new word event is processed, the most likely sentence may change. For example, the second most likely sentence based on processing of a word event could then become the most likely sentence after one or more additional word events are processed.

In some embodiments, the sentence set comprises up to 25 sentences, up to 50 words, up to 100 sentences, up to 200 sentences, up to 300 sentences, up to 400 sentences, or up to 500 sentences, or more. For example, the sentence set may include 50 sentences, 100 sentences, sentences 200 sentences, 300 sentences, 400 sentences, 500 sentences, 600 sentences, 700 sentences, 800 sentences, 900 sentences, 1000 sentences, or any number of words in between. In some embodiments, the sentence set comprises: Are you going outside; Are you tired; Bring my glasses here; Bring my glasses please; Do not feel bad; Do you feel comfortable; Faith is good; Hello how are you; Here is my computer; How do you feel; How do you like my music; I am going outside; I am not going; I am not hungry; I am not okay; I am okay; I am outside; I am thirsty; I do not feel comfortable; I feel very comfortable; I feel very hungry; I hope it is clean; I like my nurse; I need my glasses; I need you; It is comfortable; It is good; It is okay; It is right here; My computer is clean; My family is here; My family is outside; My family is very comfortable; My glasses are clean; My glasses are comfortable; My nurse is outside; My nurse is right outside; No; Please bring my glasses here; Please clean it; Please tell my family; That is very clean; They are coming here; They are coming outside; They are going outside; They have faith; What do you do; Where is it; Yes; and You are not right.

In some embodiments, the attempted speech of the subject comprises spelling out words of intended messages. The attempted speech targets may include the alphabet of any language (such as English) and/or code words representing letters of the alphabet (e.g. NATO code words such as alpha, bravo, etc.). Character probabilities can be determined by classification of the speech targets (which can use linear or non-linear (e.g., ANN) models) and processed using sequence decoding techniques (e.g., language modeling, hidden Markov modeling, Viterbi decoding, etc.) to decode full sentences from the brain activity.

In certain embodiments, the methods may further comprise decoding attempted non-speech motor movements from recorded neural activity. Non-speech motor movements may include, without limitation, imagined head, arm, hand, foot, and leg movements. Non-speech motor movements can be used in any fashion that is beneficial to the user. For example, decoding of non-speech motor movements from neural activity could be used to control a mouse cursor or otherwise interact with other devices, control error correction methods in a text decoding interface, or select high-level commands to control the system (such as “end-of-sentence” or “return to main menu” commands). A classification model may be used to identify a motor command (e.g., an imagined hand movement), which could be used to indicate to the system that the user is initiating or ending attempted speech or spelling out of an intended message.

The methods of assisting a subject with communication through decoding of neural activity associated with attempted speech, attempted spelling of words, or attempted non-speech motor movement can be combined. The techniques are complementary. In some cases, decoding of attempted spelling may enable a larger vocabulary to be used than for decoding of attempted speech. However, decoding of attempted speech may be easier and more convenient for the subject, as it allows faster, direct word decoding, which may be preferred to express frequently used words. To assist decoding, attempted non-speech motor movements may be used to signal a subject is initiating or ending attempted speech or spelling out of an intended message.

Systems and Computer Implemented Methods for Decoding Attempted Speech, Attempted Spelling, and/or Attempted Non-Speech Motor Movement from Brain Activity

The present disclosure also provides systems which find use in practicing the subject methods. In some embodiments, the system may include a) a neural recording device comprising an electrode adapted for positioning at a location in a sensorimotor cortex region of the brain of the subject to record brain electrical signal data associated with attempted speech and/or attempted spelling and/or attempted non-speech motor movement by the subject; b) a processor programmed to decode a sentence from the recorded brain electrical signal data; c) an interface in communication with a computing device, said interface adapted for positioning at a location on the head of the subject, wherein the interface receives the brain electrical signal data from the neural recording device and transmits the brain electrical signal data to the processor; and d) a display component for displaying the sentence decoded from the recorded brain electrical signal data.

For example, electrical activity in the high gamma frequency range (such as 70 Hz to 150 Hz) and/or low frequency range (e.g., 0.3 Hz to 100 Hz) from the precentral gyrus, postcentral gyrus, posterior middle frontal gyrus, posterior superior frontal gyrus, or posterior inferior frontal gyrus region, or any combination thereof may be recorded with the neural recording device using this system, wherein the interface receives the brain electrical signal data from the neural recording device and transmits the brain electrical signal data to a processor. The processor may run programming for decoding letters, words, phrases, or sentences from the recorded brain electrical signal data using one or more algorithms, as described herein.

In some embodiments, a computer implemented method is used for decoding a sentence from recorded brain electrical signal data associated with attempted speech by a subject. The processor may be programmed to perform steps of the computer implemented method comprising: a) receiving the recorded brain electrical signal data associated with the attempted speech by the subject; b) analyzing the recorded brain electrical signal data using a speech detection model to calculate the probability that attempted speech is occurring at any time point and detect onset and offset of word production during the attempted speech by the subject; c) analyzing the brain electrical signal data using a word classification model that identifies patterns of electrical signals in the recorded brain electrical signal data associated with attempted word production by the subject and calculates predicted word probabilities; d) performing sentence decoding by using the calculated word probabilities from the word classification model in combination with predicted word sequence probabilities in the sentence using a language model that provides next-word probabilities given a previous word or phrase in a sequence of words to calculate predicted word sequence probabilities and determining the most likely sequence of words in the sentence based on the predicted word probabilities determined using the word classification model and the language model; and e) displaying the sentence decoded from the recorded brain electrical signal data.

In some embodiments, a computer implemented method is used for decoding a sentence from recorded brain electrical signal data associated with attempted spelling of letters of words of an intended sentence by a subject. The processor may be programmed to perform steps of the computer implemented method comprising: a) receiving the recorded brain electrical signal data associated with the attempted spelling of letters of words of an intended sentence by the subject; b) analyzing the recorded brain electrical signal data using a speech detection model to calculate the probability that attempted spelling is occurring at any time point and detect onset and offset of letter production during the attempted spelling by the subject; c) analyzing the brain electrical signal data using a letter classification model that identifies patterns of electrical signals in the recorded brain electrical signal data associated with attempted letter production by the subject and calculates a sequence of predicted letter probabilities; d) computing potential sentence candidates based on the sequence of predicted letter probabilities and automatically inserting spaces into the letter sequences between predicted words in the sentence candidates, wherein decoded words in the letter sequences are constrained to only words within a vocabulary of a language used by the subject; e) analyzing the potential sentence candidates using a language model that provides next-word probabilities given a previous word or phrase in a sequence of words to calculate predicted word sequence probabilities and determining the most likely sequence of words in a sentence; and f) displaying the sentence decoded from the recorded brain electrical signal data.

In some embodiments, a computer implemented method is used for decoding a sentence from recorded brain electrical signal data associated with attempted speech and attempted spelling by a subject.

In certain embodiments, the system may be used not only for decoding speech or spelling information from neural activity collected during attempted speech or attempted spelling, but also for decoding attempted non-speech motor movements from recorded neural activity. Non-speech motor movements may include, without limitation, imagined head, arm, hand, foot, and leg movements. Non-speech motor movements can be used in any fashion that is beneficial to the user. For example, decoding of non-speech motor movements from neural activity could be used to control a mouse cursor or otherwise interact with other devices, control error correction methods in a text decoding interface, or select high-level commands to control the system (such as “end-of-sentence” or “return to main menu” commands). A classification model may be used to identify a motor command (e.g., an imagined hand movement), which could be used to indicate to the system that the user is initiating or ending attempted speech or spelling out of an intended message.

In some embodiments, the computer implemented method further comprises: receiving recorded brain electrical signal data associated with an attempted non-speech motor movement of the subject, wherein the subject performs the attempted non-speech motor movement to indicate the initiation or termination of the attempted speech or attempted spelling of words of an intended sentence or to control an external device; and analyzing the brain electrical signal data using a classification model that identifies patterns of electrical signals in the recorded brain electrical signal data associated with the attempted non-speech motor movement and calculates a probability that the subject attempted the non-speech motor movement.

In certain embodiments, the computer implemented method further comprises storing a user profile for the subject comprising information regarding the patterns of electrical signals in the recorded brain electrical signal data associated with attempted word production by the subject.

In some embodiments, artificial neural network (ANN) models are used for the speech detection and the letter/word classification and natural language processing techniques such as, but not limited to, a hidden Markov model (HMM) or a Viterbi decoding model are used for the sentence decoding.

In certain embodiments, the subject is limited to a specified word set for the attempted speech. In some embodiments, the processor is further programmed to calculate a probability that a word of the word set is an intended word that the subject tried to produce during the attempted speech for every word of the word set, and select the word of the word set having the highest probability of being the intended word that the subject tried to produce during the attempted speech. In some embodiments, the attempted speech of the subject may include any chosen sequence of words of the selected word set. In other embodiments, the subject is limited to a specified sentence set for the attempted speech.

In some embodiments, the processor is further programmed to calculate a probability that a sequence of words is an intended sentence that the subject tried to produce during the attempted speech. In some embodiments, the processor is programmed to calculate the probability of many possible sentences composed entirely of words from the specified word set as being the intended sentence that the subject tried to produce during the attempted speech. In some embodiments, the processor is programmed to maintain the most likely sentence as well as one or more less likely sentences composed entirely of words from the specified word set that the subject tried to produce during the attempted speech. In some embodiments, the processor is programmed to track the first, second, and third most likely sentence possibilities at any given point in time. When a new word event is processed, the most likely sentence may change. For example, the second most likely sentence based on processing of a word event at a previous round could then become the most likely sentence after one or more additional word events are processed.

In certain embodiments, the processor is further programmed to assign event labels for preparation, speech/spelling (full words, letters, or any other speech target), non-speech motor movement, and rest to time points during the recording of the brain electrical signal data. In some embodiments, the processor is further programmed to use the recorded brain electrical signal data within a time window around the detected onset of word or letter classification. For example, the processor may be programmed to use the recorded brain electrical signal data from 1 second before the detected onset up to 3 seconds after the detected onset for word or letter classification.

In certain embodiments, the processor is further programmed to assign more weight to words that occur more frequently than words that occur less frequently according to the language model.

The recorded brain electrical signal data may be processed in various ways before decoding. For example, data processing may include, without limitation, real-time sample-by-sample processing of neural feature streams, the use of common-average referencing across individual electrode channels, the use of finite impulse response (FIR) filters to perform digital signal filtering, a running sliding-window normalization procedure, e.g., using Welford's method, automatic artifact rejection, and parallelization and linear pipelining to improve computational efficiency. Processing of neural features may be performed in real-time to extract one or more feature streams for use during speech/text decoding. For a description of data processing methods, see, e.g., Moses et al. (2018) J. Neural. Eng. 15(3):036005, Moses et al. (2019) Nat. Commun. 2019 10(1):3096, Moses et al. (2021) N. Engl. J. Med. 385(3):217-227, Sun et al. (2020) J. Neural. Eng. 17(6), and Makin et al. (2020) Nature Neuroscience 23:575-582; herein incorporated by reference in their entireties.

The methods described herein can be implemented in digital electronic circuitry, or in computer software, firmware, or hardware. The disclosed and other embodiments can be implemented as one or more computer program products, i.e., one or more modules of computer program instructions encoded on a computer readable medium for execution by, or to control the operation of, a data processing apparatus. The computer readable medium can be a machine-readable storage device, a machine-readable storage substrate, a memory device, a composition of matter effecting a machine-readable propagated signal, or any combination thereof.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

In a further aspect, the system for performing the computer implemented method, as described, may include a computer containing a processor, a storage component (i.e., memory), a display component, and other components typically present in general purpose computers. The storage component stores information accessible by the processor, including instructions that may be executed by the processor and data that may be retrieved, manipulated or stored by the processor.

The storage component includes instructions. For example, the storage component includes instructions for decoding a sentence from recorded brain electrical signal data associated with attempted speech and/or attempted spelling by a subject. The computer processor is coupled to the storage component and configured to execute the instructions stored in the storage component in order to receive brain electrical signal data associated with attempted speech by the subject and analyze the data according to one or more algorithms, as described herein. The display component displays the sentence decoded from the recorded brain electrical signal data.

The storage component may be of any type capable of storing information accessible by the processor, such as a hard-drive, memory card, ROM, RAM, DVD, CD-ROM, USB Flash drive, write-capable, and read-only memories. The processor may be any well-known processor, such as processors from Intel Corporation. Alternatively, the processor may be a dedicated controller such as an ASIC or an FPGA.

The instructions may be any set of instructions to be executed directly (such as machine code) or indirectly (such as scripts) by the processor. In that regard, the terms “instructions,” “steps” and “programs” may be used interchangeably herein. The instructions may be stored in object code form for direct processing by the processor, or in any other computer language including scripts or collections of independent source code modules that are interpreted on demand or compiled in advance.

Data may be retrieved, stored or modified by the processor in accordance with the instructions. For instance, although the system is not limited by any particular data structure, the data may be stored in computer registers, in a relational database as a table having a plurality of different fields and records, XML documents, or flat files. The data may also be formatted in any computer-readable format such as, but not limited to, binary values, ASCII or Unicode. Moreover, the data may comprise any information sufficient to identify the relevant information, such as numbers, descriptive text, proprietary codes, pointers, references to data stored in other memories (including other network locations) or information which is used by a function to calculate the relevant data.

In certain embodiments, the processor and storage component may comprise multiple processors and storage components that may or may not be stored within the same physical housing. For example, some of the instructions and data may be stored on removable CD-ROM and others within a read-only computer chip. Some or all of the instructions and data may be stored in a location physically remote from, yet still accessible by, the processor. Similarly, the processor may comprise a collection of processors which may or may not operate in parallel.

The system also includes an interface capable of communication with a computing device. The interface may be implanted in the cranium or placed on the head of the subject to provide an externally accessible platform through which brain electrical signals can be acquired from the neural recording device and transmitted to a computing device for decoding. In some embodiments, the interface comprises a percutaneous pedestal connector anchored in the cranium of the subject. The interface can be connected, for example, to a computing device such as a computer or a handheld computing device (e.g., cell phone or tablet) with a detachable digital connector and cable. Alternatively, the interface may be connected to a computing device wirelessly. In some embodiments, the interface comprises a first wireless communication unit in communication with a computing device comprising a second wireless communication unit. In some embodiments, the first wireless communication unit utilizes a wireless communication protocol using an electromagnetic carrier wave (e.g., a radio wave, microwave, or an infrared carrier wave) or ultrasound to transfer data from the interface to the computing device comprising the second wireless communication unit. Brain-computer interfaces are commercially available, including the Neuroport™ system from Blackrock Microsystems (Salt Lake City, Utah), See also, e.g., Weiss et al. (2019) Brain-Computer Interfaces 6:106-117; herein incorporated by reference.

Components of systems for carrying out the presently disclosed methods are further described in the examples below.

### Kits

Kits are also provided for carrying out the methods described herein. In some embodiments, the kit comprises software for carrying out the computer implemented methods for decoding a sentence from recorded brain electrical signal data associated with attempted speech and/or attempted spelling by a subject, as described herein. In some embodiments, the kit comprises a system for assisting a subject with communication as described herein. Such a system may comprise: a neural recording device comprising an electrode adapted for positioning at a location in a sensorimotor cortex region of the subject to record brain electrical signal data associated with attempted speech and/or attempted spelling and/or non-speech motor movement by the subject; a processor programmed to decode a sentence from the recorded brain electrical signal data according to a computer implemented method described herein; an interface capable of communication with a computing device, said interface adapted for positioning at a location on the head of the subject, wherein the interface receives the brain electrical signal data from the neural recording device and transmits the brain electrical signal data to the processor; and a display component for displaying the sentence decoded from the recorded brain electrical signal data.

In addition, the kits may further include (in certain embodiments) instructions for practicing the subject methods. These instructions may be present in the subject kits in a variety of forms, one or more of which may be present in the kit. For example, instructions may be present as printed information on a suitable medium or substrate, e.g., a piece or pieces of paper on which the information is printed, in the packaging of the kit, in a package insert, and the like. Another form of these instructions is a computer readable medium, e.g., diskette, compact disk (CD), flash drive, and the like, on which the information has been recorded. Yet another form of these instructions that may be present is a website address which may be used via the internet to access the information at a removed site.

### Utility

The methods, devices, and systems of the present disclosure find use in assisting individuals with communication. In particular, methods, devices, and systems are provided for decoding words and sentences directly from neural activity of an individual. In the disclosed methods, cortical activity from a region of the brain involved in speech processing is recorded while an individual attempts to say or spell out words of an intended sentence. Deep learning computational models are used to detect and classify letters/words from the recorded brain activity. Decoding of speech from brain activity is aided by use of a language model that predicts how likely certain sequences of words are to occur. In addition, decoding of attempted non-speech motor movements from neural activity can be used to further assist communication.

The methods, devices, and systems disclosed herein may be used to assist individuals who have difficulty with communication caused by conditions and diseases including, without limitation, anarthria, strokes, traumatic brain injuries, brain tumors, amyotrophic lateral sclerosis, multiple sclerosis, Huntington's disease, Niemann-Pick disease, Friedreich's ataxia, Wilson's disease, cerebral palsy, Guillain-Barre syndrome, Tay-Sachs disease, encephalopathy, central pontine myelinolysis, and other conditions causing dysfunction or paralysis of the muscles of the head, neck, or chest resulting in anarthria. The methods disclosed herein may be used to restore communication to such individuals and improve autonomy and quality of life.

### EXAMPLES OF NON-LIMITING ASPECTS OF THE DISCLOSURE

Aspects, including embodiments, of the present subject matter described above may be beneficial alone or in combination, with one or more other aspects or embodiments. Without limiting the foregoing description, certain non-limiting aspects of the disclosure numbered 1-159 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:
1. A method of assisting a subject with communication, the method comprising:
positioning a neural recording device comprising an electrode at a location in a sensorimotor cortex region of the brain of the subject to record brain electrical signal data associated with attempted speech by the subject;
positioning an interface in communication with a computing device at a location on the head of the subject, wherein the interface is connected to the neural recording device;
recording the brain electrical signal data associated with attempted speech by the subject using the neural recording device, wherein the interface receives the brain electrical signal data from the neural recording device and transmits the brain electrical signal data to a processor of the computing device; and
decoding a word, a phrase, or a sentence from the recorded brain electrical signal data using the processor.
2. The method of aspect 1, wherein the subject has difficulty with said communication because of anarthria, a stroke, a traumatic brain injury, a brain tumor, or amyotrophic lateral sclerosis.
3. The method of aspect 1 or 2, wherein the subject is paralyzed.
4. The method of any one of aspects 1-3, wherein the location of the neural recording device is in the ventral sensorimotor cortex.
5. The method of any one of aspects 1-4, wherein the electrode is positioned on a surface of the sensorimotor cortex region or within the sensorimotor cortex region.
6. The method of aspect 5, wherein the electrode is positioned on a surface of the sensorimotor cortex region of the brain in a subdural space.
7. The method of any one of aspects 1-6, wherein the neural recording device comprises a brain-penetrating electrode array.
8. The method of any one of claims 1-7, wherein the neural recording device comprises an electrocorticography (ECoG) electrode array.
9. The method of any one of aspects 1-8, wherein the electrode is a depth electrode or a surface electrode.
10. The method of any one of aspects 1-9, wherein the electrical signal data comprises high-gamma frequency content features.
11. The method of aspect 10, wherein the electrical signal data comprises neural oscillations in a range from 70 Hz to 150 Hz.
12. The method of any one of aspects 1-11, wherein said recording the brain electrical signal data comprises recording the brain electrical signal data from a sensorimotor cortex region selected from a precentral gyrus, postcentral gyrus, posterior middle frontal gyrus, posterior superior frontal gyrus, or posterior inferior frontal gyrus region, or any combination thereof.
13. The method of any one of aspects 1-12, further comprising mapping the brain of the subject to identify an optimal location for positioning the electrode for recording the brain electrical signals associated with the attempted speech by the subject.
14. The method of any one of aspects 1-13, wherein the interface comprises a percutaneous pedestal connector attached to the subject's cranium.
15. The method of aspect 14, wherein the interface further comprises a headstage connected to the percutaneous pedestal connector.
16. The method of any one of aspects 1-15, wherein the processor is provided by a computer or handheld device.
17. The method of aspect 16, wherein the handheld device is a cell phone or a tablet.
18. The method of any one of aspects 1-17, wherein the processor is programmed to automate speech detection, word classification, and sentence decoding based on identification of a neural activity pattern of electrical signals in the recorded brain electrical signal data associated with attempted word production.
19. The method of aspect 18, wherein the processor is programmed to use a machine learning algorithm for speech detection, word classification, and sentence decoding.
20. The method of aspect 19, wherein artificial neural network (ANN) models are used for the speech detection and the word classification, and a hidden Markov model (HMM), a Viterbi decoding model, or a natural language processing technique is used for the sentence decoding.
21. The method of any one of aspects 1-20, wherein the processor is programmed to automate detection of onset and offset of word production during the attempted speech by the subject.
22. The method of aspect 21, further comprising assigning speech event labels for preparation, speech, and rest to time points during the recording of the brain electrical signal data.
23. The method of aspect 21 or 22, wherein the processor is programmed to use the recorded brain electrical signal data within a time window around the detected onset of word classification.
24 The method of any one of aspects 1-23, wherein the subject is limited to a specified word set for the attempted speech.
25. The method of aspect 24, wherein the processor is programmed to calculate a probability that a word of the word set is an intended word that the subject tried to produce during the attempted speech.
26. The method of aspect 25, wherein the processor is programmed to calculate the probability that a word of the word set is an intended word that the subject tried to produce during the attempted speech for every word of the word set.
27. The method of any one of aspects 24-26, wherein the word set comprises am, are, bad, bring, clean, closer, comfortable, coming, computer, do, faith, family, feel, glasses, going, good, goodbye, have, hello, help, here, hope, how, hungry, I, is, it, like, music, my, need, no, not, nurse, okay, outside, please, right, success, tell, that, they, thirsty, tired, up, very, what, where, yes, and you.
28. The method of any one of aspects 1-27, wherein the subject may use the words of the word set without limitation to create sentences.
29. The method of aspect 28, wherein the processor is programmed to calculate a probability that a sequence of words is an intended sentence that the subject tried to produce during the attempted speech.
30. The method of any one of aspects 1-29, wherein the processor is programmed to use a language model that provides next-word probabilities given a previous word or phrase in a sequence of words to aid the decoding by determining predicted word sequence probabilities.
31. The method of aspect 30, wherein words that occur more frequently are assigned more weight than words that occur less frequently according to the language model.
32. The method of aspect 30 or 31, wherein the processor is programmed to use a Viterbi decoding model to determine the most likely sequence of words in the intended speech of the subject given the brain electrical signal data associated with the attempted speech, the predicted word probabilities from the word classification model using the machine learning algorithm, and the word sequence probabilities using the language model.
33. The method of any one of aspects 1-32, further comprising:
recording brain electrical signal data associated with an attempted non-speech motor movement of the subject, wherein the subject performs the attempted non-speech motor movement to indicate the initiation or termination of the attempted speech or to control an external device; and
analyzing the brain electrical signal data using a non-speech motor movement classification model that identifies patterns of electrical signals in the recorded brain electrical signal data associated with the attempted non-speech motor movement and calculates a probability that the subject attempted the non-speech motor movement.
34. The method of aspect 33, wherein the attempted non-speech motor movement comprises an attempted head, arm, hand, foot, or leg movement.
35. The method of aspect 34, wherein the attempted hand movement comprises an imagined hand gesture or an imagined hand squeeze.
36. The method of any one of aspects 33-35, wherein the processor is further programmed to automate detection of an attempted non-speech motor movement of the subject signaling the end of the attempted speech by the subject based on identification of a neural activity pattern of electrical signals in the recorded brain electrical signal data associated with the attempted non-speech motor movement.
37. The method of aspect 36, wherein the processor is further programmed to assign event labels for the attempted non-speech motor movement to time points during the recording of the brain electrical signal data.
38. The method of any one of aspects 1-37, wherein the method further comprises assessing accuracy of the decoding.
39. A computer implemented method for decoding a sentence from recorded brain electrical signal data associated with attempted speech by a subject, the computer performing steps comprising:
   a) receiving the recorded brain electrical signal data associated with the attempted speech by the subject;
   b) analyzing the recorded brain electrical signal data using a speech detection model to calculate the probability that attempted speech is occurring at any time point during recording of the brain electrical signal data and detect onset and offset of word production during the attempted speech by the subject;
   c) analyzing the brain electrical signal data using a word classification model that identifies patterns of electrical signals in the recorded brain electrical signal data associated with attempted word production by the subject and calculates predicted word probabilities;
   d) performing sentence decoding by using the calculated word probabilities from the word classification model in combination with predicted word sequence probabilities in the sentence using a language model that provides next-word probabilities given a previous word or phrase in a sequence of words to calculate predicted word sequence probabilities and determining the most likely sequence of words in the sentence based on the predicted word probabilities determined using the word classification model and the language model; and
   e) displaying the sentence decoded from the recorded brain electrical signal data.
40. The computer implemented method of aspect 39, wherein a machine learning algorithm is used for speech detection, word classification, and sentence decoding.
41. The computer implemented method of aspect 40, wherein artificial neural network (ANN) models are used for the speech detection and the word classification, and a hidden Markov model (HMM), a Viterbi decoding model, or a natural language processing technique is used for the sentence decoding.
42. The computer implemented method of any one of aspects 39-41, wherein the subject is limited to a specified word set for the attempted speech.
43. The computer implemented method of aspect 42, further comprising calculating a probability that a word of the word set is an intended word that the subject tried to produce during the attempted speech for every word of the word set and select the word of the word set having the highest probability of being the intended word that the subject tried to produce during the attempted speech.
44. The computer implemented method of any one of aspects 39-43, wherein the subject may use the words of the word set without limitation to create sentences or is limited to a specified sentence set for the attempted speech.
45. The computer implemented method of any one of aspects 39-44, further comprising calculating a probability that a sequence of words is an intended sentence that the subject tried to produce during the attempted speech.
46. The computer implemented method of aspect 45, further comprising maintaining the most likely sentence and one or more less likely sentences and recalculating the probability that a sequence of words is an intended sentence that the subject tried to produce during the attempted speech after decoding of each word.
47. The computer implemented method of aspect 46, wherein the most likely sentence and the one or more less likely sentences are composed only of words from the word set used by the subject for the attempted speech.
48. The computer implemented method of any one of aspects 39-47, further comprising assigning speech event labels for preparation, speech, and rest to time points during the recording of the brain electrical signal data.
49. The computer implemented method of aspect 48, wherein only the recorded brain electrical signal data within a time window around the detected onset of word classification is used.
50. The computer implemented method of any one of aspects 39-49, wherein more weight is assigned to words that occur more frequently than words that occur less frequently according to the language model.
51. The computer implemented method of any one of aspects 39-50, further comprising storing a user profile for the subject comprising information regarding the patterns of electrical signals in the recorded brain electrical signal data associated with attempted word production by the subject.
52. The computer implemented method of any one of aspects 39-51, further comprising:
receiving recorded brain electrical signal data associated with an attempted non-speech motor movement of the subject, wherein the subject performs the attempted non-speech motor movement to indicate the initiation or termination of the attempted speech or to control an external device; and
analyzing the brain electrical signal data using a classification model that identifies patterns of electrical signals in the recorded brain electrical signal data associated with the attempted non-speech motor movement and calculates a probability that the subject attempted the non-speech motor movement.
53. The computer implemented method of aspect 52, wherein the attempted non-speech motor movement comprises an attempted head, arm, hand, foot, or leg movement.
54. The computer implemented method of aspect 53, wherein the attempted hand movement comprises an imagined hand gesture or an imagined hand squeeze.
55. The computer implemented method of any one of aspects 52-54, further comprising assigning event labels for the attempted non-speech motor movement to time points during the recording of the brain electrical signal data.
56. A non-transitory computer-readable medium comprising program instructions that, when executed by a processor in a computer, causes the processor to perform the method of any one of aspects 39-55.
57. A kit comprising the non-transitory computer-readable medium of aspect 56 and instructions for decoding brain electrical signal data associated with attempted speech by a subject.
58. A system for assisting a subject with communication, the system comprising:
a neural recording device comprising an electrode adapted for positioning at a location in a sensorimotor cortex region of the brain of the subject to record brain electrical signal data associated with attempted speech or an attempted non-speech motor movement by the subject;
a processor programmed to decode a sentence from the recorded brain electrical signal data according to the computer implemented method of any one of aspects 39-55;
an interface in communication with a computing device, said interface adapted for positioning at a location on the head of the subject, wherein the interface receives the brain electrical signal data from the neural recording device and transmits the brain electrical signal data to the processor; and
a display component for displaying the sentence decoded from the recorded brain electrical signal data.
59. The system of aspect 58, wherein the subject has difficulty with said communication because of anarthria, a stroke, a traumatic brain injury, a brain tumor, or amyotrophic lateral sclerosis.
60. The system of aspect 58 or 59, wherein the location of the neural recording device is in the ventral sensorimotor cortex.
61. The system of any one of aspects 58-60, wherein the electrode is adapted for positioning on a surface of the sensorimotor cortex region or within the sensorimotor cortex region.
62. The system of aspect 61, wherein the electrode is adapted for positioning on a surface of the sensorimotor cortex region of the brain in a subdural space.
63. The system of any one of aspects 58-62, wherein the neural recording device comprises a brain-penetrating electrode array.
64. The system of any one of aspects 58-63, wherein the neural recording device comprises an electrocorticography (ECoG) electrode array.
65. The system of any one of aspects 58-64, wherein the electrode is a depth electrode or a surface electrode.
66. The system of any one of aspects 58-65, wherein the electrical signal data comprises high-gamma frequency content features.
67. The system of aspect 66, wherein the electrical signal data comprises neural oscillations in a range from 70 Hz to 150 Hz.
68. The system of any one of aspects 58-67, wherein the interface comprises a percutaneous pedestal connector attached to the subject's cranium.
69. The system of aspect 68, wherein the interface further comprises a headstage that is connectable to the percutaneous pedestal connector.
70. The system of any one of aspects 58-69, wherein the processor is provided by a computer or handheld device.
71. The system of aspect 70, wherein the handheld device is a cell phone or tablet.
72. The system of any one of aspects 58-71, wherein a machine learning algorithm is used for speech detection, word classification, and sentence decoding.
73. The system of aspect 72, wherein artificial neural network (ANN) models are used for the speech detection and the word classification, and a hidden Markov model (HMM), a Viterbi decoding model, or a natural language processing technique is used for the sentence decoding.
74. The system of any one of aspects 58-73, wherein the processor is further programmed to assign speech event labels for preparation, speech, and rest to time points during the recording of the brain electrical signal data.
75. The system of aspect 74, wherein the processor is further programmed to use the recorded brain electrical signal data within a time window around the detected onset of word classification.
76. The system of any one of aspects 58-75, wherein the subject is limited to a specified word set for the attempted speech.
77. The system of aspect 76, wherein the processor is further programmed to calculate a probability that a word of the word set is an intended word that the subject tried to produce during the attempted speech for every word of the word set and select the word of the word set having the highest probability of being the intended word that the subject tried to produce during the attempted speech.
78. The system of aspect 76 or 77, wherein the word set comprises: am, are, bad, bring, clean, closer, comfortable, coming, computer, do, faith, family, feel, glasses, going, good, goodbye, have, hello, help, here, hope, how, hungry, I, is, it, like, music, my, need, no, not, nurse, okay, outside, please, right, success, tell, that, they, thirsty, tired, up, very, what, where, yes, and you.
79. The system of any one of aspects 76-78, wherein the subject may use any chosen sequence of words of the selected word set.
80. The system of aspect 79, wherein the processor is programmed to calculate a probability that a sequence of words is an intended sentence that the subject tried to produce during the attempted speech.
81. The system of aspect 80, wherein the processor is programmed to maintain the most likely sentence and one or more less likely sentences and recalculate the probability that a sequence of words is an intended sentence that the subject tried to produce during the attempted speech after decoding of each word.
82. The system of aspect 81, wherein the most likely sentence and the one or more less likely sentences are composed only of words from the word set used by the subject for the attempted speech.
83. The system of any one of aspects 58-82, wherein the processor is further programmed to automate detection of an attempted non-speech motor movement of the subject signaling the initiation or termination of the attempted speech by the subject based on identification of a neural activity pattern of electrical signals in the recorded brain electrical signal data associated with the attempted non-speech motor movement.
84. The system of aspect 83, wherein the processor is further programmed to assign event labels for the attempted non-speech motor movement to time points during the recording of the brain electrical signal data.
85. A kit comprising the system of any one of aspects 58-84 and instructions for using the system for recording and decoding brain electrical signal data associated with attempted speech by a subject.
86. A method of assisting a subject with communication, the method comprising:
positioning a neural recording device comprising an electrode at a location in a sensorimotor cortex region of the brain of the subject to record brain electrical signal data associated with attempted spelling of letters of words of an intended sentence by the subject;
positioning an interface in communication with a computing device at a location on the head of the subject, wherein the interface is connected to the neural recording device;
recording the brain electrical signal data associated with said attempted spelling by the subject using the neural recording device, wherein the interface receives the brain electrical signal data from the neural recording device and transmits the brain electrical signal data to a processor of the computing device; and
decoding the spelled words of the intended sentence from the recorded brain electrical signal data using the processor.
87. The method of aspect 86, wherein the subject has difficulty with said communication because of anarthria, a stroke, a traumatic brain injury, a brain tumor, or amyotrophic lateral sclerosis.
88. The method of aspect 86 or 87, wherein the subject is paralyzed.
89. The method of any one of aspects 86-88, wherein the location of the neural recording device is in the ventral sensorimotor cortex.
90. The method of any one of aspects 86-89, wherein the electrode is positioned on a surface of the sensorimotor cortex region or within the sensorimotor cortex region.
91. The method of aspect 90, wherein the electrode is positioned on a surface of the sensorimotor cortex region of the brain in a subdural space.
92. The method of any one of aspects 86-91, wherein the neural recording device comprises a brain-penetrating electrode array.
93. The method of any one of aspects 86-92, wherein the neural recording device comprises an electrocorticography (ECoG) electrode array.
94. The method of any one of aspects 86-93, wherein the electrode is a depth electrode or a surface electrode.
95. The method of any one of aspects 86-94, wherein the electrical signal data comprises high-gamma frequency content features and low frequency content features.
96. The method of aspect 95, wherein the electrical signal data comprises neural oscillations in a high-gamma frequency range from 70 Hz to 150 Hz and in a low frequency range from 0.3 Hz to 100 Hz.
97. The method of any one of aspects 86-96, wherein said recording of the brain electrical signal data comprises recording the brain electrical signal data from a sensorimotor cortex region selected from a precentral gyrus region, a postcentral gyrus region, a posterior middle frontal gyrus region, a posterior superior frontal gyrus region, or a posterior inferior frontal gyrus region, or any combination thereof.
98. The method of any one of aspects 86-97, further comprising mapping the brain of the subject to identify an optimal location for positioning the electrode for recording the brain electrical signals associated with the attempted spelling of words or attempted non-speech motor movement by the subject.
99. The method of any one of aspects 86-98, wherein the interface comprises a percutaneous pedestal connector attached to the subject's cranium.
100. The method of aspect 99, wherein the interface further comprises a headstage connected to the percutaneous pedestal connector.
101. The method of any one of aspects 86-100, wherein the processor is provided by a computer or handheld device.
102. The method of aspect 101, wherein the handheld device is a cell phone or a tablet.
103. The method of any one of aspects 86-102, wherein the processor is programmed to automate detection of the attempted spelling, letter classification, word classification, and sentence decoding based on identification of a neural activity pattern of electrical signals in the recorded brain electrical signal data associated with the attempted spelling of words by the subject.
104. The method of aspect 103, wherein the processor is programmed to use a machine learning algorithm for the speech detection, letter classification, word classification, and sentence decoding.
105. The method of aspect 104, wherein the processor is further programmed to constrain word classification from sequences of letters decoded from neural activity associated with attempted spelling of words by the subject to only words within a vocabulary of a language used by the subject.
106. The method of any one of aspects 86-105, wherein the processor is further programmed to assign event labels for preparation, attempted spelling, and rest to time points during the recording of the brain electrical signal data.
107. The method of aspect 106, wherein the processor is programmed to use the recorded brain electrical signal data within a time window around the detected onset of attempted spelling of a letter by the subject.
108. The method of any one of aspects 86-107, further comprising providing a series of go cues to the subject indicating when the subject should initiate attempted spelling of each letter of the words of the intended sentence.
109. The method of aspect 108, wherein the series of go cues are provided visually on a display.
110. The method of aspect 109, wherein each go cue is preceded by a countdown to the presentation of the go cue, wherein the countdown for the next spelled letter is provided visually on the display and automatically started after each go cue.
111. The method of any one of aspects 108-110, wherein the series of go cues are provided with a set interval of time between each go cue.
112. The method of aspect 111, wherein the subject can control the set interval of time between each go cue.
113. The method of any one of aspects 108-112, wherein the processor is programmed to use the recorded brain electrical signal data within a time window following the go cue.
114. The method of any one of aspects 86-113, wherein the processor is programmed to calculate a probability that a sequence of decoded words from a sequence of decoded letters is an intended sentence that the subject tried to produce during the attempted spelling of letters of words of an intended sentence by the subject.
115. The method of any one of aspects 86-114, wherein the processor is programmed to use a language model that provides next-word probabilities given a previous word or phrase in a sequence of words to aid the decoding by determining predicted word sequence probabilities.
116. The method of aspect 115, wherein words that occur more frequently are assigned more weight than words that occur less frequently according to the language model.
117. The method of any one of aspects 86-116, wherein the processor is further programmed to use a sequence of predicted letter probabilities to compute potential sentence candidates and automatically insert spaces into letter sequences between predicted words in the sentence candidates.
118. The method of any one of aspects 86-117, further comprising:
recording brain electrical signal data associated with an attempted non-speech motor movement of the subject, wherein the subject performs the attempted non-speech motor movement to indicate the initiation or termination of the attempted spelling of words of the intended sentence or to control an external device; and
analyzing the brain electrical signal data using a classification model that identifies patterns of electrical signals in the recorded brain electrical signal data associated with the attempted non-speech motor movement and calculates a probability that the subject attempted the non-speech motor movement.
119. The method of aspect 118, wherein the attempted non-speech motor movement comprises an attempted head, arm, hand, foot, or leg movement.
120. The method of aspect 119, wherein the attempted hand movement comprises an imagined hand gesture or an imagined hand squeeze.
121. The method of any one of aspects 118-120, further comprising assigning event labels for the attempted non-speech motor movement to time points during the recording of the brain electrical signal data.
122. The method of any one of aspects 86-121, further comprising assessing accuracy of the decoding.
123. The method of any one of aspects 86-122, further comprising:
recording brain electrical signal data associated with attempted speech by the subject using the neural recording device, wherein the interface receives the brain electrical signal data from the neural recording device and transmits the brain electrical signal data to the processor of the computing device; and
decoding a word, a phrase, or a sentence from the recorded brain electrical signal data associated with attempted speech by the subject using the processor.
124. A computer implemented method for decoding a sentence from recorded brain electrical signal data associated with attempted spelling of letters of words of an intended sentence by a subject, the computer performing steps comprising:
   a) receiving the recorded brain electrical signal data associated with the attempted spelling of letters of words of an intended sentence by the subject;
   b) analyzing the recorded brain electrical signal data using a speech detection model to calculate the probability that attempted spelling is occurring at any time point during the recording of the electrical signal data and detect onset and offset of letter production during the attempted spelling by the subject;
   c) analyzing the brain electrical signal data using a letter classification model that identifies patterns of electrical signals in the recorded brain electrical signal data associated with attempted letter production by the subject and calculates a sequence of predicted letter probabilities;
   d) computing potential sentence candidates based on the sequence of predicted letter probabilities and automatically inserting spaces into the letter sequences between predicted words in the sentence candidates, wherein decoded words in the letter sequences are constrained to only words within a vocabulary of a language used by the subject;
   e) analyzing the potential sentence candidates using a language model that provides next-word probabilities given a previous word or phrase in a sequence of words to calculate predicted word sequence probabilities and determining the most likely sequence of words in a sentence; and
   f) displaying the sentence decoded from the recorded brain electrical signal data.
125. The computer implemented method of aspect 124 wherein the recorded brain electrical signal data is only used within a time window around the detected onset of attempted spelling of a letter by the subject.
126. The computer implemented method of aspect 124 or 125, further comprising displaying a series of go cues to the subject indicating when the subject should initiate attempted spelling of each letter of the words of the intended sentence.
127. The computer implemented method of aspect 126, wherein each go cue is preceded by displaying a countdown to the presentation of the go cue, wherein the countdown for the next spelled letter is automatically started after each go cue.
128. The computer implemented method of aspect 126 or 127, wherein the series of go cues are provided with a set interval of time between each go cue.
129. The computer implemented method of aspect 128, wherein the subject can control the set interval of time between each go cue.
130. The computer implemented method of any one of aspects 122-127, wherein the recorded brain electrical signal data within a time window following the go cue is used for letter classification.
131. The computer implemented method of any one of aspects 124-130, further comprising:
receiving recorded brain electrical signal data associated with an attempted non-speech motor movement of the subject, wherein the subject performs the attempted non-speech motor movement to indicate the initiation or termination of the attempted spelling of words of the intended sentence or to control an external device; and
analyzing the brain electrical signal data using a classification model that identifies patterns of electrical signals in the recorded brain electrical signal data associated with the attempted non-speech motor movement and calculates a probability that the subject attempted the non-speech motor movement.
132. The method of aspect 131, wherein the attempted non-speech motor movement comprises an attempted head, arm, hand, foot, or leg movement.
133. The method of aspect 132, wherein the attempted hand movement comprises an imagined hand gesture or an imagined hand squeeze.
134. The computer implemented method of any one of aspects 124-133, wherein a machine learning algorithm is used for detection of attempted spelling or attempted non-speech motor movement or letter classification.
135. The computer implemented method of any one of aspects 124-134, further comprising assigning more weight to words that occur more frequently than words that occur less frequently according to the language model.
136. The computer implemented method of any one of aspects 124-135, further comprising storing a user profile for the subject comprising information regarding the patterns of electrical signals in the recorded brain electrical signal data associated with letter production during attempted spelling by the subject.
137. The computer implemented method of any one of aspects 124-136, wherein the electrical signal data comprises high-gamma frequency content features and low frequency content features.
138. The computer implemented method of aspect 137, wherein the electrical signal data comprises neural oscillations in a high-gamma frequency range from 70 Hz to 150 Hz and in a low frequency range from 0.3 Hz to 100 Hz.
139. The computer implemented method of any one of aspects 124-138, further comprising assessing accuracy of the decoding.
140. The computer implemented method of any one of aspects 124-139, further comprising decoding a sentence from recorded brain electrical signal data associated with attempted speech by the subject, the computer further performing steps comprising:
   a) receiving the recorded brain electrical signal data associated with the attempted speech by the subject;
   b) analyzing the recorded brain electrical signal data using a speech detection model to calculate the probability that attempted speech is occurring at any time point and detect onset and offset of word production during the attempted speech by the subject;
   c) analyzing the brain electrical signal data using a word classification model that identifies patterns of electrical signals in the recorded brain electrical signal data associated with attempted word production by the subject and calculates predicted word probabilities;
   d) performing sentence decoding by using the calculated word probabilities from the word classification model in combination with predicted word sequence probabilities in the sentence using a language model that provides next-word probabilities given a previous word or phrase in a sequence of words to calculate predicted word sequence probabilities and determining the most likely sequence of words in the sentence based on the predicted word probabilities determined using the word classification model and the language model; and
   e) displaying the sentence decoded from the recorded brain electrical signal data.
141. The computer implemented method of aspect 140, wherein a machine learning algorithm is used for speech detection and word classification, and sentence decoding.
142. The computer implemented method of aspect 141, wherein artificial neural network (ANN) models are used for the speech detection and the word classification, and a hidden Markov model (HMM), a Viterbi decoding model, or a natural language processing technique is used for the sentence decoding.
143. A non-transitory computer-readable medium comprising program instructions that, when executed by a processor in a computer, causes the processor to perform the method of any one of aspects 124-142.
144. A kit comprising the non-transitory computer-readable medium of aspect 143 and instructions for decoding brain electrical signal data associated with attempted spelling of letters of words of an intended sentence by a subject.
145. A system for assisting a subject with communication, the system comprising:
a neural recording device comprising an electrode adapted for positioning at a location in a sensorimotor cortex region of the brain of the subject to record brain electrical signal data associated with attempted speech, attempted spelling of letters of words of an intended sentence, or attempted non-speech motor movement by the subject, or a combination thereof;
a processor programmed to decode a sentence from the recorded brain electrical signal data according to the computer implemented method of any one of aspects 124-142;
an interface in communication with a computing device, said interface adapted for positioning at a location on the head of the subject, wherein the interface receives the brain electrical signal data from the neural recording device and transmits the brain electrical signal data to the processor; and
a display component for displaying the sentence decoded from the recorded brain electrical signal data.
146. The system of aspect 145, wherein the subject has difficulty with said communication because of anarthria, a stroke, a traumatic brain injury, a brain tumor, or amyotrophic lateral sclerosis.
147. The system of aspect 145 or 146, wherein the location of the neural recording device is in the ventral sensorimotor cortex.
148. The system of any one of aspects 145-147, wherein the electrode is adapted for positioning on a surface of the sensorimotor cortex region or within the sensorimotor cortex region.
149. The system of aspect 148, wherein the electrode is adapted for positioning on a surface of the sensorimotor cortex region of the brain in a subdural space.
150. The system of any one of aspects 145-149, wherein the neural recording device comprises a brain-penetrating electrode array.
151. The system of any one of aspects 145-150, wherein the neural recording device comprises an electrocorticography (ECoG) electrode array.
152. The system of any one of aspects 145-151, wherein the electrode is a depth electrode or a surface electrode.
153. The system of any one of aspects 145-152, wherein the electrical signal data comprises high-gamma frequency content features and low frequency content features.
154. The system of aspect 153, wherein the electrical signal data comprises neural oscillations in a high-gamma frequency range from 70 Hz to 150 Hz and in a low frequency range from 0.3 Hz to 100 Hz.
155. The system of any one of aspects 145-154, wherein the interface comprises a percutaneous pedestal connector attached to the subject's cranium.
156. The system of aspect 155, wherein the interface further comprises a headstage that is connectable to the percutaneous pedestal connector.
157. The system of any one of aspects 145-156, wherein the processor is provided by a computer or handheld device.
158. The system of aspect 157, wherein the handheld device is a cell phone or tablet.
159. A kit comprising the system of any one of aspects 145-158 and instructions for using the system for recording and decoding brain electrical signal data associated with attempted speech, attempted spelling of words, or attempted non-speech motor movement by a subject, or a combination thereof.

### EXAMPLES

As can be appreciated from the disclosure provided above, the present disclosure has a wide variety of applications. Accordingly, the following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, dimensions, etc.) but some experimental errors and deviations should be accounted for. Those of skill in the art will readily recognize a variety of noncritical parameters that could be changed or modified to yield essentially similar results.

### Example 1: A Speech Neuroprosthesis for Decoding Words in a Person with Severe Paralysis

### Introduction

Anarthria is the loss of the ability to articulate speech. It can result from a variety of conditions, including stroke, traumatic brain injury, and amyotrophic lateral sclerosis [1]. For paralyzed individuals with severe movement impairment, it hinders communication with family, friends, and caregivers, reducing self-reported quality of life [2].

Advances have been made with typing-based brain-computer interfaces that allow impaired individuals to spell out intended messages using cursor control [3-7]. However, letter-by-letter selection interfaces driven by neural signal recordings can be relatively slow and tedious. A more efficient and natural approach may be to directly decode whole words from brain areas that control speech. In the last decade, our understanding of how the speech motor cortex orchestrates the rapid articulatory movements of the vocal tract has expanded [8-13]. In parallel, engineering efforts have leveraged these findings to demonstrate that speech can be decoded from brain activity in people without speech impairments [14-17].

However, it is unclear whether speech decoding approaches will work in paralyzed individuals who cannot speak. Neural activity cannot be precisely aligned with intended speech due to the absence of speech output, posing an obstacle for training computational models [18]. In addition, it is unclear whether neural signals underlying speech control are still intact in individuals who have not spoken for years or decades. In an earlier study, a person with locked-in syndrome used an implanted two-channel microelectrode device to generate vowel sounds and phonemes through an audio-visual interface [19, 20]. It remains unknown whether it is possible to reliably decode full words from the neural activity of a person with anarthria.

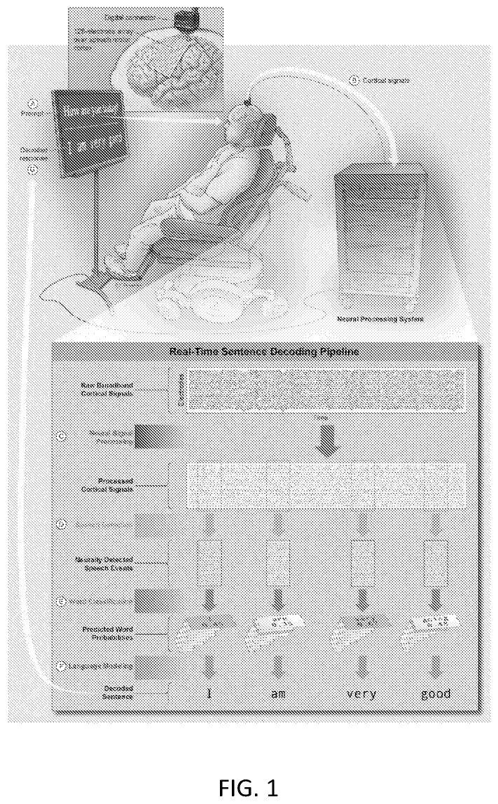

In this work, we demonstrate real-time word and sentence decoding from the neural activity of a person with severe paralysis and anarthria resulting from a remote brainstem stroke (FIG. 1). Our findings represent a proof-of-concept for long-term communication restoration through a direct speech brain-computer interface.

### METHODS

### Trial Overview

This work was performed as part of the BRAVO study (BCI Restoration of Arm and Voice function, clinicaltrials.gov, NCT03698149), which is a single-institution clinical trial that aims to evaluate the potential of electrocorticography (ECoG; a method for recording neural activity directly from the surface of the brain) and custom decoding techniques for long-term communication and movement restoration. The ECoG device used in this study received Investigational Device Exemption approval by the United States Food and Drug Administration. At the time of writing, only one clinical trial participant (“Bravo-1”; the participant in this study) has been implanted with the ECoG device.

### Participant

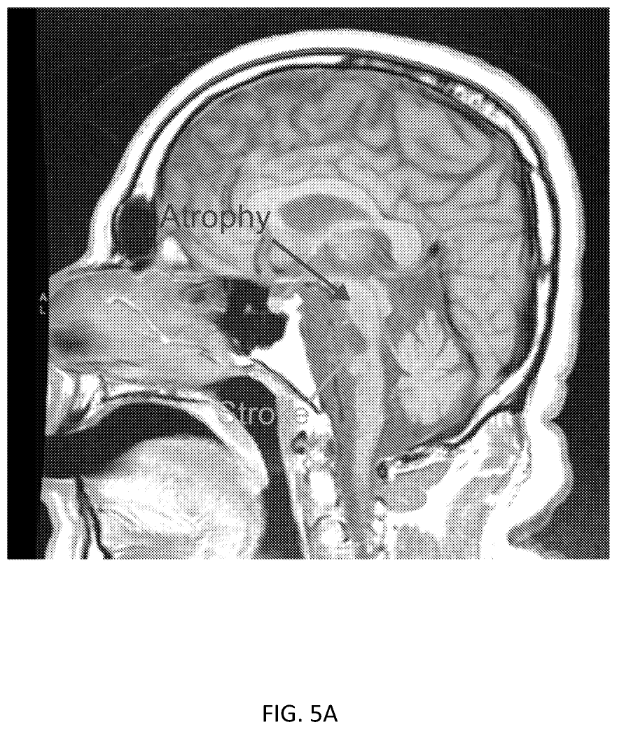

The participant is a right-handed male who was 36 years old at the start of the study. At age 20, he suffered extensive bilateral pontine strokes associated with a right vertebral artery dissection, which resulted in severe spastic quadriparesis and anarthria (diagnosed by a speech language pathologist and neurologists; FIG. 5). He is cognitively intact (assessed with the Mini-Mental Status Exam). He is able to vocalize grunts and moans but unable to produce intelligible speech. He normally communicates using an assistive computer-based typing interface controlled by his residual head movements, with typing rates at approximately 5 correct words or 18 correct characters per minute (Supplementary Method S1).

### Implant Device

The neural implant used to acquire brain signals from the participant is a customized hybrid of a high-density ECoG electrode array (PMT Corporation, MN, USA) with a pedestal connector (Blackrock Microsystems, UT, USA). The ECoG array consists of 128 flat, disc-shaped electrodes with 4-mm center-to-center spacing. During surgical implantation, the speech sensorimotor cortex was exposed via craniotomy and the array was laid on the surface of the brain in the subdural space. The dura was sutured closed, and the cranial bone flap was replaced. The percutaneous pedestal connector was placed at a separate site and anchored to the cranium with small titanium screws. This pedestal connector is an externally accessible platform through which brain signals can be acquired and transmitted to a computer via a detachable digital connector and cable (FIG. 1). The participant underwent surgical implantation of the device in early 2019. The procedure was successful, and his recovery was uneventful. The electrode coverage enabled sampling from multiple cortical regions that have been implicated in speech processing, including portions of the left precentral gyrus, postcentral gyrus, posterior middle frontal gyrus, and posterior inferior frontal gyrus [8, 10-12].

### Neural Data Acquisition and Real-Time Processing

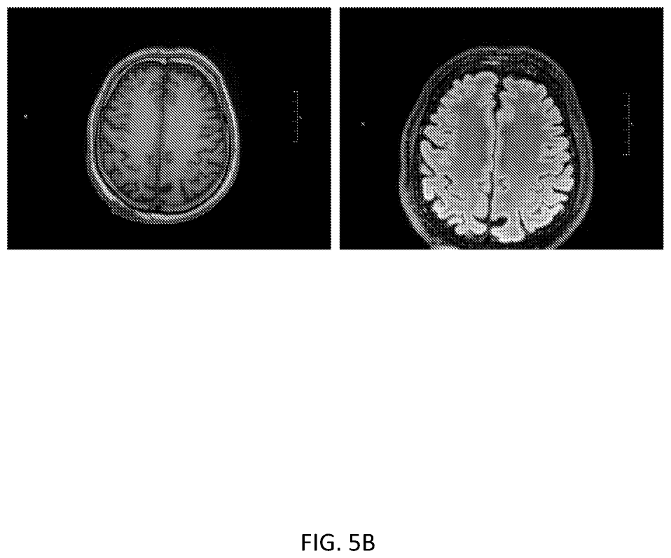

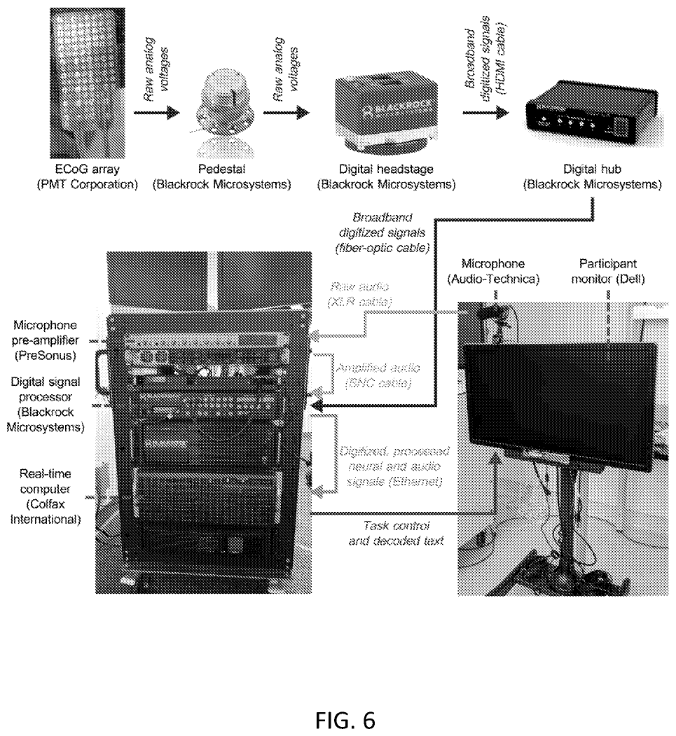

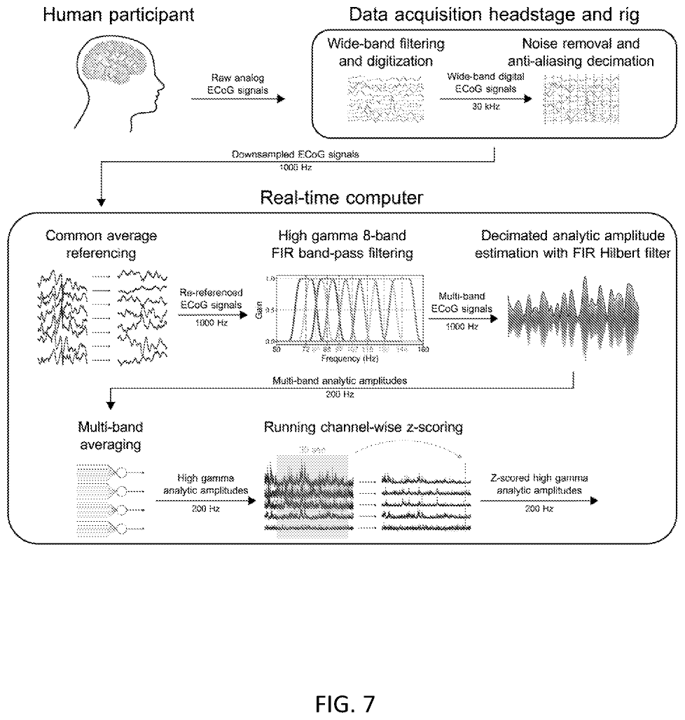

Using a digital signal processing unit and peripheral hardware (NeuroPort System, Blackrock Microsystems), signals from all 128 channels of the implant device were acquired and transmitted to a separate computer running custom software for real-time analysis (Supplementary Method S2; FIGS. 6 and 7) [16, 21]. On this computer, we measured high gamma activity (neural oscillations in the 70-150 Hz frequency range) for each channel, which we then used in all subsequent analyses and during real-time decoding.

### Task Design

The participant engaged in two tasks: an isolated word task and a sentence task (Supplementary Method S3). In each trial of each task, the participant was visually presented with a text target and then attempted to produce (say aloud) that target.

In the isolated word task, the participant attempted to produce individual words from a set of 50 English words. This word set contained common English words that can be used to create a variety of sentences, including words that are relevant to caregiving and words requested by the participant. In each trial, the participant was presented with one of these 50 words, and, after a brief delay, he attempted to produce that word when presented with a visual go cue.

In the sentence task, the participant attempted to produce word sequences from a set of 50 English sentences consisting only of words from the 50-word set (Supplementary Methods S4 and S5). In each trial, the participant was presented with a target sentence and attempted to produce the words in that sentence (in order) at the fastest rate that he was comfortably able to. Throughout the trial, the word sequence decoded from neural activity was updated in real time and displayed as feedback to the participant.

### Modeling

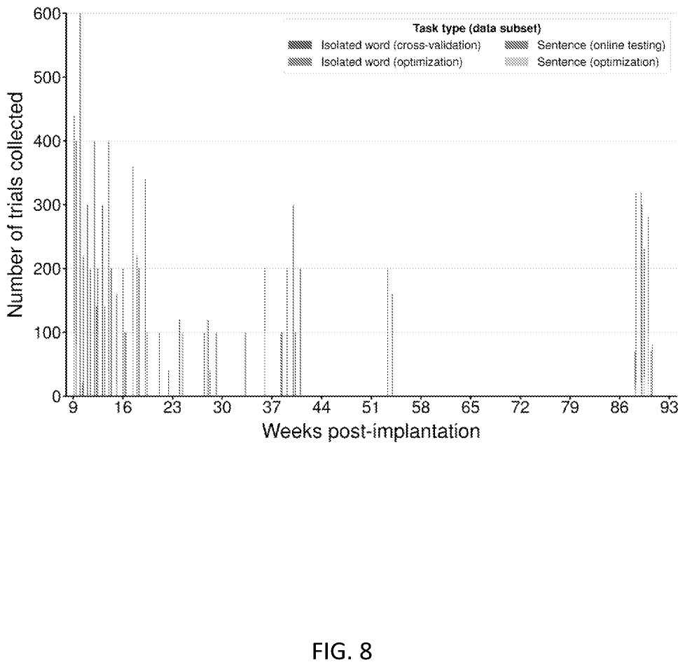

We used neural activity collected during the tasks to train, optimize, and evaluate custom models (Supplementary Methods S6 and S7; FIG. 8; Supplementary Table S1). Specifically, we created speech detection and word classification models that both leveraged deep learning techniques to make predictions from the neural activity. To decode sentences from the participant's neural activity in real time during the sentence task, we used a decoding pipeline containing these two models, a language model, and a Viterbi decoder (FIG. 1).

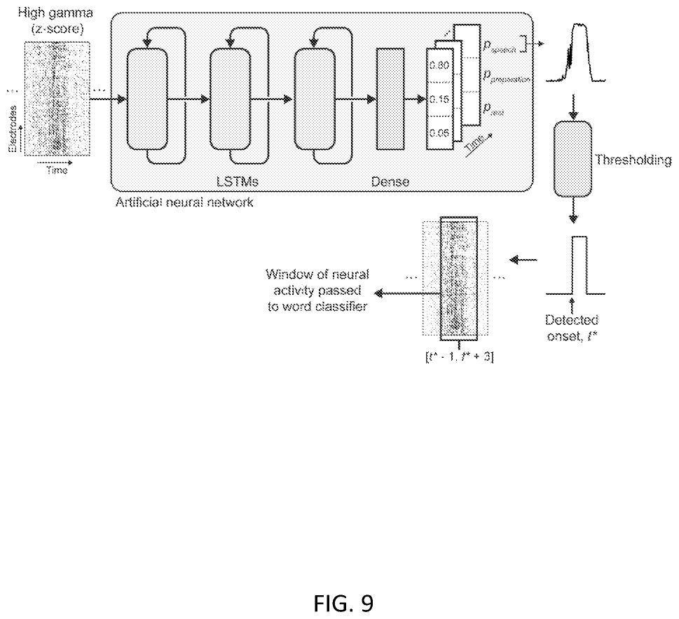

The speech detector processed each time point of neural activity during a task and detected onsets and offsets of attempted word production events in real time (Supplementary Method S8; FIG. 9). We fit this model using only neural data and task timing information from the isolated word task.

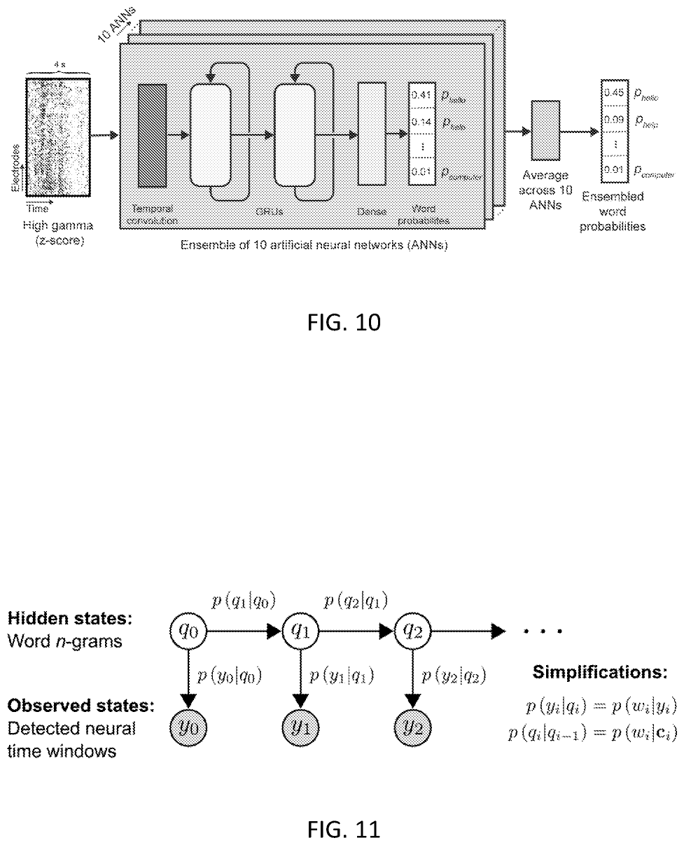

For each detected event, the word classifier predicted a set of word probabilities by processing the neural activity spanning from 1 second before to 3 seconds after the detected onset (Supplementary Method S9; FIG. 10). The predicted probability associated with each word in the 50-word set quantified how likely it was that the participant was attempting to say that word during the detected event. We fit this model using neural data from the isolated word task.

In English, certain sequences of words are more likely than others. We leveraged this underlying structure by using a language model that yielded next-word probabilities given the previous words in a sequence [22, 23] (Supplementary Method S10). We trained this model on a collection of sentences consisting only of words from the 50-word set, which was obtained using a custom task on a crowdsourcing platform (Supplementary Method S4).

We used a custom Viterbi decoder as the final component in the decoding pipeline, which is a type of model that determines the most likely sequence of words given predicted word probabilities from the word classifier and word sequence probabilities from the language model [24] (Supplementary Method S11; FIG. 11). By incorporating the language model, the Viterbi decoder was capable of decoding more plausible sentences than what would result from simply stringing together the predicted words from the word classifier.

### Evaluations

To evaluate the performance of our decoding pipeline, we analyzed the sentences that were decoded in real time using two metrics: word error rate and words per minute (Supplementary Method S12). The word error rate of a decoded sentence is defined as the edit distance (the number of word errors in that sentence) divided by the number of words in the target sentence. The words per minute metric measures how many words were decoded per minute of neural data. We also measured the latency of our system during real-time decoding.

To further characterize the detection and classification of word production attempts from the participant's neural activity, we processed the isolated word data with the speech detector and word classifier in offline analyses (see Supplementary Method S13). To assess how performance was affected by the amount of training data, we used predicted word probabilities from the word classifier to measure classification accuracy while varying the number of trials used during training. Here, classification accuracy is equal to the percent of predictions in which the word classifier correctly assigned the highest probability to the target word. We also measured the contributions that each electrode made to detection and classification by measuring the impact that each channel of neural activity had on the models' predictions [17, 25].

To investigate the clinical viability of our approach for a long-term application, we evaluated the stability of the acquired ECoG signals over time using the isolated word data (Supplementary Method S14). We first determined if the magnitude of neural responses collected during word production attempts changed over the course of the 81-week study period. We also assessed if detection and classification performance was stable throughout the study period by training and testing models using neural data sampled from four different date ranges (“Early”, “Middle”, “Late”, and “Very late”) and then comparing the resulting classification accuracies and electrode contributions.

### Statistical Analyses

The statistical tests used in this work are stated alongside the corresponding significance claims, and thorough descriptions of the tests are provided in Supplementary Method S15. Briefly, we used Wilcoxon signed-rank tests to compare decoding performance to chance and to assess the impact of the language model on performance (with the word error rate metric), linear mixed-effects modeling to assess signal stability, Fisher's exact tests and exact McNemar's tests to compare classification accuracy across different date ranges, and Wilcoxon signed-rank tests to compare electrode contributions across different date ranges. For all tests, we used an alpha level of 0.01. When the neural data used in individual statistical tests of the same type were not independent of each other, we used Holm-Bonferroni correction to account for multiple comparisons.

### Results

### Sentence Decoding

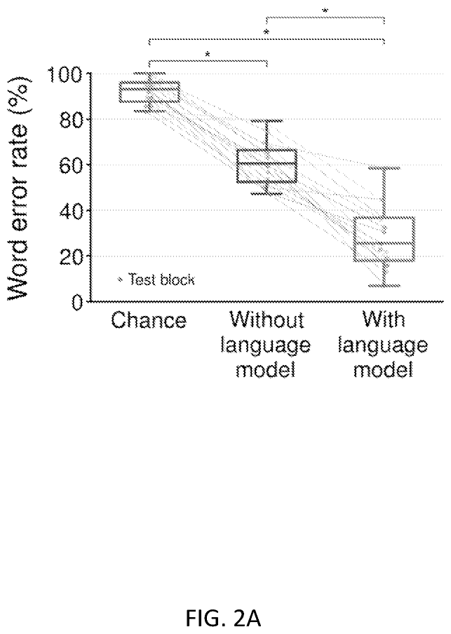

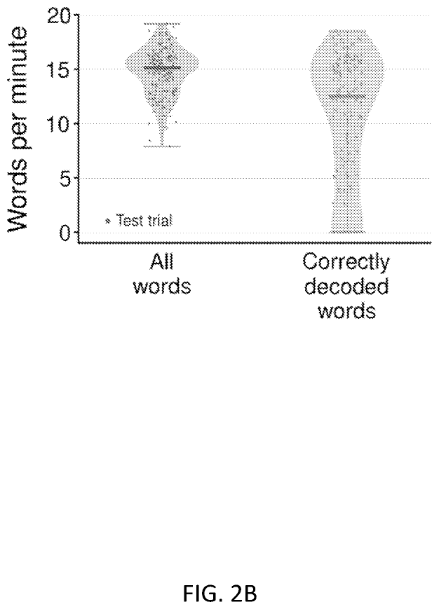

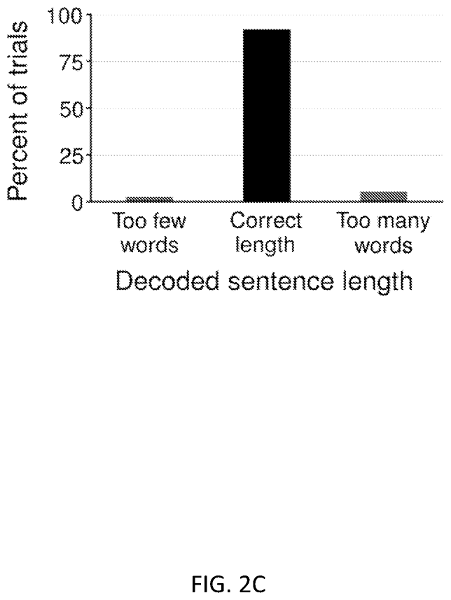

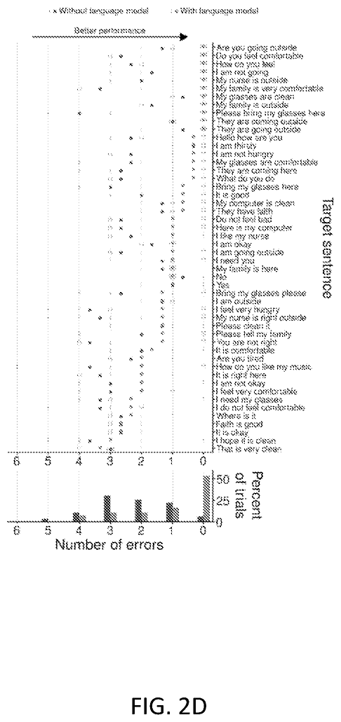

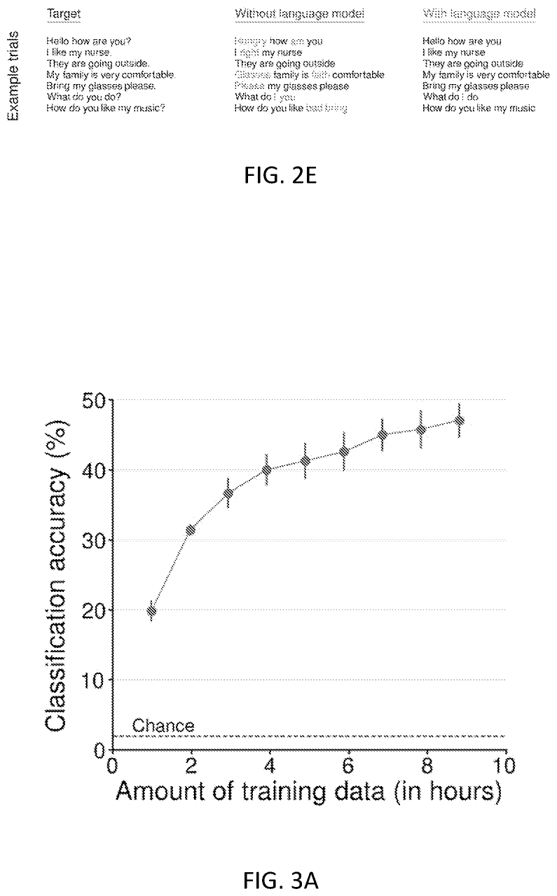

During real-time sentence decoding, the median decoded word error rate across sentence blocks (each block contained 10 trials) was 60.5% without language modeling and 25.6% with language modeling (FIG. 2A). The lowest word error rate observed for a single test block was 6.98% (with language modeling). Word error rates were significantly better than chance and were significantly reduced when incorporating the language model (P<0.001, one-tailed Wilcoxon signed-rank tests, 3-way Holm-Bonferroni correction). Across all 150 trials, the median decoding rate was 15.2 words per minute when including all decoded words and 12.5 words per minute when only including correctly decoded words (FIG. 2B). In 92.0% of trials, the number of detected words was equal to the number of words in the target sentence (FIG. 2C). The detected sentence length was at least one word too short in 2.67% of trials and as least one word too long in 5.33% of trials. Across all 15 sentence blocks, 5 speech events were erroneously detected before the first trial in the block and were excluded from real-time decoding and analysis (all other detected speech events were included). For almost every target sentence, mean edit distance decreased when the language model was used (FIG. 2D). Furthermore, over half of the sentences were decoded without error (80 out of 150 trials; with language modeling; indicated by an edit distance of zero). Use of the language model during decoding improved performance by correcting grammatically and semantically implausible word predictions (FIG. 2E). The mean latency associated with the real-time word predictions was estimated to be 4.0 s (with a standard deviation of 0.91 s).

### Word Detection and Classification

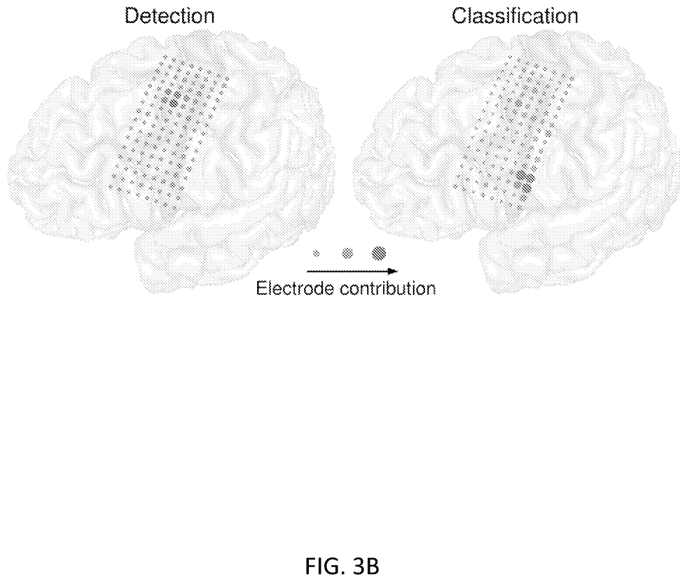

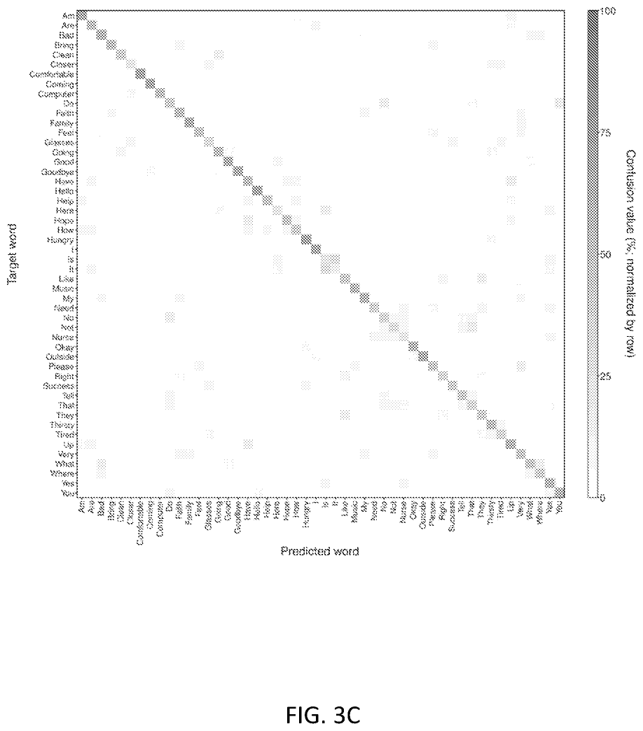

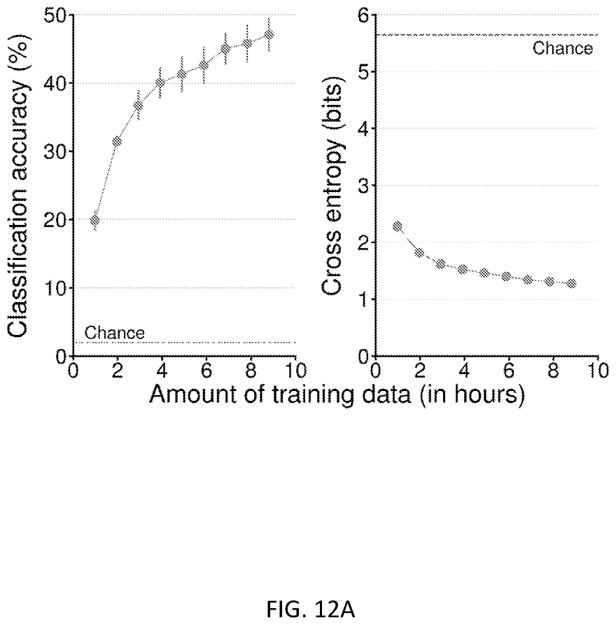

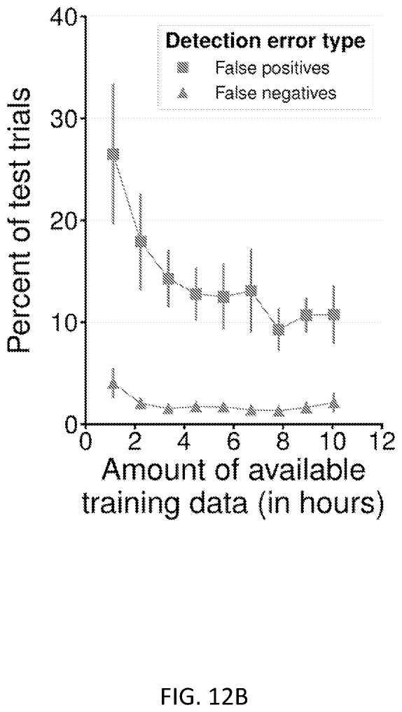

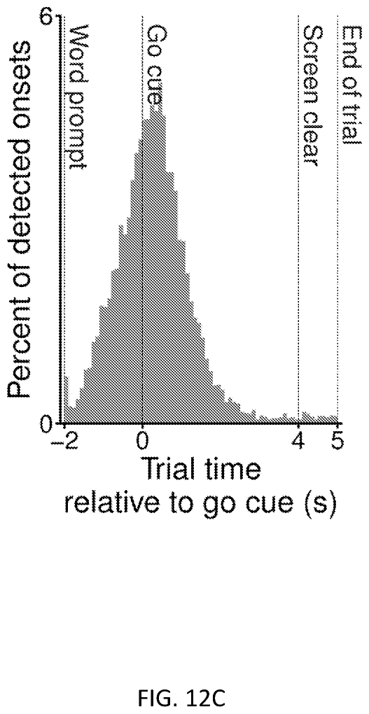

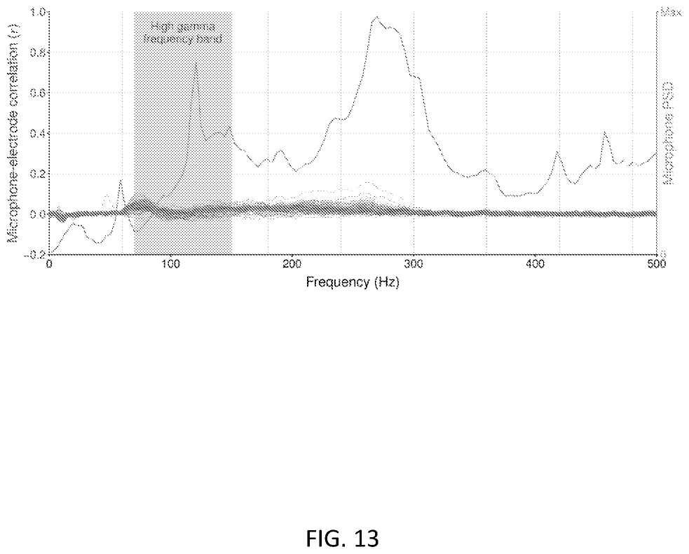

During offline analysis of the isolated word production attempts using detected time windows of cortical activity, classification accuracy increased as the amount of training data increased (up to 47.1% when using all available data; FIG. 3A). Performance improved more rapidly for the first four hours of training data and then less rapidly for the next 5 hours, although it did not plateau. Of the 9000-word production attempts in the isolated word data, 98% were successfully detected (191 attempts were not associated with a detected event), and 968 detected events were spurious (not associated with an attempt; see FIGS. 12 and 13 for additional isolated word analysis results). Electrodes contributing to word classification performance were primarily localized to the ventral-most aspect of the ventral sensorimotor cortex (vSMC), with electrodes in the dorsal aspect of the vSMC contributing to both speech detection and word classification performance (FIG. 3B). Overall, electrode contributions were more distributed for speech detection than for word classification, with over 50% of the total contributions coming from the top 37 electrodes for the word classifier and the top 50 electrodes for the speech detector. Word confusion analysis revealed consistent classification accuracy across the majority of the word targets (FIG. 3C; 47.1% mean and 14.5% standard deviation of the classification accuracy along the diagonal of the row-normalized confusion matrix).

### Long-Term Signal Stability

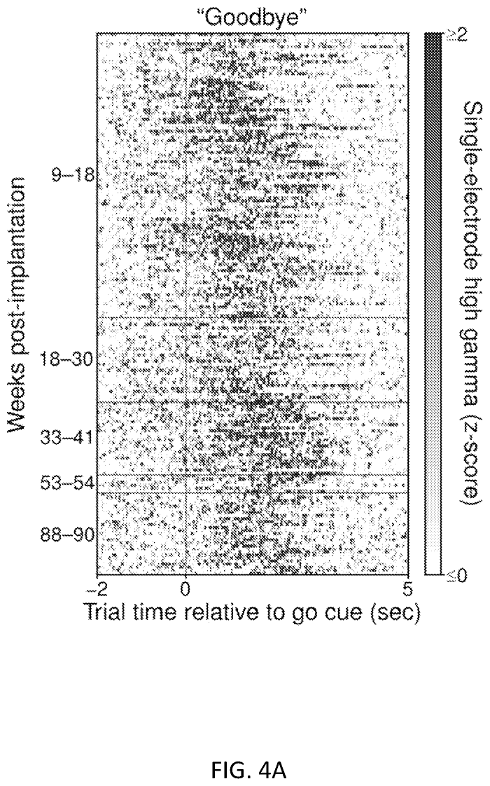

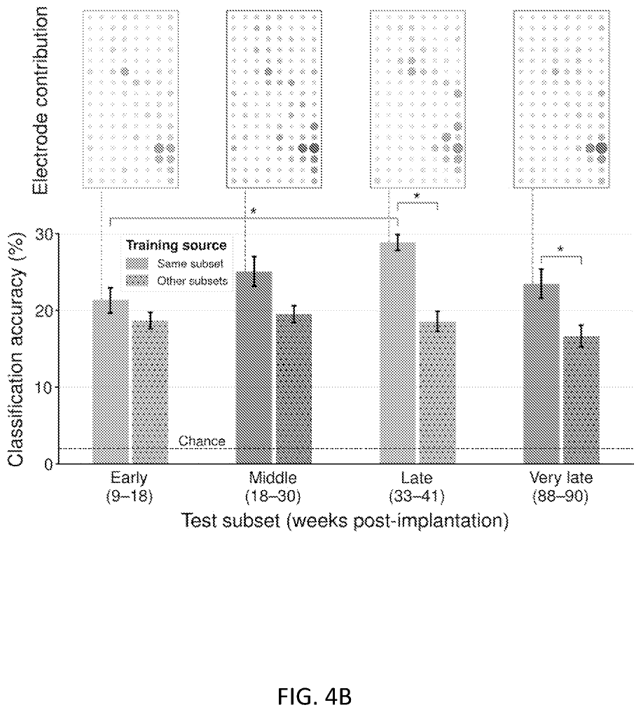

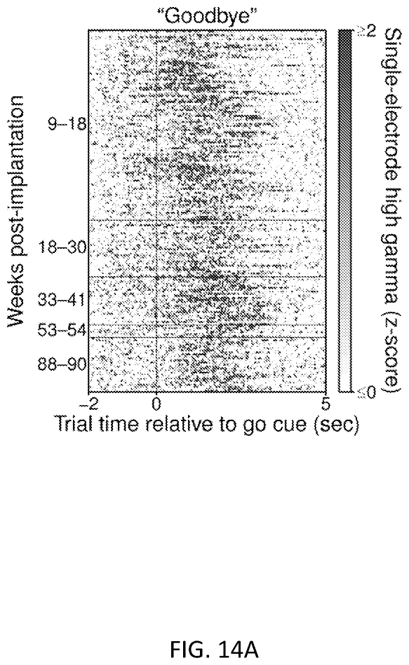

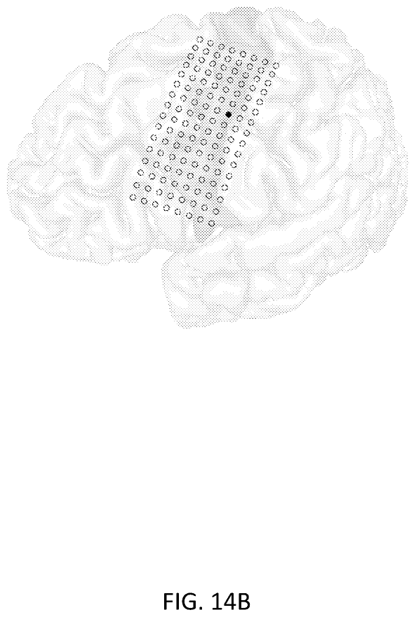

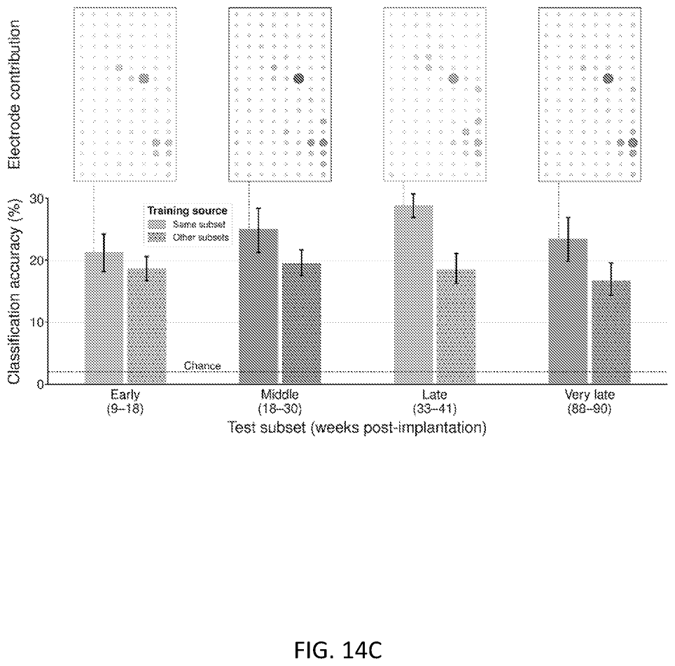

We observed relatively stable single-trial neural activity patterns during word production attempts throughout the 81-week study period (FIG. 4A). Across all electrodes and isolated word trials, there was a slightly overall negative effect of time since implantation on the magnitude of neural responses during attempted speech (slope=−0.00021, SE=0.000011, P<0.001, linear mixed-effects modeling, 129-way Holm-Bonferroni correction; FIG. 14). However, individual electrode modeling revealed significant effects in only 4 of the 128 electrodes (1 positive, 3 negative; P<0.01, linear mixed-effects modeling, 129-way Holm-Bonferroni correction).

By training and testing the speech detector and word classifier on subsets of the isolated word data from distinct date ranges, we found that classification accuracy was lowest for the earliest subset and relatively consistent across the remaining subsets (P=0.0015 for the “Early” vs. “Late” comparison, P>0.01 for all other comparisons, two-tailed Fisher's exact tests, 10-way Holm-Bonferroni correction; FIG. 4B). When evaluating the data in the two latest subsets, classification accuracy was significantly higher when training the models on data from within the same subset as opposed to data from other subsets (P<0.001 for the “Late” and “Very late” subsets and P>0.01 for the other subsets, two-tailed exact McNemar's tests, 10-way Holm-Bonferroni correction). There were no significant changes in electrode contributions across the four subsets (all P>0.32, two-tailed Wilcoxon signed-rank test, uncorrected).

### Discussion

We demonstrate that high-resolution recordings of cortical activity from a severely paralyzed person can be used to decode full words and sentences in real time. Our deep learning models were able to detect and classify word production attempts from neural activity, and we could use these models together with language modeling techniques to decode a variety of meaningful sentences. Signals recorded from the neural interface exhibited stability throughout the study period, enabling successful decoding even up to 90 weeks after surgical implantation. Together, these results have immediate practical implications for paralyzed people who may benefit from speech neuroprosthetic technology.

Previous demonstrations of word and sentence decoding from neural activity were conducted with participants who possessed intact speech and did not require assistive technology to communicate [14-17]. When decoding speech with someone who cannot speak, the lack of precise time alignment between intended speech and neural activity poses a significant challenge during model training. Here, we managed this time-alignment problem with detection techniques [16, 26, 27] and classifiers that leveraged machine learning advances, such as model ensembling and data augmentation (described in Supplementary Method S9), to increase tolerance to minor temporal variabilities [28, 29]. Additionally, our decoding models leveraged neural activity patterns in ventral sensorimotor cortex, consistent with previous work implicating this area in intact speech production [8, 11, 12]. This outcome demonstrates the persistence of functional cortical speech representations after more than 15 years of anarthria, analogous to previous findings of limb-related cortical motor representations in tetraplegic individuals years after loss of movement [30].

Despite imperfect word classification performance, incorporation of language modeling techniques enabled perfect decoding in over half of the sentence trials. This improvement was facilitated by leveraging additional probabilistic information from the word classifier (beyond simply the most likely word identity for each detected word production attempt) and allowing the decoder to correct previous errors given new inputs. These results demonstrate the benefit of integrating linguistic information when decoding speech from neural recordings. Speech decoding approaches generally become usable at word error rates below 30% [31], suggesting that our approach could be immediately applicable in clinical settings.

A fundamental consideration in designing a long-term brain-computer interface (BCI) is the choice of neural recording modality (for example, invasive versus non-invasive) and the implications that this choice has on the resolution, spatial coverage, and stability of the acquired neural signals. Previous motor control BCI studies have demonstrated that electrocorticography (ECoG, the recording modality used in this study) has relatively high signal stability over long evaluation periods compared to other recording modalities [4, 32-34], but these decoding efforts were constrained by limited channel counts and spatial coverage. With our high-density ECoG device, we leveraged broad spatial coverage and high spatial resolution to reliably decode words while observing relatively stable cortical activity throughout the study (only 3 electrodes exhibited significantly diminishing neural response magnitudes over time). Offline classification performance improved and then mostly stabilized after the first few weeks of the study, which can potentially be explained by brain tissue settling during early post-implantation healing [35, 36]. Consistent with a recent cursor-control study with this implant device and study participant [37], our results show that ECoG-based BCIs can maintain consistent speech decoding performance for months with occasional model recalibration. Overall, our findings add to the demonstrations of chronic viability, safety, and signal stability of ECoG-based interfaces for responsive neural stimulation for epilepsy [35, 36] and long-term BCI control [34, 37], extending these attributes to include speech BCIs with high-density ECoG.

Speech is typically the fastest, most natural, and most efficient communication method for healthy individuals [38]. Although our current decoding rates are far slower than natural speaking rates, which often exceed 130 words per minute [38, 39], these results demonstrate the early feasibility of direct speech decoding from cortical signals in a paralyzed person with anarthria. From this proof-of-principle, we can develop and evaluate novel decoders to enable generation of a wider variety of sentences with larger vocabularies. Ultimately, through future work to improve decoding accuracy, flexibility, and speed, we aim to realize the full communicative potential of speech-based neuroprosthetics for people suffering from severe communication disorders.

### REFERENCES

1. Beukelman D R, Fager S, Ball L, Dietz A. AAC for adults with acquired neurological conditions: A review. Augmentative and Alternative Communication 2007; 23(3):230-42.
2. Felgoise S H, Zaccheo V, Duff J, Simmons Z. Verbal communication impacts quality of life in patients with amyotrophic lateral sclerosis. Amyotrophic Lateral Sclerosis and Frontotemporal Degeneration 2016; 17(3-4):179-83.
3. Sellers E W, Ryan D B, Hauser C K. Noninvasive brain-computer interface enables communication after brainstem stroke. Science translational medicine 2014; 6(257):257re7.
4. Vansteensel M J, Pels E G M, Bleichner M G, et al. Fully Implanted Brain-Computer Interface in a Locked-In Patient with ALS. New England Journal of Medicine 2016; 375(21):2060-6.
5. Pandarinath C, Nuyujukian P, Blabe C H, et al. High performance communication by people with paralysis using an intracortical brain-computer interface. ELife 2017; 6:1-27.
6. Brumberg J S, Pitt K M, Mantie-Kozlowski A, Burnison J D. Brain-Computer Interfaces for Augmentative and Alternative Communication: A Tutorial. Am J Speech Lang Pathol 2018; 27(1):1-12.
7. Linse K, Aust E, Joos M, Hermann A, Oliver D J. Communication Matters—Pitfalls and Promise of Hightech Communication Devices in Palliative Care of Severely Physically Disabled Patients With Amyotrophic Lateral Sclerosis. 2018; 9(July):1-18.
8. Bouchard K E, Mesgarani N, Johnson K, Chang E F. Functional organization of human sensorimotor cortex for speech articulation. Nature 2013; 495(7441):327-32.
9. Lotte F, Brumberg J S, Brunner P, et al. Electrocorticographic representations of segmental features in continuous speech. Frontiers in Human Neuroscience 2015; 09(February):1-13.
10. Guenther F H, Hickok G. Neural Models of Motor Speech Control. In: Neurobiology of Language. Elsevier; 2016. p. 725-40.
11. Mugler E M, Tate M C, Livescu K, Templer J W, Goldrick M A, Slutzky M W. Differential Representation of Articulatory Gestures and Phonemes in Precentral and Inferior Frontal Gyri. The Journal of Neuroscience 2018; 4653:1206-18.
12. Chartier J, Anumanchipalli G K, Johnson K, Chang E F. Encoding of Articulatory Kinematic Trajectories in Human Speech Sensorimotor Cortex. Neuron 2018; 98(5):10421054.e4.
13. Salari E, Freudenburg Z V, Branco M P, Aamoutse E J, Vansteensel M J, Ramsey N F. Classification of Articulator Movements and Movement Direction from Sensorimotor Cortex Activity. Sci Rep 2019; 9(1):14165.
14. Herff C, Heger D, de Pesters A, et al. Brain-to-text: decoding spoken phrases from phone representations in the brain. Frontiers in Neuroscience 2015; 9(June):1-11.
15. Anumanchipalli G K, Chartier J, Chang E F. Speech synthesis from neural decoding of spoken sentences. Nature 2019; 568(7753):493-8.
16. Moses D A, Leonard M K, Makin J G, Chang E F. Real-time decoding of question-and-answer speech dialogue using human cortical activity. Nat Commun 2019; 10(1):3096.
17. Makin J G, Moses D A, Chang E F. Machine translation of cortical activity to text with an encoder-decoder framework. Nat Neurosci 2020; 23(4):575-82.
18. Martin S, Iturrate I, Millin J del R, Knight R T, Pasley B N. Decoding Inner Speech Using Electrocorticography: Progress and Challenges Toward a Speech Prosthesis. Front Neurosci 2018; 12:422.
19. Guenther F H, Brumberg J S, Wright E J, et al. A Wireless Brain-Machine Interface for Real-Time Speech Synthesis. PLoS ONE 2009; 4(12):e8218.
20. Brumberg J S, Wright E J, Andreasen D S, Guenther F H, Kennedy P R. Classification of intended phoneme production from chronic intracortical microelectrode recordings in speech-motor cortex. Front Neurosci 2011; 5:65.
21. Moses D A, Leonard M K, Chang E F. Real-time classification of auditory sentences using evoked cortical activity in humans. J Neural Eng 2018; 15(3):036005.
22. Kneser R, Ney H. Improved backing-off for M-gram language modeling. In: 1995 International Conference on Acoustics, Speech, and Signal Processing. Detroit, M I, USA: IEEE; 1995. p. 181-4.
23. Chen S F, Goodman J. An empirical study of smoothing techniques for language modeling. Computer Speech & Language 1999; 13(4):359-93.
24. Viterbi A J. Error Bounds for Convolutional Codes and an Asymptotically Optimum Decoding Algorithm. IEEE Transactions on Information Theory 1967; 13(2):260-9.
25. Simonyan K, Vedaldi A, Zisserman A. Deep Inside Convolutional Networks: Visualising Image Classification Models and Saliency Maps. In: Bengio Y, LeCun Y, editors. Workshop at the International Conference on Learning Representations. Banff, Canada: 2014.
26. Kanas V G, Mporas I, Benz H L, Sgarbas K N, Bezerianos A, Crone N E. Real-time voice activity detection for ECoG-based speech brain machine interfaces. In: 19th International Conference on Digital Signal Processing. 2014. p. 862-5.
27. Dash D, Ferrari P, Dutta S, Wang J. NeuroVAD: Real-Time Voice Activity Detection from Non-Invasive Neuromagnetic Signals. Sensors 2020; 20(8):2248.
28. Sollich P, Krogh A. Learning with ensembles: How overfitting can be useful. In: Touretzky D S, Mozer M C, Hasselmo M E, editors. Advances in Neural Information Processing Systems 8. MIT Press; 1996. p. 190-196.
29. Krizhevsky A, Sutskever I, Hinton G E. ImageNet Classification with Deep Convolutional Neural Networks. In: Pereira F, Burges C J C, Bottou L, Weinberger K Q, editors. Advances in Neural Information Processing Systems 25. Curran Associates, Inc.; 2012. p. 1097-1105.
30. Shoham S, Halgren E, Maynard E M, Normann R A. Motor-cortical activity in tetraplegics. Nature 2001; 413(6858):793-793.
31. Watanabe S, Delcroix M, Metze F, Hershey J R. New era for robust speech recognition: exploiting deep learning. Berlin, Germany: Springer-Verlag; 2017.
32. Chao Z C, Nagasaka Y, Fujii N. Long-term asynchronous decoding of arm motion using electrocorticographic signals in monkey. FrontNeuroeng 2010; 3:3.
33. Freudenburg Z V, Branco M P, Leinders S, et al. Sensorimotor ECoG Signal Features for BCI Control: A Comparison Between People With Locked-In Syndrome and Able-Bodied Controls. Front Neurosci 2019; 13:1058.
34. Pels E G M, Aarnoutse E J, Leinders S, et al. Stability of a chronic implanted brain-computer interface in late-stage amyotrophic lateral sclerosis. Clinical Neurophysiology 2019; 130(10):1798-803.
35. Rao V R, Leonard M K, Kleen J K, Lucas B A, Mirro E A, Chang E F. Chronic ambulatory electrocorticography from human speech cortex. NeuroImage 2017; 153:273-82.
36. Sun F T, Arcot Desai S, Tcheng T K, Morrell M J. Changes in the electrocorticogram after implantation of intracranial electrodes in humans: The implant effect. Clinical Neurophysiology 2018; 129(3):676-86.
37. Silversmith D B, Abiri R, Hardy N F, et al. Plug-and-play control of a brain-computer interface through neural map stabilization. Nat Biotechnol 2020.
38. Hauptmann A G, Rudnicky A I. A comparison of speech and typed input. In: Proceedings of the workshop on Speech and Natural Language—HLT '90. Hidden Valley, Pennsylvania: Association for Computational Linguistics; 1990. p. 219-24.
39. Waller A. Telling tales: unlocking the potential of AAC technologies. International Journal of Language & Communication Disorders 2019; 54(2):159-69.

### Example 2: Supplementary Methods for Word Decoding

### The Method S1. The Participant's Assistive Typing Device

### Assistive Typing Device Description

The participant often uses a commercially available touch-screen typing interface (Tobii Dynavox) to communicate with others, which he controls with a long (approximately 18-inch) plastic stylus attached to a baseball cap by using residual head and neck movement. The device displays letters, words, and other options (such as punctuation) that the participant can select with his stylus, enabling him to construct a text string. After creating the desired text string, the participant can use his stylus to press an icon that synthesizes the text string into an audible speech waveform. This process of spelling out a desired message and having the device synthesize it is the participant's typical method of communication with his caregivers and visitors.

### Typing Rate Assessment Task Design

To compare with the neural-based decoding rates achieved with our system, we measured the participant's typing rate while he used his typing interface in a custom task. In each trial of this task, we presented a word or sentence on the screen and the participant typed out that word or sentence using his typing interface. We instructed the participant to not use any of the word suggestion or completion options in his interface, but use of correction features (such as backspace or undo options) was permitted. We measured the amount of time between when the target word or sentence first appeared on the screen and when the participant entered the final letter of the target. We then used this duration and the target word or utterance to measure words per minute and correct characters per minute for each trial.

We used a total of 35 trials (25 words and 10 sentences). Punctuation was included when presented to the participant, but the participant was instructed not to type out punctuation during the task. The target words and sentences were:
1. Thirsty
2. I
3. Tired
4. Are
5. Up
6. How
7. Outside
8. You
9. Bad
10. Clean
11. Have
12. Tell
13. Hello
14. Going
15. Right
16. Closer
17. What
18. Success
19. It
20. Family
21. That
22. Help
23. Do
24. Am
25. Okay
26. It is good.
27. I am thirsty.
28. They are coming here.
29. Are you going outside?
30. I am outside.
31. Faith is good.
32. My family is here.
33. Please tell my family.
34. My glasses are comfortable.
35. They are coming outside.

### Typing Rate Results and Discussion

Across all trials of this typing task, the mean standard deviation of the participant's typing rate was 5.03±3.24 correct words per minute or 17.9±3.47 correct characters per minute.

Although these typing rates are slower than the real-time decoding rates of our approach, the unrestricted vocabulary size of the typing interface is a key advantage over our approach. Given the correct characters per minute that the participant is able to achieve with the typing interface, replacing the letters in the interface with the 50 words from this task could result in higher decoding rate and accuracy than what was achieved with our approach. However, this typing interface is less natural and appears to require more physical exertion than attempted speech, suggesting that the typing interface might be more fatiguing than our approach.

### Method S2. Neural Data Acquisition and Real-Time Processing

### Initial Data Acquisition and Preprocessing Steps

The implanted electrocorticography (ECoG) array (PMT Corporation) contains electrodes arranged in a 16-by-8 lattice formation with 4-mm center-to-center spacing. The rectangular ECoG array has a length of 6.7 cm, a width of 3.5 cm, and a thickness of 0.51 mm, and the electrode contacts are disc-shaped with 2-mm contact diameters. To process and record neural data, signals were acquired from the ECoG array and processed in several steps involving multiple hardware devices (FIG. 6 and FIG. 7). First, a headstage (a detachable digital link; Blackrock Microsystems) connected to the percutaneous pedestal connector (Blackrock Microsystems) acquired electrical potentials from the implanted electrode array. The pedestal is a male connector and the headstage is a female connector. This headstage performed band-pass filtering on the signals using a hardware-based Butterworth filter between 0.3 Hz and 7.5 kHz. The digitized signals (with 16-bit, 250-nV per bit resolution) were then transmitted through an HDMI cable to a digital hub (Blackrock Microsystems), which then sent the data through an optical fiber cable to a Neuroport system (Blackrock Microsystems). In early recording sessions, before the digital headstage was approved for use in human research, we used a human patient cable (Blackrock Microsystems) to connect the pedestal to a front-end amplifier (Blackrock Microsystems), which amplified and digitized the signals before they were sent through an optical fiber to the Neuroport system. This Neuroport system sampled all 128 channels of ECoG data at 30 kHz, applied software-based line noise cancellation, performed anti-aliasing low-pass filtering at 500 Hz, and then streamed the processed signals at 1 kHz to a separate real-time processing machine (Colfax International). The Neuroport system also acquired, streamed, and stored synchronized recordings of the relevant acoustics at 30 kHz (microphone input and speaker output from the real-time processing computer).

### Further Preprocessing and Feature Extraction

The real-time processing computer, which is a Linux machine (64-bit Ubuntu 18.04, 48 Intel Xeon Gold 6146 3.20 GHz processors, 500 GB of RAM), used a custom software package called real-time Neural Speech Recognition (rtNSR) [1, 2] to analyze and process the incoming neural data, run the tasks, perform real-time decoding, and store task data and metadata to disk. Using this software, we performed the following preprocessing steps on all acquired neural signals in real time:

We applied a common average reference to each time sample of the acquired ECoG data (across all electrodes), which is a standard technique for reducing shared noise in multi-channel data [3, 4].

We applied eight band-pass finite impulse response (FIR) filters with logarithmically increasing center frequencies in the high gamma band (at 72.0, 79.5, 87.8, 96.9, 107.0, 118.1, 130.4, and 144.0 Hz, rounded to the nearest decimal place). Each of these 390th-order filters was designed using the Parks-McClellan algorithm [5].

We computed analytic amplitude values for each band and channel using a 170th-order FIR filter designed with the Parks-McClellan algorithm to approximate the Hilbert transform. For each band and channel, we estimated the analytic signal by using the original signal (delayed by 85 samples, which is half of the filter order) as the real component and the Hilbert transform of the original signal (approximated by this FIR filter) as the imaginary component [6]. Afterwards, we obtained analytic amplitude values by computing the magnitude of each of these analytic signals. We only applied this analytic amplitude calculation to every fifth sample of the band-passed signals, yielding analytic amplitudes decimated to 200 Hz.

We computed a single high gamma analytic amplitude measure for each channel by averaging the analytic amplitude values across the eight bands.

We z-scored the high gamma analytic amplitude values for each channel using Welford's method with a 30-second sliding window [7].

We used these high gamma analytic amplitude z-score time series (sampled at 200 Hz) in all analyses and during online decoding.

### Portability and Cost of the Hardware Infrastructure

In this work, the hardware used was fairly large but still portable, with most of the hardware components residing on a mobile rack with a length and width each at around 76 cm. We performed all data collection and online decoding tasks either in the participant's bedroom or in a small office room near the participant's residence. Although we supervised all use of the hardware throughout the clinical trial, the hardware and software setup procedures required to begin recording were straightforward; it is feasible that a caregiver could, after a few hours of training and with the appropriate regulatory approval, prepare our system for use by the participant without our direct supervision. To set up the system for use, a caregiver would perform the following steps:
1. Remove and clean the percutaneous connector cap, which protects the external electrical contacts on the percutaneous connector while the system is not being used
2. Clean percutaneous connector, the digital link, and the scalp area around the percutaneous connector
3. Connect the digital link to the percutaneous connector
4. Turn on computers and start the software
5. Ensure that the screen was properly positioned in front of the participant for use
   Afterwards, to disengage the system, a caregiver would perform the following steps:
1. Close the software and turn off the computers
2. Disconnect the digital link from the percutaneous connector
3. Clean percutaneous connector, the digital link, and the scalp area around the percutaneous connector
4. Place the percutaneous connector cap back on the percutaneous connector

The full hardware infrastructure was fairly expensive, primarily due to the relatively high cost of a new Neuroport system (compared to the costs of other hardware devices used in this work). However, recent work has demonstrated that a relatively cheap and portable brain-computer interface system can be deployed without a significant decrease in system performance (compared to a typical system containing Blackrock Microsystem devices, such as the system used in this work) [8]. The demonstrations in that work suggest that future iterations of our hardware infrastructure could be made cheaper and more portable without sacrificing decoding performance.

### Computational Modeling Infrastructure

We uploaded the collected data from the real-time processing computer to our lab's computational and storage server infrastructure. Here, we fit and optimized the decoding models, using multiple NVIDIA V100 GPUs to reduce computation time. Finalized models were then downloaded to the real-time processing computer for online decoding.

### Method S3. Task Design

All data were collected as a series of “blocks”, with each block lasting about 5 or 6 minutes and consisting of multiple trials. There were two types of tasks: an isolated word task and a sentence task.

### Isolated Word Task

In the isolated word task, the participant attempted to produce individual words from a 50-word set while we recorded his cortical activity for offline processing. This word set was chosen based on the following criteria:
1. The ease with which the words could be used to create a variety of sentences.
2. The ease with which the words could be used to communicate basic caregiving needs.
3. The participant's interest in including the words. We iterated through a few versions of the 50-word set with the participant using feedback that he provided to us through his commercially-available assistive communication technology.
4. The desire to include a number of words that are large enough to create a meaningful variety of sentences but small enough to enable satisfactory neural-based classification performance. This latter criterion was informed by exploratory, preliminary assessments with the participant following device implantation (prior to collection of any of the data analyzed in this study). A list of the words contained in this 50-word set is provided at the end of this section.

To keep task blocks short in duration, we arbitrarily split this word set into three disjoint subsets, with two subsets containing 20 words each and the third subset containing the remaining 10 words. During each block of this task, the participant attempted to produce each word contained in one of these subsets twice, resulting in a total of either 40 or 20 attempted word productions per block (depending on the size of the word subset). In three blocks of the third, smaller subset, the participant attempted to produce the 10 words in that subset four times each (instead of the usual two).

Each trial in a block of this task started with a blank screen with a black background. After 1 second (or, in very few blocks, 1.5 seconds), one of the words in the current word subset was shown on the screen in white text, surrounded on either side by four period characters (for example, if the current word was “Hello”, the text “ . . . Hello . . . ” would appear). For the next 2 seconds, the outer periods on either side (the first and last characters of the displayed text string) would disappear every 500 ms, visually representing a countdown. When the final period on either side of the word disappeared, the text would turn green and remain on the screen for 4 seconds. This color transition from white to green represented the go cue for each trial, and the participant was instructed to attempt to produce the word as soon as the text turned green. Afterwards, the task continued to the next trial. The word presentation order was randomized within each task block. The participant chose this countdown-style task paradigm from a set of potential paradigm options that we presented to him during a presurgical interview, claiming that he was able to use the consistent countdown timing to better align his production attempts with the go cue in each trial.

### Sentence Task

In the sentence task, the participant attempted to produce sentences from a 50-sentence set while his neural activity was processed and decoded into text. These sentences were composed only of words from the 50-word set. These 50 sentences were selected in a semi-random fashion from a corpus of potential sentences (see Method S5). A list of the sentences contained in this 50-sentence set is provided at the end of this section. To keep task blocks short in duration, we arbitrarily split this sentence set into five disjoint subsets, each containing 10 sentences. During each block of this task, the participant attempted to produce each sentence contained in one of these subsets once, resulting in a total of 10 attempted sentence productions per block.

Each trial in a block of this task started with a blank screen divided horizontally into top and bottom halves, both with black backgrounds. After two seconds, one of the sentences in the current sentence subset was shown in the top half of the screen in white text. The participant was instructed to attempt to produce the words in the sentence as soon as the text appeared on the screen at the fastest rate that he was comfortably able to. While the target sentence was displayed to the participant, his cortical activity was processed in real time by a speech detection model. Each time an attempted word production was detected from the acquired neural signals, a set of cycling ellipses (a text string that cycled each second between one, two, and three period characters) was added to the bottom half of the screen as feedback, indicating that a speech event was detected. Word classification, language, and Viterbi decoding models were then used to decode the most likely word associated with the current detected speech event given the corresponding neural activity and the decoded information from any previous detected events within the current trial. Whenever anew word was decoded, that word replaced the associated cycling ellipses text string in the bottom half of the screen, providing further feedback to the participant. The Viterbi decoding model, which maintained the most likely word sequence in a trial given the observed neural activity, often updated its predictions for previous speech events given a new speech event, causing previously decoded words in the feedback text string to change as new information became available. After a pre-determined amount of time had elapsed since the detected onset of the most recent speech event, the sentence target text turned from white to blue, indicating that the decoding portion of the trial had ended and that the decoded sentence had been finalized for that trial. This pre-determined amount of time was either 9 or 11 seconds depending on the block type (see the following paragraph). After 3 seconds, the task continued to the next trial.

We collected two types of blocks of the sentence task: optimization blocks and testing blocks. The differences between these two types of blocks are:
1. We used the optimization blocks to perform hyperparameter optimization and the testing blocks to assess the performance of the decoding system.
2. We used intermediate (non-optimized) models when collecting the optimization blocks and finalized (optimized) models when collecting the testing blocks.
3. Although detected speech attempts and decoded word sequences were always provided to the participant as feedback during this task, during collection of optimization blocks he was instructed not to repeat a word if a speech event was missed or use the feedback to alter which words he attempted to produce. We included these instructions to protect the integrity of the data for use with the hyperparameter optimization procedure (if the participant had altered his behavior because of imperfect speech detection, discrepancies between the prompted word sequence and the word sequence that the participant actually attempted could have hindered the optimization procedure). During testing blocks, however, we encouraged the participant to take the feedback into consideration when attempting to produce the target sentence. For example, if an attempted word production was not detected, the participant could repeat the production attempt before proceeding to the next word.
4. During optimization blocks, the pre-determined amount of time that controlled when the decoded word sequence in each trial was finalized (see the previous paragraph) was set to 9 seconds. During testing blocks, this task parameter was set to 11 seconds to give the participant extra time to incorporate the provided feedback from the decoding pipeline.

We also collected a conversational variant of the sentence task to demonstrate that the decoding approach could be used in a more open-ended setting in which the participant could generate custom responses to questions from the 50 words. In this variant of the task, instead of being prompted with a target sentence to attempt to repeat, the participant was prompted with a question or statement that mimicked a conversation partner and was instructed to attempt to produce a response to the prompt. Other than the conversational prompts and this change in task instructions to the participant, this variant of the task was identical to the regular version. We did not perform any analyses with data collected from this variant of the sentence task; it was used for demonstration purposes only. This variant of the task is shown in FIG. 1 in the main text.

### Word and Sentence Lists

The 50-word set used in this work is:
1. Am
2. Are
3. Bad
4. Bring
5. Clean
6. Closer
7. Comfortable
8. Coming
9. Computer
10. Do
11. Faith
12. Family
13. Feel
14. Glasses
15. Going
16. Good
17. Goodbye
18. Have
19. Hello
20. Help
21. Here
22. Hope
23. How
24. Hungry
25. I
26. Is
27. It
28. Like
29. Music
30. My
31. Need
32. No
33. Not
34. Nurse
35. Okay
36. Outside
37. Please
38. Right
39. Success
40. Tell
41. That
42. They
43. Thirsty
44. Tired
45. Up
46. Very
47. What
48. Where
49. Yes
50. You

The 50-sentence set used in this work is:
1. Are you going outside?
2. Are you tired?
3. Bring my glasses here.
4. Bring my glasses please.
5. Do not feel bad.
6. Do you feel comfortable?
7. Faith is good.
8. Hello how are you?
9. Here is my computer.
10. How do you feel?
11. How do you like my music?
12. I am going outside.
13. I am not going.
14. I am not hungry.
15. I am not okay.
16. I am okay.
17. I am outside.
18. I am thirsty.
19. I do not feel comfortable.
20. I feel very comfortable.
21. I feel very hungry.
22. I hope it is clean.
23. I like my nurse.
24. I need my glasses.
25. I need you.
26. It is comfortable.
27. It is good.
28. It is okay.
29. It is right here.
30. My computer is clean.
31. My family is here.
32. My family is outside.
33. My family is very comfortable.
34. My glasses are clean.
35. My glasses are comfortable.
36. My nurse is outside.
37. My nurse is right outside.
38. No.
39. Please bring my glasses here.
40. Please clean it.
41. Please tell my family.
42. That is very clean.
43. They are coming here.
44. They are coming outside.
45. They are going outside.
46. They have faith.
47. What do you do?
48. Where is it?
49. Yes.
50. You are not right.

### Method S4. Collection of the Sentence Corpus

To train a domain-specific language model for the sentence task (and to obtain a set of target sentences for this task), we used an Amazon Mechanical Turk task to crowdsource an unbiased corpus of natural English sentences that only contained words from the 50-word set. A web-based interface was designed to display the 50 words, and Mechanical Turk workers (referred to as “Turkers”) were instructed to construct sentences that met the following criteria:
Each sentence should only consist of words from the 50-word set.
No duplicates should be present in the sentence responses for each individual Turker.
Each sentence should be grammatically valid.
Each sentence should have a length of 8 words or fewer.

Additionally, the Turkers were encouraged to use different words across the different sentences (while always restricting words to the 50-word set). Only Turkers from the USA were allowed for this task to restrict dialectal influences in the collected sentences. After removing spurious submissions and spammers, the corpus contained 3415 sentences (1207 unique sentences) from 187 Turkers.

### Method S5. Creation of the Sentence Target Set

To extract the set of 50 sentences used as targets in the sentence task from the Amazon Mechanical Turk corpus (refer to Method S4 for more details about this corpus), we first restricted this selection process to only consider sentences that appeared more than once in the corpus. We imposed this inclusion criterion to discourage the selection of idiosyncratic sentences for the target set. Afterwards, we randomly sampled from the remaining sentences, discarding some samples if they contained grammatical mistakes or undesired content (such as “Family is bad”). After a set of 50 sentence samples was created, a check was performed to ensure that at least 90% of the words in the 50-word set appeared at least once in this sentence set. If this check failed, we ran the sentence sampling process again until the check was passed, yielding the target sentence set for the sentence task.

During the sentence sampling procedure that ultimately yielded the 50-sentence set used in this study, the following 22 sentences were discarded:
1. Good family is success
2. Tell success
3. Bring computer
4. Tell that family
5. I going outside
6. You are hungry
7. I feel very bad
8. I need glasses
9. I need computer
10. You need my help
11. You are coming closer
12. Tell you right
13. I am closer
14. It is bad outside
15. Success is not coming
16. I like nurse
17. Family is bad
18. I tell you
19. That nurse is thirsty
20. Need help
21. They are very thirsty
22. Where is computer

The target sentence set contained 45 of the 50 possible words. The following 5 words did not appear in the target sentence set:
1. Closer
2. Goodbye
3. Help
4. Success
5. Up

However, because the word classifier was trained on isolated attempts to produce each word in the 50-word set and computed probabilities across all 50 words during inference, these 5 words could still appear in the sentences decoded from the participant's neural activity.

### Method S6. Data Organization

### Isolated Word Data: Subset Creation

In total, we collected 22 hours and 30 minutes of the isolated word task in 291 task blocks across 48 days of recording, with 196 trials (attempted productions) per word (9800 trials total). We split these blocks into 11 disjoint subsets: a single optimization subset and 10 cross-validation subsets. The optimization subset contained a total of 16 trials per word, and each cross-validation subset contained 18 trials per word.

To create subsets that were similarly distributed across time, we first ordered the blocks chronologically. Next, we assigned the blocks that occurred at evenly spaced indices within this ordered list (spanning from the earliest to the latest blocks) to the optimization subset. We then assigned the remaining blocks to the cross-validation subsets by iterating through the blocks while cycling through the cross-validation subset labels. We deviated slightly from this approach only to ensure that each subset contained the desired number of trials per word. This prevented any single subset from having an over-representation of data from a specific time period, although our irregular recording schedule prevented the subsets from containing blocks that were equally spaced in time (see FIG. 8).

We evaluated models on data in the optimization subset during hyperparameter optimization (see Method S7). We used the hyperparameter values found during this process for all isolated word analyses, unless otherwise stated.

Using the hyperparameter values found during this process, we performed 10-fold cross-validation with the 10 cross-validation subsets, fitting our models on 9 of the subsets and evaluating on the held-out subset in each fold. Unless stated otherwise, the trials in the optimization subset were not used directly during isolated word evaluations.

### Isolated Word Data: Learning Curve Scheme

To assess how quantity of training data affected performance, we used the 10 cross-validation subsets to generate a learning curve scheme. In this scheme, the speech detector and word classifier were assessed using cross-validation with nine different amounts of training data. Specifically, for each integer value of N∈[1, 9], we performed 10-fold cross-validated evaluation with the isolated word data while only training on N randomly selected subsets in each fold. Through this approach, all of the available trials were evaluated for each value of N even though the amount of training data varied, and there was no overlap between training and testing data in any individual assessment. The final set of analyses in this learning curve scheme (with N=9) was equivalent to a full 10-fold cross-validation analysis with all of the available data, and, with the exception of the learning curve results, we only used this set of analyses to compute all of the reported isolated word results (including the electrode contributions and confusion matrix shown in FIG. 3 in the main text). With 18 attempted productions per word in each subset, the nine sets of analyses using this learning curve scheme contained 18, 36, 54, 72, 90, 108, 126, 144, and 162 trials per word during training, in that order. Because the word classifier was fit using curated detected events, not every trial was evaluated in each set of analyses (see Method S13 and Method S8 for more details).

### Isolated Word Data: Stability Subsets

To assess how stable the signals driving word detection and classification were throughout the study period, we used the isolated word data to define four date-range subsets containing data collected during different date ranges. These date-range subsets, named “Early”, “Middle”, “Late”, and “Very late”, contained data collected 9-18, 18-30, 33-41, and 88-90 weeks post-implantation, respectively. Data collected on the day of the exact 18-week mark was considered to be part of the “Early” subset, not the “Middle” subset. Each of these subsets contained 20 trials for each word, randomly drawn (without replacement) from the available data in the corresponding date range. Trials were only sampled from the isolated word cross-validation subsets (not from the optimization subset). In FIG. 4 in the main text, the date ranges for these subsets are expressed relative to the start of data collection for this study (instead of being expressed relative to the device implantation date). Within each of these subsets, we further split the data into 10 disjoint subsets (referred to in this section as “pieces” to disambiguate these subsets from the four date-range subsets), each containing 2 trials of each word. Using these four date-range subsets, we defined three evaluation schemes: a within-subset scheme, an across-subset scheme, and a cumulative-subset scheme.

The within-subset scheme involved performing 10-fold cross-validation using the 10 pieces within each date-range subset. Specifically, each piece in a date-range subset was evaluated using models fit on all of the data from the remaining pieces of that date-range subset. We used the within-subset scheme to detect all of the speech events for the word classifier to use during training and testing (for each date-range subset and each evaluation scheme). The training data used within each individual cross-validation fold for each date-range subset always consisted of 18 trials per word.

The across-subset scheme involved evaluating the data in a date-range subset using models fit on data from other date-range subsets. In this scheme, the within-subset scheme was replicated, except that each piece in a date-range subset was evaluated using models fit on 6 trials per word randomly sampled (without replacement) from each of the other date-range subsets. The training data used within each individual cross-validation fold for each date-range subset always consisted of 18 trials per word.

The cumulative-subset scheme involved evaluating the data from the “Very late” subset using models fit with varying amounts of data. In this scheme, four cross-validated evaluations were performed (using the 10 pieces defined for each date-range subset). In the first evaluation, data from the “Very late” subset were analyzed by the word classifier using 10-fold cross-validation (this was identical to the “Very late” within-subset evaluation). In the second evaluation, the cross-validated analysis from the first evaluation was repeated, except that all of the data from the “Late” subset was added to the training dataset for each cross-validation fold. The third evaluation was similar except that all of the data from the “Middle” and “Late” subsets were also included during training, and in the fourth evaluation all of the data from the “Early”, “Middle”, and “Late” subsets were included during training.

Refer to Method S14 for a description of how these schemes were used to analyze signal stability.

### Sentence Data

In total, we collected 2 hours and 4 minutes of the sentence task in 25 task blocks across 7 days of recording, with 5 trials (attempted productions) per sentence (250 trials total). We split these blocks into two disjoint subsets: A sentence optimization subset and a sentence testing subset. We used the sentence optimization subset, which contained 2 trials of each sentence, to optimize our sentence decoding pipeline prior to online testing. When collecting these blocks, we used non-optimized models. Afterwards, we used the data from these blocks to optimize our models for online testing (refer to the hyperparameter optimization procedure described in Method S7). These blocks were only used for optimization and were not included in further sentence decoding analyses.

We used the outcomes of the blocks contained in the testing subset, which contained 3 trials of each sentence, to evaluate decoding performance. These blocks were collected using optimized models.

We did not fit any models directly on neural data collected during the sentence task (from either subset).

### Method S7. Hyperparameter Optimization

To find optimal values for the model hyperparameters used during performance evaluation, we used hyperparameter optimization procedures to evaluate many possible combinations of hyperparameter values, which were sampled from custom search spaces, with objective functions that we designed to measure model performance. During each hyperparameter optimization procedure, a desired number of combinations were tested, and the combination associated with the lowest (best) objective function value across all combinations was chosen as the optimal hyperparameter value combination for that model and evaluation type. The data used to measure the associated objective function values were distinct from the data that the optimal hyperparameter values would be used to evaluate (hyperparameter values used during evaluation of a test set were never chosen by optimizing on data in that test set). We used three types of hyperparameter optimization procedures to optimize a total of 9 hyperparameters (see Table S1 for the hyperparameters and their optimal values).

Speech Detection Optimization with Isolated Word Data

To optimize the speech detector with isolated word data, we used the hyperopt Python package [9], which samples hyperparameter value combinations probabilistically during the optimization procedure. We used this procedure to optimize the smoothing size, probability threshold, and time threshold duration hyperparameters (described in Method S8). Because these thresholding hyperparameters were only applied after speech probabilities were predicted, these hyperparameters did not affect training or evaluation of the artificial neural network model driving the speech detector. In each iteration of the optimization procedure, the current hyperparameter value combination was used to generate detected speech events from the existing speech probabilities. We used the objective function given in Equation S5 to measure the model performance with each hyperparameter value combination. In each detection hyperparameter optimization procedure, we evaluated 1000 hyperparameter value combinations before stopping.

As described in Method S6, we computed speech probabilities for isolated word blocks in each of the 10 cross-validation data subsets using a speech detection model trained on the data from the other 9 cross-validation subsets. To compute speech probabilities for the blocks in the optimization subset, we used a speech detection model trained on data from all 10 of the cross-validation subsets. Afterwards, we performed hyperparameter optimization with the blocks in the optimization subset, which yielded the optimal hyperparameter value combination that was used during evaluation of the data in the 10 cross-validation subsets (including learning curve and stability analyses).

To generate detected events for blocks in the optimization subset (which were used during hyperparameter optimization of the word classifier), we performed a separate hyperparameter optimization with a subset of data from the 10 cross-validation subsets. This subset, containing 16 trials of each of the 50 words, was created by randomly selecting blocks from the 10 cross-validation subsets. We then performed hyperparameter optimization with this new subset using the predicted speech probabilities that had already been computed for those blocks (as described in the previous paragraph). Afterwards, we used the resulting optimal hyperparameter value combination to detect speech events for blocks in the optimization subset.

Word Classification Optimization with Isolated Word Data

To optimize the word classifier with isolated word data, we used the Ray Python package [10], which performs parallelized hyperparameter optimization with randomly sampled hyperparameter value combinations from pre-defined search spaces. This hyperparameter optimization approach uses a scheduler based on the “Asynchronous Successive Halving Algorithm” (ASHA) [11], which performs aggressive early stopping to discard underperforming hyperparameter value combinations before they are fully evaluated. This approach has been shown to outperform Bayesian hyperparameter optimization approaches when the computational complexity associated with the evaluation of a single hyperparameter value combination is high and a large number of hyperparameter combinations are evaluated [10]. We used this approach to optimize the word classification hyperparameters because of the long computation times required to train the ensemble of deep artificial neural network models comprising each word classifier. Training a single network on an NVIDIA V100 GPU required approximately 28 seconds per epoch using our augmented dataset. Each network required, on average, approximately 25 epochs of training (although the duration of each epoch can vary due to early stopping). This approximation indicates that a single network required 700 seconds to train. Because we used an ensemble of 4 networks during hyperparameter optimization, a total GPU time of approximately 46 minutes and 40 seconds was required to train a word classifier for a single hyperparameter value combination (for the word classifiers used during evaluation and real-time prediction, which each contained an ensemble of 10 networks, the approximate training time per classifier was 1 hour, 56 minutes, and 40 seconds). To evaluate a large number of hyperparameter value combinations given these training times, it was beneficial to use a computationally efficient hyperparameter optimization algorithm (such as the ASHA algorithm used here).

We performed two different hyperparameter optimizations for the word classifier, both using cross-entropy loss on a held-out set of trials as the objective function during optimization (see Equation S6 in Method S9). Each optimization evaluated 300 different combinations of hyperparameter values. For the first optimization, we used the optimization subset as the held-out set while training on data from all 10 cross-validation subsets. We used the resulting hyperparameter value combination for the isolated word analyses. For the second optimization, we created a held-out set by randomly selecting (without replacement) 4 trials of each word from blocks collected within three weeks of the online sentence decoding test blocks. The training set for this optimization contained all of the isolated word data (from the cross-validation and optimization subsets) except for the trials in this held-out set. We used the resulting optimal hyperparameter value combination during offline optimization of other hyperparameters related to sentence decoding and during online sentence decoding.

Optimization with Sentence Data

Using the sentence optimization subset, we performed hyperparameter optimization of the threshold detection hyperparameters (see Method S8), the initial word smoothing value (for the language model; see Method S10), and the language model scaling factor (for the Viterbi decoder; see Method S11). In this procedure, we first used the speech detector (trained on all isolated word data, including the isolated word optimization subset) to predict speech probabilities for all of the sentence optimization blocks. Then, using these predicted speech probabilities, the word classifier trained and optimized on the isolated word data for use during sentence decoding, and the language model and Viterbi decoder, we performed hyperparameter optimization across all optimization sentence blocks (see Method S6). We used the mean decoded word error rate across trials (computed by evaluating the detected events in each trial with the word classifier, language model, and Viterbi decoder) as the objective function during hyperparameter optimization. Using the hyperopt Python package [9], we evaluated 100 hyperparameter value combinations during optimization. We used the resulting optimal hyperparameter value combination during collection of the sentence testing blocks with online decoding.

### Method S8. Speech Detection Model

### Data Preparation for Offline Training and Evaluation

For supervised training and evaluation of the speech detector with the isolated word data, we assigned speech event labels to the neural time points. We used the task timing information during these blocks to determine the label for each neural time point. We used three types of speech event labels: preparation, speech, and rest.

Within each isolated word trial, the target utterance appeared on the screen with the countdown animation, and 2 seconds later the utterance turned green to indicate the go cue. We labeled all neural time points collected during this 2-second window ([−2, 0] seconds relative to the go cue) as preparation. Relative to the go cue, we labeled neural time points collected between [0.5, 2] seconds as speech and points collected between [3, 4] as rest. To reduce the impact that variability in the participant's response times had on training, we excluded the time periods of [0, 0.5] and [2, 3] seconds relative to the go cue (the time periods surrounding the speech time period) from the training datasets. During evaluation, these time periods were labeled as preparation and rest, respectively.

We included the preparation label to enable the detector to neurally disambiguate attempted speech production from speech preparation. This was motivated by the assumption that neural activity related to attempted speech production would be more readily discriminable by the word classifier than activity related to speech preparation.

### Speech Detection Model Architecture and Training

We used the PyTorch 1.6.0 Python package to create and train the speech detection model [12].

The speech detection architecture was a stack of three long short-term memory (LSTM) layers with decreasing latent dimension sizes (150, 100, and 50) and a dropout of 0.5 applied at each layer. Recurrent layers are capable of maintaining an internal state through time that can be updated with new individual time samples of input data, making them well suited for real-time inference with temporally dynamic processes [13]. We use LSTMs specifically because they are better suited to model long-term dependencies compared to the original recurrent layer. The LSTMs are followed by a fully connected layer to project the last latent dimensions to probabilities across the three classes (rest, speech, and preparation). A similar model has been used to detect overt speech in a recent study [14], although our architecture was designed independently. A schematic depiction of this architecture is given in FIG. 9.

Let y denote a series of neural data windows and l denote a series of corresponding labels for those windows, with yₙ as the data window at index n in the data series and lₙ as the corresponding label at index n in the label series. The speech detection model outputs a distribution of probabilities Q(lₙ|yₙ) over the three possible values of lₙ from the set of state labels L={rest, preparation, speech}. The predicted distribution Q implicitly depends on the model parameters. We trained the speech detection model to minimize the cross-entropy loss of this distribution with respect to the true distribution using the data and label series, represented by the following equation:

$${H}_{P , Q} ( ℓ \vert y ) \approx - \frac{1}{N} {\sum }_{n}^{N} {w}_{fp , n} log Q ( {ℓ}_{n} \vert {y}_{n} ) , \qquad ( S 1 )$$

with the following definitions:
P: The true distribution of the states, determined by the assigned state labels f.
N: The number of samples.
H_{P,Q}( [symbol] |y): The cross entropy of the predicted distribution with respect to the true distribution for [symbol] .
log: The natural logarithm.

Here, we approximate the expectation of the true distribution with a sample average under the observed data with N samples.

During training, a false positive weighting of 0.75 was applied to any frame where the speech label was falsely predicted. With this modification, the cross-entropy loss from Equation S1 is redefined as:

$$\begin{matrix}{H}_{P , Q} ( ℓ \vert y ) = {𝔼}_{P} [ - log Q ( ℓ \vert y ) ] \\ \approx - \frac{1}{N} {\sum }_{n = 1}^{N} log Q ( {ℓ}_{n} \vert y ) ,\end{matrix} \qquad ( S 2 )$$

where w_{fp,n} is the false positive weight for sample n and is defined as:

$${w}_{fp , n} := \{ \begin{matrix}0.75 & if ( {ℓ}_{n} \neq speech ) and ( {argmax}_{l ∈ L} [ Q ( l \vert {y}_{n} ] = speech ) \\ 1 & otherwise\end{matrix} . \qquad ( S 3 )$$

As a result of this weighting, the loss associated with a sample that was incorrectly classified as occurring during a speech production attempt was only weighted 75% as much as the other samples. This weighting was only applied during training of speech detection models that were used to evaluate isolated word data. We applied this weighting to encourage the model to prefer detecting full speech events, which discouraged fluctuating speech probabilities during attempted speech productions that could prevent a production attempt from being detected. This effectively increased the number of isolated word trials that had an associated detected speech event during training and evaluation of the word classifier.

Typically, LSTM models are trained with backpropagation through time (BPTT), which unrolls the backpropagation through each time step of processing [15]. Due to the periodicity of our isolated word task structure, it is possible that relying only on BPTT would cause the model to learn this structure and predict events at every go cue instead of trying to learn neural indications of speech events. To prevent this, we used truncated BPTT, an approach that limits how far back in time the gradient can propagate [16, 17]. We manually implemented this by defining 500 ms sliding windows in the training data. These windows were highly overlapping, shifting by only one neural sample (5 ms) between windows. We used these windows as the yₙ values during training, with lₙ equal to the label assigned to the final time point in the window. By processing the training data in windows, this forced the gradient to only backpropagate 500 ms at a time, which was not long enough to learn the periodicity of the task (the time between each trial's go cue was typically 7 seconds). During online and offline inference, the data was not processed in windows and was instead processed time point by time point.

During training, we used the Adam optimizer to minimize the cross-entropy given in Equation S2 [18], with a learning rate of 0.001 and default values for the remaining Adam optimization parameters. When evaluating the speech detector on isolated word data, we used the 10-fold cross-validation scheme described in Method S6. When performing offline and online inference on sentence data, we used a version of the speech detector that was trained on all of the isolated word data in the 10 cross-validation subsets. During training, the training set was further split into a training set and a validation set, where the validation set was used to perform early stopping. We trained the model until model performance did not improve (if cross entropy loss on the validation set was not lower than the lowest value plus a loss tolerance value computed in a previous epoch) for 5 epochs in a row and at least 10 epochs had been completed, at which point model training was stopped and the model parameters associated with the lowest loss were saved. The loss tolerance value was set to 0.001, although it did not seem to have significant impact on model training.

### Speech Event Detection

During testing, the neural network predicted probabilities for each class (rest, preparation, speech) given the input neural data from a block. To detect attempted speech events, we applied thresholding to the predicted speech probabilities. This thresholding approach is identical to the approach we used in our previous work [2]. First, we smoothed the probabilities using a sliding window average. Next, we applied a threshold to the smoothed probabilities to binarize each frame (with a value of 1 for speech and 0 otherwise). Afterwards, we “debounced” these binarized values by applying a time threshold. This debouncing step required that a change in the presence or absence of speech (as indicated by the binarized values) be maintained for a minimum duration before the detector deemed it as an actual change. Specifically, a speech onset was only detected if the binarized value changed from 0 to 1 and remained 1 for a pre-determined number of time points (or longer). Similarly, a speech offset was only detected if the binarized value changed from 1 to 0 and remained 0 for the same pre-determined number of time points (or longer). This process of obtaining speech events from the predicted probabilities was parameterized by three detection thresholding hyperparameters: the size of the smoothing window, the probability threshold value, and the time threshold duration. We used hyperparameter optimization to determine values for these parameters (see the following section and Method S7).

### Detection Score and Hyperparameter Optimization

During hyperparameter optimization of the detection thresholding hyperparameters with the isolated word data, we used an objective function derived from a variant of the detection score metric used in our previous work [2]. The detection score is a weighted average of frame-level and event-level accuracies for each block.

The frame-level accuracy measures the speech detector's ability to predict whether or not a neural time point occurred during speech. Ideally, the speech detector would detect events that spanned the duration of the actual attempted speech event (as opposed to detecting small subsets of each actual speech event, for example). We defined frame-level accuracy a_{frame} as:

$${a}_{frame} := \frac{{w}_{p} {F}_{TP} + ( 1 - {w}_{p} ) {F}_{TN}}{{w}_{p} {F}_{P} + ( 1 - {w}_{p} ) {F}_{N}} ,$$

with the following variable definitions:
wₚ: The positive weight fraction, which we used to control the importance of correctly detecting positive frames (correctly identifying which neural time points occurred during attempted speech) relative to negative frames (correctly identifying which neural time points did not occur during attempted speech).
F_{P}: The number of actual positive frames (the number of time points that were assigned the speech label during data preparation).
F_{TP}: The number of detected true positive frames (the number of time points that were correctly identified as occurring during an attempted speech event).
F_{N}: The number of actual negative frames (the number of time points that were labeled as preparation or rest during data preparation).
F_{TN}: The number of detected true negative frames (the number of time points that were correctly identified as not occurring during an attempted speech event).

In this work, we used wₚ=0.75, which encouraged the speech detector to prefer making false positive errors to making false negative errors.

The event-level accuracy measures the detector's ability to detect a speech event during an attempted word production. We defined event-level accuracy aₑᵥₑₙₜ as:

$${a}_{event} := max ( 0 , \frac{{E}_{TP} - {E}_{FP} - {E}_{FN}}{{E}_{P}} ) , \qquad ( S 4 )$$

with the following variable definitions:
E_{TP}: The number of true positive detected events (the number of detected speech events that corresponded to an actual word production attempt).
E_{FP}: The number of false positive detected events (the number of detected speech events that did not correspond to an actual word production attempt).
E_{FN}: The number of false negative events (the number of actual word production attempts that were not associated with any detected event).
E_{P}: The number of actual word production attempts (the number of trials).

We calculated event-level accuracy after curating the detected events, which involved matching each trial with a detected event (or the absence of a detected event; see the following section for more details). The event-level accuracy ranges from 0 to 1, with a value of 1 indicating that there were no false positive or false negative detected events.

Using these two accuracy measures, we compute the detection score as:

$${s}_{detection} = {w}_{F} {a}_{frame} + ( 1 - {w}_{F} ) {a}_{event} ,$$

where w_{F} is the frame-level accuracy weight. Because the word classifier relied on fixed-duration time windows of neural activity relative to the detected onsets of the speech events, accurately predicting the detected offsets was less important than successfully detecting an event each time the participant attempted to produce a word. Informed by this, we set w_{F}=0.4 to assign more weight to the event-level accuracy than the frame-level accuracy.

During optimization of the three detection thresholding hyperparameters with the isolated word data, the primary goal was to find hyperparameter values that maximized detection score. We also included an auxiliary goal to select small values for the time threshold duration hyperparameter. We included this auxiliary goal because a large time threshold duration increases the chance of missing shorter utterances and, if the duration is large enough, adds delays to real-time speech detection. The objective function used during this hyperparameter optimization procedure, which encapsulated both of these goals, can be expressed as:

$${c}_{hp} ( Θ ) = {( 1 - {s}_{detection} )}^{2} + {λ}_{time} {θ}_{time} , \qquad ( S 5 )$$

with the following variable definitions:
cₕₚ (Θ): The value of the objective function using the hyperparameter value combination Θ.
λₜᵢₘₑ: The penalty applied to the time threshold duration.
θₜᵢₘₑ: The time threshold duration value, which is one of the three parameters contained in Θ. Here, we used λₜᵢₘₑ=0.00025.

We only used this objective function during optimization of the detection models that were used to detect speech events for the isolated word trials. We used a different objective function when preparing detection models for use with the sentence data. See Method S7 and Table S1 for more information on the hyperparameter optimization procedures.

### Detected Event Curation for Isolated Word Data

After processing the neural data for an isolated word block and detecting speech events, we curated the detected events to match each one to an actual word production attempt (and to identify word production attempts that did not have a corresponding detected event and detected events that did not correspond to a word production attempt). We used this curation procedure to measure the number of false positive and false negative event detections during calculation of the event-level accuracy (Equation S4) and to match trials to neural data during training and evaluation of the word classifier. We did not use this curation procedure with sentence data.

To curate detected events, we performed the following steps for each trial: We identified all of the detected onsets that occurred in a time window spanning from −1.5 to 3.5 seconds (relative to the go cue). Any events with detected onsets outside of this time window were considered false positive events and were included when computing the value of E_{FP}.

If there was exactly one detected onset in this time window, we assigned the associated detected event to the trial.

Otherwise, if there were no detected onsets in this time window, we did not assign a detected event to the trial (this was considered a false negative event and was included when computing the value of E_{FN}).

Otherwise, there were two or more detected onsets in this time window, and we performed the following steps to process these detected events:

If exactly one of these detected onsets occurred after the go cue, we assigned the detected event associated with that detected onset to the trial.

Otherwise, if none of these detected onsets occurred after the go cue, we assigned the detected event associated with the latest detected onset to the trial (this was the detected event that had the detected onset closest to the go cue).

Otherwise, if two or more detected onsets occurred after the go cue, we computed the length of each detected event associated with these detected onsets and assigned the longest detected event to the trial. If a tie occurred, we assigned the detected event with an onset closest to the go cue to the trial.

Each of these detected events that were not assigned to the trial were considered false positive events and were included when computing the value of E_{FP}.

Because false negatives cause some trials to not be associated with a detected event, the number of trials that actually get used in an analysis step may be less than the number of trials reported. For example, if we state that N trials of each word were used in an analysis step, the actual number of trials analyzed by the word classifier in that step may be less than N for one or more words depending on how many false negative detections there were.

### Method S9. Word Classification Model

### Data Preparation for Offline Training and Evaluation

During training and evaluation of the word classifier with the isolated word data, for each trial we obtained the time of the detected onset (if available; determined by the detection curation procedure described in Method S8). During evaluation with each trial, the word classifier predicted the probability of each of the 50 words being the target word that the participant was attempting to produce given the time window of high gamma activity spanning from −1 to 3 seconds relative to the detected onset.

To increase the number of training samples and improve robustness of the learned feature mapping to small temporal variabilities in the neural inputs, during model fitting we augmented the training dataset with additional copies of the trials by jittering the onset times, which is analogous to the well-established use of data augmentation techniques used to train neural networks for supervised image classification [19]. Specifically, for each trial, we obtained the neural time windows spanning from (−1+a) to (3+a) seconds relative to the detected onset for each a∈{−1, −0.667, −0.333, 0, 0.333, 0.667, 1}. Each of these time windows was included as a training sample and was assigned the associated target word from the trial as the label.

During offline and online training and evaluation, we downsampled the high gamma activity in each time window before passing the activity to the word classifier, which has been shown to improve speech decoding with artificial neural networks (ANNs) in our previous work [20]. We used the decimate function within the SciPy Python package to decimate the high gamma activity for each electrode by a factor of 6 (from 200 Hz to 33.3 Hz) [21]. This function applies an 8^{th}-order Chebyshev type I anti-aliasing filter before decimating the signals. After decimation, we normalized each time sample of neural activity such that the Euclidean norm across all electrodes was equal to 1.

### Word Classification Model Architecture and Training

We used the TensorFlow 1.14 Python package to create and train the word classification model [22].

Within the word classification ANN architecture, the neural data was processed by a temporal convolution with a two-sample stride and two-sample kernel size, which further downsampled the neural activity in time while creating a higher-dimensional representation of the data. Temporal convolution is a common approach for extracting robust features from time series data [23]. This representation was then processed by a stack of two bidirectional gated recurrent unit (GRU) layers, which are often used for nonlinear classification of time series data [24]. Afterwards, a fully connected (dense) layer with a softmax activation projects the latent dimension from the final GRU layer to probability values across the 50 words. Dropout layers are used between each intermediate representation for regularization. A schematic depiction of this architecture is given in FIG. 10.

Let y denote a series of high gamma time windows and w denote a series of corresponding target word labels for those windows, with yₙ as the time window at index n in the data series and wₙ as the corresponding label at index n in the label series. The word classifier outputs a distribution of probabilities Q(wₙ|yₙ) over the 50 possible values of wₙ from the 50-word set W. The predicted distribution Q implicitly depends on the model parameters. We trained the word classifier to minimize the cross-entropy loss of this distribution with respect to the true distribution using the data and label series, represented by the following equation:

$$\begin{matrix}{H}_{P , Q} ( w \vert y ) = {𝔼}_{P} [ - log Q ( w \vert y ) ] \\ \approx - \frac{1}{N} {\sum }_{n = 1}^{N} log Q ( {w}_{n} \vert {y}_{n} ) ,\end{matrix} \qquad ( S 6 )$$

with the following definitions:
P: The true distribution of the labels, determined by the assigned word labels w.
N: The number of samples.
H_{P,Q}(w|y): The cross entropy of the predicted distribution with respect to the true distribution for w.
log: The natural logarithm.

Here, we approximate the expectation of the true distribution with a sample average under the observed data with N samples.

During training, we used the Adam optimizer to minimize the cross entropy given in Equation S6 [18], with a learning rate of 0.001 and default values for the remaining Adam optimization parameters. Each training set was further split into a training set and a validation set, where the validation set was used to perform early stopping. We trained the model until model performance did not improve (if cross-entropy loss on the validation set was not lower than the lowest value computed in a previous epoch) for 5 epochs in a row, at which point model training was stopped and the model parameters associated with the lowest loss were saved. Training typically lasted between 20 and 30 epochs. When applying gradient updates to the model parameters after each epoch, if the Euclidean norm of the gradient across all of the parameter update values (before scaling these values with the learning rate) was greater than 1, then, to prevent exploding gradients, the gradient was normalized such that its Euclidean norm was equal to 1 [25].

To reduce overfitting on the training data, each word classifier contained an ensemble of 10 ANN models, each with identical architectures and hyperparameter values but with different parameter values (weights) [26]. During training, each ANN was initialized with random model parameter values and was individually fit using the same training samples, although each ANN processed the samples in a different order during stochastic gradient updates. This process yielded 10 different sets of model parameters. During evaluation, all 10 of the ensembled ANNs processed each input neural time window, and we averaged the predicted distribution Q(wₙ|yₙ) for each ANN to compute the overall predicted word probabilities for each of the 50 possible values of wₙ given the neural time window yₙ.

We used a hyperparameter optimization procedure to select values for model parameters that were not directly learned during training. We computed two different hyperparameter value combinations: one for offline isolated word analyses and one for online sentence decoding. For faster hyperparameter searching, we used ensembles of 4 ANN models when searching for hyperparameters rather than the full set of 10. See Method S7 and Table S1 for more details.

### Modifications for the Sentence Task

For online sentence decoding, we trained a modified version of the word classifier on all of the isolated word data. During hyperparameter optimization for this version of the word classifier, the held-out set contained 4 trials of each word randomly sampled from blocks collected near the end of the study period (see Method S7 for more details). After hyperparameter optimization, we then trained a word classifier with the selected hyperparameters by using this held-out set of 4 trials of each word as the validation set (used to perform early stopping) and all of the remaining isolated word data as the training set. During this training procedure, we added a single modification to the loss function used during training: We weighted each training sample by the occurrence frequency of the target word label within a corpus consisting only of words from the 50-word set. Words that occurred more frequently were assigned more weight. The corpus used to compute word occurrence frequency is the same corpus that was crowdsourced from Amazon Mechanical Turk and used to the train the language model (see Method S4). We included this modification to encourage the word classifier to focus on correctly classifying neural time windows detected during attempted production of high-frequency words (such as “I”), at the cost of classification performance for low-frequency words (such as “glasses”).

With this modification, the loss function from Equation S6 can be revised to:

$${H}_{P , Q}^{′} ( w \vert y ) \approx - \frac{1}{N} {\sum }_{n = 1}^{N} ξ ( {w}_{n} ) log Q ( {w}_{n} \vert {y}_{n} ) , \qquad ( S 7 )$$

with the following variable definitions:
H′_{P,Q} (w y): The revised cross-entropy loss function.
ξ(wₙ): The word occurrence frequency weighting function

The word occurrence frequency weighting function is defined as:

$$ξ ( {w}_{n} ) := \frac{{κ}_{{w}_{n}}}{\overline{ξ} {\sum }_{w ∈ W} {κ}_{w}} \qquad ( S 8 )$$

where K_{w}_{ₙ} is the number of times the target word label wₙ occurred in the reference corpus,

$${\sum }_{w ∈ W} {κ}_{ω}$$

is the total number of words in the reference corpus, and W is the 50 word set.

We define ξ̅ as:

$$\overline{ξ} := \frac{1}{\vert W \vert} {\sum }_{{w}_{i} ∈ W} \frac{{κ}_{{w}_{i}}}{{\sum }_{w ∈ W} {κ}_{w}} \qquad ( S 9 )$$

where W denotes the cardinality of the 50-word set (which is equal to 50). Therefore, ξ̅ acts to scale each word frequency in Equation S8 so that the mean word occurrence frequency is 1, which scales the objective function such that the loss value is comparable with the loss value resulting from Equation S6.

### Method S10. Language Modeling

### Model Fitting and Word-Sequence Probabilities

To fit a language model for use during sentence decoding, we first crowdsourced a training corpus using an Amazon Mechanical Turk task (view Method S4 for more details). This corpus contained 3415 sentences comprised only of words from the 50-word set. To discourage overfitting of the language model on the most common sentences, we only included a maximum of 15 instances of each unique sentence in the training corpus created from these responses.

Next, we extracted all n-grams with n∈{1, 2, 3, 4, 5} from each sentence in the training corpus. Here, an n-gram is a word sequence with a length of n words [27]. For example, the n-grams (represented as tuples) extracted from the sentence “I hope my family is coming” in this approach would be:
1. (I)
2. (Hope)
3. (My)
4. (Family)
5. (Is)
6. (Coming)
7. (I, Hope)
8. (Hope, My)
9. (My, Family)
10. (Family, Is)
11. (Is, Coming)
12. (I, Hope, My)
13. (Hope, My, Family)
14. (My, Family, Is)
15. (Family, Is, Coming)
16. (I, Hope, My, Family)
17. (Hope, My, Family, Is)
18. (My, Family, Is, Coming)
19. (I, Hope, My, Family, Is)
20. (Hope, My, Family, Is, Coming)

We used the n-grams extracted in this manner from all of the sentences in the training corpus to fit a 5^{th}-order interpolated Kneser-Ney n-gram language model with the nltk Python package [28, 29]. A discount factor of 0.1 was used for this model, which was the default value specified within nltk. The details of this language model architecture, along with characterizations of its ability to outperform simpler n-gram architectures on various corpus modeling tasks, can be found in existing literature [27, 28].

Using the occurrence frequencies of specific word sequences in the training corpus (as specified by the extracted n-grams), the language model was trained to yield the conditional probability of any word occurring given the context of that word, which is the sequence of (n−1) or fewer words that precede it. These probabilities can be expressed as p(wᵢ|c_{i,n}), where wᵢ is the word at position i in some word sequence, c_{i,n} is the context of that word assuming it is part of an n-gram (this n-gram is a word sequence containing n words, with wᵢ as the last word in that sequence), and n∈{1, 2, 3, 4, 5}. The context of a word wᵢ is defined as the following tuple:

$${c}_{i , n} := ( {w}_{i - ( n - 1 )} , … , {w}_{i - 1} ) . \qquad ( S 10 )$$

When n=1, the context is ( ), an empty tuple. When n=2, the context of a word wᵢ is (wᵢ−1), a single-element tuple containing the word preceding wᵢ. With the language model used in this work, this pattern continues up to n=5, where the context of a word wᵢ is (wᵢ−4, wᵢ−3, wᵢ−2, wᵢ−1), a tuple containing the four words in the sequence that precede wᵢ (in order). It was required that each wᵢ∈W, where W is the 50-word set. This requirement included the words contained in the contexts c_{i,n}.

### Sentence Independence

During the sentence task, each sentence was decoded independently of the other sentences in the task block. The contexts c_{i,n} that we used during inference with the language model could only contain words that preceded, but were also in the same sentence as, wᵢ (contexts never spanned two or more sentences). The relationship between the values i and n in the contexts we used during inference can be expressed as:

$$n = min ( i + 1 , m ) , \qquad ( S 11 )$$

where m is the order of the model (for this model, m=5) and i=0 specifies the index of the initial word in the sentence. Substituting this definition of n into the definition for c_{i,n} specified in Equation S10 yields:

$${c}_{i} := ( {w}_{i - min ( i , m - 1 )} , … , {w}_{i - 1} ) \qquad ( S 12 )$$

where cᵢ is the context of word wᵢ within a sentence trial. This substitution simplifies the form of the word probabilities obtained from the language model to p(wᵢ|cᵢ).

### Initial Word Probabilities

Because sentences were always decoded independently in this task, an empty tuple was only used as context when performing inference for w₀, the initial word in a sentence. Instead of using the values for p(w₀|c₀) yielded by the language model during inference, we instead used word counts directly from the corpus and two different types of smoothing. First, we computed the following probabilities:

$$ϕ ( {w}_{0} \vert {c}_{0} ) = \frac{{k}_{{w}_{0}} + δ}{N + δ \vert W \vert} , \qquad ( S 13 )$$

where k_{w0} is the number of times that the word w₀ appeared as the initial word in a sentence in the training corpus, N is the total number of sentences in the training corpus, and 8 is an additive smoothing factor. Here, the additive smoothing factor is a value that is added to all of the counts k_{w0} prior to normalization, which smooths (reduces the variance of) the probability distribution [27]. In this work, N=3415, 6=3, and W=50.

We then smoothed these ϕ(w₀|c₀) values to further control how flat the probability distribution over the initial word probabilities was. This can be interpreted as control over how “confident” the initial word probability predictions by the language model were (flatter probability distributions indicate less confidence). We used a hyperparameter to control the extent of this smoothing, allowing the hyperparameter optimization procedure to determine how much smoothing was optimal during testing (see Method S7 and Table S1 for a description of the hyperparameter optimization procedure). We used the following equation to perform this smoothing:

$$p ( {w}_{0} \vert {c}_{0} ) = \frac{{ϕ ( {w}_{0} \vert {c}_{0} )}^{ψ}}{{\sum }_{{w}_{j} ∈ W} {ϕ ( {w}_{j} \vert {c}_{0} )}^{ψ}} , \qquad ( S14 )$$

where ψ is the initial word smoothing hyperparameter value. When ψ>1, the variance of the initial word probabilities is increased, making them less smooth. When ψ<1, the variance of the initial word probabilities is decreased, making them smoother. When ψ=1, p(w₀|c₀)=ϕ (w₀|c₀). Note that the denominator in Equation S14 is used to re-normalize the smoothed probabilities so that they sum to 1.

The Viterbi decoding model used in this work contained a language model scaling factor (LMSF), which is a separate hyperparameter that re-scaled the p(wᵢ|cᵢ) values during the sentence decoding approach (see Method S11 for more details). The effect that this hyperparameter had on all of the language model probabilities resembles the effect that ψ had on the initial word probabilities. This should have encouraged the hyperparameter optimization procedure to find an LMSF value that optimally scaled the language model probabilities and a value for that optimally smoothed the initial word probabilities relative to the scaling that was subsequently applied to them.

### Real-Time Implementation

To ensure rapid inference during real-time decoding, we pre-computed the p(wᵢ|cᵢ) values with the language model and smoothing hyperparameter values for every possible combination of wᵢ and cᵢ and then stored these values in an hdf5 file [30]. This file served as a lookup table during real-time decoding; the values were stored in multi-dimensional arrays within the file, and efficient lookup queries to the table were fulfilled during real-time decoding using the h5py Python package [31]. In future iterations of this decoding approach requiring larger vocabulary sizes, it may be more suitable to use a more sophisticated language model that is also computationally efficient enough for real-time inference, such as the kenlm language model [32].

### Method S11. Viterbi Decoding

### Hidden Markov Model Representation of the Sentence Decoding Procedure

During a sentence trial, the relationship between the sequence of words that the participant attempted to produce and the sequence of neural activity time windows provided by the speech detector can be represented as a hidden Markov model (HMM). In this HMM, each observed state yᵢ is the time window of neural activity at index i within the sequence of detected time windows for any particular trial, and each hidden state qᵢ is the n-gram containing the words that the participant had attempted to produce from the first word to the word at index i in the sequence (FIG. 11). Here, qᵢ={wᵢ,cᵢ}, where wᵢ is the word at index i in the sequence and cᵢ is the context of that word (defined in Equation S12; see Method S10).

The emission probabilities for this HMM are p(yᵢ|qᵢ), which specify the likelihood of observing the neural time window yᵢ given the n-gram qᵢ. With the assumption that the time window of neural activity associated with the attempted production of wᵢ is conditionally independent of all of the other attempted word productions given wᵢ(yᵢ⊥wⱼ|wᵢ∀j≠i), p(yᵢ|q) simplifies to p(yᵢ|wᵢ). The word classifier provided the probabilities p(wᵢ|yᵢ), which was used directly as the values for p(yᵢ|wᵢ) by applying Bayes' theorem and assuming a flat prior probability distribution.

The transition probabilities for this HMM are p(qᵢ|qᵢ₋₁), which specify the probability that qᵢ is the n-gram at index i (the sequence of at most n words, containing wᵢ as the final word, that the participant attempted to produce) given that the n-gram at index (i−1) was qᵢ₋₁. Here, q₋₁ can be defined as an empty set, indicating that q₀ is the first word in the sequence. Because any elements in cᵢ will be contained in qᵢ₋₁ and to, is the only word in qᵢ that is not contained in qᵢ₋₁, p(qᵢ|qᵢ₋₁) simplifies to p(wᵢ|cᵢ), which were the word sequence prior probabilities provided by the language model. Implicit in this simplification is the assertion that p(qᵢ|qᵢ₋₁)=0 if qᵢ is incompatible with qᵢ₋₁ (for example, if the final word in cᵢ is not equal to the second-to-last word in qᵢ₋₁).

### Viterbi Decoding Implementation

To predict the words that the participant attempted to produce during the sentence task, we implemented a Viterbi decoding algorithm with this underlying HMM structure. The Viterbi decoding algorithm uses dynamic programming to compute the most likely sequence of hidden states given hidden-state prior transition probabilities and observed-state emission likelihoods [33, 34]. To determine the most likely hidden-state sequence, this algorithm iteratively computes the probabilities of various “paths” through the hidden-state sequence space (various combinations of qᵢ values). Here, each of these Viterbi paths was parameterized by a particular path through the hidden states (a particular word sequence) and the probability associated with that path given the neural activity. Each time a new word production attempt was detected, this algorithm created a set of new Viterbi paths by computing, for each existing Viterbi path, the probability of transitioning to each valid new word given the detected time window of neural activity and the preceding words in the associated existing Viterbi path. The creation of new Viterbi paths from existing paths can be expressed using the following recursive formula:

$${V}_{i} = \{ {v}_{i - 1} + log p ( {y}_{i} \vert {q}_{i , {v}_{i - 1}} ) + L log p ( {q}_{i , {v}_{i - 1}} \vert {q}_{i - 1 , {v}_{i - 1}} ) \vert \text{} {v}_{i - 1} ∈ {V}_{i - 1} ∧ {w}_{i} ∈ W \} , \qquad ( S15 )$$

with the following variable definitions:
Vⱼ: The set of all Viterbi paths created after the word production attempt at index j within a sentence trial.
vⱼ: A Viterbi path within Vj. Each of these Viterbi paths was parameterized by the n-grams (q₀, . . . , qⱼ) (or, equivalently, the words (w₀, . . . , wⱼ)) and the log probability of that sequence of words occurring given the neural activity, although these equations only describe the recursive computation of the log probability values (the tracking of the words associated with each Viterbi path is implicitly assumed).
q_{j,vk}: The n-gram qⱼ, containing the word wⱼ and the context of that word. This context is determined from the most recent words within the hidden state sequence of Viterbi path vₖ.
p(y|qⱼ|q_{j,vj-1}): The emission probability specifying the likelihood of the observed neural activity y₁ given the n-gram q_{j,vj-1}.
p(q_{i,vi-1}|q_{i-1,vi-1}): The transition probability specifying the prior probability of transitioning to the n-gram q_{i,vi-1}, from the n-gram q_{i-1,vi-1}.
L: The language model scaling factor, which is a hyperparameter that we used to control the weight of the transition probabilities from the language model relative to the emission probabilities from the word classifier (see Method S7 and Table S1 for a description of the hyperparameter optimization procedure).
W: The 50-word set.
log: The natural logarithm.

Using the simplifications described in the previous section, Equation S15 can be simplified to the following equation:

$${V}_{i} = \{ {v}_{i - 1} + log p ( {w}_{i} \vert {y}_{i} ) + L log p ( {w}_{i} \vert {c}_{i , {v}_{i - 1}} ) \vert \text{} {v}_{i - 1} ∈ {V}_{i - 1} ∧ {w}_{i} ∈ W \} , \qquad ( S16 )$$

where c_{j,vk} is the context of word wj determined from the Viterbi path vₖ, p(wᵢ|yᵢ) are the emission probabilities (obtained from the word classifier), and p(wᵢ|c_{i,vi-1}), are the transition probabilities (obtained from the language model). At the start of each sentence trial, the index i was reset to zero (the first word in each trial was denoted w₀), and any existing Viterbi paths from a previous trial were discarded. To initialize the recursion, we defined V₋₁ as a singleton set containing a single Viterbi path with the empty set as its hidden state sequence and an associated log probability of zero. We used log probabilities in practice for numerical stability and computational efficiency.

### Viterbi Path Pruning Via Beam Search

As specified in Equation S16, when new emission probabilities p(wᵢ|yᵢ) were obtained from the word classifier, our Viterbi decoder computed the new set of Viterbi paths Vᵢ, comprised of the paths created by transitioning each existing path within Vᵢ₋₁ to each possible next n-gram qᵢ. As a result, the number of new Viterbi paths created for index i was equal to |Vᵢ₋₁×W| (the number of existing Viterbi paths at index i−1 multiplied by 50). Without intervention, the number of Viterbi paths grows exponentially as the index increases (|Vᵢ|=|W|⁽ⁱ⁺¹⁾).

To prevent exponential growth, we applied a beam search with a beam width of β to each new Viterbi path set Vᵢ immediately after it was created. This beam search enforced a maximum size of β for each new Viterbi path set, retaining the β most likely paths (the paths with the greatest associated log probabilities) and pruning (discarding) the rest. All paths were retained if |Vᵢ|≤β. Expanding Equation S16 to include the beam search procedure yields the final set of Viterbi decoding update equations that we used in practice during sentence decoding:

$${V}_{i}^{′} = \{ {v}_{i - 1} + log p ( {w}_{i} \vert {y}_{i} ) + L log p ( {w}_{i} \vert {c}_{i , {v}_{i - 1}} ) \vert \text{} {v}_{i - 1} ∈ {V}_{i - 1} ∧ {w}_{i} ∈ W \} \qquad ( S17 )$$

$${V}_{i} = \{ {v}_{i , j} \vert j ∈ \{ 0 , … , min ( β , \vert {V}_{i}^{′} \vert ) - 1 \} \} , \qquad ( S18 )$$

where V is the set of all Viterbi paths created after the word production attempt at index i within a sentence trial (before pruning) and v_{1,j} is the element at index j of a vector created by sorting the Viterbi paths in V in order of descending log probability (ties are broken arbitrarily during sorting).

### Method S12. Sentence Decoding Evaluations

We evaluated the performance of our decoding pipeline (speech detector, word classifier, language model, and Viterbi decoder) using the online predictions made during the sentence task blocks (in the testing subset; see Method S6). Specifically, we analyzed the sentences decoded in real time from the participant's neural activity during the active phase of each trial (the portion of each trial during which the participant was instructed to attempt to produce the prompted sentence target). Offline, we counted the number of false positive speech events that were erroneously detected during inactive task phases (which were ignored during real-time decoding). These false positive events only occurred before the first trial in a block, and this count is reported in the Results section of the main text.

### Word Error Rates and Edit Distances

To measure the quality of the decoding results, we computed the word error rates (WERs) between the target and decoded sentences in each trial. WER is a commonly used metric to measure the quality of predicted word sequences, computed by calculating the edit (Levenshtein) distance between a reference (target) and decoded sentence and then dividing the edit distance by the number of words in the reference sentence. Here, the edit distance measurement can be interpreted as the number of word errors in the decoded sentence (in FIG. 2 in the main text, the edit distance is referred to as the “number of word errors” or the “error count”). It is computed as the minimum number of insertions, deletions, and substitutions required to transform the decoded sentence into the reference sentence. Below we demonstrate each type of edit operation that can be used to transform an example decoded sentence (on the left side of each arrow) into the target sentence “I am good”. In each case, the example decoded sentence has an edit distance of 1 to the target sentence.
Insertion: I good→I am good
Deletion: I am very good→I am good
Substitution: I am going→I am good

Lower edit distances and WERs indicate better performance. We computed edit distances and WERs using predictions made with and without the language model and Viterbi decoder.

To compute block-level WERs, which are shown in FIG. 2A in the main text, we first computed the edit distance for each sentence trial (which are shown in FIG. 2D in the main text). We then computed the block-level WER as the sum of the edit distances across all of the trials in a test block divided by the sum of the target-sentence word lengths across all trials. This approach to measure block-level WER was preferred to simply averaging trial-level WER values because it does not overvalue short sentences compared to long ones. For example, if we simply averaged trial-level WERs to compute a block-level WER, then one error in a trial with the target sentence “I am thirsty” would cause a greater impact on WER than one error in a trial with the target sentence “My family is very comfortable”, which was not a desired aspect of our block-level WER measurement.

To assess chance performance of our decoding approach with the sentence task, we measured WER using randomly generated sentences from the language model and Viterbi decoder (independent of any neural data). To generate these sentences, we performed the following steps for each trial:
Step 1: Start with an empty word sequence.
Step 2: Acquire the word probabilities from the language model using the current word sequence as context.
Step 3: Randomly sample a word from the 50-word set, using the word probabilities in step 2 as weights for the sampling.
Step 4: Add the word from step 3 to the current word sequence.
Step 5: Repeat steps 2-4 until the length of the current word sequence is equal to the length of the target sentence for the trial.

With the randomly generated sentence for each trial, we measured chance performance by computing block-level WERs using the method described in the preceding paragraph. Note that this method of measuring chance performance overestimates the true chance performance because it uses the language model and the same sentence length as the target sentence for each trial (which is equivalent to assuming that the speech detection model always detected the correct number of words in each trial).

### Words Per Minute and Decoded Word Correctness

To measure decoding rate, we used the words per minute (WPM) metric. For each trial, we computed a WPM value by counting the number of detected words in the trial and dividing that count by the detected trial duration. We calculated each detected trial duration as the elapsed time between the time at which the sentence prompt appeared on the participant's monitor (the go cue) and the time of the last neural time sample passed from the speech detector to the word classifier in the trial.

To measure the rate at which words were accurately decoded, we also computed WPMs while only counting correctly decoded words. To determine which words were correctly decoded in each trial, we performed the following steps:
Step 1: Start with n=1 and w=0.
Step 2: Compute the WER between the first n words in the decoded sentence and the first n words in the target sentence.
Step 3: If this WER is less than or equal to w, and if w≠1, the word at index n in the decoded sentence is deemed correct (with n=1 being the index of the first word in the sentence). Otherwise, the word at index n is deemed incorrect.
Step 4: Let w equal this WER value and increment n by 1.
Step 5: Repeat steps 2-4 until each word in the decoded sentence has been deemed correct or incorrect.

### System Latency Calculation

To estimate the latency of the decoding pipeline during real-time sentence decoding, we first randomly selected one of the sentence testing blocks to use to compute latencies. Because the infrastructure and model parameters were identical across sentence testing blocks, we made the assumption that the distribution of latencies from any block should be representative of the distribution of latencies across all blocks. This was further supported by no noticeable differences in latencies across all of the sentence testing blocks (from our perspective and from the perspective of the participant). After randomly selecting a sentence testing block, we used a video recording of the block to identify the time at which each decoded word appeared on the screen. We then computed the latency of each real-time word prediction as the difference between the word appearance time (from the video) and the time of the final neural data point contained in the detected window of neural activity associated with the word (the final time point of neural data used by the word classifier to predict probabilities for that word production attempt, obtained from the result file associated with the block). By using these differences, the computed latencies represented the amount of time the system required to predict the next word in the sequence after obtaining all of the associated neural data that would be required to make that prediction. The timing between the video and the result file timestamps were synchronized using a short beep that is played at the start of every block (speaker output was also acquired and stored in the result file during each block; see Method S2). Across all trials, there were 42 decoded words in this block.

Using this approach, we found that the mean latency associated with the real-time word predictions was 4.0 s (with a standard deviation of 0.91 s).

### Method S13. Isolated Word Evaluations

### Classification Accuracy, Cross Entropy, and Detection Errors

During offline cross-validated evaluation of the isolated word data (see Method S6), we used the word classifier to predict word probabilities from the neural data associated with the word production attempt in each trial. We computed these word probabilities using time windows of neural activity associated with curated detected events from the speech detector (see Method S8). From these predicted word probabilities, we computed classification accuracy as the fraction of trials in which the target word was equal to the word with the highest predicted probability. We also used these predicted probabilities to compute cross entropy, which measures the amount of additional information that would be required to determine the target word identities from the predicted probabilities. To compute cross entropy, we first obtained the predicted probability of the target word in each trial. The cross entropy (in bits) was then calculated as the mean of the negative log (base 2) across all of these probabilities. In addition to using the curated detected events to compute these metrics, we also used them to measure the number of detection errors made. Specifically, we measured two types of detection errors: the number of false negatives (the number of trials that were not associated with a detected event) and the number of false positives (the number of detected events were not associated with a trial). We reported these detection errors separately (classification accuracy and cross entropy were only computed with correctly detected trials and were not penalized for detection errors).

We performed these analyses using a learning curve scheme that varied the amount of data used to fit both the speech detector and word classifier (detailed in Method S6). The final set of analyses in this learning curve scheme was equivalent to using all of the available data. For every set of analyses in the learning curve scheme, the speech detector provided curated detected speech events. We used neural data aligned to the onsets of these curated detected events to fit the word classifier and predict word probabilities.

### Measuring Training Data Quantities for the Learning Curve Scheme

Because the speech detection and word classification models used different training procedures, we measured the amount of neural data used by each type of model separately for each set of analyses in the learning curve scheme. For each word classifier, we multiplied the number of detected events used to fit the model by 4 seconds (the size of the neural time window used by the classifier). Because each set of analyses in the learning curve scheme used 10-fold cross-validation, this resulted in 10 measures of the amount of training data used for each set of analyses. By computing the mean across the 10 folds, we obtained a single measure of the average amount of data used to fit the word classifier for each set of analyses.

Each speech detection model was fit with sliding windows to predict individual time points of neural activity, resulting in many more training samples per task block than trials. Here, each training sample was a single window from the sliding window training procedure, which corresponded to an individual time point in the task block. Because we used early stopping to prevent overfitting, in practice each speech detector never used all of the data available during model fitting. However, increasing the amount of data available can increase the diversity of the training data (for example, by having data from blocks that were collected across long time periods), which can also affect the number of epochs that the detector is trained for and the robustness of the trained detection model. To measure the amount of data available to each speech detector during training, we simply divided the number of available training samples by the sampling rate (200 Hz). To measure the amount of data that was actually used by each speech detector during training, we divided the number of training samples used by the sampling rate. By computing the mean across the 10 folds, we measured the average amount of data available and the average amount that was actually used to fit the speech detector for each set of analyses.

### Electrode Contributions (Saliences)

To measure how much each electrode contributed to detection and classification performance, we computed electrode contributions (saliences) with the artificial neural networks (ANNs) driving the speech detection and word classification models, respectively. We used a salience calculation method that has been demonstrated with convolutional ANNs during identification of image regions that were most useful for image classification [35]. We have also used this method in our previous work to measure which electrodes were most useful for speech decoding with a recurrent and convolutional ANN [20].

To compute electrode saliences for each type of ANN, we first calculated the gradient of the loss function for the ANN with respect to the input features. The input features were individual time samples of high gamma activity across entire blocks for the speech detector or across detected time windows for the word classifier. For each input feature, we backpropagated the gradient through the ANN to the input layer. We then computed the Euclidean norm across time (within each block or trial) of the resulting gradient values associated with each electrode. Here, we used the norm of the gradient to measure the magnitude of the sensitivity of the loss function to each input (disregarding the direction of the sensitivity). Next, we computed the mean across blocks or trials of the Euclidean norm values, yielding a single salience value for each electrode. Finally, we normalized each set of electrode saliences so that they summed to 1.

We computed these saliences during the final set of analyses in the learning curve scheme, using 10-fold cross-validated evaluation of the speech detector and word classifier. We used the blocks and trials that were evaluated in the test set of each fold to compute the gradients. We also computed saliences during the signal stability analyses (see Method S14).

### Information Transfer Rate

The information transfer rate (ITR) metric, which measures the amount of information that a system communicates per unit time, is commonly used to evaluate brain-computer interfaces [36]. Similar to formulations described in existing literature [2, 36, 37], we used the following formula to compute ITRs in this work:

$$ITR = \frac{1}{T} [ {log}_{2} N + P {log}_{2} P + ( 1 - P ) {log}_{2} ( \frac{1 - P}{N - 1} ) ] , \qquad S19$$

where N is the number of unique targets, P is the prediction accuracy, and T is the average time duration for each prediction. In this work, N=50 (the size of the word set) and T=4 seconds (the size of the neural time window that the classifier uses to compute word probabilities). We set P equal to the mean classification accuracy for the full cross-validation analysis with the isolated word data (from the final set of analyses in the learning curve scheme). This formula makes the following assumptions:

On average, all possible word targets had the same prior probability (that is, the probability independent of the neural data) of being the actual word target in any trial. This is reasonable because there was an equal number of isolated word trials collected for each word target.

The classification accuracy used for P was representative of the overall accuracy of the word classifier (given the amount of training data) and is consistent across trials. This should be a valid assumption because our cross-validated analysis enabled us to evaluate performance across all collected trials.

On average, each incorrect word target had the same probability of being assigned the highest probability value in any trial. Although this is not exactly true in practice for our results (as is evident by the confusion matrix shown in FIG. 3, which shows that some words are predicted slightly more often than others on average), it is typically not exactly true in other studies that have used this formula, and it is generally regarded as an acceptable simplifying assumption.

Using Equation S19, we computed the ITR and reported the result in the caption for FIG. 12.

The ITR was only computed for the isolated word predictions from the word classifier (which used the detected neural windows from the speech detector). Calculation of the ITR of the full decoding pipeline (including the language model) on sentence data would be significantly more complicated because the word-sequence probabilities from the language model will violate assumptions (1) and (3) from the list provided above [38]. The fact that some decoded sentences differed in word length from the corresponding target sentence also makes ITR computation more difficult. For simplicity, we decided to only report ITR using the word classifier outputs. This ITR measurement can also be more easily compared to the performance of the discrimination models reported in other brain-computer interface applications (independent of our specific language-modeling approach).

### Investigating Potential Acoustic Contamination

In recent work, Roussel and colleagues have demonstrated that acoustic signals can directly “contaminate” electrophysiological recordings, causing the spectrotemporal content of signals recorded via an electrophysiological recording methodology to strongly correlate with simultaneously occurring acoustic waveforms [39]. To assess whether or not acoustic contamination was present in our neural recordings, we applied the contamination identification methods described in [39] to our dataset (with some minor procedural deviations, which are noted below).

First, we randomly selected a set of 24 isolated word task blocks (which were chronologically distributed across the 81-week study period) to consider in this analysis. From each block, we obtained the neural activity recorded at 1 kHz (which was not processed using re-referencing against a common average or high gamma feature extraction) and the microphone signals recorded at 30 kHz. These microphone signals were already synchronized to the neural signals (as described in Method S2). We then downsampled the microphone signals to 1000 Hz to match the neural data. Next, as was performed in [39], we “centered” the microphone signal by subtracting from the signal at each time point its mean value over the preceding one second.

We then computed spectrograms for the neural activity recorded from each electrode channel and the recorded microphone signal. We computed the spectrograms as the absolute value of the short-time Fourier transform. For computational efficiency, we slightly departed from [39] to use powers of two in our approach. We computed the Fourier transform within sliding windows of 256 samples (with each window containing 256 ms of data), as opposed to the 200 ms windows used in [39], resulting in 129 frequency bands with evenly spaced center frequencies between 0 and 500 Hz. Each sliding window was spaced 32 time samples apart, yielding spectrogram samples at approximately 31 Hz, as opposed to the 50 Hz rate used in [39]. Because inclusion of a large amount of “silent” task segments (segments during which the participant was not attempting to speak) would bias the analysis against finding acoustic contamination, we clipped periods of time corresponding to inter-trial silence out of the spectrograms. Specifically, we only retained the spectrograms computed from data that occurred between 0.5 seconds before and 3.5 seconds after the go cue in each trial. Although these time periods still contained samples recorded while the participant was silent, this approach drastically reduced the overall proportion of silence in the considered data.

We then measured the across-time correlations (within individual frequency bands) between each microphone spectrogram and the corresponding spectrograms for each electrode. Small correlations between a neural channel and the microphone signal are not definitive evidence of acoustic contamination; there are many factors that could influence correlation, including the presence of shared electrical noise and the characteristics of purely physiological neural responses evoked during attempted speech production. By computing correlations within narrow frequency bands, the resulting correlations are more likely (but not guaranteed) to be indicative of acoustic contamination; for example, spectral power at 300 Hz in the acoustic signal would not be expected to correlate strongly with neural oscillations at that frequency in electrophysiological signals. We aggregated the correlation matrices across spectrograms to obtain an overall correlation matrix across all the considered data, which contained one element for each electrode and frequency band. This procedure was equivalent to concatenating together the (clipped) neural and acoustic spectrograms from each block and then computing a single correlation matrix across all of the data.

To further characterize any potential acoustic contamination, we compared the correlations between the neural and acoustic spectrograms as a function of frequency against the power spectral density (PSD) of the microphone. We expected correlations to be non-zero because a core hypotheses in this work is that the neural activity recorded from the implanted electrodes is causally related to attempted speech production. However, strong correlations between the neural and acoustic spectrograms that also increase and decrease with this PSD would be strong evidence of acoustic contamination. Here, we computed the microphone PSD as the mean of the microphone spectrogram (along the frequency dimension) across all spectrogram samples and blocks (yielding a single value per frequency band).

### Method S14. Stability Evaluations

To assess the stability of the neural signals recorded during word production attempts, we computed classification accuracies and electrode contributions (saliences) with the speech detector and word classifier while varying the date ranges from which the data used to train and test the models were sampled. We performed these analyses using the four date-range subsets (“Early”, “Middle”, “Late”, and “Very late”) and the three evaluation schemes (within-subset, across-subset, and cumulative-subset) defined in Method S6.

First, to yield curated detected times for each subset, the speech detection model used the within-subset training scheme. As a result, all curated detected events for a subset were obtained from a speech detection model fit only with data from the same subset. The percent of trials excluded from further analysis in each subset because they were not associated with a detected event during the detected event curation procedure was 2.3%, 3.8%, 0.8%, and 1.5% for the “Early”, “Middle”, “Late”, and “Very late” subsets, respectively. The word classifier was trained and tested using neural data aligned to the onsets of these curated detected events.

To determine if the neural signals recorded during each date range contained similar amounts of discriminatory information (and to assess the likelihood of a degradation in overall recording quality over time), we compared the classification accuracies from different date-range subsets computed using the within-subset evaluation scheme. To assess the stability of the spatial maps learned by the classification models, we also computed electrode saliences (contributions) for each date-range subset using the within-subset evaluation scheme.

To determine if the temporal proximity of training and testing data affected classification performance (and assess whether or not there were significant changes in the underlying neural activity between date-range subsets even if all of the within-subset accuracies were similar), we compared the within-subset and across-subset classification accuracies individually for each subset. The within-subset and across-subset comparisons are shown in FIG. 14.

To assess whether cortical activity collected across months of recording could be accumulated to improve model performance without frequent recalibration, we computed classification accuracies on the “Very late” subset while varying the amount of training data using the cumulative-subset evaluation scheme (shown in FIG. 4 in the main text). To measure training data quantities for this evaluation scheme, we used the same method as the one described in Method S13 to measure training data quantities for the word classifier in the learning curve analyses.

### Method S15. Statistical Testing

### Word Error Rate Confidence Intervals

To compute 95% confidence intervals for the word error rates (WERs), we performed the following steps for each set of results (chance, without language model, and with language model):
1. Compile the block-level WERs into a single array (with 15 elements, one for each block).
2. Randomly sample (with replacement) 15 WER values from this array and then compute and store the median WER from these values.
3. Repeat step 2 until one million median WER values have been computed.
4. Compute the confidence interval as the 2.5 and 97.5 percentiles of the collection of median WER values from step 3.

### Classification Accuracy Confidence Intervals

To compute 95% confidence intervals for the classification accuracies obtained during the signal stability analyses, we performed the following steps for each date-range subset (“Early”, “Middle”, “Late”, and “Very late”) and each evaluation scheme (within-subset, across-subset, and cumulative-subset):
1. Compile the classification accuracies from each cross-validation fold into a single array (with 10 elements, one for each fold).
2. Randomly sample (with replacement) 10 classification accuracies from this array and then compute and store the mean classification accuracy from these values.
3. Repeat step 2 until one million mean classification accuracies have been computed.
4. Compute the confidence interval as the 2.5 and 97.5 percentiles of the collection of mean classification accuracies from step 3.

SUPPLEMENTARY TABLE S1
Hyperparameter definitions and values.

| | Hyperparameter | Search | Value | Optimal |
|---|---|---|---|---|
| Model | description | space type | range | values¹ |
| Speech | Smoothing size | Uniform (integer) | [1, 80] | (8, 5, 22) |
| Detector | Probability threshold | Uniform | [0.1, 0.9] | (0.297, 0.319, 0.592) |
| | Time threshold | Uniform (integer) | [25, 150] | (79, 82, 93) |
| | duration | | | |
| Word | Number of GRU layers | Uniform (integer) | [1, 3] | (2, 2) |
| Classifier | Nodes per GRU layer | Uniform (integer) | [64, 512] | (434, 420) |
| | Dropout fraction | Uniform | [0.5, 0.95] | (0.704, 0.646) |
| | Convolution kernel | Uniform (integer) | [1, 2] | (2, 2) |
| | size and skip | | | |
| Language | Initial word | Logarithmically | [0.001, 1000] | 0.576 |
| model | smoothing (ψ) | uniform | | |
| Viterbi | Language model | Logarithmically | [0.1, 10] | 0.913 |
| decoder | scaling factor (L) | uniform | | |

¹For the speech detection hyperparameters, three values are listed: the first is the optimal value found when optimizing the detector on the isolated word optimization subset (used to detect word production attempts in the cross-validation subsets for evaluation by the word classifier), the second is the optimal value found when optimizing the detector on a subset of the pooled cross-validation subsets (used to detect word production attempts in the isolated word optimization subset for use during hyperparameter optimization of the word classifier), and the third is the optimal value found during hyperparameter optimization of the decoding pipeline with the sentence optimization subset (the value used during online sentence decoding). For the word classification hyperparameters, two values are listed: the first is the optimal value found when optimizing the classifier on the isolated word optimization subset (the value used for all isolated word evaluations) and the second is the optimal value found when optimizing the classifier on a small subset of isolated word trials near the end of the study period (the value used for offline sentence optimization and online sentence decoding). For the language modeling and Viterbi decoding hyperparameters, the optimal value listed was found when optimizing the decoding pipeline with the sentence optimization subset (the value used for online sentence decoding).

### SUPPLEMENTARY REFERENCES

1. Moses D A, Leonard M K, and Chang E F. Real-time classification of auditory sentences using evoked cortical activity in humans. Journal of Neural Engineering 2018; 15:036005.
2. Moses D A, Leonard M K, Makin J G, and Chang E F. Real-time decoding of question-and-answer speech dialogue using human cortical activity. Nature Communications 2019; 10.
3. Ludwig K A, Miriani R M, Langhals N B, Joseph M D, Anderson D J, and Kipke D R. Using a common average reference to improve cortical neuron recordings from microelectrode arrays. Journal of neurophysiology 2009; 101:1679-89.
4. Williams A J, Trumpis M, Bent B, Chiang C H, and Viventi J. A Novel iECoG Electrode Interface for Comparison of Local and Common Averaged Referenced Signals. In:
2018 40th Annual International Conference of the IEEE Engineering in Medicine and Biology Society (EMBC). Honolulu, H I: IEEE, 2018:5057-60.
5. Parks T W and McClellan J H. Chebyshev Approximation for Nonrecursive Digital Filters with Linear Phase. IEEE Transactions on Circuit Theory 1972; 19:189-94.
6. Romero D E T and Jovanovic G. Digital FIR Hilbert Transformers:
Fundamentals and Efficient Design Methods. In: MATLAB—A Fundamental Tool for Scientific Computing and Engineering Applications—Volume 1. 2012:445-82.
7. Welford B P. Note on a Method for Calculating Corrected Sums of Squares and Products. Technometrics 1962; 4:419-9.
8. Weiss J M, Gaunt R A, Franklin R, Boninger M L, and Collinger J L. Demonstration of a portable intracortical brain-computer interface. Brain-Computer Interfaces 2019; 6:106-17.
9. Bergstra J, Yamins D L K, and Cox D D. Making a Science of Model Search: Hyper-parameter Optimization in Hundreds of Dimensions for Vision Architectures. Icml 2013:115-23.
10. Liaw R, Liang E, Nishihara R, Moritz P, Gonzalez J E, and Stoical. Tune: A Research Platform for Distributed Model Selection and Training. arXiv:1807.05118 2018.
11. Li L, Jamieson K, Rostamizadeh A, et al. A System for Massively Parallel Hyperparam-eter Tuning. arXiv:1810.05934 2020.
12. Paszke A, Gross S, Massa F, et al. PyTorch: An Imperative Style, High-Performance Deep Learning Library. In: Advances in Neural Information Processing Systems 32. Ed. by Wallach H, Larochelle H, Beygelzimer A, d'Alch'e-Buc F, Fox E, and Garnett R. Curran Associates, Inc., 2019:8024-35.
13. Hochreiter S and Schmidhuber J. Long Short-Term Memory. Neural Computation 1997; 9:1735-80.
14. Dash D, Ferrari P, Dutta S, and Wang J. NeuroVAD: Real-Time Voice Activity Detection from Non-Invasive Neuromagnetic Signals. Sensors 2020; 20:2248.
15. Werbos P. Backpropagation through time: what it does and how to do it. Proceedings of the IEEE 1990; 78:1550-60.
16. Elman J L. Finding Structure in Time. Cognitive Science 1990; 14:179-211.
17. Williams R J and Peng J. An Efficient Gradient-Based Algorithm for On-Line Training of Recurrent Network Trajectories. Neural Computation 1990; 2:490-501.
18. Kingma D P and Ba J. Adam: A Method for Stochastic Optimization. arXiv:1412.6980 2017.
19. Krizhevsky A, Sutskever I, and Hinton G E. ImageNet Classification with Deep Convolutional Neural Networks. In: Advances in Neural Information Processing Systems 25. Ed. by Pereira F, Burges C J C, Bottou L, and Weinberger K Q. Curran Associates, Inc., 2012:1097-105.
20. Makin J G, Moses D A, and Chang E F. Machine translation of cortical activity to text with an encoder-decoder framework. Nature Neuroscience 2020; 23:575-82.
21. Virtanen P, Gommers R, Oliphant T E, et al. SciPy 1.0: Fundamental Algorithms for Scientific Computing in Python. Nature Methods 2020; 17:261-72.
22. Martin Abadi, Ashish Agarwal, Paul Barham, et al. TensorFlow: Large-Scale Machine Learning on Heterogeneous Systems. Software available from tensorflow.org. 2015.
23. Zhang Y, Chan W, and Jaitly N. Very Deep Convolutional Networks for End-to-End Speech Recognition. arXiv:1610.03022 2016.
24. Cho K, Merrienboer B van, Gulcehre C, et al. Learning Phrase Representations using RNN Encoder-Decoder for Statistical Machine Translation. arXiv:1406.1078 2014.
25. Pascanu R, Mikolov T, and Bengio Y. On the difficulty of training recurrent neural networks. In: Proceedings of the 30th International Conference on Machine Learning. Ed. by Dasgupta S and McAllester D. Vol. 28. Proceedings of Machine Learning Research. Atlanta, Georgia, USA: PMLR, 2013:1310-8.
26. Sollich P and Krogh A. Learning with ensembles: How overfitting can be useful. In: Advances in Neural Information Processing Systems 8. Ed. by Touretzky D S, Mozer M C, and Hasselmo M E. MIT Press, 1996:190-6.
27. Chen S F and Goodman J. An empirical study of smoothing techniques for language modeling. Computer Speech & Language 1999; 13:359-93.
28. Kneser R and Ney H. Improved backing-off for M-gram language modeling. In: 1995 International Conference on Acoustics, Speech, and Signal Processing. Vol. 1. Detroit, M I, USA: IEEE, 1995:181-4.
29. Bird S, Klein E, and Loper E. Natural language processing with Python: analyzing text with the natural language toolkit. O'Reilly Media, Inc., 2009.
30. Group T H. Hierarchical Data Format. 1997.
31. Collette A. Python and HDF5: unlocking scientific data. “O'Reilly Media, Inc.”, 2013.
32. Heafield K. KenLM: Faster and Smaller Language Model Queries. In:
Proceedings of the Sixth Workshop on Statistical Machine Translation. WMT '11. Association for Computational Linguistics, 2011:187-97.
33. Viterbi A J. Error Bounds for Convolutional Codes and an Asymptotically Optimum Decoding Algorithm. IEEE Transactions on Information Theory 1967; 13:260-9.
34. Jurafsky D and Martin J H. Speech and language processing: an introduction to natural language processing, computational linguistics, and speech recognition. 2nd. Upper Saddle River, New Jersey: Pearson Education, Inc., 2009.
35. Simonyan K, Vedaldi A, and Zisserman A. Deep Inside Convolutional Networks: Visualising Image Classification Models and Saliency Maps. In: Workshop at the International Conference on Learning Representations. Ed. by Bengio Y and LeCun Y. Banff, Canada, 2014.
36. Wolpaw J R, Birbaumer N, McFarland D J, Pfurtscheller G, and Vaughan T M. Brain-computer interfaces for communication and control. Clinical neurophysiology: official journal of the International Federation of Clinical Neurophysiology 2002; 113:767-91.
37. Mugler E M, Patton J L, Flint R D, et al. Direct classification of all American English phonemes using signals from functional speech motor cortex. Journal of neural engineering 2014; 11:35015-15.
38. Speier W, Arnold C, and Pouratian N. Evaluating True BCI Communication Rate through Mutual Information and Language Models. PLoS ONE 2013; 8. Ed. by Wennekers T:e78432.
39. Roussel P, Godais G L, Bocquelet F, et al. Observation and assessment of acoustic contamination of electrophysiological brain signals during speech production and sound perception. Journal of Neural Engineering 2020; 17:056028.

### Example 3: Generalizable Spelling Using a Speech Neuroprosthesis in a Paralyzed Person

### Introduction

Devastating neurological conditions such as stroke and amyotrophic lateral sclerosis can lead to anarthria, the loss of ability to communicate through speech¹. Anarthric patients can have intact language skills and cognition, but paralysis may inhibit their ability to operate assistive devices, severely restricting communication with family, friends, and caregivers and reducing self-reported quality of life².

Brain-computer interfaces (BCIs) have the potential to restore communication to such patients by decoding neural activity into intended messages^{3,4}. Existing communication BCIs typically rely on decoding imagined arm and hand movements into letters to enable spelling of intended sentences^{5,6}. Although implementations of this approach have exhibited promising results, decoding natural attempts to speak directly into speech or text may offer faster and more natural control over a communication BCI. Indeed, a recent survey of prospective BCI users suggests that many patients would prefer speech-driven neuroprostheses over arm- and hand-driven neuroprostheses⁷. Additionally, there have been several recent advances in the understanding of how the brain represents vocal-tract movements to produce speech⁸⁻¹¹ and demonstrations of text decoding from the brain activity of able speakers¹²⁻¹⁵, suggesting that decoding attempted speech from brain activity could be a viable approach for communication restoration.

To assess this, we recently developed a speech neuroprosthesis to directly decode full words in real time from the cortical activity of a person with anarthria and paralysis as he attempted to speak¹⁶. This approach exhibited promising decoding accuracy and speed, but as an initial study focused on a preliminary 50-word vocabulary. While direct word decoding with a limited vocabulary has immediate practical benefit, expanding access to a larger vocabulary of at least 1,000 words would cover over 85% of the content in natural English sentences¹⁷ and enable effective day-to-day use of assistive-communication technology¹⁸. Hence, a powerful complementary technology could expand current speech-decoding approaches to enable users to spell out intended messages from a large and generalizable vocabulary while still allowing fast, direct word decoding to express frequent and commonly used words. Separately, in this prior work the participant was controlling the neuroprosthesis by attempting to speak aloud, making it unclear if the approach would be viable for potential users who cannot produce any vocal output whatsoever.

Here, we demonstrate that real-time decoding of silent attempts to say 26 alphabetic code words from the NATO phonetic alphabet can enable highly accurate and rapid spelling in a participant with paralysis and anarthria. During training sessions, we cued the participant to attempt to produce individual code words and a hand-motor movement, and we used the simultaneously recorded cortical activity from an implanted 128-channel electrocorticography (ECoG) array to train classification and detection models. After training, the participant performed spelling tasks in which he spelled out sentences in real time with a 1,152-word vocabulary using attempts to silently say the corresponding alphabetic code words. A beam-search algorithm used predicted code-word probabilities from a classification model to find the most likely sentence given the neural activity while automatically inserting spaces between decoded words. To initiate spelling, the participant silently attempted to speak, and a speech-detection model identified this start signal directly from ECoG activity. After spelling out the intended sentence, the participant attempted the hand-motor movement to disengage the speller. When the classification model identified this hand-motor command from ECoG activity, a large neural network-based language model rescored the potential sentence candidates from the beam search and finalized the sentence. In post-hoc simulations, our system generalized well across large vocabularies of over 9,000 words.

### Results

### Overview of the Real-Time Spelling Pipeline

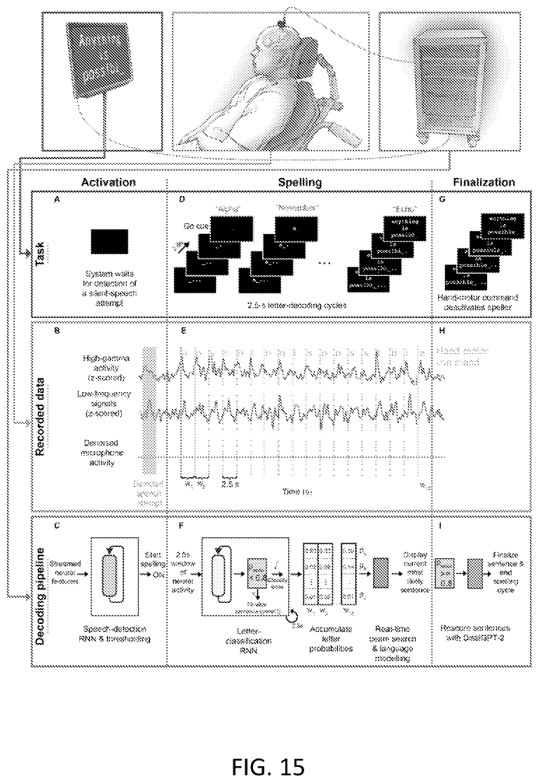

We designed a sentence-spelling pipeline that enabled a participant with anarthria and paralysis to silently spell out messages using signals acquired from a high-density electrocorticography (ECoG) array implanted over his sensorimotor cortex (FIG. 15). We tested the spelling system under copy-typing and conversational task conditions. In each trial of the copy-typing task condition, the participant was presented with a target sentence on a screen and then attempted to replicate that sentence. In the conversational task condition, there were two types of trials: Trials in which the participant spelled out volitionally chosen responses to questions presented to him and trials in which he spelled out arbitrary, unprompted sentences. Prior to real-time testing, no day-of recalibration occurred; model parameters and hyperparameters were fit using data exclusively from preceding sessions.

When the participant was ready to begin spelling a sentence, he attempted to silently say an arbitrary word (FIG. 15A). We define silent-speech attempts as volitional attempts to articulate speech without vocalizing. Meanwhile, the participant's neural activity was recorded from each electrode and processed to simultaneously extract high-gamma activity (HGA; between 70-150 Hz) and low-frequency signals (LFS; between 0.3-100 Hz; FIG. 15B). To initiate spelling, a speech-detection model processed each time point of data in the combined feature stream (containing HGA+LFS features; FIG. 15C) to detect this initial silent-speech attempt.

Once an attempt to speak was detected, the paced spelling procedure began (FIG. 15D). In this procedure, an underline followed by three dots appeared on the screen in white text. The dots disappeared one by one, representing a countdown. After the last dot disappeared, the underline turned green to indicate a go cue, at which time the participant attempted to silently say the NATO code word corresponding to the first letter in the sentence. The time window of neural features from the combined feature stream obtained during the 2.5-second interval immediately following the go cue was passed to a neural classifier (FIG. 15E). Shortly after the go cue, the countdown for the next letter automatically started. This procedure was then repeated until the participant volitionally disengaged it (described later in this section).

The neural classifier processed each time window of neural features to predict probabilities across the 26 alphabetic code words (FIG. 15F). A beam-search algorithm used the sequence of predicted letter probabilities to compute potential sentence candidates, automatically inserting spaces into the letter sequences where appropriate and using a language model to prioritize linguistically plausible sentences. During real-time sentence spelling, the beam search only considered sentences composed of words from a predefined 1,152-word vocabulary, which contained common words that are relevant for assistive-communication applications. The most likely sentence at any point in the task was always visible to the participant (FIG. 15D). We instructed the participant to continue spelling even if there were mistakes in the displayed sentence, since the beam search could correct the mistakes after receiving more predictions.

After attempting to silently spell out the entire sentence, the participant was instructed to attempt to squeeze his right hand to disengage the spelling procedure (FIG. 15H). The neural classifier predicted the probability of this attempted hand-motor movement from each 2.5-second window of neural features, and if this probability was greater than 80%, the spelling procedure was stopped and the decoded sentence was finalized (FIG. 15I). To finalize the sentence, sentences with incomplete words were first removed from the list of potential candidates, and then the remaining sentences were rescored with a separate language model. The most likely sentence was then updated on the participant's screen (FIG. 15G). After a brief delay, the screen was cleared and the task continued to the next trial.

To train the detection and classification models prior to real-time testing, we collected data as the participant performed an isolated-target task. In each trial of this task, a NATO code word appeared on the screen, and the participant was instructed to attempt to silently say the code word at the corresponding go cue. In some trials, an indicator representing the hand-motor command was presented instead of a code word, and the participant was instructed to imagine squeezing his right hand at the go cue for those trials.

### Decoding Performance

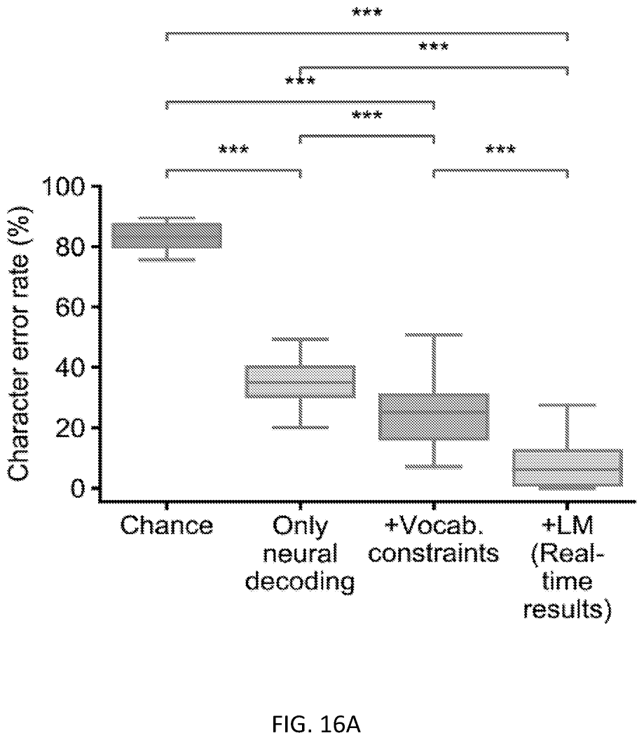

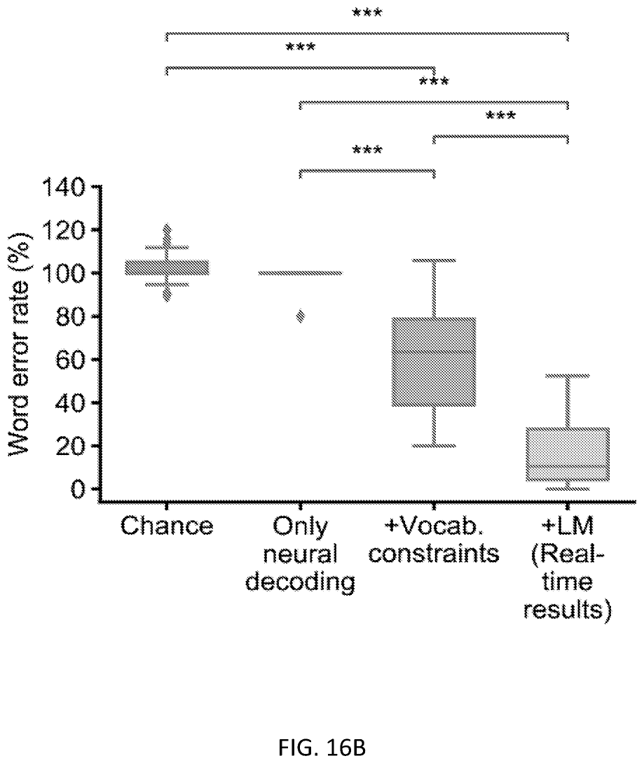

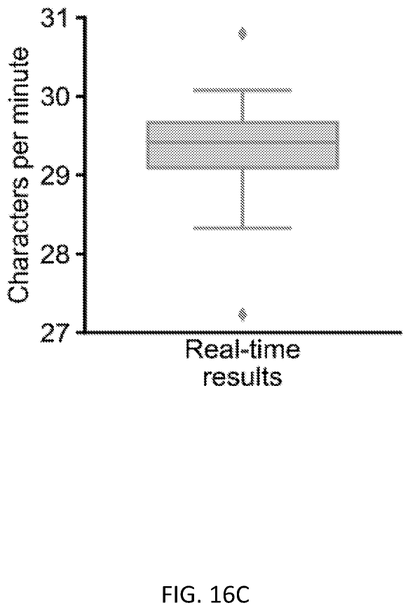

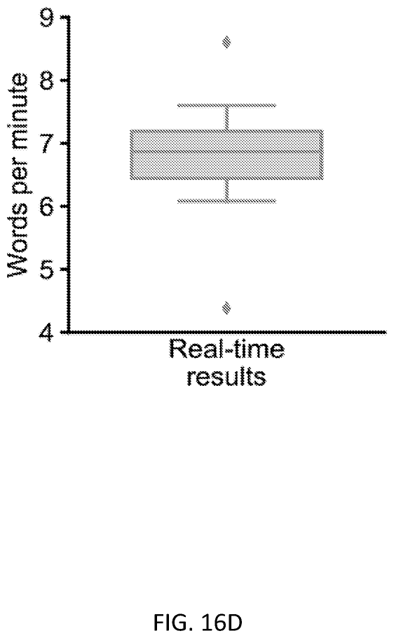

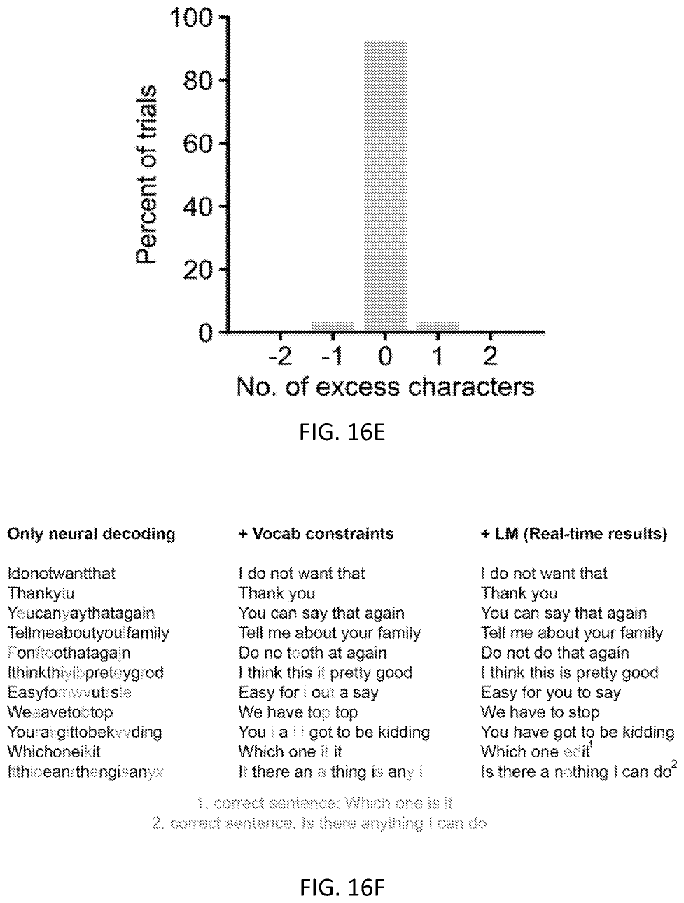

To evaluate the performance of the spelling system, we decoded sentences from the participant's neural activity in real time as he attempted to spell out 150 sentences (two repetitions each of 75 unique sentences selected from an assistive-communication corpus; see Table S1) during the copy-typing task. We evaluated the decoded sentences using word error rate (WER), character error rate (CER), words per minute (WPM), and characters per minute (CPM) metrics (FIG. 16). For characters and words, the error rate is defined as the edit distance, which is the minimum number of character or word deletions, insertions, and substitutions required to convert the predicted sentence to the target sentence that was displayed to the participant, divided by the total number of characters or words in the target sentence, respectively. These metrics are commonly used to assess the decoding performance of automatic speech recognition systems¹⁹ and brain-computer interface applications^{6,16}.

We observed a median CER of 6.13% and median WER of 10.53% (99% confidence interval (CI) [2.25, 11.6] and [5.76, 24.8]) across the real-time test blocks (each block contained multiple sentence-spelling trials; FIG. 16A, 16B). Across 150 sentences, 105 (70%) were decoded without error, and 69 of the 75 sentences (92%) were decoded perfectly at least one of the two times they were attempted. Additionally, across 150 sentences, 139 (92.7%) sentences were decoded with the correct number of letters, enabled by high classification accuracy of the attempted hand squeeze (FIG. 16E). We also observed a median CPM of 29.41 and median WPM of 6.86 (99% CI [29.1, 29.6] and [6.54, 7.12]) across test blocks, with spelling rates in individual blocks as high as 30.79 CPM and 8.60 WPM (FIGS. 16C, 16D). These rates are higher than the median rates of 17.37 CPM and 4.16 WPM (99% CI [16.1, 19.3] and [3.33, 5.05]) observed with the participant as he used his commercially available Tobii Dynavox assistive-typing device (as measured in our previous work¹⁶)

To understand the individual contributions of the classifier, beam search, and language model to decoding performance, we performed offline analyses using data collected during these real-time copy-typing task blocks (FIGS. 16A, 16B). To examine the chance performance of the system, we replaced the model's predictions with randomly generated values while continuing to use the beam search and language model. This resulted in a CER and WER that was significantly worse than the real-time results (z=7.09, P=8.08×10⁻¹² and z=7.09, P=8.08×10⁻¹². This demonstrates that the classification of neural signals was critical to system performance and that system performance was not just relying on a constrained vocabulary and language-modeling techniques.

To assess how well the neural classifier alone could decode the attempted sentences, we compared character sequences composed of the most likely letter for each individual 2.5-second window of neural activity using only the neural classifier to the corresponding target character sequences. All whitespace characters were ignored during this comparison (during real-time decoding, these characters were inserted automatically by the beam search). This resulted in a median CER of 35.1% (99% CI [30.6, 38.5]), which is significantly lower than chance (z=7.09, P=8.08×10⁻¹², two-sided Wilcoxon Rank-Sum test with 6-way Holm-Bonferroni correction), and shows that time windows of neural activity during silent code-word production attempts were discriminable. This corresponds to a classifier accuracy rate of 64.9%. The median WER was 100% (99% CI [100.0, 100.0]) for this condition; without language modeling or automatic insertion of whitespace characters, the predicted character sequences rarely matched the corresponding target character sequences.

To measure how much decoding was improved by the beam search, we passed the neural classifier's predictions into the beam search and constrained character sequences to be composed of only words within the vocabulary without incorporating any language modeling. This significantly improved CER and WER over only using the most likely letter at each timestep (z=4.51, P=6.37×10⁻⁶ and z=6.61, P=1.19×10⁻¹⁰ respectively, two-sided Wilcoxon Rank-Sum test with 6-way Holm-Bonferroni correction). As a result of not using language modeling, which incorporates the likelihood of word sequences, the system would sometimes predict nonsensical sentences, such as “Do no tooth at again” instead of “Do not do that again” (FIG. 16F). Hence, including language modeling to complete the full real-time spelling pipeline significantly improved median CER to 6.13% and median WER to 10.53% over using the system without any language modeling (z=5.53, P=6.34×10⁻⁸ and z=6.11, P=2.01×10⁻⁹ respectively, two-sided Wilcoxon Rank-Sum test with 6-way Holm-Bonferroni correction), illustrating the benefits of incorporating the natural structure of English during decoding.

### Discriminatory Content in High-Gamma Activity and Low-Frequency Signals

Previous efforts to decode speech from brain activity have typically relied on content in the high-gamma frequency range (between 70-170 Hz, but exact boundaries vary) during decoding^{12,13,24} However, recent studies have demonstrated that low-frequency content (between 0-40 Hz) can also be used for spoken- and imagined-speech decoding^{14,15,25-27} although the differences in the discriminatory information contained in each frequency range remain poorly understood.

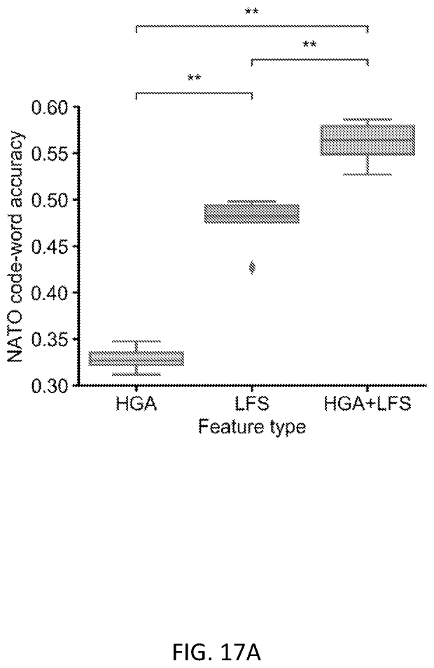

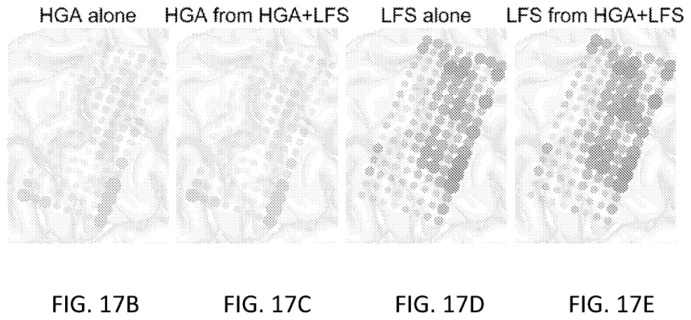

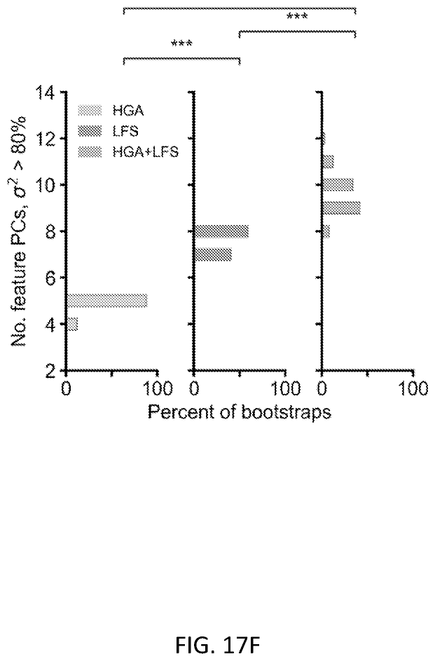

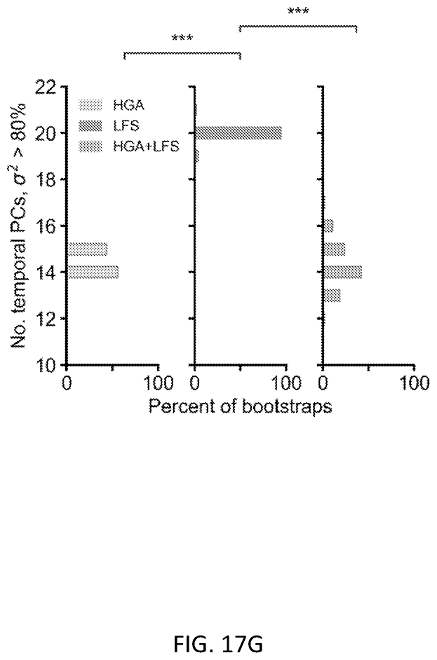

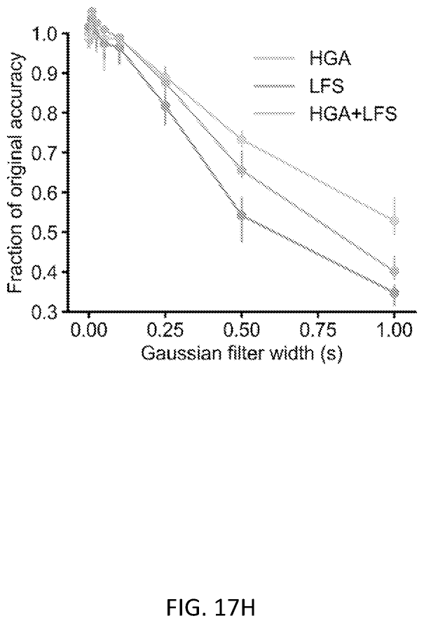

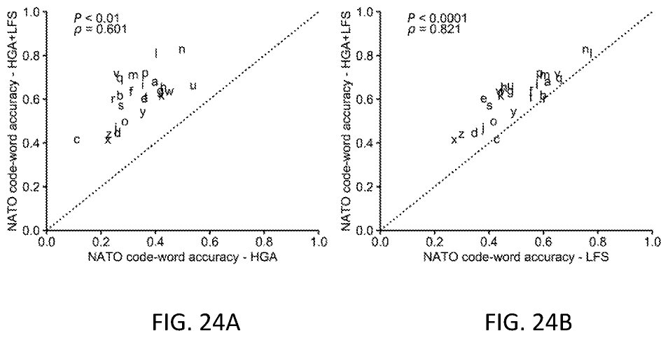

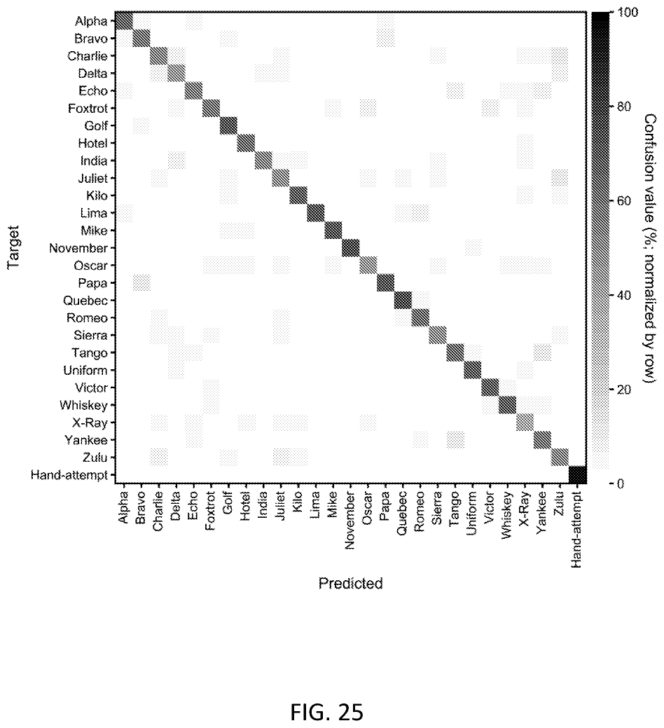

Although previous efforts to decode speech from brain activity typically only used high-gamma activity (HGA)^{12,13,15} our spelling system also used low-frequency signals (LFS) during decoding. Because inputs to the classifier were downsampled (with an anti-aliasing filter) to 33.33 Hz prior to classification, LFS used during classification only contained signal components between 0.3 to 16.67 Hz. Using the most recent 9,132 trials of the isolated-word task (in each of these trials, the participant attempted to silently say a code word), we trained 10-fold cross-validated models using only HGA, only LFS, and with both feature types. Models using only LFS demonstrated higher code-word classification accuracy than models using only HGA, and models using both feature types (HGA+LFS) outperformed the other two models (P<0.001 for all comparisons, two-sided Mann-Whitney U test with 3-way Holm-Bonferroni correction; FIGS. 17A, 24), achieving a median classification accuracy of 56.4% (FIG. 25).

We then investigated the relative contributions of each electrode and feature type to the neural classification models trained using HGA, LFS, and HGA+LFS. For each model, we first computed each electrode's contribution to classification by measuring the effect that small changes to the electrode's values had on the model's predictions²⁸. Electrode contributions for the HGA model were primarily localized to the ventral portion of the grid, corresponding to the ventral sensorimotor cortex (vSMC), pars opercularis, and pars triangularis (FIG. 17B). Contributions for the LFS model were much more diffuse, covering more dorsal and posterior parts of the grid corresponding to dorsal aspects of the vSMC in the pre- and postcentral gyri (FIG. 17D). Contributions from the HGA model and the LFS model were moderately correlated with a Spearman rank correlation of 0.501 (n=128 electrode contributions per feature type, P<0.01). The separate contributions from HGA and LFS in the HGA+LFS model were highly correlated with the contributions for HGA-only and LFS-only models, respectively (n=128 electrode contributions per feature type, (P<0.01 for both Spearman rank correlations of 0.922 and 0.963, respectively; FIGS. 17C, 17E). These findings indicate that the information contained in the two feature types that was most useful during decoding was not redundant and was recorded from relatively distinct cortical areas.

To further characterize HGA and LFS features, we investigated whether the LFS had increased feature or temporal dimensionality, which could contribute to increased decoding accuracy. First, we performed principal component analysis (PCA) on the feature dimension for HGA, LFS, and HGA+LFS feature sets. The resulting principal components (PCs) captured the spatial variability (across electrode channels) for the HGA and LFS feature sets and the spatial and spectral variabilities (across electrode channels and feature types, respectively) for the HGA+LFS feature set. We then calculated the minimum number of principal components (PCs) needed to explain more than 80% of the variance. To explain more than 80% of the variance, LFS required significantly more feature PCs than HGA (z=12.2, P=7.57×10⁻³⁴, two-sided Wilcoxon Rank-Sum test with 3-way Holm-Bonferroni correction; FIG. 17F). The combined HGA+LFS feature set required significantly more feature PCs than the individual HGA or LFS features (P=6.20×10⁻³⁸ and P=1.60×10⁻³³, respectively, two-sided Wilcoxon Rank-Sum test with 3-way Holm-Bonferroni correction; FIG. 17F), suggesting that LFS did not simply replicate HGA at each electrode but instead added unique feature variance.

To assess the temporal content of the features, we first used a similar PCA approach to measure temporal dimensionality. We observed that the LFS features required significantly more temporal PCs than both the HGA and HGA+LFS feature sets (P=2.72×10⁻³⁹ and P=1.37×10⁻³⁸, respectively, FIG. 17G; two-sided Mann-Whitney U test with 3-way Holm-Bonferroni correction). We observed that the LFS features required significantly more temporal PCs than both the HGA and HGA+LFS feature sets to explain more than 80% of the variance (z=12.2, P=7.57×10⁻³⁴ and z=12.2, P=7.57×10⁻¹4, respectively, FIG. 17G; two-sided Wilcoxon Rank-Sum test with 3-way Holm-Bonferroni correction). Because the inherent temporal dimensionality for each feature type remained the same within the HGA+LFS feature set, the required number of temporal PCs to explain this much variance for the HGA+LFS features was in between the corresponding numbers for the individual feature types. Then, to assess how the temporal resolution of each feature type affected decoding performance, we temporally smoothed each feature time series with Gaussian filters of varying width. A wider Gaussian filter causes a greater amount of temporal smoothing, effectively temporally blurring the signal and hence lowering temporal resolution. Temporally smoothing the

LFS features decreased the classification accuracy significantly more than smoothing the HGA or HGA+LFS features (Wilcoxon signed-rank statistic=737.0, P=4.57×10⁻⁵ and statistic=391.0, P=1.13×10⁻⁸, two-sided Wilcoxon signed-rank test with 3-way Holm-Bonferroni correction; FIG. 17H). (Wilcoxon signed-rank statistic=1460.0, P=0.443). This is largely consistent with the outcomes of the temporal-PCA comparisons. Together, these results indicate that the temporal content of LFS had higher variability and contained more speech-related discriminatory information than HGA.

### Differences in Neural Discriminability Between NATO Code Words and Letters

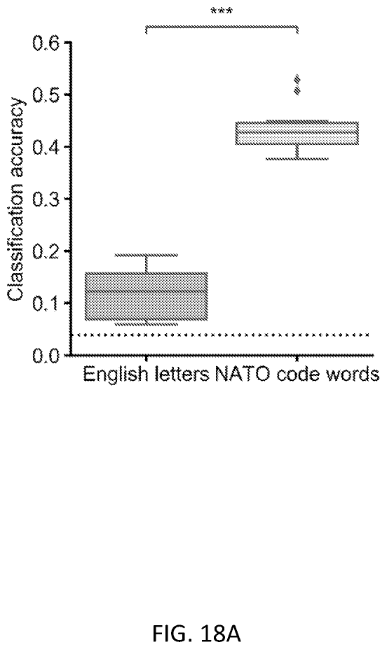

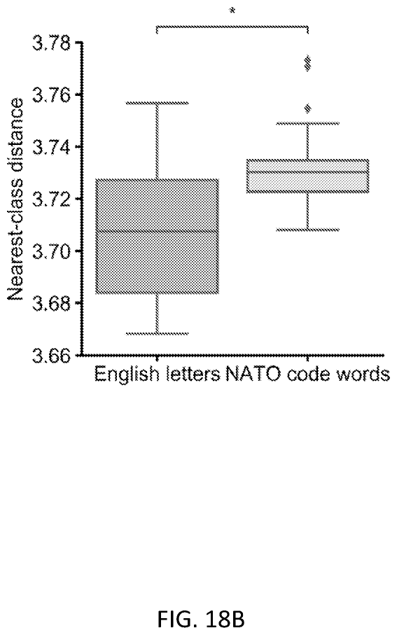

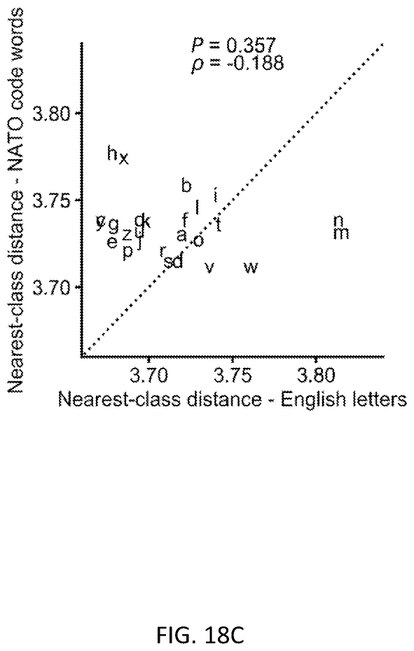

During control of our system, the participant attempted to silently say NATO code words to represent each letter (“alpha” instead of “a”, “beta” instead of “b”, and so forth) rather than simply saying the letters themselves. We hypothesized that neural activity associated with attempts to produce code words would be more discriminable than letters due to increased phonetic variability and longer utterance lengths. To test this, we first collected data using a modified version of the isolated-target task in which the participant attempted to say each of the 26 English letters instead of the NATO code words that represented them. Afterwards, we trained and tested classification models using HGA+LFS features from the most recent 29 attempts to silently say each code word and each letter in 10-fold cross-validated analyses. Indeed, code words were classified with significantly higher accuracy than the letters (z=3.78, P=1.57×10⁻⁴, two-sided Wilcoxon Rank-Sum test; FIG. 18A).

To perform a model-agnostic comparison between the neural discriminability of each type of utterance (either code words or letters), we computed nearest-class distances for each utterance using the HGA+LFS feature set. Here, each utterance represented a single class, and distances were only computed between utterances of the same type. A larger nearest-class distance for a code word or letter indicates that that utterance is more discriminable in neural feature space because the neural activation patterns associated with silent attempts to produce it are more distinct from other code words or letters, respectively. We found that nearest-class distances for code words were significantly higher overall than for letters (z=2.98, P=2.85×10⁻³, two-sided Wilcoxon Rank-Sum test; FIG. 18B), although not all characters had a higher nearest-class distance when using code words instead of letters (FIG. 18C).

### Distinctions in Evoked Neural Activity Between Silent- and Overt-Speech Attempts

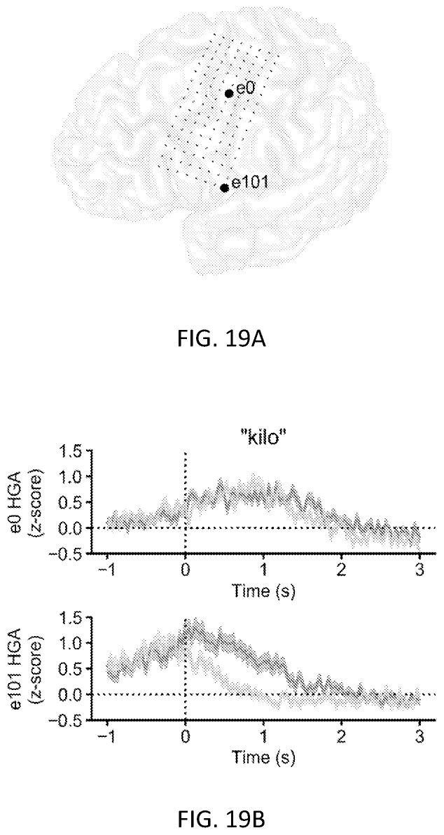

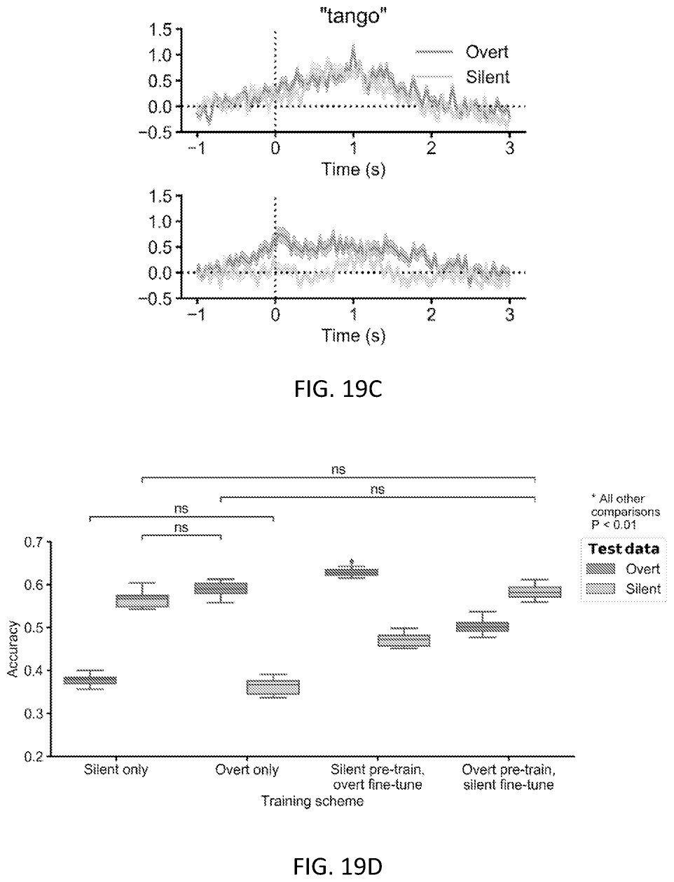

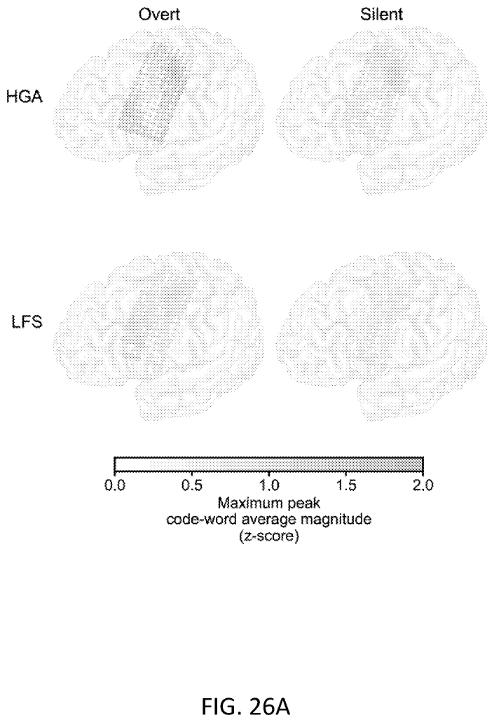

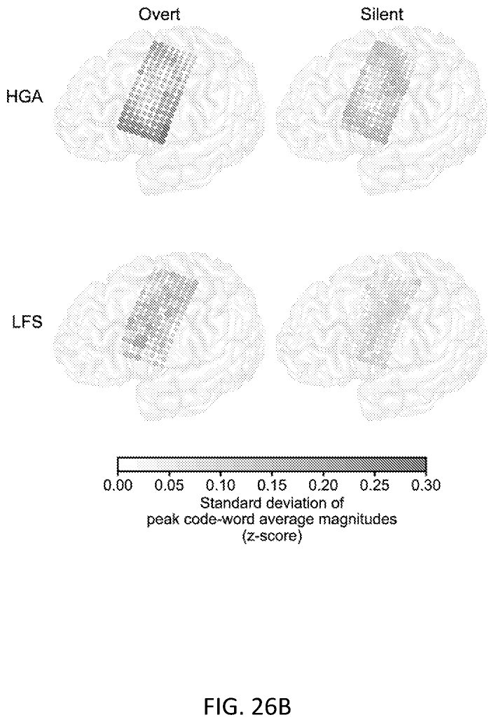

The spelling system was controlled by silent-speech attempts, differing from our previous work in which the same participant used overt-speech attempts (attempts to speak aloud) to control a similar speech-decoding system¹⁶. To assess differences in neural activity and decoding performance between the two types of speech attempts, we collected a version of the isolated-target task in which the participant was instructed to attempt to say the code words aloud (overtly instead of silently). To visualize the differences between overt and silent speech attempts, we compared the evoked HGA for different code words and electrodes. The spatial patterns of evoked neural activity for the two types of speech attempts exhibited similarities, and inspections of evoked HGA for two electrodes suggest that some neural populations respond similarly for each speech type while others do not (FIGS. 19B, 19C; FIG. 26). To compare the discriminatory neural content between silent- and overt-speech attempts, we performed 10-fold cross-validated classification analyses using the HGA+LFS features associated with the speech attempts (FIG. 19D). First, for each speech type (silent or overt), we trained a classification model using data collected with that speech type. To determine if the classification models could leverage similarities in the neural representations associated with each speech type to improve performance, we also created models by pre-training on one speech type and then fine-tuning on the other speech type. We then tested each classification model on held-out data associated with each speech type and compared all 28 combinations of pairs of results. Models trained solely on silent data but tested on overt data and vice versa resulted in classification accuracies that were above chance (median accuracies of 36.3%, 99% CI [35.0, 37.5] and 33.5%, 99% CI [31.0, 35.0], respectively; chance accuracy is 3.85%). However, for both speech types, training and testing on the same type resulted in significantly higher performance (P<0.01, two-sided Wilcoxon Rank-Sum test, 28-way Holm-Bonferroni correction). Pre-training models using the other speech type led to increases in classification accuracy, though the increase was more modest and not significant for the overt speech type (median accuracy increasing by 2.33%, z=2.65, P=0.033 for overt, median accuracy increasing by 10.4%, z=3.78, P=4.40×10⁻³ for silent, two-sided Wilcoxon Rank-Sum test, 28-way Holm-Bonferroni correction). Together, these results suggest that the neural activation patterns evoked during silent and overt attempts to speak shared some similarities but were not identical.

### Generalizability to Larger Vocabularies and Alternative Tasks

Although the 1,152-word vocabulary enabled communication of a wide variety of common sentences, we also assessed how well our approach can scale to larger vocabulary sizes. Specifically, we simulated the copy-typing spelling results using three larger vocabularies selected based on their words' frequency in large-scale English corpora with sizes of 3,303, 5,249, and 9,170 words. For each vocabulary, we retrained the language model used during the beam search to incorporate the new words. The large language model used when finalizing sentences was not altered for these analyses because it was designed to generalize to any English text.

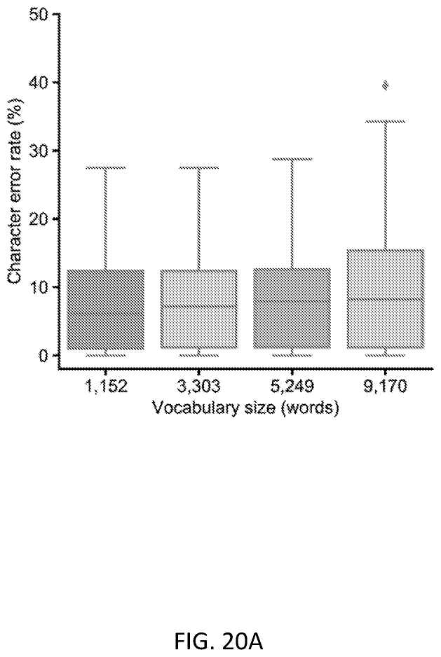

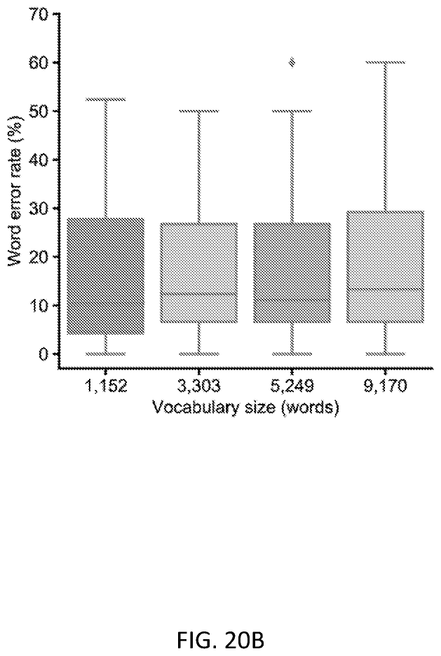

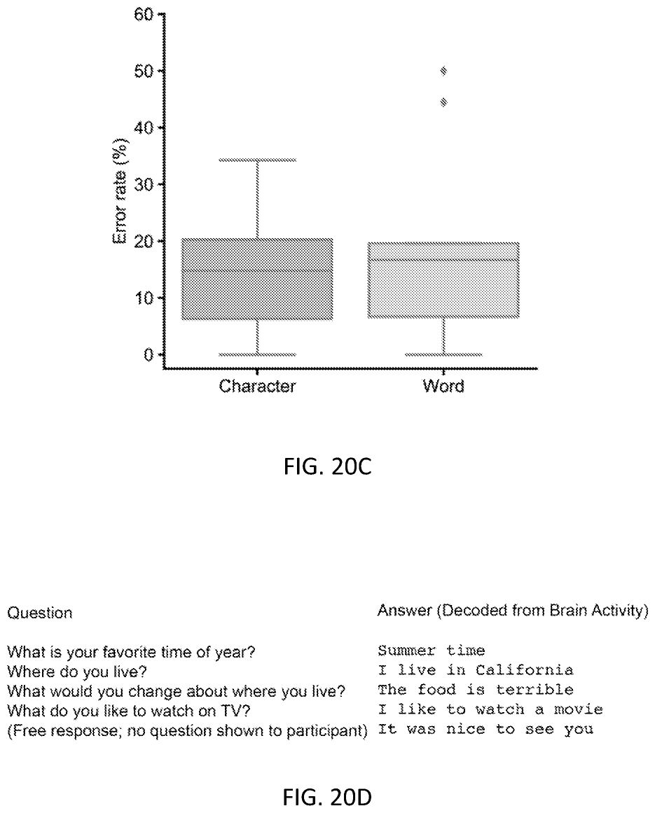

High performance was maintained with each of the new vocabularies, with median character error rates (CERs) of 7.18% (99% CI [2.25, 11.6]), 7.93% (99% CI [1.75, 12.1]), and 8.23% (99% CI [2.25, 13.5]) for the 3,303-, 5,249-, and 9,170-word vocabularies, respectively (FIG. 20A; median real-time CER was 6.13%(99% CI [2.25, 11.6]) with the original vocabulary containing 1,152 words). Median word error rates (WERs) were 12.4% (99% CI [8.01, 22.7]), 11.1% (99% CI [8.01, 23.1]), and 13.3% (99% CI [7.69, 28.3]), respectively (FIG. 20B; WER was 10.53% (99% CI [5.76, 24.8]) for the original vocabulary). Overall, no significant differences were found between the CERs or WERs with any two vocabularies (P>0.01 for all comparisons, two-sided Wilcoxon Rank-Sum test with 6-way Holm-Bonferroni correction), illustrating the generalizability of our spelling approach to larger vocabulary sizes that enable fluent communication.

Finally, to assess the generalizability of our spelling approach to behavioral contexts beyond the copy-typing task structure, we measured performance as the participant engaged in a conversational task condition. In each trial of this condition, the participant was either presented with a question (as text on a screen) or was not presented with any stimuli. He then attempted to spell out a volitionally chosen response to the presented question or any arbitrary sentence if no stimulus was presented. To measure the accuracy of each decoded sentence, we asked the participant to nod his head to indicate if the sentence matched his intended sentence exactly. If the sentence was not perfectly decoded, the participant used his commercially available assistive-communication device to spell out his intended message. Across 28 trials of this real-time conversational task condition, the median CER was 14.8% (99% CI [0.00, 29.7]) and the median WER was 16.7% (99% CI [0.00, 44.4]) (FIGS. 20C, 20D). We observed a slight increase in decoding error rates compared to the copy-typing task, potentially due to the participant responding using incomplete sentences (such as “going out” and “summer time”) that would not be well represented by the language models. Nevertheless, these results demonstrate that our spelling approach can enable a user to generate responses to questions as well as unprompted, volitionally chosen messages.

### Discussion

Here, we demonstrated that a paralyzed person with anarthria could control a neuroprosthesis to spell out intended messages in real time using attempts to silently speak. With phonetically rich code words to represent individual letters and an attempted hand movement to indicate an end-of-sentence command, we used deep-learning and language-modeling techniques to decode sentences from electrocorticographic (ECoG) signals. These results significantly expand our previous word-decoding findings with the same participant²⁰ by enabling completely silent control, leveraging both high- and low-frequency ECoG features, including a non-speech motor command to finalize sentences, facilitating large-vocabulary sentence decoding through spelling, and demonstrating continued stability of the relevant cortical activity beyond 128 weeks since device implantation.

Previous implementations of spelling brain-computer interfaces (BCIs) have demonstrated that users can type out intended messages by visually attending to letters on a screen^{29,30} or by using motor imagery to control a two-dimensional computer cursor^{4,5} or attempt to handwrite letters⁶. BCI performance using penetrating microelectrode arrays in motor cortex has steadily improved over the past 20 years³¹⁻³³, recently achieving spelling rates as high as 90 characters per minute with a single participant⁶, although this participant was able to speak normally. Our results extend the list of immediately practical and clinically viable control modalities for spelling-BCI applications to include silently attempted speech with an implanted ECoG array, which may be preferred for daily use by some patients due to the relative naturalness of speech⁷ and may be more chronically robust across patients through the use of less invasive, non-penetrating electrode arrays with broader cortical coverage.

In post-hoc analyses, we showed that decoding performance improved as more linguistic information was incorporated into the spelling pipeline. This information helped facilitate real-time decoding with a 1,152-word vocabulary, allowing for a wide variety of general and clinically relevant sentences as possible outputs. Furthermore, through offline simulations, we validated this spelling approach with vocabularies containing over 9,000 common English words, which exceeds the estimated lexical-size threshold for basic fluency and enables general communication^{34,35}. These results add to consistent findings that language modeling can significantly improve neural-based speech decoding^{12,15,20} and demonstrates the immediate viability of speech-based spelling approaches for a general-purpose assistive-communication system.

In this study, we showed that neural signals recorded during silent-speech attempts by an anarthric person can be effectively used to drive a speech neuroprosthesis. Supporting the hypothesis that these signals contained similar speech-motor representations to signals recorded during overt-speech attempts, we showed that a model trained solely to classify overt-speech attempts can achieve above-chance classification of silent-speech attempts, and vice versa. Additionally, the spatial localization of electrodes contributing most to classification performance was similar for both overt and silent speech, with many of these electrodes located in the ventral sensorimotor cortex, a brain area that is heavily implicated in articulatory speech-motor processing 8-10,36

Overall, these results further validate silently attempted speech as an effective alternative behavioral strategy to imagined speech and expand findings from our previous work involving the decoding of overt-speech attempts with the same participant²⁰, indicating that the production of residual vocalizations during speech attempts is not necessary to control a speech neuroprosthesis. These findings illustrate the viability of attempted-speech control for individuals with complete vocal-tract paralysis (such as those with locked-in syndrome), although future studies with these individuals are required to further our understanding of the neural differences between overt-speech attempts, silent-speech attempts, and purely imagined speech as well as how specific medical conditions might affect these differences. We expect that the approaches described here, including recording methodology, task design, and modeling techniques, would be appropriate for both speech-related neuroscientific investigations and BCI development with patients regardless of the severity of their vocal-tract paralysis, assuming that their speech-motor cortices are still intact and that they are mentally capable of attempting to speak.

In addition to enabling spatial coverage over the lateral speech-motor cortical brain regions, the implanted ECoG array also provided simultaneous access to neural populations in the hand-motor (“hand knob”) cortical area that is typically implicated during executed or attempted hand movements 37. Our approach is the first to combine the two cortical areas to control a BCI. This ultimately enabled our participant to use an attempted hand movement, which was reliably detectable and highly discriminable from silent-speech attempts with 98.43% classification accuracy (99% CI [95.31, 99.22]), to indicate when he was finished spelling any particular sentence. This may be a preferred stopping mechanism compared to previous spelling BCI implementations that terminated spelling for a sentence after a pre-specified time interval had elapsed or extraneously when the sentence was completed 5 or required a head movement to terminate the sentence 6. By also allowing a silent-speech attempt to initiate spelling, the system could be volitionally engaged and disengaged by the participant, which is an important design feature for a practical communication BCI. Although attempted hand movement was only used for a single purpose in this first demonstration of a multimodal communication BCI, separate work with the same participant suggests that non-speech motor imagery could be used to indicate several distinct commands 38.

In future communication neuroprostheses, it may be possible to use a combined approach that enables rapid decoding of full words or phrases from a limited, frequently used vocabulary²⁰ as well as slower, generalizable spelling for out-of-vocabulary items. Transfer-learning methods could be used to cross-train differently purposed speech models using data aggregated across multiple tasks and vocabularies, as validated in previous speech-decoding work¹³. Although clinical and regulatory guidelines concerning the implanted percutaneous connector prevented the participant from being able to use the current spelling system independently, development of a fully implantable ECoG array and a software application to integrate the decoding pipeline with an operating system's accessibility features could allow for autonomous usage. Facilitated by deep-learning techniques, language modeling, and the signal stability and spatial coverage afforded by ECoG recordings, future communication neuroprostheses could enable users with severe paralysis and anarthria to control assistive technology and personal devices using naturalistic silent-speech attempts to generate intended messages and attempted non-speech motor movements to issue high-level, interactive commands.

### Methods

### Clinical Trial Overview

This study was conducted as part of the BCI Restoration of Arm and Voice (BRAVO) clinical trial (ClinicalTrials.gov; NCT03698149). The goal of this single-institution clinical trial is to determine if ECoG and custom decoding methods can enable assistive neurotechnology to restore communication and mobility. The Food and Drug Administration approved an investigational device exemption for the neural implant used in this study. The study protocol was approved by the Committee on Human Research at the University of California, San Francisco. The data safety monitoring board agreed to the release of results in the manuscript prior to the completion of the trial. The participant gave his informed consent to participate in this study after the details concerning the neural implant, experimental protocols, and medical risks were thoroughly explained to him.

### Participant

The participant, who was 36 years old at the start of the study, was diagnosed with severe spastic quadriparesis and anarthria by neurologists and a speech-language pathologist after experiencing an extensive pontine stroke. He is fully cognitively intact. Although he retains the ability to vocalize grunts and moans, he is unable to produce intelligible speech, and his attempts to speak aloud are abnormally effortful due to his condition (according to self-reported descriptions). He typically relies on assistive computer-based interfaces that he controls with residual head movements to communicate. This participant has participated in previous studies as part of this clinical trial^{16,20}, although neural data from those studies were not used in the present study

### Neural Implant

The neural implant device consisted of a high-density electrocorticography (ECoG) array (PMT) and a percutaneous connector (Blackrock Microsystems). The ECoG array contained 128 disk-shaped electrodes arranged in a lattice formation with 4-mm center-to-center spacing. The array was surgically implanted on the pial surface of the left hemisphere of the brain over cortical regions associated with speech production, including the dorsal posterior aspect of the inferior frontal gyrus, the posterior aspect of the middle frontal gyrus, the precentral gyrus, and the anterior aspect of the postcentral gyrus^{8,10,32}. The percutaneous connector was implanted in the skull to conduct electrical signals from the ECoG array to a detachable digital headstage and cable (NeuroPlex E; Blackrock Microsystems), minimally processing and digitizing the acquired brain activity and transmitting the data to a computer. The device was implanted in February 2019 without any surgical complications. More details on the device and surgical procedure can be found in our previous work with the same device and participant¹⁶.

### Data Acquisition and Preprocessing

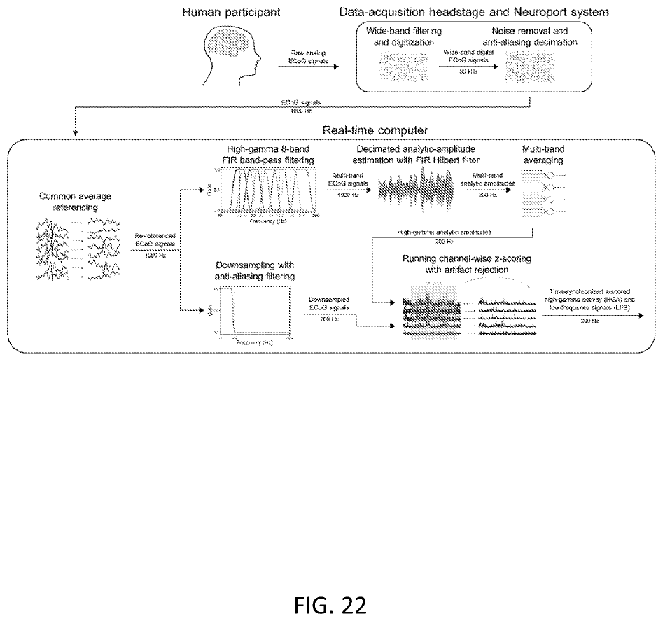

We acquired neural features from the implanted ECoG array using a pipeline involving several hardware components and processing steps (see FIG. 22). We connected a headstage (a detachable digital connector; NeuroPlex E, Blackrock Microsystems) to the percutaneous pedestal connector, which digitized neural signals from the ECoG array and transmitted them through an HDMI connection to a digital hub (Blackrock Microsystems). The digital hub then transmitted the digitized signals through an optical fiber cable to a Neuroport system (Blackrock Microsystems), which applied noise cancellation and an anti-aliasing filter to the signals before streaming them at 1 kHz through an Ethernet connection to a separate real-time computer (Colfax International).

On the real-time processing computer, we used a custom Python software package (rtNSR) to process and analyze the ECoG signals, execute the real-time tasks, perform real-time decoding, and store the data and task metadata^{16,33,34}. Using this software package, we first applied a common average reference (across all electrode channels) to each time sample of the ECoG data. Common average referencing is commonly applied to multi-channel datasets to reduce shared noise^{35,36}. These re-referenced signals were then processed in two parallel processing streams to extract high-gamma activity (HGA) and low-frequency signal (LFS) features using digital finite impulse response (FIR) filters designed using the Parks-McClellan algorithm 37 (see FIG. 22). Briefly, we used these FIR filters to compute the analytic amplitude of the signals in the high-gamma frequency band (70-150 Hz) and an anti-aliased version of the signals (with a cutoff frequency at 100 Hz). We combined the time-synchronized high-gamma analytic amplitudes and downsampled signals into a single feature stream at 200 Hz. Next, we z-scored the values for each channel and each feature type using a 30-second sliding window to compute running statistics. Finally, we implemented an artifact-rejection approach that identified neural time points containing at least 32 features with z-score magnitudes greater than 10, replacing each of these time points with the z-score values from the preceding time point and ignoring these time points when updating the running z-score statistics. During real-time decoding and in offline analyses, we used the z-scored high-gamma analytic amplitudes as the HGA features and the z-scored downsampled signals as the LFS features (and the combination of the two as the HGA+LFS feature set). The neural classifier further downsampled these feature streams by a factor of 6 before using them for inference (using an anti-aliasing filter with a cutoff frequency at 16.67 Hz), but the speech detector did not.

We performed all data collection and real-time decoding tasks in a small office room near the participant's residence. We uploaded data to our lab's server infrastructure and trained the decoding models using NVIDIA V100 GPUs hosted on this infrastructure. Additional information regarding the recording hardware, task-setup procedures with the participant, and clinical trial protocol are provided in our previous work¹⁶.

### Task Design

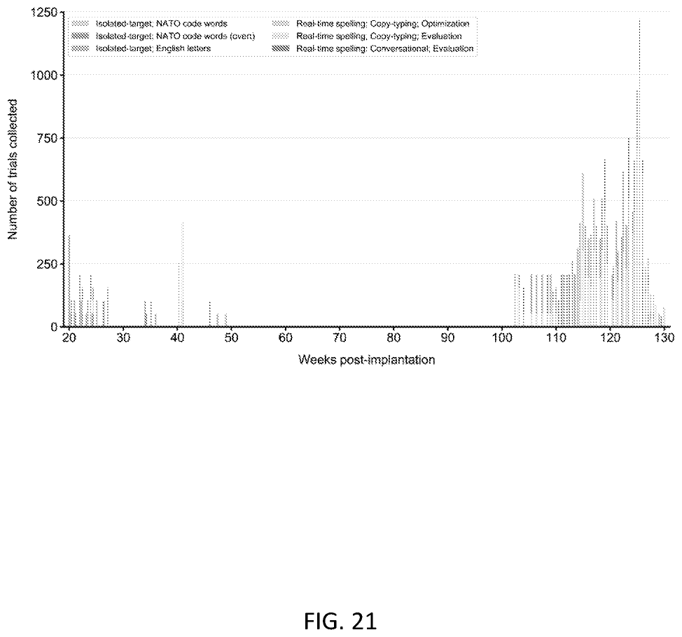

We recorded neural data with the participant during two general types of tasks: an isolated-target task and a sentence-spelling task (FIG. 21). In each trial of the isolated-target task, a text target appeared on the screen along with 4 dots on either side. Dots on both sides disappeared one by one until no dots remained, at which point the text target turned green to represent a go cue. At this go cue, the participant either attempted to say the target (silently or aloud, depending on the current task instructions) if it was either a NATO code word or an English letter. If the target was a text string containing the word “Right” and an arrow pointing right, the participant instead attempted to squeeze his right hand. We used the neural data collected during the isolated-target task to train and optimize the detection and classification models and to evaluate classifier performance (see Method S1).

The sentence-spelling task is described in the start of the Results section and in FIG. 15. Briefly, the participant used the full spelling pipeline (described in the following sub-section) to either spell sentences presented to him as targets in a copy-typing task condition or to spell arbitrary sentences in a conversational task condition. We did not implement functionality to allow the participant to retroactively alter the predicted sentence, although the language model could alter previously predicted words in a sentence after receiving additional character predictions. Data collected during the sentence-spelling task were used to optimize beam-search hyperparameters and evaluate the full spelling pipeline.

### Modeling

We fit detection and classification models using data collected during the isolated-target task as the participant attempted to produce code words and the hand-motor command. After fitting these models offline, we saved the trained models to the real-time computer for use during real-time testing. In addition to these two models, we also used language models to enable sentence spelling. We used hyperparameter optimization procedures on held-out validation datasets to choose values for model hyperparameters (see Table S2).

### Speech Detection

To determine when the participant was attempting to engage the spelling system, we developed a real-time silent-speech detection model. Similar to a previous implementation, this model used long short-term memory layers, a type of recurrent neural network layer, to process neural activity in real time and detect attempts to silently speak¹⁶. This model used both LFS and HGA features (a total of 256 individual features) at 200 Hz.

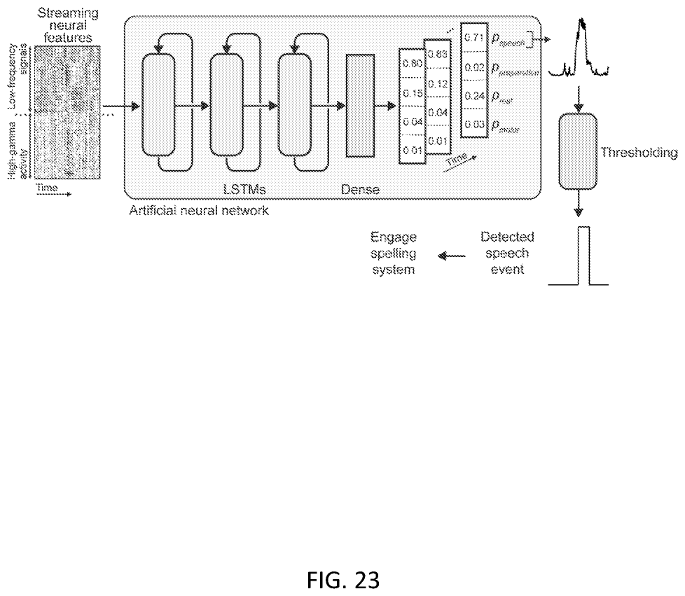

The speech-detection model was trained using supervised learning and truncated backpropagation through time. For training, we labeled each time point in the neural data as one of four classes depending on the current state of the task at that time: ‘rest’, ‘speech preparation’, ‘motor’, and ‘speech.’ Though only the speech probabilities were used during real-time evaluation to engage the spelling system, the other labels were included during training to help the detection model disambiguate attempts to speak from other behavior. See Method S2 and FIG. 23 for further details about the speech-detection model.

### Classification

We trained an artificial neural network (ANN) to classify the attempted code word or hand-motor command yᵢ from the time window of neural activity xᵢ associated with an isolated-target trial or 2.5-s letter-decoding cycle i. The training procedure was a form of maximum likelihood estimation, where given an ANN classifier parameterized by 0 and conditioned on the neural activity xᵢ, our goal during model fitting was to find the parameters θ* that maximized the probability of the training labels. This can be written as the following optimization problem:

$${θ}^{*} = arg {max}_{θ} {\prod }_{i} {p}_{θ} ( {y}_{i} \vert {x}_{i} ) = arg {max}_{θ} {\sum }_{i} log {p}_{θ} ( {y}_{i} \vert {x}_{i} ) = arg {min}_{θ} - {\sum }_{i} log {p}_{θ} ( {y}_{i} \vert {x}_{i} )$$

We approximated the optimal parameters θ* using stochastic gradient descent and the Adam optimizer³⁸.

To model the temporal dynamics of the neural time-series data, we used an ANN with a one-dimensional temporal convolution on the input layer followed by two layers of bidirectional gated recurrent units (GRUs)³⁹, for a total of three layers. We multiplied the final output of the last GRU layer by an output matrix then applied a softmax function to yield the estimated probability of each of the 27 labels ŷᵢ given xᵢ. See Method S3 for further details about the data-augmentation, hyperparameter-optimization, and training procedures used to fit the neural classifier.

Classifier ensembling for sentence spelling: During sentence spelling, we used model ensembling to improve classification performance by reducing overfitting and unwanted modeling variance caused by random parameter initializations 4. Specifically, we trained 10 separate classification models using the same training dataset and model architecture but with different random parameter initializations. Then, for each time window of neural activity xᵢ, we averaged the predictions from these 10 different models together to produce the final prediction ŷᵢ.

### Incremental Classifier Recalibration for Sentence Spelling

To improve sentence-spelling performance, we trained the classifiers used during sentence spelling on data recorded during sentence-spelling tasks from preceding sessions (in addition to data from the isolated-target task). In an effort to only include high-quality sentence-spelling data when training these classifiers, we only used data from sentences that were decoded with a character error rate of 0.

### Beam Search

During sentence spelling, our goal was to compute the most likely sentence text s* given the neural data X. We used the formulation from Hannun et al.¹⁹ to find s* given its likelihood from the neural data and its likelihood under an adjusted language-model prior, which allowed us to incorporate word-sequence probabilities with predictions from the neural classifier. This can be expressed formulaically as:

$${s}^{*} = arg {max}_{s} {p}_{nc} ( s \vert X ) {{p}_{lm} ( s )}^{α} {\vert s \vert}^{β}$$

Here, p_{nc}(s|X) is the probability of s under the neural classifier given each window of neural activity, which is equal to the product of the probability of each letter ins given by the neural classifier for each window of neural activity xᵢ. pₗₘ) is the probability of the sentence sunder a language-model prior. Here, we used an n-gram language model to approximate pₗₘ). Our n-gram language model, with n=3, provides the probability of each word given the preceding two words in a sentence. The probability under the language model of a sentence is then taken as the product of the probability of each word given the two words that precede it (see Method S5).

As in Hannun et al. 1, we assumed that the n-gram language-model prior was too strong and downweighted it using a hyperparameter a. We also included a word-insertion bonus β to encourage the language model to favor sentences containing more words, counteracting an implicit consequence of the language model that causes the probability of a sentence under it pₗₘ(s) to decrease as the number of words in s increases. |s| denotes the cardinality of s, which is equal to the number of words in s. If a sentence s was partially completed, only the words preceding the final whitespace character in s were considered when computing pₗₘ(s) and Isl.

We then used an iterative beam-search algorithm as in Hannun et al.¹⁹ to approximate s*at each timepoint t=τ. We used a list of the B most likely sentences from t=τ−1 (or a list containing a single empty-string element if t=1 as a set of candidate prefixes, where B is the beam width. Then, for each candidate prefix l and each English letter c with p_{nc}(c|x_{τ})>0.001, we constructed new candidate sentences by considering l followed by c. Additionally, for each candidate prefix l and each text string c⁺, composed of an English letter followed by the whitespace character, with p_{nc}(c⁺|x_{τ})>0.001, we constructed more new candidate sentences by considering l followed by c+. Here and throughout the beam search, we considered p_{nc}(c⁺|x_{τ})=p_{nc}(c⁺|x_{τ}) for each c and corresponding c⁺. Next, we discarded any resulting candidate sentences that contained words or partially completed words that were not valid given our constrained vocabulary. Then, we rescored each remaining candidate sentence I with p(l̅)=p_{nc}(l̅|X_{1:τ})pₗₘ(l̅)^{α}|l̅|^{β}. The most likely candidate sentence, s*, was then displayed as feedback to the participant.

We chose values for α, β, and B using hyperparameter optimization (See Method S4 for more details).

If at any time point t the probability of the attempted hand-motor command (the sentence-finalization command) was greater than 80%, the B most likely sentences from the previous iteration of the beam search were processed to remove any sentence with incomplete or out-of-vocabulary words. The probability of each remaining sentence \hat{l} was then recomputed as

$$p ( \hat{l} ) = {p}_{nc} ( \hat{l} \vert {X}_{1 : t - 1} ) {{p}_{lm} ( \hat{l} )}^{α} {\vert \hat{l} \vert}^{β} {{p}_{gpt 2} ( \hat{l} )}^{{α}_{gpt 2}}$$

Here, p_{gpt2}(l̅) denotes the probability of \hat{l} under the DistilGPT-2 language model, a low-parameter variant of GPT-2 (see Method S5 for more details), and α_{gpt2} represents a scaling hyperparameter that was set through hyperparameter optimization. The most likely sentence \hat{l} given this formulation was then displayed to the participant and stored as the finalized sentence.

See Method S4 for further details about the beam-search algorithm.

### Performance Evaluation

### Character Error Rate (CER) and Word Error Rate (WER):

Because CER and WER are overly influenced by short sentences, as in previous studies^{6,16} we reported CER and WER as the sum of the character or word edit distances between each of the predicted and target sentences in a sentence-spelling block and then divided this number by the total number of characters or words across all target sentences in the block. Each block contained between two to five sentence trials.

### Assessing Performance During the Conversational Task Condition:

To obtain ground truth sentences to calculate CERs and WERs for the conversational condition of the sentence-spelling task, after completing each block we reminded the participant of the questions and the decoded sentences from that block, and then, for each decoded sentence, he either confirmed that the decoded sentence was correct or typed out the intended sentence using his commercially available assistive-communication device. Each block used for evaluation contained between two to four sentence trials.

### Characters and Words Per Minute:

We calculated the characters per minute and words per minute rates for each sentence-spelling (copy-typing) block as follows:

$$rate = \frac{{\sum }_{i} {N}_{i}}{{\sum }_{i} {D}_{i}} .$$

Here, i indexes each trial, Nᵢ denotes the number of words or characters (including whitespace characters) decoded for trial i, and Dᵢ denotes the duration of trial i (in minutes; computed as the difference between the time at which the window of neural activity corresponding to the final code word in trial i ended and the time of the go cue of the first code word in trial i).

### Electrode Contributions

To compute electrode contributions using data recorded during the isolated-target task, we computed the derivative of the classifier's loss function with respect to the input features across time as in Simonyan et al. 4, yielding a measure of how much the predicted model outputs were affected by small changes to the input feature values for each electrode and feature type (HGA or LFS) at each time point. Then, we calculated the L2-norm of these values across time and averaged the resulting values across all isolated-target trials, yielding a single contribution value for each electrode and feature type for that classifier.

### Cross-Validation

For each fold, we used stratified cross-validation folds of the isolated-target task. We split each fold into a training set containing 90% of the data and a held-out testing set containing the remaining 10%, 10% of the training dataset was then selected as a validation set.

### Analyzing Neural-Feature Principal Components

To characterize the HGA and LFS neural features, we used bootstrapped principal component analyses. First, for each NATO code word, we randomly sampled (with replacement) cue-aligned time windows of neural activity (spanning from the go cue to 2.5 seconds after the go cue) from the first 318 silently attempted isolated-target trials for that code word. To clearly understand the role of each feature stream for classification, we downsampled the signals by a factor of 6 to obtain the signals used by the classifier. Then, we trial averaged the data for each code word, yielding 26 trial averages across time for each electrode and feature set (HGA, LFS, and HGA+LFS). We then arranged this into a matrix with dimensionality N×TC, where N is the number of features (128 for HGA and for LFS; 256 for HGA+LFS), T is the number of time points in each 2.5-second window, and C is the number of NATO code words (26), by concatenating the trial-averaged activity for each feature. We then performed principal component analysis along the feature dimension of this matrix. Additionally, we arranged the trial-averaged data for each code word into a matrix with dimensionality T×NC. We then performed principal component analysis along the temporal dimension. For each analysis, we performed the measurement procedure 100 times to obtain a representative distribution of the minimum number of principal components required to explain more than 80% of the variance.

### Nearest-Class Distance Comparison

To compare nearest-class distances for the code words and letters, we first calculated averages across 1,000 bootstrap iterations of the combined HGA+LFS feature set across 47 silently attempted isolated-target trials for each code word and letter. We then computed the Frobenius norm of the difference between each pairwise combination. For each code word, we used the smallest computed distance between that code word and any other code word as the nearest-class distance. We then repeated this process for the letters.

### Generalizability to Larger Vocabularies

During real-time sentence spelling, the participant created sentences composed of words from a 1,152-word vocabulary that contained common words and words relevant to clinical caregiving. To assess the generalizability of our system, we tested the sentence-spelling approach in offline simulations using three larger vocabularies. The first of these vocabularies was based on the ‘Oxford 3000’ word list, which is composed of 3,000 core words chosen based on their frequency in the Oxford English Corpus and relevance to English speakers⁴². The second was based on the ‘Oxford 5000’ word list, which is the ‘Oxford 3000’ list augmented with an additional 2,000 frequent and relevant words. The third was a vocabulary based on the most frequent 10,000 words in Google's Trillion Word Corpus, a corpus of over 1 trillion words of text⁴³. To eliminate non-words that were included in this list (such as “f”, “gp”, and “ooo”), we excluded words composed of 3 or fewer characters if they did not appear in the ‘Oxford 5000’ list. After supplementing each of these three vocabularies with the words from the original 1,152-word vocabulary that were not already included, the three finalized vocabularies contained 3,303, 5,249, and 9,170 words (these sizes are given in the same order that the vocabularies were introduced).

For each vocabulary, we retrained the n-gram language model used during the beam-search procedure with n-grams that were valid under the new vocabulary (see Method S5) and used the larger vocabulary during the beam search. We then simulated the sentence-spelling experiments offline using the same hyperparameters that were used during real-time testing.

### Trial Rejection

During the copy-typing condition of the sentence-spelling task, the participant was instructed to attempt to silently spell each intended sentence regardless of how accurate the decoded sentence displayed as feedback was. However, during a small number of trials, the participant self-reported making a mistake (for example, by using the wrong code word or forgetting his place in the sentence) and sometimes stopped his attempt. This mostly occurred during initial sentence-spelling sessions while he was still getting accustomed to the interface. To focus on evaluating the performance of our system rather than the participant's performance, we excluded these trials (13 trials out of 163 total trials) from performance-evaluation analyses, and we had the participant attempt to spell the sentences in these trials again in subsequent sessions to maintain the desired amount of trials during performance evaluation (2 trials for each of the 75 unique sentences). Including these rejected sentences when evaluating performance metrics only modestly increased the median CER and WER observed during real-time spelling blocks to 8.52% (99% CI [3.20, 15.1]) and 13.75% (99% CI [8.71, 29.9]), respectively.

During the conversational condition of the sentence-spelling task, trials were rejected if the participant self-reported making a mistake (as in the copy-typing condition) or if an intended word was outside of the 1,152 word vocabulary. For some blocks, the participant indicated that he forgot one of his intended responses when we asked him to report the intended response after the block concluded. Because there was no ground truth for this conversational task condition, we were unable to use the trial for analysis. Of 39 original conversational sentence-spelling trials, the participant got lost on 2 trials, tried to use an out-of-vocabulary word during 6 trials, and forgot the ground-truth sentence during 3 trials (leaving 28 trials for performance evaluation). Incorporating blocks where the participant used intended words outside of the vocabulary only modestly raised CER and WER to median values of 15.7% (99% CI [6.25, 30.4]) and 17.6%, (99% CI [12.5, 45.5]) respectively.

### Statistical Testing

The statistical tests used in this work are all described in the figure captions and text. In brief, we used two-sided Wilcoxon Rank-Sum tests to compare any two groups of observations. When the observations were paired, we instead used a two-sided Wilcoxon signed-rank test. We used Holm-Bonferroni correction for comparisons in which the underlying neural data were not independent of each other. We considered P-values less than 0.01 as significant. We computed P-values for Spearman rank correlations using permutation testing. For each permutation, we randomly shuffled one group of observations and then determined the correlation. We computed the p-value as the fraction of permutations that had a correlation value with a larger magnitude than the Spearman rank correlation computed on the non-shuffled observations. For any confidence intervals around a reported metric, we used a bootstrap approach to estimate the 99% confidence interval. On each iteration (of a total of 2000 iterations), we randomly sampled the data (such as accuracy per cross-validation fold) with replacement and calculated the desired metric (such as the median). The confidence interval was then computed on this distribution of the bootstrapped metric.

### BIBLIOGRAPHY

1. Beukelman, D. R., Fager, S., Ball, L. & Dietz, A. AAC for adults with acquired neurological conditions: A review. Augment. Altem. Commun. 23, 230-242 (2007).
2. Felgoise, S. H., Zaccheo, V., Duff, J. & Simmons, Z. Verbal communication impacts quality of life in patients with amyotrophic lateral sclerosis. Amyotroph. Lateral Scler. Front. Degener. Amyotroph. Lateral Scler. Front. Degener. 17, 179-183 (2016).
3. Brumberg, J. S., Pitt, K. M., Mantie-Kozlowski, A. & Burnison, J. D. Brain-Computer Interfaces for Augmentative and Alternative Communication: A Tutorial. Am. J. Speech Lang. Pathol. 27, 1-12 (2018).
4. Vansteensel, M. J. et al. Fully Implanted Brain-Computer Interface in a Locked-In Patient with ALS. N. Engl. J. Med. 375, 2060-2066 (2016).
5. Pandarinath, C. et al. High performance communication by people with paralysis using an intracortical brain-computer interface. eLife 6, 1-27 (2017).
6. Willett, F. R., Avansino, D. T., Hochberg, L. R., Henderson, J. M. & Shenoy, K. V. High-performance brain-to-text communication via handwriting. Nature 593, 249-254 (2021).
7. Branco, M. P. et al. Brain-Computer Interfaces for Communication: Preferences of Individuals With Locked-in Syndrome. Neurorehabil. Neural Repair 35, 267-279 (2021).
8. Bouchard, K. E., Mesgarani, N., Johnson, K. & Chang, E. F. Functional organization of human sensorimotor cortex for speech articulation. Nature 495, 327-332 (2013).
9. Carey, D., Krishnan, S., Callaghan, M. F., Sereno, M. I. & Dick, F. Functional and Quantitative MRI Mapping of Somatomotor Representations of Human Supralaryngeal Vocal Tract. Cereb. Cortex 27, 265-278 (2017).
10. Chartier, J., Anumanchipalli, G. K., Johnson, K. & Chang, E. F. Encoding of Articulatory Kinematic Trajectories in Human Speech Sensorimotor Cortex. Neuron 98, 1042-1054.e4 (2018).
11. Lotte, F. et al. Electrocorticographic representations of segmental features in continuous speech. Front. Hum. Neurosci. 09, 1-13 (2015).
12. Herff, C. et al. Brain-to-text: decoding spoken phrases from phone representations in the brain. Front. Neurosci. 9, 1-11 (2015).
13. Makin, J. G., Moses, D. A. & Chang, E. F. Machine translation of cortical activity to text with an encoder-decoder framework. Nat. Neurosci. 23, 575-582 (2020).
14. Mugler, E. M. et al. Direct classification of all American English phonemes using signals from functional speech motor cortex. J. Neural Eng. 11, 035015-035015 (2014).
15. Sun, P., Anumanchipalli, G. K. & Chang, E. F. Brain2Char: a deep architecture for decoding text from brain recordings. J. Neural Eng. 17, 066015 (2020).
16. Moses, D. A. et al. Neuroprosthesis for Decoding Speech in a Paralyzed Person with Anarthria. N. Engl. J. Med. 385, 217-227 (2021).
17. Adolphs, S. & Schmitt. Lexical Coverage of Spoken Discourse. Appl. Linguist. 24, 425-438 (2003).
18. van Tilborg, A. & Deckers, S. R. J. M. Vocabulary Selection in AAC: Application of Core Vocabulary in Atypical Populations. Perspect. ASHA Spec. Interest Groups 1, 125-138 (2016).
19. Hannun, A. Y., Maas, A. L., Jurafsky, D. & Ng, A. Y. First-Pass Large Vocabulary Continuous Speech Recognition using Bi-Directional Recurrent DNNs. ArXiv14082873 Cs (2014).
20. Silversmith, D. B. et al. Plug-and-play control of a brain-computer interface through neural map stabilization. Nat. Biotechnol. 39, 326-335 (2020).
21. Rezeika, A. et al. Brain-Computer Interface Spellers: A Review. Brain Sci. 8, 57 (2018).
22. Sellers, E. W., Ryan, D. B. & Hauser, C. K. Noninvasive brain-computer interface enables communication after brainstem stroke. Sci. Transl. Med. 6, 257re7-257re7 (2014).
23. Gilja, V. et al. A high-performance neural prosthesis enabled by control algorithm design. Nat. Neurosci. 15, 1752-1757 (2012).
24. Kawala-Sterniuk, A. et al. Summary of over Fifty Years with Brain-Computer Interfaces-A Review. Brain Sci. 11, 43 (2021).
25. Serruya, M. D., Hatsopoulos, N. G., Paninski, L., Fellows, M. R. & Donoghue, J. P. Instant neural control of a movement signal. Nature 416, 141-142 (2002).
26. Wolpaw, J. R., McFarland, D. J., Neat, G. W. & Fomeris, C. A. An EEG-based brain-computer interface for cursor control. Electroencephalogr. Clin. Neurophysiol. 78, 252-259 (1991).
27. Laufer, B. What percentage of text-lexis is essential for comprehension. Spec. Lang. Hum. Think. Think. Mach. 316323, (1989).
28. Webb, S. & Rodgers, M. P. H. Vocabulary Demands of Television Programs. Lang. Learn. 59, 335-366 (2009).
29. Nourski, K. V. et al. Sound identification in human auditory cortex: Differential contribution of local field potentials and high gamma power as revealed by direct intracranial recordings. Brain Lang. 148, 37-50 (2015).
30. Conant, D. F., Bouchard, K. E., Leonard, M. K. & Chang, E. F. Human sensorimotor cortex control of directly-measured vocal tract movements during vowel production. J. Neurosci. 38, 2382-17 (2018).
31. Gerardin, E. et al. Partially Overlapping Neural Networks for Real and Imagined Hand Movements. Cereb. Cortex 10, 1093-1104 (2000).
32. Guenther, F. H. & Hickok, G. Neural Models of Motor Speech Control. in Neurobiology of Language 725-740 (Elsevier, 2016).
33. Moses, D. A., Leonard, M. K. & Chang, E. F. Real-time classification of auditory sentences using evoked cortical activity in humans. J. Neural Eng. 15, (2018).
34. Moses, D. A., Leonard, M. K., Makin, J. G. & Chang, E. F. Real-time decoding of question-and-answer speech dialogue using human cortical activity. Nat. Commun. 10, 3096 (2019).
35. Ludwig, K. A. et al. Using a common average reference to improve cortical neuron recordings from microelectrode arrays. J. Neurophysiol. 101, 1679-89 (2009).
36. Williams, A. J., Trumpis, M., Bent, B., Chiang, C.-H. & Viventi, J. A Novel pECoG Electrode Interface for Comparison of Local and Common Averaged Referenced Signals. in 2018 40th Annual International Conference of the IEEE Engineering in Medicine and Biology Society (EMBC) 5057-5060 (IEEE, 2018). doi:10.1109/EMBC.2018.8513432.
37. Parks, T. W. & McClellan, J. H. Chebyshev Approximation for Nonrecursive Digital Filters with Linear Phase. IEEE Trans. Circuit Theory 19, 189-194 (1972).
38. Kingma, D. P. & Ba, J. Adam: A Method for Stochastic Optimization. ArXiv14126980 Cs (2017).
39. Cho, K. et al. Learning Phrase Representations using RNN Encoder-Decoder for Statistical Machine Translation, in 1724-1734 (2014). doi:http://dx.doi.org/10.3115/v1/D14-1179.
40. Fort, S., Hu, H. & Lakshminarayanan, B. Deep Ensembles: A Loss Landscape Perspective. ArXiv191202757 Cs Stat (2020).
41. Simonyan, K., Vedaldi, A. & Zisserman, A. Deep Inside Convolutional Networks: Visualising Image Classification Models and Saliency Maps. ArXiv13126034 Cs (2014).
42. About the Oxford 3000 and 5000 word lists at Oxford Learner's Dictionaries. https://www.oxfordleamersdictionaries.com/us/about/wordlists/oxford3000-5000.
43. Brants, Thorsten & Franz, Alex. Web iT 5-gram Version 1. 20971520 KB (2006) doi:10.35111/CQPA-A498.

### Example 4: Participant Survey on Overt- Versus Silent-Speech Attempts

We asked the participant the following questions about controlling the spelling system using either silent or overt attempts to speak. The participant's responses are provided after each question.
1. How long do you think you could comfortably use the spelling system for communication with overt-speech attempts? Response: 15 minutes
2. How long do you think you could comfortably use the spelling system for communication with silent-speech attempts? Response: 30 minutes
3. Can you please rank your comfort using the spelling system with overt-speech attempts on a scale from 1-10? Response: 5
4. Can you please rank your comfort using the spelling system with silent-speech attempts on a scale from 1-10? Response: 8
5. What is the minimum amount of time you need between go cues to use the spelling system with overt-speech attempts? Response: 4 seconds
6. What is the minimum amount of time you need between go cues to use the spelling system with silent-speech attempts? Response: 2.5 seconds
7. How does using silent-speech attempts compare to using overt speech attempts to control the speller device?
(a) Silent is much easier than overt
(b) Silent is easier than overt
(c) Silent is the same as overt
(d) Silent is harder than overt
(e) Silent is much harder than overt
   Response: (a) Silent is Much Easier than Overt

The participant's responses are summarized below. Overall, the participant vastly prefers silent-speech attempts to control the spelling neuroprosthesis.

| Question | Overt | | Silent | |
|---|---|---|---|---|
| How long could you comfortably use the | 15 | minutes | 30 | minutes |
| device? | | | | |
| What is your comfort using the device for | 5 | | 8 | |
| communication (1-10)? | | | | |
| What is the smallest amount of time you | 4 | seconds | 2.5 | seconds |
| need between go cues? | | | | |

| | | |
|---|---|---|
| How much easier is using silent speech | (n/a) | Much easier |
| attempts than overt? | | |

### Example 5: Data Re-Normalization

To promote neural-feature consistency across recording sessions, we used a running 30-second z-score on all neural features (see FIG. 22). However, the neural activity recorded during the participant's attempts to squeeze his right hand typically differed in signal magnitude when compared to activity recorded during silent-speech attempts. As a result, when using a running z-score, some isolated-target task blocks with only speech content (letter and NATO code-word trials) or only attempted hand-movement trials had different neural-feature baselines than isolated-target blocks with both speech and hand-movement trials.

To mitigate this, we jointly re-normalized letter and NATO code-word isolated-target blocks and attempted hand-movement isolated-target blocks that were recorded on the same day. For each recording day, and independently for each speech type (silent or overt), we combined all attempted speech trials and attempted hand-movement trials that were recorded on that day by concatenating (along the time dimension) time windows of neural features (high-gamma activity and low-frequency signals without z-score normalization) associated with these trials. These time windows of neural features ranged from 2 seconds before to 3.5 seconds after the go cue for each trial. To reduce the effect of potential signal artifacts in these un-normalized signals, we clipped the signal magnitude for each feature (each electrode channel for each feature type) to be within the 1st and 99th percentiles of the signal magnitudes recorded for that feature. Then, we re-normalized the neural features for each trial recorded on that day by subtracting the feature-wise mean and dividing by the feature-wise standard deviation of the concatenated data matrix. Note that some task blocks containing only attempted speech or only attempted hand-movements were not re-normalized in this manner (if there were not both types of data recorded on the same day). Additionally, because some attempted hand-movement blocks were recorded on days where both overtly and silently attempted NATO code-word isolated-target were also recorded, this meant that there were three possible types of attempted hand-movement blocks: blocks that were not re-normalized (these blocks were not recorded on the same day as blocks containing only attempted speech), blocks that were re-normalized with blocks that only contained overt-speech attempts, and blocks that were re-normalized with blocks that only contained silent-speech attempts. Data from task blocks that were not re-normalized used the running 30-second z-score normalization procedure and automatic artifact rejection described in FIG. 22.

### Example 6: Supplementary Information for Spelling Decoding

### Section S1. Isolated-Target Task

We recorded the participant's neural activity as he silently (or sometimes overtly) attempted to say prompted utterances or perform prompted motor movements during an isolated-target task. As described in the Methods section of the main text, each trial of the isolated-target task began with the textual presentation of a single speech or motor target on the participant's screen with 4 dots on either side of the text. These dots disappeared one at a time (simultaneously on each side of the text) at a constant rate, providing task timing to the participant. As the final dot disappeared, the text target turned green, representing a go cue. At this go cue, the participant was instructed to attempt to produce the target. The text target remained on the participant's screen for a brief interval before the screen was cleared and the next trial began.

We collected the following four utterance sets with the isolated-target paradigm for training the speech detection and neural classification models:
1. 26 English letters
2. 26 NATO code words
3. 26 NATO code words and attempted hand squeeze
4. Attempted hand squeeze and 3 other attempted motor movements

Within each block of the isolated-target task, the rate at which the countdown dots disappeared τₚ and the duration that the target text remained on the screen after the go cue it was identical across trials within a single block. However, these two task-interval parameters did vary across blocks. For the attempted motor movement blocks, we used τₚ∈[0.35, 0.5] seconds per dot and τₜ=4.0 seconds. For all other isolated-target blocks, we used τₚ∈[0.45, 1.5] seconds per dot and τₜ∈[0.45, 6.0] seconds.

### Section S2. Speech-Detection Model

We designed a speech-detection model to analyze the neural features in real time to identify when a silently attempted speech event occurred. We used this speech detector to enable volitional engagement of the spelling system during real-time sentence spelling. All data used to train and evaluate the speech detector was either trials of attempted hand squeezes or of silently attempted speech (no overtly attempted speech data was used).

### Data Preparation

We trained the speech detector using data from isolated-target task blocks containing trials of the 26 NATO code words, blocks containing trials of the 26 NATO code words and the attempted right-hand squeeze, and blocks containing a variety of attempted motor movements including the attempted hand squeeze (from which we only used the attempted hand squeeze). We used four categories to label each time point of neural-feature data to train the speech detector: “speech preparation”, “speech”, “motor”, and “rest”. Time points between the appearance of a target NATO code word on the participant's screen and the associated go cue were labeled as speech preparation. Time points between a go cue and 1 second after that go cue for NATO code-word attempts were labeled as speech. Time points between a go cue and 2 seconds after that go cue for attempted hand squeezes were labeled as motor. Time points between the end of the allotted time period for an attempt (1 second after the go cue for speech or 2 seconds for hand-squeezes) and the end of that trial (when the screen cleared for an inter-trial interval) were not trained on. Training data for the speech detector included blocks of the attempted motor isolated target task. For blocks containing only attempted motor movements, time points during attempted motor trials that were not the attempted hand squeeze were ignored. All other time points were labeled as rest.

The speech detector used both low-frequency signals (LFS) and high-gamma activity (HGA) as features at 200 Hz. Note that this is different than the classifier, which also used these features but further downsampled them to 33.3 Hz.

### Model Architecture and Training

We used Python 3.6.6 and PyTorch 1.6.0 to create and train the speech detector [1]. The speech detector contained a stack of 3 long short-term memory (LSTM) layers with 100, 50, and 50 nodes, respectively. The LSTM layers were followed by a single fully connected layer that projected the latent dimensions to probabilities across the four classes (speech preparation, speech, rest, and motor). The model processed each time point continuously from the feature stream, outputting a continuous stream of probabilities (one predicted probability vector per neural-feature time point at 200 Hz). A schematic of the model is shown in FIG. 23.

The speech-detection model was trained to minimize a modified cross-entropy loss. Cross-entropy loss is originally defined as:

$$\begin{matrix}{H}_{P , Q} ( ℓ \vert y ) = {𝔼}_{P} [ - log Q ( ℓ \vert y ) ] \\ \approx - \frac{1}{N} {\sum }_{n = 1}^{N} log Q ( {ℓ}_{n} \vert {y}_{n} )\end{matrix} , \qquad ( S1 )$$

where:
P: The true distribution of the classes, determined by the assigned class labels [symbol] .
N: The number of samples.
H_{P,Q}( [symbol] |y): The cross entropy of the predicted distribution with respect to the true distribution for [symbol] .
log: The natural logarithm.

We modified this loss to add an extra penalty on 3 types of incorrect predictions: time points that were labeled as motor but predicted to be speech, time points that were labeled as speech but predicted to be motor, and time points that were labeled as rest but predicted to be speech. In practice, we defined wₙ as 1.1. With these modifications, the cross-entropy loss defined in Equation S1 is redefined as:

$${H}_{P , Q} ( ℓ \vert y ) \approx - \frac{1}{N} {\sum }_{n}^{N} {w}_{n} log Q ( {ℓ}_{n} \vert {y}_{n} ) , \qquad ( S2 )$$

where wₙ is the penalty weight for sample n and is defined as:

$${w}_{n} := \{ \begin{matrix}1.1 & if ( {ℓ}_{n} = motor ) and ( {arg max}_{l ∈ L} [ Q ( l \vert {y}_{n} ) ] = speech ) \\ 1.1 & if ( {ℓ}_{n} = speech ) and ( {arg max}_{l ∈ L} [ Q ( l \vert {y}_{n} ) ] = motor ) \\ 1.1 & if ( {ℓ}_{n} = rest ) and ( {arg max}_{l ∈ L} [ Q ( l \vert {y}_{n} ) ] = speech ) \\ 1 & otherwise\end{matrix} . \qquad ( S3 )$$

We used this penalty modification to reduce the likelihood that the speech detector would make false-positive mistakes (such as erroneously detecting an attempted-speech event when the participant was actually attempting to squeeze his hand).

As previously described in [2], we used truncated backpropagation through time (BPTT) to train the speech detector. In brief, we manually implemented BPTT by only letting the speech-detection model backpropagate 500 ms at a time to prevent the model from relying on task periodicity to make predictions. We used the Adam optimizer to minimize the cross-entropy loss given in Equation S2 [3], with a learning rate of 0.001 and default values for the remaining optimization parameters. To prevent overfitting, we used early stopping on a held-out validation set and a dropout of 0.5 on each LSTM layer except for the final layer. For all training steps, we balanced classes between (included the same number of training examples for) the 4 possible classes.

### Event Detection

During real-time sentence spelling, the speech detector continuously processed time points of LFS and HGA and yielded a stream of silent-speech probabilities. We identified silent-speech events from this stream of probabilities using the same approach described in Supplementary Section S8 of [2]. In brief, the speech probabilities were first temporally smoothed using a moving window average. Then, we binarized the smoothed probabilities using a probability threshold. Finally, we “de-bounced” these binarized values by requiring that a change in binary state (from absence of speech to presence of speech, or vice versa) must last for longer than a certain duration of time before the change is deemed a speech onset or offset. These 3 parameter values were chosen via hyperparameter optimization and are listed in Table S2.

### Hyperparameter Optimization

The hyperparameter optimization process is identical to our previous work [2]. In brief, we used the hyperopt Python package [4] to optimize the 3 detection hyperparameters by minimizing a cost function based on a detection score. As defined in Supplementary Section S8 of [2]), the detection score is a measure encompassing both how accurately individual time points were predicted as speech or non-speech and how accurately the detector identified attempted-speech events in general. The cost function used to optimize the hyperparameters seeks to maximize the detection score while minimizing the time-threshold parameter (because we wanted to minimize the amount of time required to detect a silent-speech attempt. The cost function was defined as:

$${c}_{hp} ( ⊖ ) = {( 1 - {s}_{detection} )}^{2} + {λ}_{time} {θ}_{time} , \qquad ( S4 )$$

where:
cₕₚ (Θ): The value of the objective function using the hyperparameter value combination Θ.
λₜᵢₘₑ: The penalty applied to the time-threshold duration.
θₜᵢₘₑ: The time-threshold duration value, which is one of the three parameters contained in &.

Here, we used λₜᵢₘₑ=0.00025.

Because we only optimized the detection parameters that were applied to speech probabilities, we were able to compute the speech probability across a set of task blocks from a trained model and use the speech probabilities from these blocks to evaluate the hyperparameter combinations. After training a model on isolated-target blocks, we used the model to predict the speech probabilities for 12 held-out blocks of the isolated-target task containing NATO code-word silent-speech attempts and attempted hand squeezes. We chose to optimize over blocks containing both the silent-speech attempts and attempted hand squeezes because the real-time sentence-spelling task involved both of these types of attempts. After 1000 optimization iterations, we selected the final hyperparameters from the optimization run with the lowest cost value.

### Section S3. Classification Model

### Data Preparation

We trained the classifier using data from isolated-target task blocks containing trials of the 26 NATO code words, blocks containing trials of the 26 NATO code words and the attempted right-hand squeeze, and blocks containing a variety of attempted motor movements including the attempted hand squeeze (from which we only used the attempted hand squeeze). For the classifiers used during the feature-type, speech-type, and utterance-set comparisons, only data from isolated-target task blocks were used.

During training of the classifiers for real-time sentence spelling (and associated offline analyses), we also included sentence-spelling (copy-typing) trials in which the decoded sentence had a 0.0 character error rate (CER). These sentence-spelling trials constituted 3.060 of the data for overt-speech attempts (preliminary sentence-spelling trials with overt-speech attempts were collected but not used during evaluation) and 22.7% of the data for silent-speech attempts. For these classifiers, we also used a transfer-learning approach to pre-train on overt-speech attempts and then fine-tune on silent-speech attempts (except where otherwise noted; more details are provided later in this section). We never included sentence-spelling trials during classifier training that were recorded during the same session as (or, for associated offline analyses, a proceeding session of) any trials that were used during testing; classifiers were not recalibrated or updated during an evaluation session. The usages of certain datasets for certain evaluations are described in the table below.

| | Isolated- | Isolated- | Sentence- | Sentence- |
|---|---|---|---|---|
| Evaluation & associated | target | target | Spelling | Spelling |
| figure(s) in the main text | (overt) | (silent) | (overt) | (silent) |
| Real-time sentence- | Pre-train | Fine-tune | Pre-train | Fine-tune |
| spelling performance | | | | & Test |
| evaluation and related | | | | |
| offline analyses (beam- | | | | |
| search, language-model, | | | | |
| vocabulary-set, and task- | | | | |
| condition assessments) | | | | |
| [FIGS. 16 & 20] | | | | |
| Feature-type (HGA | | Train | | |
| versus LFS) comparisons | | & Test | | |
| [FIG. 17] | | | | |
| Offline speech-type | Train* | Train* | | |
| (silent vs. overt) | & Test | & Test | | |
| comparisons | | | | |
| [FIG. 18] | | | | |
| Offline utterance-set | | Train* | | |
| (letters vs. code words) | | & Test | | |
| [FIG. 19] | | | | |

*= used for pre-training and fine-tuning where applicable (see FIG. 18 for details)

There was no overlap between data used for evaluation and data used for hyperparameter optimization.

For each isolated-target trial, we defined the relevant time window of neural features (high-gamma activity (HGA) and low-frequency signal (LFS) features at 200 Hz) as 2 seconds before the go cue to 4 seconds after. This window of neural features was larger than the windows actually used for training and testing (detailed below in the “Architecture and training” sub-section) because we employed a time-jittering data augmentation, where smaller windows are pulled from this larger trial-relevant window. We then decimated the neural activity by a factor of 6 to 33.33 Hz with a 16.67 Hz anti-aliasing filter applied prior to decimation. We normalized each time sample to have an l2-norm of 1 across all neural features (each electrode channel and separately for the HGA and LFS feature types). For real-time inference and for offline evaluations, we used the combined (concatenated) HGA+LFS features during relevant time windows of neural activity. Thus, for each training example, we had a matrix of neural activity xᵢ of shape (T, C), where T is the number of time steps and C refers to the 256 features (2 features from each of the 128 electrodes). If only one feature stream was being used for a particular analysis, C would be equal to 128.

### Modeling Architecture and Training

To model the temporal and spatial dynamics of the participant's neural activity during silent-speech attempts, we trained artificial neural networks to classify which NATO code word (or the imagined hand squeeze) the participant had produced given a 2.5-second window of neural features after the associated go cue. We used gated-recurrent unit (GRU) layers [5], which have been shown to outperform other recurrent architectures (such as long-short term memory networks) [6] on sequence tasks [7].

In the classifier, neural features were first processed by a 1-dimensional convolutional layer parameterized by weights Wand bias term b. This results in an output representation hₙ (the output of hidden layer n) defined as:

$${h}_{1 , j} = {b}_{j} + {\sum }_{k = 0}^{C - 1} W [ j , k ] * {x}_{i} [ : , k ] , \qquad ( S5 )$$

where h_{1,j} is element j of the output of hidden layer 1, * denotes the valid cross-correlation operator, and C refers to the number of neural features in the input matrix xᵢ.

This representation was then passed into a stack of n GRU layers. Each unit was parameterized by Wᵢ, bᵢ, Wₕ, and bₕ, which are weights and biases that acted on the input and hidden states, respectively. Portions of each matrix were dedicated to a reset gate rₜ, an update gate zₜ, and a new gate nt.

At each time point t, the GRU computed:

$${r}_{t} = σ ( {W}_{ir} {x}_{t} + {b}_{ir} + {W}_{hr} h {(}_{t - 1 )} + {b}_{hr} ) ,$$

$${z}_{t} = σ ( {W}_{iz} {x}_{t} + {b}_{iz} + {W}_{hz} {h}_{( t - 1 )} + {b}_{hz} ) ,$$

$${n}_{t} = tanh ( {W}_{in} {x}_{t} + {b}_{in} + {r}_{t} * ( {W}_{hn} {h}_{( t - 1} ) + {b}_{hn} ) ) ,$$

$${h}_{t} = ( 1 - {z}_{t} ) * {n}_{t} + {z}_{t} * {h}_{( t - 1 )} ,$$

where * denotes the Hadamard product, σ denotes the sigmoid function, and ht is the output at each time point t for this layer. Basically, the GRU decided at each time point how much to update the hidden state from its previous value given the new activity (with the reset function incorporated) using zₜ. Each layer's output hₙ is used as the input to the next layer. During training, to minimize overfitting, we used dropout [8] to randomly set elements of hₙ to 0.0 with probability p_{dropout}, which we determined through hyperparameter optimization.

To improve accuracy, we used bidirectional GRU layers. This means that at each GRU, the input was copied, flipped backwards, and then used as an input to the network. This enabled us to learn forward and backward representations and use them as context when predicting class probabilities.

To compute a predicted probability distribution over the 26 NATO code words and imagined hand squeeze given the final time point of the final GRU layer, we multiplied this by a matrix Wₒᵤₜ and add a bias term bout, where Wₒᵤₜ has shape (Nₕₙ, 27), with Nₕₙ corresponding to the number of hidden units in the final GRU layer. We then applied a softmax function to these activations, giving the value of the output vector ŷ for each window i and each element (class) k to be:

$${\hat{y}}_{i , k} = \frac{exp ( {( {W}_{out} {h}_{n} )}_{k} )}{{\sum }_{j} ( exp ( {( {W}_{out} {h}_{n} )}_{j} )} , \qquad ( S6 )$$

where ŷᵢ can be thought of as a multinomial distribution over the possible output classes given sample xᵢ and the parameters of our neural-network model θ.

The goal during training was to maximize the likelihood of our labeled training data given the neural activity and θ, which can be written as the optimization problem:

$$\begin{matrix}{θ}^{*} = {arg max}_{θ} {\prod }_{i} {p}_{θ} ( {y}_{i} \vert {x}_{i} ) \\ = {arg max}_{θ} {\sum }_{i} log ( {p}_{θ} ( {y}_{i} \vert {x}_{i} ) ) .\end{matrix} \qquad ( S7 )$$

We approximated the solution to this problem using mini-batch stochastic gradient descent to solve the equivalent optimization problem:

$${θ}^{*} = {arg max}_{θ} {\sum }_{i} - log ( {p}_{θ} ( {y}_{i} \vert {x}_{i} ) ) . \qquad ( S8 )$$

Specifically, we use the Adam optimizer [9], which incorporates adaptive estimates of the mean and un-centered variance of the gradient to improve rates of convergence. We implemented the neural-network models and optimization procedures using PyTorch 1.6.0 [10]. We early stopped models after 5 epochs with no improvement in validation set accuracy and used the model parameters corresponding to the highest validation-set accuracy.

For real-time inference, we ensembled models by averaging 10 model predictions to improve performance, as in [2].

We used models that were trained using a 2.69-second window of neural features then tested using 2.5-second windows. This discrepancy was caused by a change made to task timing before collection of the sentence-spelling evaluation blocks; specifically, we had originally planned to use 2.69-second letter-decoding cycles during sentence spelling and trained the classifiers accordingly, but ultimately we decided to use 2.5-second letter-decoding cycles for a faster pacing. Because the classifier was designed to perform inference on inputs with flexible window lengths, we were able to evaluate the 2.5-second windows seamlessly and without any noticeable performance degradation.

### Augmentations

To bolster classifier performance, we used data augmentations, which have been shown to improve generalization and reduce overfitting for both images [11, 12] and neural activity [13, 14]. The following augmentations were applied sequentially to each trial of neural activity xᵢ during training (but not testing), without changing the associated label yᵢ:
1. Time jittering: shift the neural features by a time shift τ, such that:

$${x}_{i} ( t ) = {x}_{i} ( t - τ ) ,$$

$$τ ∼ U ( - j , j ) ,$$

where j is a hyperparameter.
2. Temporal masking: set some time points of the neural features to 0, such that:

$${x}_{i} [ {t}_{0} : {t}_{1} ] = ( 1 - {δ}_{p} ) , {t}_{1} = {t}_{0} + s$$

$$s ∼ U ( 0 , b ) ,$$

where t₀ is a randomly drawn time point within xᵢ and p is the probability of δₚ being one, and the time points being set to 0. Both b and p are hyperparameters.
3. Scaling: scale the magnitude of the neural features, such that:

$${x}_{i} = α {x}_{i} ,$$

$$α ∼ U [ {α}_{min} , {α}_{max} ] ,$$

where αₘᵢₙ and αₘₐₓ are hyperparameters.
4. Additive noise: add a matrix of random Gaussian noise to the neural features xᵢ, such that:

$${x}_{i} = {x}_{i} + N ( 0 , {σ}_{n}^{2} ) ,$$

where σₙ is a hyperparameter.
5. Channel-wise noise: offset the neural features by a value randomly sampled from a Gaussian distribution to each channel c, such that:

$${x}_{i} [ : , c ] = {x}_{i} + N ( 0 , {σ}_{c h}^{2} ) ,$$

where σ_{ch} is a hyperparameter and is shared across all features.

### Model Pre-Training and Fine-Tuning

When training the ensemble of classifiers used for real-time sentence spelling, which were also subsequently used during offline analyses to evaluate the effect of the beam search, the language model, and different vocabulary sizes on the real-time copy-typing results, we first pre-trained models on overt-speech attempts and then fine-tuned them on silent-speech attempts. Specifically, we trained classifiers on an initial dataset containing overt-speech attempts with a learning rate of 10⁻³. We split this initial dataset into training and validation sets, and we early stopped models after the accuracy on the validation set did not improve for 5 epochs in a row and reset the model parameters to those corresponding to the highest validation accuracy. Then, starting from those parameters, we fine-tuned the model on a second dataset containing silent-speech attempts, which involved training the pre-trained model on the new dataset with the same early-stopping process but with a smaller learning rate of 10⁻⁴.

### Hyperparameter Optimization

For the classifiers, we optimized the number of layers, number of hidden nodes in each layer, kernel size, stride, dropout rate, and augmentation hyperparameters using the Asynchronous Hyperband (ASH) method [15] with the Ray software package. We used the Hyperopt software package to suggest the next set of hyperparameters after each evaluation run [16]. The search space and final values are detailed in S2, and we searched 300 possible sets of hyperparameters.

We used all of the neural data from the overtly and silently attempted trials from isolated-target blocks recorded before collecting any sentence-spelling task blocks as the held-out validation dataset during hyperparameter optimization. We used the remaining isolated-target trials as training data during this process. During each evaluation run in the hyperparameter search, we initialized a new model using a set of hyperparameters determined by the algorithm and then began training the model. Because we performed model pre-training before fine-tuning, we first trained and evaluated the model on data recorded during overt-speech attempts. After each epoch of training, we evaluated the model accuracy on these overtly attempted trials with the current hyperparameter set. Because ASH uses the accuracy at each step to terminate underperforming hyperparameter combinations early, we scaled the accuracy by 0.1 during this pre-training process to prevent it from terminating prematurely if accuracy decreased once fine-tuning began.

We early stopped models as usual, re-instating the parameters corresponding to the highest accuracy. Then, starting from those parameters, we fine-tuned (and evaluated) the model on the silently attempted portion of the dataset with a learning rate of 10⁻³. Here, we purposefully used a greater learning rate than what was used during the final training procedure (which was 10⁻⁴) to evaluate hyperparameter combinations more quickly. ASH monitored the un-scaled accuracy values during the fine-tuning process. We terminated hyperparameter-optimization iterations after the accuracy on the hyperparameter-optimization dataset did not improve for 5 epochs in a row, and we kept the best accuracy as the score for that set of hyperparameters.

We used the resulting optimal neural-classifier hyperparameters for all of the real-time sentence-spelling blocks and analyses, and the blocks used for hyperparameter optimization were excluded from being used as evaluation blocks in all analyses.

Before each real-time sentence-spelling evaluation session, we trained 10 neural classifier models on all the data available prior to that day, including any previously recorded data from copy-typing sentence-spelling trials in which the decoded sentence had a CER of 0.0. Because our recording sessions were not back-to-back days, the most recent data available for training a new classifier was always at least 3 days prior to a given session (e.g. if the next recording session was on day 4, the most recent data would be from day 1, with no recording on days 2 and 3). We never updated models mid-session; we performed all real-time sentence-spelling evaluations without day-of model recalibration.

### Section S4. Adapted Beam Search

As described in the Methods section of the main text, we used an adapted prefix beam search as in [17] to find the transcription l* containing the sequence of characters (including whitespace characters) that maximizes

$${p}_{n c} ( ℓ \vert X ) {p}_{l m} ( ℓ ) , \qquad ( S9 )$$

over the set of possible transcriptions [symbol] Here X is the set of windows of neural activity x₁, . . . , X_{T}, p_{nc}( [symbol] |X) is the probability under the neural classifier of [symbol] given X, and pₗₘ( [symbol] ) is the probability of transcription [symbol] under a language-model prior. As in [17] we postulated that a language-model prior from an n-gram language model is too constrained, so we deemphasized it using a weighting parameter (α) and added a word-insertion bonus β to make up for the implicit decreasing of the probability of a sentence [symbol] as the number of words increases, revising the expression that the beam search tries to maximize to

$${P}_{n c} ( ℓ \vert X ) {{p}_{l m} ( ℓ )}^{a} {\vert ℓ \vert}^{β} , \qquad ( S10 )$$

where [symbol] is the cardinality of the word sequence yielded from transcription [symbol] . Both α and β were hyperparameters found via hyperparameter optimization on held-out sentence-spelling data. We used an n-gram language model to approximate pₗₘ( [symbol] ). The full algorithm is detailed in Algorithm 1:

### Sentence Finalization

If the probability of the attempted hand movement (the sentence-finalization command) was greater than 80%, the predicted sentence was finalized. Specifically, we pruned the current list of candidate sentences (from the beam search) to remove sentences that contained incomplete or out-of-vocabulary words. We then updated the probability of each remaining candidate sentence [symbol] as follows:

$${p}_{finalized} ( ℓ ) = p ( ℓ ) {{p}_{gpt 2} ( ℓ )}^{{α}_{gpt 2}} , \qquad ( S11 )$$

where p_{finalized}( [symbol] ) is the finalized probability of sentence [symbol] , p( [symbol] ) is the probability of the sentence [symbol] under equation S10, p_{gpt2}( [symbol] ) is the probability of [symbol] using Distil-GPT2 [18], and α_{gpt2} is a scaling parameter found through hyperparameter optimization. We then used the most likely sentence [symbol] as the finalized sentence.

### Hyperparameter Optimization

To find the optimal hyperparameters α, β, α_{gpt2}, and B, we collected an optimization dataset containing copy-typing sentence-spelling data recorded across 3 sessions to tune these parameters prior to performance evaluation of the spelling system. During these 3 sessions, the participant attempted to spell 35 of the 75 copy-typing sentences. Of these 35 sentences, there were 15 randomly selected sentences that the participant attempted 10 times, 5 sentences that the participant attempted 9 times, and 15 sentences that the participant attempted once. The remaining 40 sentences were unseen by the participant prior to real-time evaluation. We then used these sentences offline to optimize α, β, a_{gpt2}, and B.

Algorithm 1 Constrained beam search. Given T windows of neural activity and p(c|x₁:T) (where c is a character), this algorithm finds the most likely sentence [symbol] * composed of words within a constrained vocabulary V. After a character is added to [symbol] to give [symbol] ⁺, we check that the final word in [symbol] ⁺ is in Vₚₐᵣₜᵢₐₗ, which is composed of every possible word and partial word∈V. The function w_{final} extracts all the characters after the final space. To automatically insert spaces, the vocabulary considers every text string in A⁺, where A⁺=A∪A_{space}, A is the set of text strings containing a single English letters (“a”, “b”, “c”, . . . , “z”), and Aspace is the same set as A but with the whitespace character appended after each letter (“a”, “b”, “c”, . . . , “z”). We set the probability for a character c with a space equal to p(c|xᵢ) (the probability of that character without the space). Here, let the function W( [symbol] ) segment the sequence of characters [symbol] at each space and truncate any characters trailing the last space, yielding a list of completed words in t. Let pₗₘ(W( [symbol] ⁺)|W( [symbol] )) give the probability of the last word in [symbol] ⁺ given the n−1 preceding words, enabling the use of an n-gram language model. The probability threshold for characters to be considered in the beam search was set to 10⁻³. B is the beam width (the number of beams used in the beam search).

```
sents = {(Ø, 0)}
for i = 1, . . . ,T do
  new_sents = {}
  for [symbol]    , log p( [symbol]   ) ϵ sents do
    for c in (A⁺) do
      if p(c|xᵢ) < 0.001 then
        continue to next character
      end if [symbol]
      ⁺ ← append c to [symbol]
      if w_{final}( [symbol]   ⁺) ϵ [symbol]    ₚₐᵣₜᵢₐₗ then
        if c ϵ A_{space} then
          log p( [symbol]   ⁺) ← log p( [symbol]   ) + α log pₗₘ (W( [symbol]   ⁺) | W ( [symbol]   )) + log p(c | xᵢ)+
            β log|W ( [symbol]   ⁺)| − β log(max(1, |W( [symbol]   )|))
        else
          log p( [symbol]   ⁺) ← log p( [symbol]   ) + log p(c | xᵢ)
        end if
        add ( [symbol]   ⁺, log p( [symbol]   ⁺)) to new_sents
      end if
  end for
  end for
  sents ← B most probable prefixes in new sents
end for
return most probable prefix in sents
```

As with the classifier, we used the Asynchronous Hyperband method [15] with the Ray package [16], using Hyperopt to suggest the next set of hyperparameters after each iteration. We searched 500 sets of hyperparameters and chose the set that produced the best word error rate to use for the first day of real-time sentence-spelling evaluation. After that first day of evaluation, we re-ran the hyperparameter optimization procedure using only the data collected during that day. We used the hyperparameter values found during this second optimization run during all proceeding real-time sentence-spelling evaluation sessions.

### No-Beam Edge Case

For 3 of the copy-typing sentence-spelling trials recorded during the real-time evaluation sessions, the beam search ran out of valid sentences. This occurred if the participant made a mistake such that no letter sequence that could make valid sentence candidates surpassed the threshold for consideration by the beam search.

On the first day of the real-time evaluation sessions, if this occurred, we would simply output the most likely letters obtained from the neural classifier (without any spaces). Before the second day of real-time evaluation, we modified the beam-search algorithm to output the most likely sentence candidate at that point (immediately before the beam search contained no valid sentence candidates) and then subsequently output the most likely letters obtained from the neural classifier for the remainder of the trial. Additionally, for the first day of the real-time evaluation sessions, the probability threshold for a letter to be considered in the beam search (see Algorithm 1) was set to 10⁻³. For the second day of real-time evaluation, we kept the threshold the same, but modified the beam-search algorithm so that if less than 3 letters (and their counterparts with spaces) had probability >10⁻³, we considered the 13 most likely letters (and their counterparts with spaces) to avoid running out of valid beams.

Section S5. Language Modeling n-Gram Modeling

During the beam-search process, as we were updating each beam with a new character, we used a trigram language model because it was reliable while also being capable of producing predictions more quickly than a large neural network-based language model.

The basic n-gram formulation is defined as having the probability of a word wₖ in position k as:

$$p ( {w}_{k} \vert {w}_{k - 1} , … , {w}_{k - n + 1} ) = \frac{C ( {w}_{k} , … , {w}_{k - n + 1} )}{C ( {w}_{k - 1} , … , {w}_{k - n + 1} )} , \qquad ( S12 )$$

where C is a function that counts the number of times each n-gram happens in a corpus.

Improved n-gram modeling can be achieved with back-off and discounting [19]. Back-off refers to using lower-order n-gram models to estimate the probability of higher-order n-grams, since high-order n-grams can be sparse. The n-gram probability p(wᵢ|wᵢ₋ₙ₊₁ⁱ⁻¹) directly depends on the lower-order n-gram p(wᵢ|wᵢ₋ₙ₊₂ⁱ⁻¹) (i.e. trigram probabilities depend on bigram and unigram probabilities), as shown in Equation S13. Discounting is a form of regularization of the n-gram probability distribution in which a constant number is removed from the count of each n-gram prior to computing the n-gram probabilities, and the probability mass that was removed in this manner is redistributed through a weighted lower-order n-gram model. For more details, see [20].

We used the following formulation to implement back-off with discounting:

$$p ( {w}_{i} \vert {w}_{i - n + 1}^{i - 1} ) = \frac{max ( C ( {w}_{i - n + 1}^{i} ) - δ , 0}{{\sum }_{{w}_{i}} C ( {w}_{i - n + 1}^{i} )} + α ( {w}_{i - n + 1}^{i - 1} ) p ( {w}_{i} \vert {w}_{i - n + 2}^{i - 1} ) . \qquad ( S13 )$$

Here, δ is the discount factor and α(wᵢ₋ₙ₊₁ⁱ⁻¹) is defined as:

$$α ( {w}_{i - n + 1}^{i - 1} ) = \frac{δ {N}_{1} + ( {w}_{i - n + 1}^{i - 1} )}{{\sum }_{{w}_{i}} C ( {w}_{i - n + 1}^{i} )} , \qquad ( S14 )$$

where N₁₊ represents the number of unique words that appear after the preceding n−1 words (the number of times the max selects something non-zero in equation S13). Whenever Σ_{w}_{ᵢ}C(wᵢ₋ₙ₊₁ⁱ)=0, we use the lower-order model probability directly to avoid division by 0.

We also used Kneser-Ney smoothing ([21]) to improve the unigram model implicit in S13, replacing it with word fertility, which represents the number of distinct context types that a word occurs in. Using word context fertility, we can write the following proportion:

$$p ( w ) ∝ \vert \{ {w}^{′} : C ( {w}^{′} , w ) > 0 \} \vert , \qquad ( S15 )$$

where w′ is the word fertility and refers to the cardinality operation.

We can now rewrite our unigram model as:

$$p ( w ) = \frac{\vert \{ {w}^{′} : C ( {w}^{′} , w ) > 0 \} \vert + {α}_{kn}}{{\sum }_{w ∈ 𝒱} \vert \{ {w}^{′} : C ( {w}^{′} , w ) > 0 \} \vert + N {α}_{kn}} , \qquad ( S16 )$$

where V is the set of words in the training vocabulary, Nis the total number of words in the vocabulary, and αₖₙ is a smoothing hyperparameter that prevents unseen words from having a probability of 0 and infrequent words from being penalized too heavily. In practice, we defined a fixed discount factor 8=0.9 and a fixed Kneser-Ney smoothing factor αₖₙ=0.003.

We used two corpora to train the language model: nltk's Twitter corpus [22] and the Cornell movies corpus [23]. We selected these two corpora because of the casual and conversational nature of their speech content. With any given vocabulary, we trained the n-gram model on all of the trigrams from both corpora that were composed solely of words from that vocabulary. Before training, we inserted two start-of-sentence tokens before the start of each sentence in both corpora to enable modeling of sentence starts during inference.

### Sentence-Finalization Language Model

To score sentences after finalization during sentence spelling, we used the DistilGPT-2 neural network-based language model [18], which is based on OpenAI's GPT-2 language model [24] but has fewer parameters.

### SUPPLEMENTARY REFERENCES

1. Paszke A, Gross S, Massa F, et al. PyTorch: An Imperative Style, High-Performance Deep Learning Library. In: Advances in Neural Information Processing Systems 32. Ed. by Wallach H, Larochelle H, Beygelzimer A, d'Alch'e-Buc F, Fox E, and Garnett R. Curran Associates, Inc., 2019:8024-35.
2. Moses D A, Metzger S L, Liu J R, et al. Neuroprosthesis for Decoding Speech in a Paralyzed Person with Anarthria. New England Journal of Medicine 2021; 385:217-27.
3. Kingma D P and Ba J. Adam: A Method for Stochastic Optimization. arXiv:1412.6980 2017.
4. Bergstra J, Yamins D L K, and Cox D D. Making a Science of Model Search: Hyper-parameter Optimization in Hundreds of Dimensions for Vision Architectures. Icml 2013:115-23.
5. Cho K, Van Merrienboer B, Bahdanau D, and Bengio Y. On the properties of neural machine translation: Encoder-decoder approaches. arXiv preprint arXiv:1409.1259 2014.
6. Hochreiter S and Schmidhuber J. Long short-term memory. Neural computation 1997; 9:1735-80.
7. Chung J, Gulcehre C, Cho K, and Bengio Y. Empirical evaluation of gated recurrent neural networks on sequence modeling. arXiv preprint arXiv:1412.3555 2014.
8. Hinton G E, Srivastava N, Krizhevsky A, Sutskever I, and Salakhutdinov R R. Improving neural networks by preventing c₀-adaptation of feature detectors. arXiv preprint arXiv:1207.0580 2012.
9. Kingma D P and Ba J. Adam: A method for stochastic optimization. arXiv preprint arXiv:1412.6980 2014.
10. Paszke A, Gross S, Massa F, et al. PyTorch: An Imperative Style, High-Performance Deep Learning Library. In: Advances in Neural Information Processing Systems 32. Ed. by Wallach H, Larochelle H, Beygelzimer A, d'Alch'e-Buc F, Fox E, and Garnett R. Curran Associates, Inc., 2019:8024-35. (papers.neurips.cc/paper/9015-pytorch-an-imperative-style-high-performance-deep-learning-library.pdf)
11. Krizhevsky A, Sutskever I, and Hinton G E. Imagenet classification with deep convolutional neural networks. Advances in neural information processing systems 2012; 25:1097-105.
12. Reed C J, Metzger S, Srinivas A, Darrell T, and Keutzer K. Selfaugment: Automatic augmentation policies for self-supervised learning. In: Proceedings of the IEEE/CVF Conference on Computer Vision and Pattern Recognition. 2021:2674-83.
13. Willett F R, Avansino D T, Hochberg L R, Henderson J M, and Shenoy K V. High-performance brain-to-text communication via handwriting. Nature 2021; 593:249-54.
14. Moses D A, Metzger S L, Liu J R, et al. Neuroprosthesis for decoding speech in a paralyzed person with anarthria. New England Journal of Medicine 2021; 385:217-27.
15. Li L, Jamieson K, Rostamizadeh A, et al. Massively parallel hyperparameter tuning. 2018.
16. Moritz P, Nishihara R, Wang S, et al. Ray: A distributed framework for emerging {AI} applications. In: 13th {USENIX} Symposium on Operating Systems Design and Implementation ({OSDI} 18). 2018:561-77.
17. Hannun A Y, Maas A L, Jurafsky D, and Ng A Y. First-pass large vocabulary continuous speech recognition using bi-directional recurrent dnns. arXiv preprint arXiv:1408.2873 2014.
18. Sanh V, Debut L, Chaumond J, and Wolf T. DistilBERT, a distilled version of BERT: smaller, faster, cheaper and lighter. 2020. arXiv: 1910.01108 [cs.CL].
19. Chen S F and Goodman J. An empirical study of smoothing techniques for language modeling. Computer Speech & Language 1999; 13:359-94.
20. Jurafsky D and Martin J H. Speech and language processing. Vol. 3. U S: Prentice Hall 2014.
21. Kneser R and Ney H. Improved backing-off for m-gram language modeling. In: 1995 international conference on acoustics, speech, and signal processing. Vol.
1. IEEE. 1995:181-4.
22. Bird S, Klein E, and Loper E. Natural language processing with Python: analyzing text with the natural language toolkit. “O'Reilly Media, Inc.”, 2009.
23. Danescu-Niculescu-Mizil C and Lee L. Chameleons in imagined conversations: A new approach to understanding coordination of linguistic style in dialogs. In:
Proceedings of the Workshop on Cognitive Modeling and Computational Linguistics, A C L 2011. 2011.
24. Radford A, Wu J, Child R, Luan D, Amodei D, Sutskever I, et al. Language models are unsupervised multitask learners. OpenAI blog 2019; 1:9.
25. Romero D E T and Jovanovic G. Digital FIR Hilbert Transformers: Fundamentals and Efficient Design Methods. In: MATLAB—A Fundamental Tool for Scientific Computing and Engineering Applications—Volume 1. 2012:445-82. (intechopen.com/books/matlab-a-fundamental-tool-for-scientific-computing-and-engineering-applications-volume-1/digital-fir-hilbert-transformers-fundamentals-and-efficient-design-methods)
26. Welford B P. Note on a Method for Calculating Corrected Sums of Squares and Products. Technometrics 1962; 4:419-9.
27. Moses D A, Leonard M K, Makin J G, and Chang E F. Real-time decoding of question-and-answer speech dialogue using human cortical activity. Nature Communications 2019; 10:3096.

SUPPLEMENTARY TABLE S1
Copy-typing task sentences.

| Target sentence | Decoded sentence in first trial | Decoded sentence in second trial |
|---|---|---|
| good morning | good morning | good for legs |
| you have got to be kidding | you have got to be kidding a | you have got to be kidding |
| what do you mean | what do you mean | what do you mean |
| good to see you | i do i leave you | good to see you |
| i think this is pretty good | i think this is pretty good | i think they is pretty good |
| i will check | i will check | i will the it |
| thank you | thank you | thank you |
| please sit down | please sit down | please believe |
| we have to stop | we have to stop | we have to stop |
| hand that to me please | hand that time please | have that time always |
| i know what you mean | i know what you mean | i know what you mean |
| what time is it | what time is it | what time is it |
| sit over here with me | sit over here with me | sit over here with me |
| no thanks | no thanks | not happen |
| you never know | you never know | you never know |
| great to see you again | great to show my case in | great to stay in town |
| forget about it | forget about it | forget about it |
| could you repeat what you said | dog lie on repeat what you said | could you repeat what you said |
| where do you live | where do you live | where do you live |
| do not be afraid to ask me questions | do not be afraid to ask me questions | do not be afraid to ask me questions |
| i cannot believe it | i can not believe it | i can not believe it |
| thanks for telling me | thank for reading me | thanks for telling me |
| i do not want that | i do not want that | i do not want that |
| that is wonderful | that is work from a | that is wonderful |
| what do you think about that | what do you think about that | what do you think about that |
| thank you very much | though it very much | thank you very much |
| i am glad you are here | i am glad you are here | i am glad you are here |
| how are you doing | how are you doing | how are you doing |
| i agree | i agree | i agree |
| i am okay | i am okay | i am okay |
| tell me what you are doing | tell me what your telling | tell me what you are doing |
| how long did it take | how long did it take | how long did it take |
| is there anything i can do | is there a nothing i can do | is there anything i can do |
| how are things going for you | how are things gives for you | how are things going for you |
| do you know what he did | do you know on the ice | do you know what he did |
| was there something else | was there to be a high else | was there something else |
| where are you going | while are you doing | where are you going |
| who is that | who is that | why is that |
| tell me about your family | tell me about your family | tell me about your family |
| i could probably do better | i could probably do better | i could probably do better |
| you can say that again | you can say that again | you can say that open |
| i am sorry to hear that | i am to get to hear that | i am sorry to hear that |
| will i see you later | will i see you later | well i keep by later |
| i am doing well | i am doing well | i am doing fine |
| can that wait until another time | can that wait until another time | can that wait until another time |
| how much more is there | how much more is there | how much were in there |
| come talk with me | come talk with me | some take with me |
| that will be fun | that will be fun | that will be fun |
| how often do you do this | how often do you do this | how often do you do this |
| how much will it cost | how much will it cost | how much will it cost |
| bring that over here | clinic hat for hat | bring that ever here |
| turn it off | turn it off | turn it off |
| i remember the last time i did that | i remember the last time i did that | i remember to plan new me i did that |
| i was just kidding | i was mike kidding | i was just kidding |
| i will meet you there | i will meet you there | i will meet you to eat |
| i do not really remember | i do not really remember | ddonoyrballyrlrefbhrh |
| i feel cold | i feel weird | i feel cold |
| excuse me for interrupting | excuse me for interrupt any | excuse me for interrupting |
| you are not going to believe this | you plan to go in on a bit love this | ypuaranpdggingloavlinesoeb |
| do you understand what i mean | do you understand what i mean | do you understand what i mean |
| what are you talking about | what are you talking about | what are you talking about |
| which one is it | which one edit | which one is it |
| would you like to go with me | a all i was like the white me | would you like to go with me |
| i do not understand | i do not understand | i do not understand |
| of course i do | of course its | of course him |
| anything is possible | anything is possible | anything is possible |
| do not do that again | do not do that again | do not do that again |
| let me see that | let me see that | let me see that |
| what have you been doing | what have you been doing | what have you been doing |
| i had a great time | i had a great time | what a great time |
| easy for you to say | easy for you to say | easy for you to say |
| i want to go | i want to go | i want to go |
| how do you feel | how do you feel | how do you feel |
| that is all right | that is all right | that is all right |
| i told you i do not know | i told you i do not know | i told you i do not know |

SUPPLEMENTARY TABLE S2
Hyperparameter definitions and values

| | Hyperparameter | Search- | Value | Optimal |
|---|---|---|---|---|
| Model | description | space type¹ | range | values² |
| Speech | Smoothing size | Uniform (int) | [1, 80] | 78 |
| detector | Probability threshold | Uniform | [0.1, 0.9] | 0.304 |
| | Time threshold duration | Uniform (int) | [25, 150] | 105 |
| Word | Number of GRU layers | Uniform (int) | [1, 4] | 2 |
| classifier | Nodes per GRU laver | Uniform (int) | [128, 512] | 274 |
| | Dropout fraction | Uniform | [0.3, 0.8] | 0.545 |
| | Convolution kernel | Uniform (int) | [1, 10] | 4 |
| | size and skip | | | |
| | Jitter amount (seconds), i | Uniform | [0.0, 2.0] | 0.474 |
| | Additive noise level, σₙ | Uniform | [0.0, 1.0] | 0.0027 |
| | Scale min., αₘᵢₙ | Uniform | [0.8, 1.0] | 0.955 |
| | Scale max., αₘₐₓ | Uniform | [1.0, 1.2] | 1.07 |
| | Max. temporal-masking | Uniform | [0.00, 1.35] | 0.871 |
| | length (seconds), b | | | |
| | Temporal masking probability, p | Uniform | [0.0, 0.5] | 0.0478 |
| | Channel-wise noise, σ_{c} | Uniform | [0.0, 1.0] | 0.0283 |
| Beam | Language-model scaling factor, α | Uniform | [0.01, 1.0] | (0.642, 0.744) |
| search | Word-insertion weight, β | Uniform | [0.0, 30.0] | (4.03, 10.5) |
| | Number of beams maintained, B | Uniform (int) | [0, 750] | (457, 739) |
| | Distil-GPT2 scaling factor, α_{gpt2} | Uniform | [0.0, 100.0] | (1.53, 1.13) |

¹“Uniform (int)” indicates that hyperparameter values were forced to be integers.
²For the language modeling and beam-search hyperparameters, two values are listed: the first is the optimal value found when optimizing on the copy-typing sentence-spelling trials prior to the first day of sentence-spelling evaluations (used during this first day), and the second is the optimal value found when optimizing on the copy-typing sentence-spelling trials from the first day of sentence-spelling evaluations (used for the second day and all subsequent days).

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims. It is also to be understood that the terminology used herein is for the purpose of describing particular embodiments only, and is not intended to be limiting, since the scope of the present invention will be limited only by the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. A method of assisting a subject with communication, the method comprising:
positioning a neural recording device comprising an electrode at a location in a sensorimotor cortex region of the brain of the subject to record brain electrical signal data associated with attempted speech by the subject;
positioning an interface in communication with a computing device at a location on the head of the subject, wherein the interface is connected to the neural recording device;
recording the brain electrical signal data associated with attempted speech by the subject using the neural recording device, wherein the interface receives the brain electrical signal data from the neural recording device and transmits the brain electrical signal data to a processor of the computing device; and
decoding a word, a phrase, or a sentence from the recorded brain electrical signal data using the processor.

2. The method of claim 1, wherein the subject has difficulty with said communication because of anarthria, a stroke, a traumatic brain injury, a brain tumor, or amyotrophic lateral sclerosis.

3. The method of claim 1 or 2, wherein the subject is paralyzed.

4. The method of any one of claims 1-3, wherein the location of the neural recording device is in the ventral sensorimotor cortex.

5. The method of any one of claims 1-4, wherein the electrode is positioned on a surface of the sensorimotor cortex region or within the sensorimotor cortex region.

6. The method of claim 5, wherein the electrode is positioned on a surface of the sensorimotor cortex region of the brain in a subdural space.

7. The method of any one of claims 1-6, wherein the neural recording device comprises a brain-penetrating electrode array.

8. The method of any one of claims 1-7, wherein the neural recording device comprises an electrocorticography (ECoG) electrode array.

9. The method of any one of claims 1-8, wherein the electrode is a depth electrode or a surface electrode.

10. The method of any one of claims 1-9, wherein the electrical signal data comprises high-gamma frequency content features.

11. The method of claim 10, wherein the electrical signal data comprises neural oscillations in a range from 70 Hz to 150 Hz.

12. The method of any one of claims 1-11, wherein said recording the brain electrical signal data comprises recording the brain electrical signal data from a sensorimotor cortex region selected from a precentral gyrus, postcentral gyrus, posterior middle frontal gyrus, posterior superior frontal gyrus, or posterior inferior frontal gyrus region, or any combination thereof.

13. The method of any one of claims 1-12, further comprising mapping the brain of the subject to identify an optimal location for positioning the electrode for recording the brain electrical signals associated with the attempted speech by the subject.

14. The method of any one of claims 1-13, wherein the interface comprises a percutaneous pedestal connector attached to the subject's cranium.

15. The method of claim 14, wherein the interface further comprises a headstage connected to the percutaneous pedestal connector.

16. The method of any one of claims 1-15, wherein the processor is provided by a computer or handheld device.

17. The method of claim 16, wherein the handheld device is a cell phone or a tablet.

18. The method of any one of claims 1-17, wherein the processor is programmed to automate speech detection, word classification, and sentence decoding based on identification of a neural activity pattern of electrical signals in the recorded brain electrical signal data associated with attempted word production.

19. The method of claim 18, wherein the processor is programmed to use a machine learning algorithm for speech detection, word classification, and sentence decoding.

20. The method of claim 19, wherein artificial neural network (ANN) models are used for the speech detection and the word classification, and a hidden Markov model (HMM), a Viterbi decoding model, or a natural language processing technique is used for the sentence decoding.

21. The method of any one of claims 1-20, wherein the processor is programmed to automate detection of onset and offset of word production during the attempted speech by the subject.

22. The method of claim 21, further comprising assigning speech event labels for preparation, speech, and rest to time points during the recording of the brain electrical signal data.

23. The method of claim 21 or 22, wherein the processor is programmed to use the recorded brain electrical signal data within a time window around the detected onset of word classification.

24. The method of any one of claims 1-23, wherein the subject is limited to a specified word set for the attempted speech.

25. The method of claim 24, wherein the processor is programmed to calculate a probability that a word of the word set is an intended word that the subject tried to produce during the attempted speech.

26. The method of claim 25, wherein the processor is programmed to calculate the probability that a word of the word set is an intended word that the subject tried to produce during the attempted speech for every word of the word set.

27. The method of any one of claims 24-26, wherein the word set comprises am, are, bad, bring, clean, closer, comfortable, coming, computer, do, faith, family, feel, glasses, going, good, goodbye, have, hello, help, here, hope, how, hungry, I, is, it, like, music, my, need, no, not, nurse, okay, outside, please, right, success, tell, that, they, thirsty, tired, up, very, what, where, yes, and you.

28. The method of any one of claims 1-27, wherein the subject may use the words of the word set without limitation to create sentences.

29. The method of claim 28, wherein the processor is programmed to calculate a probability that a sequence of words is an intended sentence that the subject tried to produce during the attempted speech.

30. The method of any one of claims 1-29, wherein the processor is programmed to use a language model that provides next-word probabilities given a previous word or phrase in a sequence of words to aid the decoding by determining predicted word sequence probabilities.

31. The method of claim 30, wherein words that occur more frequently are assigned more weight than words that occur less frequently according to the language model.

32. The method of claim 30 or 31, wherein the processor is programmed to use a Viterbi decoding model to determine the most likely sequence of words in the intended speech of the subject given the brain electrical signal data associated with the attempted speech, the predicted word probabilities from the word classification model using the machine learning algorithm, and the word sequence probabilities using the language model.

33. The method of any one of claims 1-32, further comprising:
recording brain electrical signal data associated with an attempted non-speech motor movement of the subject, wherein the subject performs the attempted non-speech motor movement to indicate the initiation or termination of the attempted speech or to control an external device; and
analyzing the brain electrical signal data using a non-speech motor movement classification model that identifies patterns of electrical signals in the recorded brain electrical signal data associated with the attempted non-speech motor movement and calculates a probability that the subject attempted the non-speech motor movement.

34. The method of claim 33, wherein the attempted non-speech motor movement comprises an attempted head, arm, hand, foot, or leg movement.

35. The method of claim 34, wherein the attempted hand movement comprises an imagined hand gesture or an imagined hand squeeze.

36. The method of any one of claims 33-35, wherein the processor is further programmed to automate detection of an attempted non-speech motor movement of the subject signaling the end of the attempted speech by the subject based on identification of a neural activity pattern of electrical signals in the recorded brain electrical signal data associated with the attempted non-speech motor movement.

37. The method of claim 36, wherein the processor is further programmed to assign event labels for the attempted non-speech motor movement to time points during the recording of the brain electrical signal data.

38. The method of any one of claims 1-37, wherein the method further comprises assessing accuracy of the decoding.

39. A computer implemented method for decoding a sentence from recorded brain electrical signal data associated with attempted speech by a subject, the computer performing steps comprising:
a) receiving the recorded brain electrical signal data associated with the attempted speech by the subject;
b) analyzing the recorded brain electrical signal data using a speech detection model to calculate the probability that attempted speech is occurring at any time point during recording of the brain electrical signal data and detect onset and offset of word production during the attempted speech by the subject;
c) analyzing the brain electrical signal data using a word classification model that identifies patterns of electrical signals in the recorded brain electrical signal data associated with attempted word production by the subject and calculates predicted word probabilities;
d) performing sentence decoding by using the calculated word probabilities from the word classification model in combination with predicted word sequence probabilities in the sentence using a language model that provides next-word probabilities given a previous word or phrase in a sequence of words to calculate predicted word sequence probabilities and determining the most likely sequence of words in the sentence based on the predicted word probabilities determined using the word classification model and the language model; and
e) displaying the sentence decoded from the recorded brain electrical signal data.

40. The computer implemented method of claim 39, wherein a machine learning algorithm is used for speech detection, word classification, and sentence decoding.

41. The computer implemented method of claim 40, wherein artificial neural network (ANN) models are used for the speech detection and the word classification, and a hidden Markov model (HMM), a Viterbi decoding model, or a natural language processing technique is used for the sentence decoding.

42. The computer implemented method of any one of claims 39-41, wherein the subject is limited to a specified word set for the attempted speech.

43. The computer implemented method of claim 42, further comprising calculating a probability that a word of the word set is an intended word that the subject tried to produce during the attempted speech for every word of the word set and select the word of the word set having the highest probability of being the intended word that the subject tried to produce during the attempted speech.

44. The computer implemented method of any one of claims 39-43, wherein the subject may use the words of the word set without limitation to create sentences or is limited to a specified sentence set for the attempted speech.

45. The computer implemented method of any one of claims 39-44, further comprising calculating a probability that a sequence of words is an intended sentence that the subject tried to produce during the attempted speech.

46. The computer implemented method of claim 45, further comprising maintaining the most likely sentence and one or more less likely sentences and recalculating the probability that a sequence of words is an intended sentence that the subject tried to produce during the attempted speech after decoding of each word.

47. The computer implemented method of claim 46, wherein the most likely sentence and the one or more less likely sentences are composed only of words from the word set used by the subject for the attempted speech.

48. The computer implemented method of any one of claims 39-47, further comprising assigning speech event labels for preparation, speech, and rest to time points during the recording of the brain electrical signal data.

49. The computer implemented method of claim 48, wherein only the recorded brain electrical signal data within a time window around the detected onset of word classification is used.

50. The computer implemented method of any one of claims 39-49, wherein more weight is assigned to words that occur more frequently than words that occur less frequently according to the language model.

51. The computer implemented method of any one of claims 39-50, further comprising storing a user profile for the subject comprising information regarding the patterns of electrical signals in the recorded brain electrical signal data associated with attempted word production by the subject.

52. The computer implemented method of any one of claims 39-51, further comprising:
receiving recorded brain electrical signal data associated with an attempted non-speech motor movement of the subject, wherein the subject performs the attempted non-speech motor movement to indicate the initiation or termination of the attempted speech or to control an external device; and
analyzing the brain electrical signal data using a classification model that identifies patterns of electrical signals in the recorded brain electrical signal data associated with the attempted non-speech motor movement and calculates a probability that the subject attempted the non-speech motor movement.

53. The computer implemented method of claim 52, wherein the attempted non-speech motor movement comprises an attempted head, arm, hand, foot, or leg movement.

54. The computer implemented method of claim 53, wherein the attempted hand movement comprises an imagined hand gesture or an imagined hand squeeze.

55. The computer implemented method of any one of claims 52-54, further comprising assigning event labels for the attempted non-speech motor movement to time points during the recording of the brain electrical signal data.

56. A non-transitory computer-readable medium comprising program instructions that, when executed by a processor in a computer, causes the processor to perform the method of any one of claims 39-55.

57. A kit comprising the non-transitory computer-readable medium of claim 56 and instructions for decoding brain electrical signal data associated with attempted speech by a subject.

58. A system for assisting a subject with communication, the system comprising:
a neural recording device comprising an electrode adapted for positioning at a location in a sensorimotor cortex region of the brain of the subject to record brain electrical signal data associated with attempted speech or an attempted non-speech motor movement by the subject;
a processor programmed to decode a sentence from the recorded brain electrical signal data according to the computer implemented method of any one of claims 39-55;
an interface in communication with a computing device, said interface adapted for positioning at a location on the head of the subject, wherein the interface receives the brain electrical signal data from the neural recording device and transmits the brain electrical signal data to the processor; and
a display component for displaying the sentence decoded from the recorded brain electrical signal data.

59. The system of claim 58, wherein the subject has difficulty with said communication because of anarthria, a stroke, a traumatic brain injury, a brain tumor, or amyotrophic lateral sclerosis.

60. The system of claim 58 or 59, wherein the location of the neural recording device is in the ventral sensorimotor cortex.

61. The system of any one of claims 58-60, wherein the electrode is adapted for positioning on a surface of the sensorimotor cortex region or within the sensorimotor cortex region.

62. The system of claim 61, wherein the electrode is adapted for positioning on a surface of the sensorimotor cortex region of the brain in a subdural space.

63. The system of any one of claims 58-62, wherein the neural recording device comprises a brain-penetrating electrode array.

64. The system of any one of claims 58-63, wherein the neural recording device comprises an electrocorticography (ECoG) electrode array.

65. The system of any one of claims 58-64, wherein the electrode is a depth electrode or a surface electrode.

66. The system of any one of claims 58-65, wherein the electrical signal data comprises high-gamma frequency content features.

67. The system of claim 66, wherein the electrical signal data comprises neural oscillations in a range from 70 Hz to 150 Hz.

68. The system of any one of claims 58-67, wherein the interface comprises a percutaneous pedestal connector attached to the subject's cranium.

69. The system of claim 68, wherein the interface further comprises a headstage that is connectable to the percutaneous pedestal connector.

70. The system of any one of claims 58-69, wherein the processor is provided by a computer or handheld device.

71. The system of claim 70, wherein the handheld device is a cell phone or tablet.

72. The system of any one of claims 58-71, wherein a machine learning algorithm is used for speech detection, word classification, and sentence decoding.

73. The system of claim 72, wherein artificial neural network (ANN) models are used for the speech detection and the word classification, and a hidden Markov model (HMM), a Viterbi decoding model, or a natural language processing technique is used for the sentence decoding.

74. The system of any one of claims 58-73, wherein the processor is further programmed to assign speech event labels for preparation, speech, and rest to time points during the recording of the brain electrical signal data.

75. The system of claim 74, wherein the processor is further programmed to use the recorded brain electrical signal data within a time window around the detected onset of word classification.

76. The system of any one of claims 58-75, wherein the subject is limited to a specified word set for the attempted speech.

77. The system of claim 76, wherein the processor is further programmed to calculate a probability that a word of the word set is an intended word that the subject tried to produce during the attempted speech for every word of the word set and select the word of the word set having the highest probability of being the intended word that the subject tried to produce during the attempted speech.

78. The system of claim 76 or 77, wherein the word set comprises: am, are, bad, bring, clean, closer, comfortable, coming, computer, do, faith, family, feel, glasses, going, good, goodbye, have, hello, help, here, hope, how, hungry, I, is, it, like, music, my, need, no, not, nurse, okay, outside, please, right, success, tell, that, they, thirsty, tired, up, very, what, where, yes, and you.

79. The system of any one of claims 76-78, wherein the subject may use any chosen sequence of words of the selected word set.

80. The system of claim 79, wherein the processor is programmed to calculate a probability that a sequence of words is an intended sentence that the subject tried to produce during the attempted speech.

81. The system of claim 80, wherein the processor is programmed to maintain the most likely sentence and one or more less likely sentences and recalculate the probability that a sequence of words is an intended sentence that the subject tried to produce during the attempted speech after decoding of each word.

82. The system of claim 81, wherein the most likely sentence and the one or more less likely sentences are composed only of words from the word set used by the subject for the attempted speech.

83. The system of any one of claims 58-82, wherein the processor is further programmed to automate detection of an attempted non-speech motor movement of the subject signaling the initiation or termination of the attempted speech by the subject based on identification of a neural activity pattern of electrical signals in the recorded brain electrical signal data associated with the attempted non-speech motor movement.

84. The system of claim 83, wherein the processor is further programmed to assign event labels for the attempted non-speech motor movement to time points during the recording of the brain electrical signal data.

85. A kit comprising the system of any one of claims 58-84 and instructions for using the system for recording and decoding brain electrical signal data associated with attempted speech by a subject.

86. A method of assisting a subject with communication, the method comprising:
positioning a neural recording device comprising an electrode at a location in a sensorimotor cortex region of the brain of the subject to record brain electrical signal data associated with attempted spelling of letters of words of an intended sentence by the subject;
positioning an interface in communication with a computing device at a location on the head of the subject, wherein the interface is connected to the neural recording device;
recording the brain electrical signal data associated with said attempted spelling by the subject using the neural recording device, wherein the interface receives the brain electrical signal data from the neural recording device and transmits the brain electrical signal data to a processor of the computing device; and
decoding the spelled words of the intended sentence from the recorded brain electrical signal data using the processor.

87. The method of claim 86, wherein the subject has difficulty with said communication because of anarthria, a stroke, a traumatic brain injury, a brain tumor, or amyotrophic lateral sclerosis.

88. The method of claim 86 or 87, wherein the subject is paralyzed.

89. The method of any one of claims 86-88, wherein the location of the neural recording device is in the ventral sensorimotor cortex.

90. The method of any one of claims 86-89, wherein the electrode is positioned on a surface of the sensorimotor cortex region or within the sensorimotor cortex region.

91. The method of claim 90, wherein the electrode is positioned on a surface of the sensorimotor cortex region of the brain in a subdural space.

92. The method of any one of claims 86-91, wherein the neural recording device comprises a brain-penetrating electrode array.

93. The method of any one of claims 86-92, wherein the neural recording device comprises an electrocorticography (ECoG) electrode array.

94. The method of any one of claims 86-93, wherein the electrode is a depth electrode or a surface electrode.

95. The method of any one of claims 86-94, wherein the electrical signal data comprises high-gamma frequency content features and low frequency content features.

96. The method of claim 95, wherein the electrical signal data comprises neural oscillations in a high-gamma frequency range from 70 Hz to 150 Hz and in a low frequency range from 0.3 Hz to 100 Hz.

97. The method of any one of claims 86-96, wherein said recording of the brain electrical signal data comprises recording the brain electrical signal data from a sensorimotor cortex region selected from a precentral gyrus region, a postcentral gyrus region, a posterior middle frontal gyrus region, a posterior superior frontal gyrus region, or a posterior inferior frontal gyrus region, or any combination thereof.

98. The method of any one of claims 86-97, further comprising mapping the brain of the subject to identify an optimal location for positioning the electrode for recording the brain electrical signals associated with the attempted spelling of words or attempted non-speech motor movement by the subject.

99. The method of any one of claims 86-98, wherein the interface comprises a percutaneous pedestal connector attached to the subject's cranium.

100. The method of claim 99, wherein the interface further comprises a headstage connected to the percutaneous pedestal connector.

101. The method of any one of claims 86-100, wherein the processor is provided by a computer or handheld device.

102. The method of claim 101, wherein the handheld device is a cell phone or a tablet.

103. The method of any one of claims 86-102, wherein the processor is programmed to automate detection of the attempted spelling, letter classification, word classification, and sentence decoding based on identification of a neural activity pattern of electrical signals in the recorded brain electrical signal data associated with the attempted spelling of words by the subject.

104. The method of claim 103, wherein the processor is programmed to use a machine learning algorithm for the speech detection, letter classification, word classification, and sentence decoding.

105. The method of claim 104, wherein the processor is further programmed to constrain word classification from sequences of letters decoded from neural activity associated with attempted spelling of words by the subject to only words within a vocabulary of a language used by the subject.

106. The method of any one of claims 86-105, wherein the processor is further programmed to assign event labels for preparation, attempted spelling, and rest to time points during the recording of the brain electrical signal data.

107. The method of claim 106, wherein the processor is programmed to use the recorded brain electrical signal data within a time window around the detected onset of attempted spelling of a letter by the subject.

108. The method of any one of claims 86-107, further comprising providing a series of go cues to the subject indicating when the subject should initiate attempted spelling of each letter of the words of the intended sentence.

109. The method of claim 108, wherein the series of go cues are provided visually on a display.

110. The method of claim 109, wherein each go cue is preceded by a countdown to the presentation of the go cue, wherein the countdown for the next spelled letter is provided visually on the display and automatically started after each go cue.

111. The method of any one of claims 108-110, wherein the series of go cues are provided with a set interval of time between each go cue.

112. The method of claim 111, wherein the subject can control the set interval of time between each go cue.

113. The method of any one of claims 108-112, wherein the processor is programmed to use the recorded brain electrical signal data within a time window following the go cue.

114. The method of any one of claims 86-113, wherein the processor is programmed to calculate a probability that a sequence of decoded words from a sequence of decoded letters is an intended sentence that the subject tried to produce during the attempted spelling of letters of words of an intended sentence by the subject.

115. The method of any one of claims 86-114, wherein the processor is programmed to use a language model that provides next-word probabilities given a previous word or phrase in a sequence of words to aid the decoding by determining predicted word sequence probabilities.

116. The method of claim 115, wherein words that occur more frequently are assigned more weight than words that occur less frequently according to the language model.

117. The method of any one of claims 86-116, wherein the processor is further programmed to use a sequence of predicted letter probabilities to compute potential sentence candidates and automatically insert spaces into letter sequences between predicted words in the sentence candidates.

118. The method of any one of claims 86-117, further comprising:
recording brain electrical signal data associated with an attempted non-speech motor movement of the subject, wherein the subject performs the attempted non-speech motor movement to indicate the initiation or termination of the attempted spelling of words of the intended sentence or to control an external device; and
analyzing the brain electrical signal data using a classification model that identifies patterns of electrical signals in the recorded brain electrical signal data associated with the attempted non-speech motor movement and calculates a probability that the subject attempted the non-speech motor movement.

119. The method of claim 118, wherein the attempted non-speech motor movement comprises an attempted head, arm, hand, foot, or leg movement.

120. The method of claim 119, wherein the attempted hand movement comprises an imagined hand gesture or an imagined hand squeeze.

121. The method of any one of claims 118-120, further comprising assigning event labels for the attempted non-speech motor movement to time points during the recording of the brain electrical signal data.

122. The method of any one of claims 86-121, further comprising assessing accuracy of the decoding.

123. The method of any one of claims 86-122, further comprising:
recording brain electrical signal data associated with attempted speech by the subject using the neural recording device, wherein the interface receives the brain electrical signal data from the neural recording device and transmits the brain electrical signal data to the processor of the computing device; and
decoding a word, a phrase, or a sentence from the recorded brain electrical signal data associated with attempted speech by the subject using the processor.

124. A computer implemented method for decoding a sentence from recorded brain electrical signal data associated with attempted spelling of letters of words of an intended sentence by a subject, the computer performing steps comprising:
a) receiving the recorded brain electrical signal data associated with the attempted spelling of letters of words of an intended sentence by the subject;
b) analyzing the recorded brain electrical signal data using a speech detection model to calculate the probability that attempted spelling is occurring at any time point during the recording of the electrical signal data and detect onset and offset of letter production during the attempted spelling by the subject;
c) analyzing the brain electrical signal data using a letter classification model that identifies patterns of electrical signals in the recorded brain electrical signal data associated with attempted letter production by the subject and calculates a sequence of predicted letter probabilities;
d) computing potential sentence candidates based on the sequence of predicted letter probabilities and automatically inserting spaces into the letter sequences between predicted words in the sentence candidates, wherein decoded words in the letter sequences are constrained to only words within a vocabulary of a language used by the subject;
e) analyzing the potential sentence candidates using a language model that provides next-word probabilities given a previous word or phrase in a sequence of words to calculate predicted word sequence probabilities and determining the most likely sequence of words in a sentence; and
f) displaying the sentence decoded from the recorded brain electrical signal data.

125. The computer implemented method of claim 124 wherein the recorded brain electrical signal data is only used within a time window around the detected onset of attempted spelling of a letter by the subject.

126. The computer implemented method of claim 124 or 125, further comprising displaying a series of go cues to the subject indicating when the subject should initiate attempted spelling of each letter of the words of the intended sentence.

127. The computer implemented method of claim 126, wherein each go cue is preceded by displaying a countdown to the presentation of the go cue, wherein the countdown for the next spelled letter is automatically started after each go cue.

128. The computer implemented method of claim 126 or 127, wherein the series of go cues are provided with a set interval of time between each go cue.

129. The computer implemented method of claim 128, wherein the subject can control the set interval of time between each go cue.

130. The computer implemented method of any one of claims 122-127, wherein the recorded brain electrical signal data within a time window following the go cue is used for letter classification.

131. The computer implemented method of any one of claims 124-130, further comprising:
receiving recorded brain electrical signal data associated with an attempted non-speech motor movement of the subject, wherein the subject performs the attempted non-speech motor movement to indicate the initiation or termination of the attempted spelling of words of the intended sentence or to control an external device; and
analyzing the brain electrical signal data using a classification model that identifies patterns of electrical signals in the recorded brain electrical signal data associated with the attempted non-speech motor movement and calculates a probability that the subject attempted the non-speech motor movement.

132. The method of claim 131, wherein the attempted non-speech motor movement comprises an attempted head, arm, hand, foot, or leg movement.

133. The method of claim 132, wherein the attempted hand movement comprises an imagined hand gesture or an imagined hand squeeze.

134. The computer implemented method of any one of claims 124-133, wherein a machine learning algorithm is used for detection of attempted spelling or attempted non-speech motor movement or letter classification.

135. The computer implemented method of any one of claims 124-134, further comprising assigning more weight to words that occur more frequently than words that occur less frequently according to the language model.

136. The computer implemented method of any one of claims 124-135, further comprising storing a user profile for the subject comprising information regarding the patterns of electrical signals in the recorded brain electrical signal data associated with letter production during attempted spelling by the subject.

137. The computer implemented method of any one of claims 124-136, wherein the electrical signal data comprises high-gamma frequency content features and low frequency content features.

138. The computer implemented method of claim 137, wherein the electrical signal data comprises neural oscillations in a high-gamma frequency range from 70 Hz to 150 Hz and in a low frequency range from 0.3 Hz to 100 Hz.

139. The computer implemented method of any one of claims 124-138, further comprising assessing accuracy of the decoding.

140. The computer implemented method of any one of claims 124-139, further comprising decoding a sentence from recorded brain electrical signal data associated with attempted speech by the subject, the computer further performing steps comprising:
a) receiving the recorded brain electrical signal data associated with the attempted speech by the subject;
b) analyzing the recorded brain electrical signal data using a speech detection model to calculate the probability that attempted speech is occurring at any time point and detect onset and offset of word production during the attempted speech by the subject;
c) analyzing the brain electrical signal data using a word classification model that identifies patterns of electrical signals in the recorded brain electrical signal data associated with attempted word production by the subject and calculates predicted word probabilities;
d) performing sentence decoding by using the calculated word probabilities from the word classification model in combination with predicted word sequence probabilities in the sentence using a language model that provides next-word probabilities given a previous word or phrase in a sequence of words to calculate predicted word sequence probabilities and determining the most likely sequence of words in the sentence based on the predicted word probabilities determined using the word classification model and the language model; and
e) displaying the sentence decoded from the recorded brain electrical signal data.

141. The computer implemented method of claim 140, wherein a machine learning algorithm is used for speech detection and word classification, and sentence decoding.

142. The computer implemented method of claim 141, wherein artificial neural network (ANN) models are used for the speech detection and the word classification, and a hidden Markov model (HMM), a Viterbi decoding model, or a natural language processing technique is used for the sentence decoding.

143. A non-transitory computer-readable medium comprising program instructions that, when executed by a processor in a computer, causes the processor to perform the method of any one of claims 124-142.

144. A kit comprising the non-transitory computer-readable medium of claim 143 and instructions for decoding brain electrical signal data associated with attempted spelling of letters of words of an intended sentence by a subject.

145. A system for assisting a subject with communication, the system comprising:
a neural recording device comprising an electrode adapted for positioning at a location in a sensorimotor cortex region of the brain of the subject to record brain electrical signal data associated with attempted speech, attempted spelling of letters of words of an intended sentence, or attempted non-speech motor movement by the subject, or a combination thereof;
a processor programmed to decode a sentence from the recorded brain electrical signal data according to the computer implemented method of any one of claims 124-142;
an interface in communication with a computing device, said interface adapted for positioning at a location on the head of the subject, wherein the interface receives the brain electrical signal data from the neural recording device and transmits the brain electrical signal data to the processor; and
a display component for displaying the sentence decoded from the recorded brain electrical signal data.

146. The system of claim 145, wherein the subject has difficulty with said communication because of anarthria, a stroke, a traumatic brain injury, a brain tumor, or amyotrophic lateral sclerosis.

147. The system of claim 145 or 146, wherein the location of the neural recording device is in the ventral sensorimotor cortex.

148. The system of any one of claims 145-147, wherein the electrode is adapted for positioning on a surface of the sensorimotor cortex region or within the sensorimotor cortex region.

149. The system of claim 148, wherein the electrode is adapted for positioning on a surface of the sensorimotor cortex region of the brain in a subdural space.

150. The system of any one of claims 145-149, wherein the neural recording device comprises a brain-penetrating electrode array.

151. The system of any one of claims 145-150, wherein the neural recording device comprises an electrocorticography (ECoG) electrode array.

152. The system of any one of claims 145-151, wherein the electrode is a depth electrode or a surface electrode.

153. The system of any one of claims 145-152, wherein the electrical signal data comprises high-gamma frequency content features and low frequency content features.

154. The system of claim 153, wherein the electrical signal data comprises neural oscillations in a high-gamma frequency range from 70 Hz to 150 Hz and in a low frequency range from 0.3 Hz to 100 Hz.

155. The system of any one of claims 145-154, wherein the interface comprises a percutaneous pedestal connector attached to the subject's cranium.

156. The system of claim 155, wherein the interface further comprises a headstage that is connectable to the percutaneous pedestal connector.

157. The system of any one of claims 145-156, wherein the processor is provided by a computer or handheld device.

158. The system of claim 157, wherein the handheld device is a cell phone or tablet.

159. A kit comprising the system of any one of claims 145-158 and instructions for using the system for recording and decoding brain electrical signal data associated with attempted speech, attempted spelling of words, or attempted non-speech motor movement by a subject, or a combination thereof.
